(19) United States
(12) Patent Application Publication
(10) Pub. No.: US 2026/0057573 A1
(43) Pub. Date: Feb. 26, 2026

(54) SYSTEMS FOR UPPER LIP LIFT PROCEDURES AND METHODS OF THEIR USE

(71) Applicant: CUPID LIFT, INC, BEVERLY HILLS, CA (US)

(72) Inventor: BENJAMIN TALEI, BEVERLY HILLS, CA (US)

(21) Appl. No.: 19/377,891

(22) Filed: Nov. 3, 2025

Related U.S. Application Data
(63) Continuation-in-part of application No. PCT/US24/27580, filed on May 3, 2024.
(60) Provisional application No. 63/499,915, filed on May 3, 2023.

(51) Int. Cl. G06T 11/00; A61B 34/10; G06T 7/73; G06V 10/77; G06V 10/774; G06V 20/20; G06V 40/16; G16H 30/40

(52) U.S. Cl. CPC G06T 11/00; A61B 34/10; G06T 7/74; G06V 10/7715; G06V 10/7747; G06V 20/20; G06V 40/171; G16H 30/40; A61B 2034/105; G06T 2200/24; G06T 2207/20092; G06T 2207/30201; G06T 2210/41; G06T 2210/44

(57) ABSTRACT

The disclosure herein provides marking system(s) for use in guiding upper lip lift procedure(s) optionally accompanied by corner lip lift(s) or ancillary procedures such as nasal base suspension or muscle suspension procedure(s). Marking system(s) herein simplify complex and artistic decision-making process(es) related to upper lip lift and corner lift procedures by applying mathematical guidelines and principles to establish natural, aesthetically pleasing balanced appearance of the upper lip. The disclosure further provides method(s) of establishing marking system(s) and method(s) of their use in procedures such as upper lip lifts, e.g., combined deep plane release subnasal and corner lip lifts, utilizing a bull's horn surgical incision design. In certain aspects, the disclosure provides automated system(s) comprising programmed algorithms describing marking system(s) or from which marking system(s) are established or otherwise derived. In aspects, the disclosure provides system(s) comprising technology(ies) capable of reliably predicting aesthetic outcome(s) of procedure(s) incorporating marking system(s).

### RELATED APPLICATIONS/PRIORITY

This patent application is a continuation-in-part of PCT Application No. PCT/US24/27580 filed May 3, 2024, entitled “SYSTEMS FOR UPPER LIP LIFT PROCEDURES AND METHODS OF THEIR USE, which claims priority to U.S. Provisional Patent Application No. 63/499,915 filed May 3, 2023, entitled “MARKING SYSTEMS FOR UPPER LIP LIFT PROCEDURES AND METHODS OF THEIR USE.” This application incorporates the content of the above-referenced priority applications by reference and claims the benefit of priority to all of the above-referenced priority applications.

### FIELD OF THE INVENTION

This disclosure primarily relates to aesthetic modification surgery(ies), specifically upper lip lift procedure(s), and guides for use therein, wherein such guide(s) increase the predictability of the aesthetic outcome(s) resulting from the procedure(s).

### BACKGROUND OF THE INVENTION

Cosmetic modification of human appearance dates back thousands of years, with the earliest documented procedures directed to cosmetic repair dating back to as early as 2000 to 3000 B.C. Procedures to fix identified disfigurements began to take hold and expand approximately 200 years ago. According to certain sources, the first cosmetic surgery was performed by John Peter Mettauer in 1817. Beginning in the 1970s, elective cosmetic surgeries became far more regular and commonplace. Since this time, as cultural emphasis on aesthetic appearance has grown, the forms and methods of aesthetic modification have ballooned, and millions of procedures have been performed.

As technology has developed, numerous forms of aesthetic modification and related procedures have been introduced, ranging from e.g. relatively simple superficial aesthetic modification of the skin to the use of fillers to far more advanced and complex surgical interventions such as facelifts.

A commonly desired modification sought by patients is a modification of the appearance of a subject's lips. The origins of injectable lip fillers can be traced back to the early 19^{th} century, with the earliest of such products comprising fat. Lip augmentation using silicone fillers began in the 1960s, with bovine collagen fillers launching in the 1980s. However, while far less invasive than other surgical procedures, in recent years, there has been an emergence of patients suffering the negative effects of fillers.

One alternative to lip fillers is the surgical procedure referred to as an upper lip lift, an aesthetic modification procedure that has been performed for over four decades. Patients seeking such a procedure often present with excess lip length and drooping, complaining of looking tired and aged. Common patient complaints include chronically long or heavy upper lip, poor tooth show/dental hooding, overfilled lips/poor filler results, filler complications, postsurgical drooping (rhinoplasty/orthognathic), a poorly defined or thin upper lip, asymmetry, buried or drooping corners of the mouth, and upper lip incompetence. An elongated upper lip can lengthen the appearance of the entire midface. Lack of tooth show can exaggerate an aged appearance and can dramatically diminish sensuality. As the lip lengthens, it also tends to display diminished function while losing character and definition of the lip feature identified in the art as Cupid's bow.

An upper lip lift procedure requires the surgeon(s) to make an independent assessment regarding the optimal degree of lift and adjustment of each region of the upper lip. Unfortunately, lip lift techniques must address complicated obstacles in order to achieve such targeted effect(s). Traditional lip lift techniques have been criticized and often avoided due to fear of visible scarring, which can negatively impact the resulting appearance. Improperly performed procedures can leave patients with a significantly “artificial” look, scaring many patients away from such procedures. While many modified versions of the upper lip lift have been presented in attempts to resolve such challenges and provide more natural-looking results, very few of the numerous variations of the procedure considered and attempted consistently provide aesthetically natural and reproducible results.

The bullhorn subnasal lip lift, described in 1971 by Cardoso and Sperli, was one of the first techniques to take hold as providing the degree and efficacy of aesthetic modification targeted by practitioners and patients. Several renditions of the bullhorn have since followed, each with the goal of reducing incision length, limiting scarring, and enhancing the degree of lift. Unfortunately, however, most of these techniques have a tendency toward nasal base effacement and scarring that is difficult to repair. Furthermore, the results of said procedures have failed to provide consistent and predictable patient results.

In 2014, Dr. Benjamin Talei reported on a technique in which he modified the bullhorn subnasal lip lift, resulting in significant improvements in results and consistency. The technique is described in “The Modified Upper Lip Lift—Advanced Approach with Deep-Plane Release and Secure Suspension: 823-Patient Series” (See: Facial Plast Surg Clin N Am 27 (2019) 385-398.) The modified upper lip lift is based on the classic bullhorn incision with a focus on deeper and more extensive release of the upper lip and more definitive suspension. The modified upper lip lift is a centrally vectored deep-plane advancement flap technique focused on releasing tension in the skin and uniformly redistributing the skin once tethering is released. A limited group of short, preoperative radial vector markings was designed and depicted by the author in the above-referenced 2019 publication for use in guiding generally uniform excision(s) in the procedure. The reference marking system disclosed in the 2019 publication comprises markings based on the classic bullhorn lip lift technique and includes an upper incision reference, a lower incision reference, and a total of 9 radially spaced short reference hashes. The markings are described on pages 391 and 392 of the 2019 publication and, for ease of reference, are provided as FIGS. 1A, 1B, and 1C in the present application. Radially spaced hash markers of the previously presented marking system extend from the nasal base to the mid-upper lip. A portion of the lip, notably uniform in height, is identified by the marking system(s) described therein. See specifically FIG. 22 of the 2019 publication. As relevant procedure(s) were described in this publication, the publication is incorporated herein by reference in its entirety.

In early applications of the modified subnasal lip lift technique, the limited markings accompanying the procedure were identified as essential to optimizing outcomes. Unfortunately, however, while some procedures are deemed successful by both the subject and the practitioner, Dr. Talei observed that such methods did not address all of the issues sought to be overcome by such methods. Application of the procedure and its accompanying marking system sometimes yielded inconsistent or unpredictable results. As the stakes and costs of such procedures are high, the risk-benefit analysis of the procedure, as previously described, is in question. Thus, in sum, including the present inventor's previous work, no alternative upper lip lift techniques have led to a satisfactory solution for addressing inconsistent and unpredictable results and accompanying complications, such as, e.g., increased scarring, related to the traditional bullhorn upper lip lift technique. Patients are left to risk unpredictable results or to live with an aesthetic appearance that is functionally problematic or deemed physically unappealing.

Accordingly, there is a need in the art for alternative approaches to this issue. As described here, many modifications of the traditional bullhorn technique have been considered and applied with no apparent success in addressing the aforementioned issues, thus demonstrating the degree of difficulty related to addressing the aforementioned issues. Resolution of such matters requires inventive ingenuity and deep familiarity and experience with the nuances of such procedures.

### SUMMARY OF THE INVENTION

This document includes a section entitled “CONSTRUCTION PRINCIPLES AND DESCRIPTION OF SELECT TERMS” that readers are encouraged to consult to help properly interpret the disclosure provided in this section and elsewhere here. That section includes acronyms that may be present in this disclosure.

This “Summary of the Invention” section (“Summary”) briefly describes the elements and characteristics of selected illustrative embodiment(s) of this disclosure. The brief summaries of such embodiments provided here are primarily intended to illustrate the nature of systems, methods, and the like provided herein, which may be used to define claimed/claimable inventions, and, accordingly, the content of this Summary is not intended to be all-inclusive, and the scope of the disclosure herein or any invention claimed therefrom is not limited to, or by, the exemplary aspects of the disclosure provided in this section. Any of the aspects of the disclosure described in this section can be combined with any other aspect described in this or any other aspect of this disclosure.

In aspects, the disclosure herein provides marking system(s) for use in guiding upper lip lift procedure(s), e.g., subnasal lip lift procedure(s) optionally accompanied by corner lip lift(s) (e.g., subnasal and corner lift procedures), further optionally accompanied by one or more additional procedures such as a nasal base suspension, muscle suspension, or both, wherein the marking system(s) simplify(ies) complex and artistic decision-making process(es) related to upper lip lift and corner lift procedures, wherein mathematical guide(s) and principle(s) are applied in the establishment of a natural, aesthetically pleasing balance of the upper lip which optimizes muscle function and determines whether corner lift procedure(s) are appropriate for concomitant performance with a subnasal upper lip lift in order to achieve target result(s). In aspects, marking system(s) and method(s) of their use herein elucidate aesthetic design elements related to upper lift aesthetic modification procedure(s) and address concern(s) related to long-term resistance to aging. In aspects, the disclosure provides system(s) and method(s) of establishing such marking system(s) and method(s) of their use in procedures such as, e.g., upper lip lifts, e.g., subnasal upper lip lifts, e.g., deep plane release subnasal upper lip lifts, e.g., combined deep plane release subnasal and corner lip lifts, e.g., those provided here utilizing a bull's horn surgical incision design, wherein such procedure(s) can, in aspects, be accompanied by ancillary procedure(s) such as a nasal base suspension, muscle suspension, or both.

According to aspects, the disclosure herein provides marking system(s)/method(s) for use in guiding upper lip lift procedure(s), e.g., subnasal lip lift procedure(s) optionally accompanied by corner lip lift(s) (e.g., subnasal and corner lift procedures), further optionally accompanied by one or more additional procedures such as a nasal base suspension, muscle suspension, or both, wherein the marking system(s) provide for controlled, precise, identification of excision(s) associated with an upper lip lift procedure(s) which is/are often characterizable as non-uniform (e.g., height(s) of a plurality of area(s) of a bullhorn excision are not uniform between the left- and right sides of the upper lip), yet yield(s) targeted, predictable aesthetic appearance characteristic(s) of the upper lip, such as, e.g., vermillion slope and tooth show.

In certain specific aspects, the disclosure provides a marking system for use on a human subject in the preparation for, execution of, or both preparation and execution of, an aesthetic modification of the upper lip by an upper lip lift procedure, the marking system comprising at least 7 radially spaced markings (markers) located between the nasal base which can in aspects be indicated by an upper excision boundary (002) and the upper vermillion border (020). In aspects, each of the at least 7 radial markings comprises at least 3 different points within its length. In aspects, the at least 3 different points within their lengths identify a first upper incision point along the nasal base (e.g., collectively defining an upper excision boundary (002)), a second lower incision point, and a third point located on the upper vermillion border (020) along each marker. In aspects, each of the at least 7 radial markings progresses from at least substantially vertical at the midline point of the Cupid's bow trough with gradual radial rotation laterally towards the angle of the nasolabial fold(s) (016A) and (016B) (respectively), following the lip's natural relaxed tension lines, pores, or both. In aspects, the at least 7 radially spaced markings (markers) comprise at least (1) a first radial marking (“radial marking 1” (labeled in figure(s) as (006)) positioned in the middle of the columella and philtral dimple, at the lowest point of the trough of Cupid's bow; (2) 2 radial markings (“radial marking 2A” (008A) and “radial marking 2B” (008B)) positioned lateral to and on either side of radial marking 1 (006), respectively, each located lateral to the columella at the peaks of Cupid's bow and at the point where the medial crural footplates diverge into the nose at the junction with the alar sill; (3) 2 radial markings (“radial marking 3A” (010A) and “radial marking 3B” (010B)) positioned lateral to each of radial markings 2A (008A) and 2B (008B), respectively, such that the two radial markings 3A (010A) and 3B (010B) are positioned centrally between radial markings 2A (008A) and 4A (012A) and between radial markings 2B (008B) and 4B (012B), respectively; and (4) 2 radial markings (“radial marking 4A” (012A) and “radial marking 4B” (012B)) positioned lateral to each of radial markings 3A (010A) and 3B (010B), respectively, from the internal angle of the alar recurvature extending radially downward and following the natural curvature and natural tension lines, radial angulation of the pores of the upper lip, or combination thereof. In aspects, radial marking(s) 4A and 4B are at least substantially diagonal line(s). All markings to a first lateral side of the first radial marking (radial marking 1 (006)) are identifiable as a first group of markings (e.g., “group A markings”), and all markings to a second lateral side of the first radial marking are identifiable as a second group of markings (e.g., “group B markings”). In aspects, a predetermined target aesthetic shape of the upper lip defines a distance between the second lower incision point and the third point located on the upper vermillion border (020) along each of the at least 7 radial markings. In aspects, the defined distance between the second and third points along each of the at least 7 radial markings defines a final height of the upper lip at the location of such marking (“final height portion” of such marking(s)). In aspects, the remaining distance along each of the at least 7 radial markings between the second point lower incision point and the first upper incision point define the excision height at the location of such marking (“excision height portion” of such marking(s)) and identifies the shape of a region to be excised (001) in the upper lip lift procedure resulting in the target aesthetic appearance. According to aspects, the target aesthetic shape is a target slope of the upper vermillion of between about 1 and about 3 such as about 2 between radial marker(s) 2A and 3A; 2B and 3B; 3A and 4A; and 3B and 4B. In aspect, the target aesthetic shape is a target incisor display of between about 1 mm and about 4 mm, such as about 2 mm-about 3 mm. In aspects, the target aesthetic shape is derived by length(s) of marking(s) yielding a height differential between the trough of the subject's Cupid's bow and the peaks of the subject's Cupid's bow of between about 1 mm and about 3 mm, such as about 2 mm; a height differential between the dip of the columella beneath the nose of the subject and the points where the medial crural footplates diverge into the nose at the junction with the alar sill of the subject of between about 1 mm and about 3 mm, such as about 2 mm; or both. According to still more specific aspects, the marking system(s) provided herein comprise additional marking(s) made lateral to one or both of markings 4A (012A) and 4B (012B), described herein as radial markers 5A and 5B, each essentially following the relaxed skin tension lines (RSTLs), extending downward from the apex of each lateral end of the nasal base.

In certain aspects, “marking system(s)” as they have been described here are virtual marking system(s) and are generated within the automated system(s) provided by this disclosure. In such embodiment(s), “marker(s)” or “marking(s)” represents the location(s) on a human face as described, wherein their characteristic(s) (such as length(s)) exist as manipulatable data within an automated system. In aspects, such system(s) are capable of being used to manipulate such data to generate and present morphed image(s), predicting the result(s) of aesthetic modification procedure(s) such as, e.g., upper lip lift procedure(s), e.g., deep plane release upper lip lift procedure(s), and optionally ancillary procedure(s) such as corner lip lift procedure(s). In aspects, the predetermined aesthetic shape of lip(s) discussed above is/are generated by automated system(s) and presented to the system user(s) for consideration prior to the conduct of an aesthetic modification procedure.

In certain aspects, the disclosure provides automated system(s) comprising programmed algorithms describing marking system(s) or from which marking system(s) are established or otherwise derived. In aspects, the disclosure provides system(s) comprising technology(ies) capable of predicting aesthetic outcome(s) of procedure(s) incorporating marking system(s) or result(s) generated by the definition(s) or constraint(s) making up such marking system(s). In aspects, the disclosure provides landmark recognition and image morphing technology(ies) providing subject(s) and practitioner(s) the ability to visualize ahead of time, and plan, outcome(s) of procedure(s) based on a selection of parameter(s) related to marking system(s) and their related algorithms.

In aspects, the disclosure provides an automated system for generating a plurality of measurements associated with at least 7 locations associated with the upper lip of a human face, the plurality of measurements being measurements which, when established by application of an upper lip lift aesthetic modification procedure to the human face provide an aesthetic appearance predicted by the automated system, the automated system comprising (1) processing and memory functionality; (2) landmark detection functionality; (3) visual guide(s) & measurement functionality; (4) image morphing functionality; (5) artificial intelligence-based calculation capability; and (6) data capture and retrieval functionality, wherein the automated system is designed to analyze an original image of at least the upper lip of a human face; apply landmark detection functionality to identify at least 7 specific locations on the image associated with dimensions of the upper lip of the human face; apply measurement functionality to the identified locations to obtain measurement(s) associated with the original image; morph the original image according to input(s), rule(s), or setting(s) to establish a morphed image representing a target aesthetic appearance; and present data to the automated system user associated with modification(s) at one or more of the at least about 7 specific location(s) to be applied in the upper lip lift aesthetic modification procedure in order to obtain the target aesthetic appearance predicted by the morphed image. In aspects, such as ˜7 (5, 6, or 7) specific location(s) described here are location(s) associated with facial feature(s) identified by marker(s) of marking system(s) of the present disclosure; e.g., marking(s) 1, 2A, 2B, 3A, 3B, 4A, 4B, 5A, 5B, H-A, H-B, LE-A, LE-B, an upper excision boundary, and, e.g., the upper vermillion border. In aspects, the upper vermillion border has a slope between 2A and 3A; 2B and 3B; 3A and 4A; 3B and 4B, 4A and 5A; and 4B and 5B of between about 1-3, such as, e.g., about 2. In aspects, marker(s) 4A and 4B establish a boundary wherein within such boundary (between such marker(s)), the upper lip is particularly sensitive to lift, while outside of such boundary (lateral to such marker(s)) the subfacial lip and lateral mouth is less sensitive to lift.

In certain aspects, the disclosure provides marked-tip adjustable spacing devices (MT-ASDs) specifically designed for establishing marking(s) of the marking system(s) provided herein. In one specific aspect, the disclosure provides a marked-tip adjustable spacing device (MT-ASD) (600) for use in marking human skin comprising a minimum marking distance and a maximum marking distance, the MT-ASD comprising (1) two frame elements (640), (655), each having at least approximately the same size and shape, wherein (a) one frame element is movable (655) relative to the other (640) about a fixed point (680), and (b) the two frame elements (640), (655) exist in a device plane; (2) a marking end (650), (665) of each of the two frame elements (640), (655), respectively, wherein each marking end (650), (665) comprises a marking tip (685), (690), respectively, extending from each of the two marking ends (650) and (665) of the frame elements (640), (655), respectively, to form a pair of marking tips, wherein each marking tip (a) has a length; and (b) extends from each of the two frame elements (640), (655), respectively in a direction perpendicular the device plane, and wherein (c) the pair of marking tips (685), (690) are positioned parallel to one another at all times and are in physical contact with one another across the entirety of their length when the MT-ASD (600) is established at its minimum marking distance position, and further wherein marking tip(s) (685), (690), or both are coated with, covered by, or are made of a non-metal colorant application material (695); or marking end(s) (650), (665), or both, and marking tip(s) (685), (690), or both are coated with, covered by, or are made of a non-metal colorant application material (695), wherein the non-metal colorant application material is capable of maintaining an effective amount of a colorant (710) such that when the colorant (710) is present on one or both of marking tip(s) (685) and (690), the MT-ASD (600) applies visible markings to human skin when the MT-ASD (600) is in use.

In certain aspects, the disclosure provides kit(s) comprising MT-ASD(s) provided herein and one or more sources of colorant, such as, e.g., container(s) of colorant, such as container(s) comprising free, liquid colorant, liquid colorant maintained by a matrix or substrate such that the liquid colorant cannot be easily spilled from a container, or both.

Relevant procedure(s) were described in, “CUPID Lift: Advanced Lip Design Using the Deep Plane Upper Lip Lift and Simplified Corner Lift,” authored by the present inventor (see Talei B, Pearlman S J. CUPID Lip Lift: Advanced Lip Design Using the Deep Plane Upper Lip Lift and Simplified Corner Lift. Aesthet Surg J. 2022 Dec. 14; 42(12):1357-1373. doi: 10.1093/asj/sjac126. PMID: 35583637). Accordingly, this reference is also incorporated by reference herein in its entirety. Modifications made to the bullhorn subnasal lip lift technique set forth herein allow a significantly wider application(s) of such a procedure (e.g., in terms of suitable patients), the procedure previously having been reserved primarily for elderly patients with light skin. The procedure as modified here can now be performed on patients of a wide variety of ages, skin types, genders, and ethnicities. Further, this procedure can be performed on patients with existing adequate “tooth show” who wish to improve upper lip height, character, and volume. Using the modified technique, even a conservative upper lip lift can significantly alter appearance and make the lip more receptive to filler augmentation. As described herein, defined mathematical relationships between marker(s)/marking(s) of marking system(s) provided herein provide a detectably or significantly improved control and precision in the procedure(s) in which they are applied, yielding detectably or significantly more reproducible and positive outcome(s) of such procedure(s).

### BRIEF DESCRIPTION OF THE DRAWINGS/FIGURES

The drawing(s)/figure(s) provided here, and the associated following brief description of figures are intended to exemplify certain aspect(s) and principle(s) of the disclosure without limiting its scope.

FIGS. 1A, 1B, and 1C each illustrate element(s) of a prior art marking system introduced by the present inventor. FIGS. 1A, 1B, and 1C are provided as black-and-white photographs.

FIGS. 2A, 2B, 2C, 2D, 2E, 2F, 2G, 2H, 2I, 2J, 2K, 2L, and 2M provide exemplary marking(s) of marking system(s) of the present disclosure. FIG. 2N provides an illustration identifying areas of the upper vermillion border for which particular slope(s) is/are generated according to characteristic(s) of marking(s) of marking system(s) of the present disclosure, such as defined characteristic(s) of the area to be excised.

FIG. 2O provides an illustration identifying height differences between adjacent points along the upper vermillion border where marker(s) of marking system(s) disclosed herein meet the upper vermillion border.

FIGS. 3A and 3B illustrate a first embodiment of the marking system(s) of the present disclosure, identifying exemplary radial marking(s) thereof. FIG. 3A is provided as a line-drawn version of the black and white photograph provided in FIG. 3B.

FIG. 4 illustrates a second embodiment of the marking system(s) of the present disclosure, identifying exemplary length(s) of various radial marking(s) thereof.

FIG. 5 illustrates one embodiment of corner lift marking(s) provided in one embodiment of the marking system(s) of the present disclosure.

FIG. 6 illustrates one embodiment of corner lift marking(s) provided in one embodiment of the marking system(s) of the present disclosure.

FIG. 7 illustrates an embodiment of marking system(s) provided by the disclosure comprising application/consideration of mathematical analysis in the establishment of the length(s) of radial marker(s) or segment(s) thereof.

FIGS. 8A and 8B provide before photographs of a patient having received lip fillers (front (8A) and slight profile (8B) views).

FIGS. 9A and 9B provide photographs of the same patient shown in FIGS. 8A and 8B resulting from a classically designed, uniform excision deep plane lip lift (front (9A) and slight profile (9B) views).

FIGS. 10A and 10B provide photographs of the same patient shown in FIGS. 8A, 8B, 9A, and 9B resulting from the application of procedure(s) comprising the marking system(s) provided by the present disclosure (front (10A) and slight profile (10B) views).

FIG. 11 provides a view of a marked-tip adjustable spacing device provided by the disclosure herein.

FIG. 12 provides an alternative view of a marked-tip adjustable spacing device provided by disclosure herein.

FIG. 13 provides yet another alternative view of a marked-tip adjustable spacing device provided by the disclosure herein.

FIG. 14A provides an exemplary marking tip of marked-tip adjustable spacing device(s) provided by the disclosure herein.

FIG. 14B provides an exemplary marking tip of the marked-tip adjustable spacing device(s) provided by the disclosure herein (shown only on the tip of the marked-tip adjustable spacing device).

FIG. 14C provides an exemplary marking end of frame elements of marked-tip adjustable spacing device(s) provided by the disclosure herein.

FIG. 14D provides an exemplary perspective of marked-tip adjustable spacing device(s) provided by the disclosure herein.

FIG. 15A provides an exemplary colorant container comprising colorant provided by the disclosure herein.

FIG. 15B provides an exemplary colorant container comprising a matrix for maintaining colorant provided by the disclosure herein.

FIG. 16 provides an exemplary use flow of an exemplary automated system in the form of a mobile application capable of generating marking system(s) or characteristic(s) thereof described herein.

FIG. 17 provides an exemplary method of achieving a target aesthetic result comprising the use of automated system(s) capable of generating marking system(s) or characteristic(s) thereof described herein.

FIG. 18 provides an exemplary method of achieving a target aesthetic result comprising the use of an automated system(s) capable of generating marking system(S) or characteristic(s) thereof described herein.

FIG. 19 provides an embodiment of feature(s) and function(s) which may be present in automated system(s) provided by this disclosure.

FIG. 20 provides an embodiment of a process of querying automated system(s) provided by this disclosure to retrieve data saved by such system(s).

FIG. 21 provides an example of the application of landmark recognition technology to an image and the ability to modify predicted outcome(s) to generate morphed image(s) within the automated system(s) provided by this disclosure.

FIG. 22 provides an example of comparison tool(s) provided in embodiment(s) of automated system(s) described herein.

FIG. 23 is a flow chart of an exemplary method according to the disclosure.

FIG. 24 is a flow chart of a variation of the method of FIG. 23.

FIG. 25 is a flow chart of steps of a method in which a user can perform an analysis for two or more proposed surgical procedures.

FIG. 26 is a flow chart of a variation of the steps/sub-method shown in FIG. 25, in which the method/sub-method includes the use of artificial intelligence models for evaluating the performance of certain steps of the method.

FIG. 27 provides a flow chart of further steps that can be performed to the methods of FIG. 23 or 24, in which a medical professional performs a plastic surgery technique based on the output of the other steps of the method.

FIG. 28 provides a schematic/map of an exemplary system of the disclosure.

### EXEMPLARY ASPECTS OF THE INVENTION

Disclosed here are several different but related exemplary aspects (variations) of systems, methods, and other disclosed subject matter provided in this disclosure that can be used to characterize claimed/claimable inventions. The following is a non-limiting list of exemplary aspects of the disclosure, which illustrates embodiments of the disclosure in a summary form to aid readers in quickly understanding the overall scope of the disclosure. Similar to patent claims, listed aspects described in the paragraphs of this section may make reference to (depend on/from) one or more other paragraphs. Readers will understand that such references mean that the features/characteristics or steps of such referenced aspects are incorporated into/combined with the referring aspect. For example, if an aspect in a paragraph (e.g., a paragraph indicated by text at the end of the paragraph as aspect 2) refers to another aspect by one or more aspect numbers (e.g., aspect 1 or “any one of aspects 1-3”), it will be understood to include the elements, steps, or characteristics of such referenced aspects (e.g., aspect 1) in addition to those of the aspect in which the reference is made (e.g., if aspect 2 refers to aspect 1, it provides a description of a composition, method, system, device, etc., including the features of both aspect 1 and aspect 2).

Lists of aspects describing specific exemplary embodiments of the disclosure are sometimes employed to aid the reader in understanding the disclosure. Such aspects can, within them, reference other exemplary aspects, either individually or as groups of aspects (e.g., via reference to a range within a list of numbered aspects when such aspects are provided as a numbered list). Reference to ranges of aspects should be interpreted as referencing all such aspects individually, each as unique embodiments of the disclosure, and in combination with one another as unique embodiment(s) of the disclosure, according to the presentation provided of such aspects unless such an aspect within such a referenced range is either contradictory or non-sensical. If contradicted, reference to the contradictory aspect should be excluded.

It is intended that these listed exemplary aspects begin with the first listed aspect (ASPECT 1) (or aspect 1) and thereafter be numbered sequentially and incrementally by the inclusion of a reference placed near or at the end of the listed aspect (ASPECT 2, ASPECT, 3, etc.). In case of a missing aspect reference or repeated aspect reference, the order of placement of the actual recited aspect in the list that is associated with the repeated aspect reference or missing aspect reference will control (e.g., if there is an unlabeled aspect located between a first aspect labeled ASPECT 1 and a third aspect labeled ASPECT 2, the unlabeled aspect should be treated as ASPECT 2, and the aspect labeled as ASPECT 2 treated as ASPECT 3, etc.), and all numbering in the list (including all references to aspects in the list) be interpreted as accordingly modified (e.g., if the fourth aspect in such list was labeled as ASPECT 3, it should be interpreted as being labeled as ASPECT 4, and if such aspect referred to “any one or both of aspect 1 or aspect 2,” it should be read as referring to “any one or more of aspects 1-3”). Similarly, if an aspect is misnumbered (e.g., by a number in the sequence being skipped or otherwise missing), readers will similarly construe this list of aspects according to the order of placement of the recited aspects, over the numerical references. Further, if one or more of the listed exemplary aspects of the disclosure in this section fails to reference any other aspects of the disclosure, such aspect, uncontradicted, should be interpreted as applying to or as capable of being incorporated into any one or more other exemplary aspect(s) provided in this section.

In some cases, readers will recognize, particularly in view of the rest of this disclosure, that there may be minor variations in terminology use or that similar terminology is used to refer to a similar element or the same element in this listing of ASPECTS. Accordingly, readers will understand where similar terminology for elements is used below, or a different term is used for elements, steps, etc., where a corresponding but differently named element may be used in other aspects, uncontradicted, this disclosure implicitly provides support for replacing the explicitly stated term with the recognizably related or similar term.

Aspects of the technology include the following:

### Marking System(s) and Related Method(s)

According to aspects, the technology herein provides a marking system for use on a human subject for use in the preparation for, execution of, or both, an upper lip modification procedure. In aspects, the marking system comprises a plurality of, e.g., at least about 5, 6, or at least about 7 (or more) identified location(s), herein such location(s) being identified as, e.g., marker(s) of such location(s), whereby particular relationship(s) between the markers are used to detectably or significantly improve upon the predictability of a surgical outcome, provide a detectably or significantly more consistent surgical result (e.g., in view of a targeted aesthetic appearance), or both. In aspects, a targeted aesthetic appearance comprises a specific relationship between two or more specific locations(s) related to the human face, e.g., the upper lip of a human face. In aspects, the technology(ies) herein, such as, e.g., marking system(s) herein, provide(s) a detectably or significantly improved ability to predict the outcome of a procedure aiming to achieve such a targeted aesthetic appearance, provide a detectably or significantly improved reproducibility of the surgical procedure(s) performed with the aim of achieving such a target appearance (across a plurality of such surgical procedures), or both.

In aspects, the disclosure (the technology) provides a marking system for use on a human subject in the preparation for, execution of, or both, an aesthetic modification of the upper lip by an upper lip lift procedure, the marking system comprising at least 7 radially spaced markings (markers) located between the nasal base which can in aspects be indicated by an upper excision boundary (002) and the upper vermillion border (020), each of the at least 7 radial markings comprising at least 3 different points within its length wherein the at least 3 different points within its length identify a first upper incision point along the nasal base (e.g., collectively defining an upper excision boundary (002)), a second lower incision point, and a third point located on the upper vermillion border (020), the at least 7 radial markings progressing from at least substantially vertical at the midline point of the Cupid's bow trough with gradual radial rotation laterally towards the angle of the nasolabial fold(s) (016A) and (016B) (respectively), following the lip's natural relaxed tension lines, pores, or both, wherein the at least 7 radially spaced markings (markers) comprise at least (1) a first radial marking (“radial marking 1” (labeled in figure(s) as (006)) positioned in the middle of the columella and philtral dimple, at the lowest point of the trough of Cupid's bow; (2) 2 radial markings (“radial marking 2A” (008A) and “radial marking 2B” (008B)) positioned lateral to and on either side of radial marking 1 (006), respectively, each located lateral to the columella at the peaks of Cupid's bow and at the point where the medial crural footplates diverge into the nose at the junction with the alar sill; (3) 2 radial markings (“radial marking 3A” (010A) and “radial marking 3B” (010B)) positioned lateral to each of radial markings 2A (008A) and 2B (008B), respectively, such that the two radial markings 3A (010A) and 3B (010B) are positioned centrally between radial markings 2A (008A) and 4A (012A) and between radial markings 2B (008B) and 4B (012B), respectively; and (4) 2 radial markings (“radial marking 4A” (012A) and “radial marking 4B” (012B)) positioned lateral to each of radial markings 3A (010A) and 3B (010B), respectively, from the internal angle of the alar recurvature extending radially downward and following the natural curvature and natural tension lines, radial angulation of the pores of the upper lip, or combination thereof and wherein radial marking(s) 4A and 4B are at least substantially diagonal line(s), wherein all markings to a first lateral side of the first radial marking (radial marking 1 (006)) are identifiable as a first group of markings (e.g., “group A markings”), and all markings to a second lateral side of the first radial marking are identifiable as a second group of markings (e.g., “group B markings”), and wherein a predetermined target aesthetic shape of the upper lip, a target lope(s) of the upper vermillion of between about 1 and about 3 such as about 2, target tooth show, or combination(s) thereof, define a distance between the second lower incision point and the third point located on the upper vermillion border (020) along each of the at least 7 radial markings, and wherein the defined distance between the second and third points along each of the at least 7 radial markings defines a final height of the upper lip at the location of such marking (“final height portion” of such marking(s)), and further wherein the remaining distance along each of the at least 7 radial markings between the second point lower incision point and the first upper incision point define the excision height at the location of such marking (“excision height portion” of such marking(s)) identifies the shape of a region to be excised (001) in the upper lip lift procedure resulting in the target aesthetic appearance. (ASPECT 1.)

In aspects, the disclosure provides the marking system of aspect 1, wherein the marking system comprises a marking along the nasal base indicating the upper excision boundary (002) and comprising the upper incision point of each of the radial markings, the upper excision boundary (002) following at least substantially horizontally along the subnasal, perialar crease. (ASPECT 2.)

In aspects, the disclosure provides the marking system of aspect 2, wherein the upper excision boundary marking (002) comprises two peaks on either side of the midline at the divergence of the medial crural footplate and the nasal sill. (ASPECT 3).

In aspects, the disclosure provides the marking system of any one or more of aspects 1-3, wherein the marking system comprises a mid-line marking identifiable as the lower excision boundary (004) between the nasal sill and the upper vermillion border (020) and comprising the lower incision point of each of the radial markings and wherein the mid-line marking is not established prior to establishing the target final height of the upper lip along each of the at least 7 radial markings. (ASPECT 4.)

In aspects, the disclosure provides the marking system of any one or more of aspects 1-4, wherein the marking system comprises two or more markings delineating at least a first defined region of the upper lip from at least a second defined region of the upper lip, wherein the at least first and second defined regions of the upper lip differ from one another in their sensitivity to the lift effect of an upper lip lift procedure. (ASPECT 5.)

In aspects, the disclosure provides the marking system of any one or more of aspects 1-5, wherein radial markings 4A (012A) and 4B (012B) each respectively determine the lateral extent of the lift effect on the upper lip caused by the upper lip lift. (ASPECT 6.)

In aspects, the disclosure provides the marking system of any one or both of aspect 5 and aspect 6, wherein radial markings 4A (012A) and 4B (012B) delineate the at least first defined region of the upper lip from the at least second defined region of the upper lip, and wherein the at least first and second defined regions of the upper lip differ from one another in their sensitivity to the lift effect of an upper lip lift procedure. (ASPECT 7.)

In aspects, the disclosure provides the marking system of any one or more of aspects 5-7, wherein the at least first defined region of the upper lip is the region of the lip which is detectably or significantly more densely attached to the pyriform than the at least second defined region and is detectably or significantly supported by the subject's front teeth (“subnasal lip”). (ASPECT 8.)

In aspects, the disclosure provides the marking system of any one or all of aspects 5-8, wherein an at least second defined region of the upper lip is the region of the lip which hangs from the anterior cheek and face (“subfacial lip & lateral mouth”). (ASPECT 9.)

In aspects, the disclosure provides the marking system of any one or more of aspects 1-9, wherein radial markings 4A (012A) and 4B (012B) each respectively mark the border between a first region of the upper lip identified as the subnasal lip as it transitions to a second region of the upper lip identified as the subfacial lip & lateral mouth. (ASPECT 10.)

In aspects, the disclosure provides the marking system of any one or more of aspects 1-10, wherein the marking system provides identification of an at least first defined region of the upper lip (“subnasal lip”) and an at least second defined region of the upper lip (“subfacial lip & lateral mouth”) delineated by radial markings 4A (012A) and 4B (012B), wherein the location of other radial marking(s) within a defined region indicates the degree of the effect of the subnasal lip lift at the location of such radial marking(s). (ASPECT 11.)

In aspects, the disclosure provides the marking system of any one or more of aspects 1-11, wherein a subnasal lip region and subfacial lip & lateral mouth regions are demarcated by the 2 bilateral radial markings 4A (012A) and 4B (012B), the subnasal lip region being located between or medial to radial markings 4A (012A) and 4B (012B) and the subfacial lip & lateral mouth regions being located lateral to each of radial markings 4A (012A) and 4B (012B), respectively. (ASPECT 12).

In aspects, the disclosure provides the marking system of any one or more of aspects 1-12, wherein the marking system identifies at least 7 independent target locations for modification of the upper lip lift such that each of the at least 7 independent target locations can be independently modified to attain one or more target aesthetic effect(s). (ASPECT 13.)

In aspects, the disclosure provides the marking system of any one or more of aspects 1-13, wherein the marking system is a guide for use before, during, or both before and during an upper lip lift procedure and establishes lip shape resulting from the upper lip lift procedure. (ASPECT 14.)

In aspects, the disclosure provides the marking system of any one or more of aspects 1-14, wherein the length of the portion of (segment of) one or more radial markings representing the final height of the upper lip, the length of the portion of (segment of) one or more radial markings representing the excision height, or a combination thereof establishes the lip shape resulting from the upper lip lift procedure. (ASPECT 15.)

In aspects, the disclosure provides the marking system of any one or more of aspects 1-15, wherein the length of the portion of (segment of) one or more radial markings representing the final height of the upper lip, the length of the portion of (segment of) one or more radial markings representing the excision height, or a combination thereof establishes the shape of the upper vermillion resulting from the upper lip lift procedure. (ASPECT 16.)

In aspects, the disclosure provides the marking system of any one or more of aspects 1-16, wherein the length of the portion of (segment of) one or more radial markings representing the final height of the upper lip, the length of the portion of (segment of) one or more radial markings representing the excision height, or a combination thereof establishes the slope of the upper vermillion border (020) resulting from the upper lip lift procedure, wherein the slope of the upper vermillion border (020) is the vertical descent of the upper vermillion border (020) from the Cupid's peak following laterally toward the oral commissure when viewing the human subject face-on. (ASPECT 17.)

In aspects, the disclosure provides the marking system of any one or more of aspects 1-17, wherein the marking system establishes a slope of the upper vermillion border (020) yielding an about 1 mm to an about 3 mm height difference between points along the vermillion border (020) associated with radial markings 2A (008A) and 3A (010A), 2B (008B) and 3B (010B), 3A (010A) and 4A (012A), and 3B (010B) and 4B (012B), respectively. (ASPECT 18.)

In aspects, the disclosure provides the marking system of any one or more of aspects 1-18, wherein the marking system establishes a slope of the upper vermillion border (020) yielding an about 2 mm height difference between points along the upper vermillion border (020) associated with radial markings 2A (008A) and 3A (010A), 2B (008B) and 3B (010B), 3A (010A) and 4A (012A), and 3B (010B) and 4B (012B), respectively. (ASPECT 19.)

In aspects, the disclosure provides the marking system of any one or more of aspects 1-19, wherein the length of the radial markings of the marking system establish a height differential between the trough of the subject's Cupid's bow and the peaks of the subject's Cupid's bow of between about 1 mm and about 3 mm; a height differential between the dip of the columella beneath the nose of the subject and the points where the medial crural footplates diverge into the nose at the junction with the alar sill of the subject of between about 1 mm and about 3 mm; or both a height differential between the trough of the Cupid's bow and the peaks of Cupid's bow and a height differential between the dip of the columella beneath the nose and the points where the medial crural footplates diverge into the nose at the junction with the alar sill of about 1 mm to about 3 mm. (ASPECT 20.)

In aspects, the disclosure provides the marking system of any one or more of aspects 1-20, wherein the length of the radial markings of the marking system establish a height differential between the trough of the subject's Cupid's bow and the peaks of the subject's Cupid's bow of about 2 mm; a height differential between the dip of the columella beneath the nose of the subject and the points where the medial crural footplates diverge into the nose at the junction with the alar sill of the subject of about 2 mm; or both a height differential between the trough of the Cupid's bow and the peaks of Cupid's bow and a height differential between the dip of the columella beneath the nose and the points where the medial crural footplates diverge into the nose at the junction with the alar sill of about 2 mm. (ASPECT 21.)

In aspects, the disclosure provides the marking system of any one or more of aspects 1-21, wherein the portion of (segment of) the length of radial marking 1 (006) indicating the final height of the upper lip and the portions of (segments of) the lengths of markings 2A (008A) and 2B (008B) indicating the final height of the upper lip are at least essentially equal, and wherein the portion of (segment of) the length of radial marking 1 (006) indicating the excision height and the portions of (segments of) the lengths of radial markings 2A (008A) and 2B (008B) indicating the excision heights are established by the location of the second lower excision point along the length of each of radial markings 1, 2A (008A), and 2B (008B). (ASPECT 22.)

In aspects, the disclosure provides the marking system of any one or more of aspects 1-22, wherein the portion of (segment of) the length of radial marking 1 (006) indicating the final height of the upper lip and the portions of (segments of) the lengths of radial markings 2A (008A) and 2B (008B) indicating the final height of the upper lip are at least essentially equal, and wherein the portion of (segment of) the length of radial marking 1 (006) indicating the final height of the upper lip and the portions of (segments of) the lengths of radial markings 2A (008A) and 2B (008B) indicating the final heights of the upper lip are established by the location of the second lower excision point along the length of each of the radial markings 1, 2A (008A), and 2B (008B). (ASPECT 23.)

In aspects, the disclosure provides the marking system of any one or more of aspects 1-23, wherein the portion of (segment of) one or more of radial markings 2A (008A), 3A (010A), 2B (008B), 3B (010B), 4A (012A), and 4B (012B) identifying the final height of the upper lip at the point of such radial markings provides a post-excision slope of the upper vermillion border (020) yielding a height differential of between about 1 mm and about 3 mm between the points on the upper vermillion border (020) established by 2A (008A) and 3A (010A), 2B (008B) and 3B (010B), 3A (010A) and 4A (012A), and 3B (010B) and 4B (012B), respectively. (ASPECT 24.)

In aspects, the disclosure provides the marking system of any one or more of aspects 1-24, wherein the portion of (segment of) one or more of radial markings 2A (008A), 3A (010A), 2B (008B), 3B (010B), 4A (012A), and 4B (012B) identifying the final height of the upper lip at the point of such radial markings provides a post-excision slope of the upper vermillion border (020) yielding a height differential of about 2 mm between the points on the upper vermillion border (020) established by 2A (008A) and 3A (010A), 2B (008B) and 3B (010B), 3A (010A) and 4A (012A), and 3B (010B) and 4B (012B). (ASPECT 25.)

In aspects, the disclosure provides the marking system of any one or more of aspects 1-25, wherein lengths of the portion of (segments of) each radial marking of the marking system identifying the final height of the upper lip establish the excision height of a bull's horn-shaped excision comprising the excision height portion of each radial marking. (ASPECT 26).

In aspects, the disclosure provides the marking system of any one or more of aspects 1-26, wherein the nasal base, upper lip, or both, of the human subject possess(es) one or more asymmetries and one or more radial markings of group A is different from one or more of the corresponding radial markings of group B to vary the excision height, e.g., vary the excision design of a bull's horn-shaped excision, from side-to-side of the subject's upper lip. (ASPECT 27.)

In aspects, the disclosure provides the marking system of any one or more of aspects 1-27, wherein the marking system comprises 2 additional (e.g., eighth and ninth) radial markings (“radial marking 5A” (014A) and “radial marking 5B” (014B)) positioned lateral to each of radial markings 4A (012A) and 4B (012B), respectively, and each of radial marking 5A (014A) and radial marking 5B (014B) at least essentially follow the relaxed skin tension lines (RSTLs), extending downward from the apex of the lateral end of the upper excision to be made at the nasal base. (ASPECT 28.)

In aspects, the disclosure provides the marking system of aspect 28, wherein the marking system identifies at least 9 independent target locations for modification of the upper lip lift such that each of the at least 9 independent target locations can be independently modified to attain one or more target aesthetic effect(s), however wherein the sensitivity to modification at location(s) medial to at least one location is identified as being higher than modification at location(s) lateral to the at least one location identified by the marking system. (ASPECT 29.)

In aspects, the disclosure provides the marking system of any one or both of aspect 28 and aspect 29, wherein the sensitivity to modification at locations lateral to radial marking(s) 4A (012A) and 4B (012B) is higher than that at locations medial to radial marking(s) 4A (012A) and 4B (012B). (ASPECT 30.)

In aspects, the disclosure provides the marking system of any one or more of aspects 1-30, wherein the marking system comprises one or more horizontal lateral extension markings(s) (“lateral extension marking LE-A” (018A) and “lateral extension marking LE-B” (018B)) as horizontal extension(s) of the point along radial line 4A (012A), 4B (012B), or both indicating the lower excision boundary marker (004) laterally toward radial marking 5A (014A), 5B (014B), or both. (ASPECT 31.)

In aspects, the disclosure provides the marking system of -aspect 31, wherein lateral extension marking LE-A (018A), lateral extension marking LE-B (018B), or both establish one or more locations applicable to the conduct of a corner lift procedure performed on the subject in addition to, e.g., concomitantly with, the upper lip lift procedure. (ASPECT 32.)

In aspects, the disclosure provides the marking system of any one or more of aspects 1-32, wherein an additional one or more radial markings (“intermedial radial markings”) are present between any two neighboring radial markings described in one or more of aspects 1-32, further dividing the space between any two markings described in one or more of aspects 1-32. (ASPECT 33.)

In aspects, the disclosure provides the marking system of aspect 33, wherein the additional one or more radial markings present between any two neighboring radial markings described in one or more of aspects 1-32 detectably or significantly increase(s) the level of control provided by the marking system to a practitioner for attaining targeted aesthetic design feature(s) resulting from the upper lip procedure. (ASPECT 34.)

In aspects, the disclosure provides the marking system of any one or both of aspect 33 and aspect 34, wherein the space between radial markings 1 (006) and 2A (008A), 1 (006) and 2B (008B), 2A (008A) and 3A (010A), 2B (008B) and 3B (010B), 3A (010A) and 4A (012A), 3B (010B) and 4B (012B), or any two or more thereof is/are divided approximately in half with an additional radial marking line, and wherein the target slope of the upper vermillion border (020) established by the marking system comprises an about 1 mm height difference between the point(s) of any new radial marking line along the upper vermillion border (020) and an immediately neighboring one or two of the points along the upper vermillion border (020) associated with radial markings 2A (008A), 3A (010A), 2B (008B), 3B (010B), 4A (012A), and 4B (012B). (ASPECT 35.)

In aspects, the disclosure provides the marking system of any one or more of aspects 1-35, wherein the marking system further comprises at least one independent marking used to facilitate a corner lip lift procedure accompanying the upper lift procedure (e.g., performed concomitantly with the upper lip lift procedure), the at least one corner lip marking comprising either (1) an at least substantially elliptical or fusiform shape and ending laterally at the oral commissure (“Type I”), or (2) an enclosed shape extending past the commissure and comprising a detectably or significantly upward inflection (“Type II”), wherein Type I markings ((042A) or (042B) or both) are a guide in a corner lip procedure when exposure and lifting the lateral vermillion is to be performed, and wherein Type II markings ((044A) or (044B) or both) are a guide in a corner lip procedure when lifting the commissure, neutralizing down-pointing rhytids extending inferior to the chelion is to be performed, or when both lifting the commissure and neutralizing down-pointing rhytids extending inferior to the chelion are to be performed. (ASPECT 36.)

In aspects, the disclosure provides the marking system of aspect 36, wherein the point(s) on the upper vermillion border (020) of radial marking lines 4A (012A) and 4B (012B), as applicable to the side(s) of the subject's mouth undergoing the corner lip procedure, is/are used as a starting point for a straight horizontal marking line drawn laterally from such point(s) (“horizontal marking H-A” (046A) and “horizontal marking H-B” (046B), respectively) to establish a reference point for use in performing a corner lift incision. (ASPECT 37.)

In aspects, the disclosure provides the marking system of any one or both of aspect 36 and aspect 37, wherein the marking system comprises at least two additional, e.g., eighth and ninth, radial markings (“radial marking 5A” (014A) and “radial marking 5B” (014B)) positioned lateral to each of radial markings 4A (012A) and 4B (012B), respectively, wherein radial markings 5A (014A) and 5B (014B) follow the relaxed skin tension lines (RSTLs) and extend downward from the apex of the lateral end of the upper excision to be made at the nasal base, and wherein the second lower incision point along radial markings 4A (012A) and 5A (014A), along radial markings 4B (012B) and 5B (014B), or along 4A (012A), 5A (014A), 4B (012B), and 5B (014B) as applicable to the side(s) of the subject's mouth undergoing the corner lip procedure are located such that the portion of (segment of) each of applicable radial markings 4A (012A) and 5A (014A), 4B (012B) and 5B (014B), or both representing the final lip height (segment of each marking between the second lower incision point and a third point located on the upper vermillion border (020)) are at least essentially equal. (ASPECT 38.)

In aspects, the disclosure provides the marking system of any one or both of aspects 36-38, wherein the marking system comprises at least two additional, e.g., eighth and ninth, radial markings (“radial marking 5A” (014A) and “radial marking 5B” (014B)) positioned lateral to each of radial markings 4A (012A) and 4B (012B), respectively, wherein radial markings 5A (014A) and 5B (014B) follow the relaxed skin tension lines (RSTLs) and extend downward from the apex of the lateral end of the upper excision to be made at the nasal base, and wherein the length of the portions of (segments of) radial markings 4A (012A) and 5A (014A) representing the excision height, the length of the portions of (segments of) radial markings 4B (012B) and 5B (014B) representing the excision height, or the length of the portions of (segments of) both radial markings 4A (012A) and 5A (014A) and 4B (012B) and 5B (014B) (as applicable to the side(s) of the subject's mouth undergoing the corner lip procedure) representing the excision height are detectably or significantly different. (ASPECT 39.)

In aspects, the disclosure provides the marking system of any one or more of aspects 36-39, wherein radial markings 4A (012A) and 4B (012B) demarcate locations medial to which the corner lift procedure is initiated (as applicable to the side(s) of the subject's mouth undergoing the corner lip procedure) to shorten the final lip height (portion of (segment of) each marking between the second lower incision point and a third point located along the upper vermillion border (020)) of radial marking(s) 4A (012A) and 4B (012B) (as applicable to the side(s) of the subject's mouth undergoing the corner lip procedure). (ASPECT 40.)

In aspects, the disclosure provides the marking system of any one or more of aspects 1-40, wherein Pythagoras' theorem a²+b²=c² is considered in the determination of the appropriate length of the portion (segment) identified as excision height of one or more radial markings given a determined lateral distance between two points along the upper vermillion of two radial markers expected to result in a height difference between the points of at least two neighboring radial markings along the upper vermillion border (020) of between about 1 mm and about 3 mm. (ASPECT 41.)

In aspects, the disclosure provides the marking system of aspect 41, wherein Pythagoras' theorem a²+b²=c² is considered in the determination of the appropriate length of the portion (segment) identified as excision height of one or more radial markings given a determined lateral distance between two points along the upper vermillion of two radial markers expected to result in a height difference between the points of at least two neighboring radial markings along the upper vermillion border (020) of about 2 mm. (ASPECT 42.)

In aspects, the disclosure provides the marking system of any one or more of aspects 1-42, wherein Pythagoras' theorem a²+b²=c² is considered in the determination of the appropriate length of the portion (segment) identified as excision height of one or more of radial markings 3A (010A), 4A (012A), 3B (010B), and 4B (012B) given a determined lateral distance between points along the upper vermillion of radial markers 1, 2A (008A), 2B (008B), 3A (010A), 4A (012A), 3B (010B), and 4B (012B) as applicable, to result in a height difference between the points of 3A (010A) and 4A (012A) along the upper vermillion border (020), the height difference between the points of 3B (010B) and 4B (012B) along the upper vermillion border (020), or to result in a height difference between the points of 3A (010A) and 4A (012A) and 3B (010B) and 4B (012B), of between about 1 mm and about 3 mm. (ASPECT 43.)

In aspects, the disclosure provides the marking system of aspect 43, wherein Pythagoras' theorem a²+b²=c² is considered in the determination of the appropriate length of the portion (segment) identified as excision height of one or more of radial markings 3A (010A), 4A (012A), 3B (010B), and 4B (012B) given a determined lateral distance between points along the upper vermillion of radial markers 1, 2A (008A), 2B (008B), 3A (010A), 4A (012A), 3B (010B), and 4B (012B) as applicable, to result in a height difference between the points of 3A (010A) and 4A (012A) along the upper vermillion border (020), the height difference between the points of 3B (010B) and 4B (012B) along the upper vermillion border (020), or to result in a height difference between the points of 3A (010A) and 4A (012A) and 3B (010B) and 4B (012B), of about 2 mm. (ASPECT 44.)

In aspects, the disclosure provides the marking system of any one or more of aspects 1-44, wherein the upper lip lift procedure is a deep plane release subnasal upper lip aesthetic modification procedure. (ASPECT 45.)

In aspects, the disclosure provides the marking system of any one or more of aspects 1-45, wherein the upper lip lift procedure encompasses the use of a bull's horn surgical technique. (ASPECT 46.)

In aspects, the disclosure provides the marking system of any one or more of aspects 1-46, wherein one or more markings of the marking system is/are used in one or more procedures ancillary to the upper lip lift comprising corner lip lift(s), a nasal base suspension, a muscle suspension, or a combination of any or all thereof. (ASPECT 47.)

In aspects, the disclosure provides the marking system of any one or more of aspects 1-47, wherein, one or more markings of the marking system is/are a guide for an upper lip lift procedure and optionally one or more additional ancillary procedures comprising corner lip lift(s), nasal base suspension, muscle suspension, or a combination of any or all thereof. (ASPECT 48.)

In aspects, the disclosure provides a procedure for the aesthetic modification of the face of a human subject, the procedure comprising the incorporation of the marking system of any one or more of aspects 1-48. (ASPECT 49.)

In aspects, the disclosure provides a method of aesthetically modifying the face of a human subject, the method comprising the use of the marking system of any one or more of aspects 1-48. (ASPECT 50.)

In aspects, the disclosure provides an upper lip lift procedure, the procedure comprising incorporation of the marking system of any one or more of aspects 1-48. (ASPECT 49.)

In aspects, the disclosure provides a method of performing an upper lip lift procedure, the method comprising the use of the marking system of any one or more of aspects 1-48. (ASPECT 52.)

In aspects, the disclosure provides a deep plane release upper lip aesthetic modification procedure, the procedure comprising incorporation of the marking system of any one or more of aspects 1-48. (ASPECT 53.)

In aspects, the disclosure provides a method of performing a deep plane release upper lip aesthetic modification procedure, the method comprising the use of the marking system of any one or more of aspects 1-48. (ASPECT 54.)

In aspects, the disclosure provides a corner lip lift procedure comprising incorporation of one or more markings of the marking system of any one or more of aspects 1-48. (ASPECT 55.)

In aspects, the disclosure provides a method of performing a corner lip lift procedure, the method comprising the use of the marking system of any one or more of aspects 1-48. (ASPECT 56.)

In aspects, the disclosure provides a nasal base suspension procedure comprising incorporation of one or more markings of the marking system of any one or more of aspects 1-48. (ASPECT 57.)

In aspects, the disclosure provides a method of performing a nasal base suspension procedure, the method comprising the use of the marking system of any one or more of aspects 1-48. (ASPECT 58.)

In aspects, the disclosure provides a muscle suspension procedure comprising incorporation of one or more markings of the marking system of any one or more of aspects 1-48. (ASPECT 59.)

In aspects, the disclosure provides a method of performing a muscle suspension procedure, the method comprising the use of the marking system of any one or more of aspects 1-48. (ASPECT 60.)

In aspects, the disclosure provides a method of (1) assessing the face of a human subject in preparation for aesthetic modification of the subject's upper lip by application of an upper lip lift procedure to identify a target aesthetic appearance and (2) characterizing one or more surgical requirements required in the upper lip lift procedure such that the procedure results in a target aesthetic appearance, the method comprising (A) evaluating one or more features of the human subject's face; (B) identifying a target aesthetic appearance; (C) identifying one or more surgical modifications required to be made in an upper lip lift procedure to achieve the target aesthetic appearance; and (D) establishing the marking system of any one or more of aspects 1-48 wherein the characteristic(s) of the marking system, when applied in the aesthetic modification, result in achieving the target aesthetic appearance. (ASPECT 61.)

In aspects, the disclosure provides a method of assessing a human subject in preparation for aesthetic modification of the upper lip and accordingly characterizing surgical requirements for an upper lip lift, the method comprising (1) marking the contour of the nasal base to establish a first generally horizontal indicator (“LEVEL 1 indicator”) (e.g., “upper excision boundary marker” (provided in the figures as (002)), wherein a third generally horizontal indicator (“LEVEL 3 indicator”) is formed by the upper vermillion border (020); (2) marking the anatomy of the upper lip of the human subject with at least 7 radially-spaced (radial) markings, wherein the at least 7 radial markings progress from at least substantially vertical at the midline point of the Cupid's bow trough with gradual radial rotation laterally towards the angle of the nasolabial fold(s) (016A) and (016B) (respectively), following the lip's natural relaxed tension lines, pores, or both, and wherein the group of at least 7 radial markings comprise at least (a) a first radial marking (“radial marking 1” (006)) positioned in the middle of the columella and philtral dimple, at the lowest point of the trough of Cupid's bow; (b) 2 radial markings (“radial marking 2A” (008A) and “radial marking 2B” (008B)) positioned lateral to and on either side of radial marking 1 (006), respectively, each located lateral to the columella, at the peaks of Cupid's bow and at the point where the medial crural footplates diverge into the nose at the junction with the alar sill; (c) 2 radial markings (“radial marking 4A” (012A) and “radial marking 4B” (012B)) positioned lateral to each of radial markings 3A (010A) and 3B (010B), respectively, each following the natural tension lines, radial angulation of the pores of the upper lip, or both; and (d) 2 radial markings (“radial marking 3A” (010A) and “radial marking 3B” (010B)) positioned lateral to each of radial markings 2A (008A) and 2B (008B), respectively, such that the two radial markings 3A (010A) and 3B (010B) are positioned centrally between radial markings 2A (008A) and 4A (012A) and between radial markings 2B (008B) and 4B (012B), respectively, each of the at least 7 radial indicators comprising a discrete anatomical location along the nasal base, e.g., collectively forming a marking in aspects identified as an upper excision boundary (002) (LEVEL 1 indicator) and a corresponding discrete anatomical location along the upper vermillion border (020) (LEVEL 3 indicator); and wherein if each first and second discrete anatomical locations along the LEVEL 1 and LEVEL 3 indicators associated with each of the at least 7 radial markings 1, 2A (008A), 2B (008B), 3A (010A), 3B (010B), 4A (012A), and 4B (012B), respectively were to be connected, each of the resulting 7 connecting lines would intersect with the LEVEL 1 indicator, LEVEL 3 indicator, and a second generally horizontal indicator (“LEVEL 2 indicator”) (e.g., “lower excision boundary marker” (004)) positioned between the LEVEL 1 and LEVEL 3 indicators, and further wherein the distance between the LEVEL 1 and LEVEL 2 indicators along each of the radial markings establishes an excision height at the location of the radial marking and the distance between the LEVEL 2 and LEVEL 3 indicators along each of the radial markings establishes a final upper lip height at the location of the radial marking; (3) evaluating one or more anatomical features of the human subject to establish a target aesthetic appearance, e.g., to establish target aesthetic effect(s);
(4) establishing the location along the path of each of the at least 7 connecting (present or virtual) lines where the LEVEL 2 indicator is positioned to achieve the target aesthetic appearance and positioning the same, such that when the area between the LEVEL 1 and LEVEL 2 indicators is excised in the lip lift procedure, the result is an at least essentially uniform slope or difference in height between the discrete anatomical locations along the upper vermillion border (020) of/between radial markings 2A (008A) and 3A (010A); 2B (008B) and 3B (010B); 3A (010A) and 4A (012A), 3B (010B) and 4B (012B), or any combination thereof. (ASPECT 62.)

In aspects, the disclosure provides the method of aspect 62, wherein the at least substantially uniform difference in height between the discrete anatomical locations along the upper vermillion border (020) of 2A (008A) and 3A (010A); 2B (008B) and 3B (010B); 3A (010A) and 4A (012A); 3B (010B) and 4B (012B); or combination thereof is between about 1 mm to about 3 mm. (ASPECT 63.)

In aspects, the disclosure provides the method of aspect 63, wherein the at least substantially uniform difference in height between the discrete anatomical locations along the upper vermillion border (020) of 2A (008A) and 3A (010A); 2B (008B) and 3B (010B); 3A (010A) and 4A (012A); 3B (010B) and 4B (012B); or combination thereof is about 2 mm. (ASPECT 64.)

In aspects, the disclosure provides the method of any one or more of aspects 61-64, wherein the method comprises consideration of the lateral spacing along the upper vermillion border (020) between at least two radial markings and the application of Pythagoras' theorem a²+b²=c² to determine the appropriate excision height at the location of at least one radial marking required to attain an at least essentially constant about 1 mm to about 3 mm height difference between the discrete anatomical locations along the upper vermillion border (020) of two neighboring radial markings. (ASPECT 65.)

In aspects, the disclosure provides the method of any one or more of aspects 61-65, wherein the method comprises consideration of the lateral spacing along the upper vermillion border (020) between at least two radial markings and the application of Pythagoras' theorem a²+b²=c² to determine the appropriate excision height at the location of at least one of the radial markings required to attain an at least essentially constant about 2 mm height difference between the discrete anatomical locations along the upper vermillion border (020) of two neighboring radial markings. (ASPECT 66.)

In aspects, the disclosure provides the method of any one or both of aspect 65 and aspect 66, wherein the method comprises the consideration of the lateral spacing along the upper vermillion border (020) between radial marking 4A (012A) and at least one other radial marking, between radial marking 4B (012B) and at least one other radial marking, or both. (ASPECT 67.)

In aspects, the disclosure provides the method of any one or more of aspects 61-67, wherein the upper/superior end of radial markings 2A (008A) and 2B (008B) identify the upper/superior peak of the subnasal incision to be made in the upper lip lift procedure. (ASPECT 68.)

In aspects, the disclosure provides the method of any one or more of aspects 61-68, wherein one or more additional (e.g., intermediary) radial markings (indicators) is/are added between any two or more of radial markings (indicators) 1 and 2A (008A), 1 and 2B (008B), 2A (008A) and 3A (010A), 2B (008B) and 3B (010B), 3A (010A) and 4A (012A), 3B (010B) and 4B (012B), or is/are added lateral to 4A (012A), 4B, or both, and where in the additional (intermediary) radial indicator(s) provide a mechanism for a detectable or significant amount of additional control in establishing the at least essentially similar difference in height between the discrete anatomical locations along the upper vermillion border (020) of radial markers 2A (008A) and 3A (010A), 2B (008B) and 3B (010B), 3A (010A) and 4A (012A), 3B (010B) and 4B (012B), or any combination thereof. (ASPECT 69.)

In aspects, the disclosure provides the method of aspect 69, wherein the additional (intermediary) radial indicator(s) are positioned between radial markers (indicators) 2A (008A) and 3A (010A), 2B (008B) and 3B (010B), 3A (010A) and 4A (012A), 3B (010B) and 4B (012B), or in any two or more such locations, and wherein the difference in height between the discrete anatomical locations along the upper vermillion border (020) of the additional radial indicator(s) and any adjacent radial indicator(s) is about 1 mm. (ASPECT 70.)

In aspects, the disclosure provides the method of any one or more of aspects 61-70, wherein the excision height within a radial marker on one lateral side of radial marker 1 (006) is detectably or significantly different than the excision height within the corresponding radial marker on the opposite lateral side of radial marker 1 (006), but wherein the final lip height within the radial marker on the one lateral side of radial marker 1 (006) is at least essentially the same as the final lip height within the corresponding radial marker on the opposite lateral side of radial marker 1 (006). (ASPECT 71.)

In aspects, the disclosure provides the method of any one or more of aspects 61-71, wherein the at least essentially uniform difference in height between the discrete anatomical locations along the upper vermillion border (020) of radial markers 2A (008A) and 3A (010A), 2B (008B) and 3B (010B), 3A (010A) and 4A (012A), 3B (010B) and 4B (012B), or any combination thereof is a height difference of between about 1 mm and about 3 mm. (ASPECT 72.)

In aspects, the disclosure provides the method of any one or more of aspects 61-72, wherein the at least essentially uniform difference in height between the discrete anatomical locations along the upper vermillion border (020) of radial markings 2A (008A) and 3A (010A), 2B (008B) and 3B (010B), 3A (010A) and 4A (012A), 3B (010B) and 4B (012B), or any combination thereof is a height difference of about 2 mm. (ASPECT 73.)

In aspects, the disclosure provides the method of any one or more of aspects 61-73, wherein the method further comprises evaluating the points of termination along the upper vermillion of radial markings 4A (012A) and 4B (012B) and the shape of the subfacial cheek lip and cheek lateral thereto, respectively, and determining whether the lateral lip slope requires adjustment by a corner lift procedure to maintain the slope of the upper vermillion targeted between the points along the upper vermillion of radial markers 2A (008A) and 3A (010A), 2B (008B) and 3B (010B), 3A (010A) and 4A (012A), and 3B (010B) and 4B (012B), or combinations thereof established in/by the method, the slope being represented by the at least essentially uniform height difference between the points along the upper vermillion of radial markers 2A (008A) and 3A (010A), 2B (008B) and 3B (010B), 3A (010A) and 4A (012A), and 3B (010B) and 4B (012B). (ASPECT 74.)

In aspects, the disclosure provides the method of any one or more of aspects 61-74, wherein 2 additional radial markings (“radial marking 5A” (014A) and “radial marking 5B” (014B)) are established and positioned lateral to each of radial markings 4A (012A) and 4B (012B), respectively, following each of the relaxed skin tension lines (RSTLs), extending downward from the apex of the lateral end of the upper excision to be made at the nasal base (upper excision boundary (002)) on either side of the upper lip. (ASPECT 75.)

In aspects, the disclosure provides the method of aspect 75, wherein the application of the method results in the at least essentially uniform difference in height between the discrete anatomical locations along the upper vermillion border (020) of radial markers 2A (008A) and 3A (010A); 2B (008B) and 3B (010B); 3A (010A) and 4A (012A); 3B (010B) and 4B (012B); 4A (012A) and 5A (014A); 4B (012B) and 5B (014B); or any combination thereof is a height difference of between about 1 mm and about 3 mm, such as about 2 mm. (ASPECT 76.)

In aspects, the disclosure provides the method of any one or more of aspects 61-76, wherein two additional markings, “lateral extension marking LE-A” (018A) and “lateral extension marking LE-B” (018B), are established as horizonal extension(s) of the point along radial line 4A (012A), 4B (012B), or both indicating the lower excision boundary marker (004) laterally toward radial marking 5A (014A), 5B (014B), or both, establishing one or more locations applicable to the conduct of a corner lift procedure performed on the subject in addition to, e.g., concomitantly with, the upper lip lift procedure. (ASPECT 77.)

In aspects, the disclosure provides the method of aspect 77, wherein lateral extension marking(s) LE-A (018A), LE-B (018B), or both, are used in the conduct of a corner lip lift procedure. (ASPECT 78.)

In aspects, the disclosure provides the method of one or both of aspect 76 and aspect 77, wherein lateral extension marking(s) LE-A (018A), LE-B (081B), or both are used to remind a practitioner to place lateral internal mattress sutures during the upper lip lift procedure. (ASPECT 79.)

In aspects, the disclosure provides the method of any one or more of aspects 61-79, wherein the method is applied in one or more surgical technique(s) wherein the alar sill and nasal base are maintained in an intact anatomic subunit and not significantly crossed or violated. (ASPECT 80.)

In aspects, the disclosure provides the method of any one or more of aspects 61-80, wherein the method further comprises the use of preoperative photography to aid in the assessment of any requirement for asymmetric excision to be utilized in the upper lip lift procedure and optionally one or more ancillary procedure(s) performed in association with the upper lip lift procedure. (ASPECT 81.)

In aspects, the disclosure provides the method of any one or more of aspects 61-81, wherein the method further comprises viewing the profile view of the subject for slope and projection as step(s) used in establishing the target aesthetic appearance and characterization of one or more surgical requirement(s) required to attain the target aesthetic appearance (e.g., target aesthetic effect(s)). (ASPECT 82.)

In aspects, the disclosure provides the method of any one or more of aspects 61-82, wherein the final upper lip height indicated by any one or more of the radial marking(s) is at least about 10 mm. (ASPECT 83.)

In aspects, the disclosure provides the method of any one or more of aspects 61-83, wherein the final upper lip height indicated by any one or more of the radial marking(s) is at least about 11 mm. (ASPECT 84.)

In aspects, the disclosure provides the method of any one or more of aspects 61-84, wherein the method comprises the identification of at least two radial markings demarcating a first defined region identifiable as the subnasal lip region having a detectably or significantly greater sensitivity to an upper lip lift technique from a second defined region (e.g., a second region comprising two portions, the two portions divided by the subnasal lip region) each portion of the second defined region identifiable as the subfacial lip region(s) and having a detectable or significantly decreased sensitivity to an upper lip lift technique, wherein the upper lip lift technique is accordingly modified at the location of one or more other radial marker(s) according to the position of the other radial marker(s) relative to each of the two defined region(s) and, accordingly, the respective sensitivity(ies) to upper lip lift technique(s) at such radial marker location(s). (ASPECT 85).

In aspects, the disclosure provides the method of any one or more of aspects 61-85, wherein the method provides the ability to detectably or significantly control, e.g., detectably or significantly predict, or detectably or significantly consistently attain, targeted lip shape during and resulting from the upper lip lift procedure or from multiple upper lip lift procedures when a plurality of procedure(s) is performed in accordance with the method. (ASPECT 86.)

In aspects, the disclosure provides the method of any one or more of aspects 61-86, wherein the method results in the establishment of a guide for use before, during, or both before and during an upper lip lift procedure and wherein the guide serves to establish the shape of the upper vermillion resulting from the upper lip lift procedure. (ASPECT 87.)

In aspects, the disclosure provides the method of any one or more of aspects 61-87, wherein the application of the method in an upper lip lift procedure results in the ability to detectably or significantly control the shape of the upper vermillion resulting from the upper lip lift procedure. (ASPECT 88.)

In aspects, the disclosure provides the method of any one or more of aspects 61-88, wherein the conduct of the method results in the establishment of a guide for use before, during, or both before and during an upper lip lift procedure and wherein the guide serves to establish the slope of the upper vermillion border (020) resulting from the upper lip lift procedure, wherein the slope of the upper vermillion border (020) is the vertical descent of the upper vermillion border (020) from the Cupid's peak following laterally toward the oral commissure when viewing the human subject face-on. (ASPECT 89.)

In aspects, the disclosure provides the method of any one or more of aspects 61-89, wherein the application of the method in an upper lip lift procedure results in the ability to control the slope of the upper vermillion border (020) resulting from the upper lip lift procedure, wherein the slope of the upper vermillion border (020) is the vertical descent of the upper vermillion border (020) from the Cupid's peak following laterally toward the oral commissure when viewing the human subject face-on. (ASPECT 90.)

In aspects, the disclosure provides the method of any one or more of aspects 61-90, wherein the method comprises the use of two or more markings of the marking system identifying two or more regions of the upper lip differing in their sensitivity to the effect of an upper lip lift procedure to achieve a target slope of the upper vermillion border (020) such that each of the at least two regions is independently considered and modified according to their sensitivity to the upper lip lift procedure, and wherein the slope of the upper vermillion border (020) is the vertical descent of the upper vermillion border (020) from the Cupid's peak following laterally toward the oral commissure when viewing the human subject face-on. (ASPECT 91.)

In aspects, the disclosure provides the method of any one or more of aspects 61-91, wherein the nasal base, upper lip, or both, of the human subject possess(es) one or more asymmetry(ies), and one or more radial marking(s) on a first side of the first radial marking (“Group A” markings) comprise(s) a different total length at a specific location which is different from the total length of the corresponding radial marking(s) located on a second side of the first radial marking (corresponding “Group B” marking(s)) and wherein the method provides identification of an excision height which varies from side-to-side of the subject's upper lip at the two corresponding locations resulting in a final lip height at the two corresponding locations which is at least substantially or at least essentially the same. (ASPECT 92.)

In aspects, the disclosure provides the method of any one or more of aspects 61-92, wherein the application of the method in a population of patients having an upper lip lift procedure incorporating the method detectably or significantly reduces the frequency of an undesirable exaggerated appearance compared to the frequency of an undesirable exaggerated appearance resulting in a population of patients having an upper lip lift procedure not incorporating the method as determined by the professional(s) performing the procedure(s), the subject(s) undergoing the procedure(s), a group of medical professionals trained in the art tasked with assessing the outcome of the procedure(s), one or more proactive or retroactive studies performed by or overseen by one or more skilled professionals in art or any combination of any or all thereof. (ASPECT 93.)

In aspects, the disclosure provides the method of any one or more of aspects 61-93, wherein the application of the method results in a detectable or significant reduction in the frequency of an undesirable exaggeration in the slope of the upper vermillion border (020) yielding an unsatisfactory result in a population of patients having an upper lip lift procedure incorporating the use of the method compared to the frequency of an exaggeration in the slope of the upper vermillion border (020) yielding an unsatisfactory result in a population of patients having an upper lip lift procedure not incorporating the use of the method as determined by the professional(s) performing the procedure(s), the subject(s) undergoing the procedure(s), a group of medical professionals trained the art tasked with assessing the outcome of the procedure(s), one or more proactive or retroactive studies performed by or overseen by one or more skilled professionals in art, or any combination of any or all thereof. (ASPECT 94.)

In aspects, the disclosure provides the method of any one or more of aspects 61-94, wherein the method comprises establishing an excision height segment length, a final height segment length, or both, of radial marking(s) 3A (010A), 3B (010B), or both yielding an application of a lift along radial marking(s) 3A (010A), 3B (010B), or both which is detectably or significantly lower relative to the lift along one or more other radial marking(s). (ASPECT 95.)

In aspects, the disclosure provides the method of any one or more of aspects 61-95, wherein establishing an excision height segment length, a final height segment length, or both, of radial marking(s) 3A (010A), 3B (010B), or both to have length(s) yielding an application of a lift along them which is detectably or significantly less than the lift applied or present along one or more other radial marking(s) results in the upper lip lift procedure detectably or significantly relieving an appearance characterizable as angry, scowling, or snarled. (ASPECT 96.)

In aspects, the disclosure provides the method of any one or more of aspects 61-94, wherein establishing radial marking(s) 3A (010A), 3B (010B), or both to have an excision height segment length(s), final lip height segment length(s), or both, yielding an application of a lift along them which is detectably or significantly greater relative to the lift applied or present along one or more other radial marking(s) results in the upper lip lift procedure detectably or significantly increasing or creating an appearance characterizable as angry, scowling, or snarled. (ASPECT 97.)

In aspects, the disclosure provides the method of any one or more of aspects 61-97, wherein the application of the method in an upper lip lift procedure yields a lip design resulting from the upper lip lift procedure which detectably or significantly ages better than an upper lip lift procedure performed without the application of the method, wherein the degree of aging is assessed by measuring the relaxed skin tension lines (RSTLs) and the extent of drooping around the upper alveolus over time, measuring the degree of the subfacial lip(s) and lateral mouth hanging from the anterior cheek and face relative to the central subnasal lip more densely attached to the pyriform and supported by the front teeth over time, or both, and wherein such a result is determined by the professional(s) performing the procedure(s), the subject(s) undergoing the procedure(s), a group of medical professionals trained in the art tasked with assessing the outcome of the procedure(s), one or more proactive or retroactive studies performed by or overseen by one or more skilled professionals in the art, or any combination of any or all thereof over a period of time lasting from between about 5 years to about 70 years after the performance of the method. (ASPECT 98.)

In aspects, the disclosure provides the method of any one or more of aspects 61-98, wherein the method establishes a guide yielding an incisor display of the human subject resulting from the upper lip lift procedure of between about 1 mm and about 8 mm. (ASPECT 99.)

In aspects, the disclosure provides the method of any one or more of aspects 61-99, wherein the method establishes a guide yielding an incisor display of the human subject resulting from the upper lip lift procedure of between about 1 mm and about 5 mm. (ASPECT 100.)

In aspects, the disclosure provides the method of any one or more of aspects 61-100, wherein the method establishes a guide yielding an incisor display of the human subject resulting from the upper lip lift procedure of less than 4 mm. (ASPECT 101.)

In aspects, the disclosure provides the method of any one or more of aspects 61-101, wherein the method further comprises evaluating the subject for the need to perform a concomitant corner lip lift (e.g., to one or both corners of the mouth), neurotoxin injection(s) into depressor(s), volumization of a triangle inferior to the commissure, facelift, corner lip lift, nasal base suspension, muscle suspension, or a combination of any or all thereof, in order to attain a target aesthetic appearance established as a step of the method. (ASPECT 102.)

In aspects, the disclosure provides the method of aspect 102, wherein the method comprises establishing one or more parameters related to a concomitant corner lip lift, neurotoxin injection(s) into depressor(s), volumization of a triangle inferior to the commissure, facelift, corner lip lift, nasal base suspension, muscle suspension, or a combination of any or all thereof, as is determined required by the evaluation described in aspect 102. (ASPECT 103.)

In aspects, the disclosure provides the method of any one or more of aspects 61-103, wherein the method further comprises marking at least one independent marking used to facilitate a corner lip lift procedure accompanying the upper lift procedure, the at least one corner lip marking comprising either (1) an at least substantially elliptical or fusiform shape and ending laterally at the oral commissure (“Type I”) ((042A) or (042B)), or (2) an enclosed shape extending past the commissure and comprising an upward inflection (“Type II”), wherein Type I markings (042A) or (042B) are used as a guide in a corner lip procedure to expose and lift the lateral vermillion, and wherein Type II markings ((044A) or (044B)) are used as a guide in a corner lip procedure to lift the commissure, neutralize down-pointing rhytids extending inferior to the chelion, or both lift the commissure and neutralize down-pointing rhytids extending inferior to the chelion, as determined is needed by the evaluation of the subject performed as a step in the method. (ASPECT 104.)

In aspects, the disclosure provides the method of any one or more of aspects 61-104, wherein the point(s) on the upper vermillion border (020) of radial marking lines 4A (012A) and 4B (012B), as applicable to the side(s) of the subject's mouth determined to require a corner lip procedure according to an evaluation performed (or as described) in aspect 102, is/are used as a starting point for marking at least substantially straight, at least substantially horizontal, or at least both substantially straight and horizontal marking line(s) drawn laterally from such point(s) toward radial marking lines 5A (014A) and 5B (014B), respectively (“horizontal marking H-A” (046A) and “horizontal marking H-B” (046B), respectively) wherein the, e.g., at least substantially straight and horizontal marking line(s) establish a reference point for use in a corner lift incision. (ASPECT 105.)

In aspects, the disclosure provides the method of any one or more of aspects 61-105, wherein the method comprises performing an ancillary corner lift procedure in addition to the upper lip lift procedure, and wherein the final upper lip height of radial markings 5A (014A), 5B (014B), or 5A (014A) and 5B (014B) (as applicable to the corner(s) of a/the mouth being lifted by a corner lift procedure) are established such that they are at least substantially similar to the final upper lip height of corresponding radial markings 4A (012A), 4B (012B), or 4A (012A) and 4B (012B) (as applicable to the corner(s) of a/the mouth being lifted by a corner lift procedure). (ASPECT 106.)

In aspects, the disclosure provides the method of any one or more of aspects 102-106, wherein the method comprises performing an ancillary nasal base suspension procedure in addition to the upper lip lift procedure, and wherein the method comprises (1) use of the location of radial markings 4A (012A), 4B (012B), or both; (2) the degree of laxity of the lip at location(s) indicated by radial markings 3A (010A), 3B (010B), or both; or (3) both (1) and (2) in the nasal base suspension procedure to place one or more suture(s), select the path of at least one of the one or more suture(s), or both. (ASPECT 107.)

In aspects, the disclosure provides the method of any one or more of aspects 102-107, wherein the method comprises performing an ancillary muscle suspension procedure in addition to the upper lip lift procedure, and wherein the method comprises use of the location of radial markings 1, 2A (008A), 2B (008B), 4A (012A), 4B (012B), or a combination thereof to identify the location of the placement of one or more sutures associated with the muscle suspension procedure. (ASPECT 108.)

In aspects, the disclosure provides the method of any one or more of aspects 61-108, wherein the method further comprises evaluating the presence of lip filler(s). (ASPECT 109.)

In aspects, the disclosure provides the method of aspect 109, wherein the method comprises dissolving at least generally all lip filler(s) present at least about 2 weeks before a lip-lift procedure performed in association with the method. (ASPECT 110.)

In aspects, the disclosure provides the method of any one or more of aspects 62-110, wherein the method comprises the use of a marking system having any one or more of the marking system characteristic(s) described in any one or more of aspects 1-48. (ASPECT 111.)

In aspects, the disclosure provides the method of any one or more of aspects 61-111 wherein the method is performed prior to the start of any excision performed as part of the upper lip lift procedure. (ASPECT 112.)

In aspects, the disclosure provides a method of marking a human face in preparation for a subnasal upper lip lift procedure and optionally one or more related procedures, the method comprising marking the human face with the marking system of any one or more of aspects 1-48. (ASPECT 113.)

In aspects, the disclosure provides a method of establishing a visual guide comprising a set of at least 7 markings on the face of a human subject for use in guiding a subnasal upper lip lift procedure and, optionally, one or more ancillary procedures comprising a corner lift procedure, nasal base suspension, muscle suspension, or any combination of any or all thereof, the method comprising (1) marking the upper boundary of a portion of the upper lip to be excised in the subnasal upper lip lift procedure, the upper boundary following horizontally along the subnasal, perialar crease, wherein the upper boundary of the portion of the upper lip to be excised comprises two peaks on either side of the midline at the divergence of the medial crural footplate and the nasal sill; (2) marking a central marking line (radial marking line 1 (006)) vertically down the middle of the philtral trough; (3) marking 2 paramedian lines (radial marking lines 2A (008A) and 2B (008B), on the first and second lateral sides of radial marking line 1 (006), respectively) along the subnasal perialar crease (e.g., the upper boundary), wherein each of the paramedial lines are on the first and second philtral columns, respectively; (4) marking 2 radial marking lines (radial marking lines 4A (012A) and 4B (012B), on the lateral sides of each of radial marking lines 2A (008A) and 2B (008B), respectively) along the subnasal perialar crease (e.g., the upper boundary), each demarcating the lateral extent of vertical lift effect of subnasal lip lift on the upper vermillion border (020); (5) marking a radial marking line between each of radial marking lines 2A (008A) and 4A (012A) and 2B (008B) and 4B (012B) (radial marking lines 3A (010A) and 3B (010B), respectively), along the subnasal perialar crease (e.g., the upper boundary) from the nasal sill and extending to the upper vermillion border (020); (6) determining one or more aesthetic result(s) to be achieved comprising, e.g., a visual balance of two or more facial features including the size of the upper lip to the nose, the size of the upper lip to the chin, the size of the upper lip to the remainder of the face, or a combination thereof; a target incisor display; a target final lip height; a target final philtral height; the accent (slope) and definition of the Cupid's bow of the subject, the upper vermillion border (020) of the subject, or both; a target volume show of the subject's red vermillion; a target height differential along the upper vermillion border (020) between two or more of radial markers 2A (008A) and 3A (010A), 2B (008B) and 3B (010B), 3A (010A) and 4A (012A), 3B (010B) and 4B (012B), or a combination thereof; or any combination thereof; (7) determining the height of the excision across the portion of the upper lip to be excised in the subnasal upper lip lift procedure required to achieve the target aesthetic result(s); (8) marking the location between the nasal sill and the upper vermillion border (020) along the projected course of each of radial marking lines 1, 2A (008A), 2B (008B), 3A (010A), 3B (010B), 4A (012A), and 4B (012B) which indicates the lower excision boundary required to achieve the target aesthetic result(s) and which establishes a length between the given location and the point along the upper vermillion border (020) identified by the projected course of each of the radial marking lines 1, 2A (008A), 2B (008B), 3A (010A), 3B (010B), 4A (012A), and 4B (012B) representing the target final lip height at the location of that radial marking(s); (9) completing the remainder of each of the radial marking lines 1, 2A (008A), 2B (008B), 3A (010A), 3B (010B), 4A (012A), and 4B (012B) from the point of the lower excision boundary marker (004) to its corresponding point along the upper vermillion border (020); (10) optionally marking 2 radial markings (“radial marking 5A” (014A) and “radial marking 5B” (014B)) (which may be referred to herein as “Outer lines” or “O lines”) positioned lateral to each of radial markings 4A (012A) and 4B (012B) respectively and following the RSTL extending downward from the apex of the upper boundary of the excision; and (11) optionally marking (a) one or more horizontal lateral line marking(s) (“horizontal marking H-A” (046A) and “horizontal marking H-B” (046B)) as horizontal extension(s) of the point along the upper vermillion border (020) of radial line 4A (012A), 4B (012B), or both, toward radial marking 5A (014A), 5B (014B), or both, respectively as applicable; (b) one or more horizontal lateral extension markings(s) (“lateral extension marking LE-A” (018A) and “lateral extension marking LE-B” (018B)) as horizonal extension(s) of the point along radial line 4A (012A), 4B (012B), or both indicating the lower excision boundary marker (004) laterally toward radial marking 5A (014A), 5B (014B), or both; or (c) optionally marking both (a) and (b). (ASPECT 114.)

In aspects, the disclosure provides the method of aspect 114, wherein optional step (10), optional step (11), or both optional steps (10) and (11) are performed after the performance of a subnasal upper lip lift is performed using the guide established by steps (1)-(9) of the method. (ASPECT 115.)

In aspects, the disclosure provides the method of any one or more of aspects 113-115, wherein the upper boundary of the portion of the upper lip to be excised (upper excision boundary (002) does not extend beyond the natural ending of the subnasal, perialar crease. (ASPECT 116.)

In aspects, the disclosure provides the method of any one or more of aspects 113-116, wherein the upper boundary of the portion of the upper lip to be excised (upper excision boundary (002) ends where the vector of the marking becomes vertical. (ASPECT 117.)

In aspects, the disclosure provides the method of any one or more of aspects 113-117, wherein the upper boundary of the portion of the upper lip to be excised (upper excision boundary (002) follows the border of the nasal skin and at least substantially avoids the mixture of nasal and labial skin via excision of nasal skin and transposition of lip skin into the nose during the subnasal upper lip lift procedure. (ASPECT 118.)

In aspects, the disclosure provides the method of any one or more of aspects 113-118, wherein radial marking line 1 (006) is made while holding the upper lip on a gentle stretch. (ASPECT 119.)

In aspects, the disclosure provides the method of any one or more of aspects 113-119, wherein one or more radial marking line(s), e.g., one or both of radial marking line(s) 2A (008A) and 2B (008B), are made with a marked-tip adjustable spacing device (MT-ASD) while gently stretching the lip. (ASPECT 120.)

In aspects, the disclosure provides the method of aspect 120, wherein the MT-ASD is specially designed for use in the method and comprises one or more of the characteristics of MT-ASDs described herein. (ASPECT 121.)

In aspects, the disclosure provides the method of any one or more of aspects 113-121, wherein if a target aesthetic effect is a detectable or significant improvement in the asymmetry of the philtral column height, markings of the guide are made by (1) measuring the residual height of a side of the philtral column identified as comprising a desirable height, and (2) applying the applicable marking to the contralateral side to establish the guide such that it yields the desired target symmetry without the placement of tension on the lip. (ASPECT 122.)

In aspects, the disclosure provides the method of any one or more of aspects 113-122, wherein step (7) of the method comprises (1) consideration of the lateral spacing along the upper vermillion border (020) between two or more neighboring radial markings and (2) the application of Pythagoras' theorem a²+b²=c² to determine the appropriate length of the excision height of one or more radial marking lines given the lateral distance between the two or more neighboring radial markings required to attain a constant target slope of the upper vermillion border (020). (ASPECT 123.)

In aspects, the disclosure provides the method of aspect 123, wherein the target slope is established by a height difference between the points of the at least two neighboring radial markings along the upper vermillion border (020) of between about 1 mm and about 3 mm. (ASPECT 124.)

In aspects, the disclosure provides the method of any one or both of aspect 123 and aspect 124, wherein the target slope is established by a height difference between the points of the at least two neighboring radial markings along the upper vermillion border (020) of about 2 mm. (ASPECT 125.)

In aspects, the disclosure provides the method of any one or more of aspects 113-125, wherein a target aesthetic effect is a targeted length of incisor display (“tooth show”), and wherein the targeted length of incisor display is between about 1 mm and about 3 mm. (ASPECT 126.)

In aspects, the disclosure provides the method of aspect 126, wherein the targeted length of the incisor display is about 2 mm. (ASPECT 127.)

In aspects, the disclosure provides the method of any one or more of aspects 113-127, wherein the method is performed with the patient maintained in an upright position. (ASPECT 128.)

In aspects, the disclosure provides the method of any one or more of aspects 113-128, wherein the method comprises assessing the vermillion body of the human subject for shape, previous scarring, volume, or any combination of any or all thereof prior to the application of any facial marking. (ASPECT 129.)

### Marked-Tip Adjustable Spacing Device (MT-AST)

According to certain aspects, the technology herein provides device(s) or other types of tool(s) for use in the performance of surgical procedure(s) requiring the marking of skin, e.g., human skin, e.g., the skin of the face, e.g., the upper lip of a human face. In aspects, the technology provides tool(s)/device(s) which allow for (in other words are adapted for/adapted to or configured for/configured to) the application of precisely placed, precisely measured, or precisely placed and precisely measured mark(s)/marking(s) on human skin (e.g., markings having the relationship(s) described herein, placement(s) described herein, in pattern(s) described herein, etc.). According to aspects, tool(s)/device(s) herein are designed for ease of use, allowing for the positioning of such tool(s)/device(s) in such a way that provides for a clear view of the applicable surgical target location for the tool/device user, allowing for the comfortable maintenance of the tool/device in the user's hand, allowing for precise measurement control, allowing for accurate measurement, allowing for clearly applied mark(s) on the skin, and combination(s) of any or all thereof (e.g., by having precise measurement indicators and positioning means for marking position(s) with respect to other position(s)). In aspects, tool(s)/device(s) herein can be used in procedure(s) such as aesthetic modification procedure(s), e.g., upper lip lift procedure(s), such as, e.g., specifically upper lip lift procedure(s) described in detail herein. In aspects, tool(s)/device(s) of the technology herein are suitable for use in other types(s) of aesthetic modification procedures (e.g., facial reconstruction procedures of various types) as well as, e.g., non-aesthetic surgical procedures, such as, e.g., corrective procedures, or procedures performed to address illness or injury. In aspects, such a device comprises, is, or is similar to a surgical caliper. In aspects, the caliper is specifically adapted for use in procedures described herein (e.g., by measurements, indicators, and the like described herein).

In aspects, the disclosure provides a marked-tip adjustable spacing device (MT-ASD) (600) for use in marking human skin, comprising a minimum marking distance and a maximum marking distance, the MT-ASD comprising
(1) two frame elements (640), (655), each having at least approximately the same size and shape, wherein (a) the frame elements (640), (655) are movable relative to one another about a fixed point (680), and (b) the two frame elements (640), (655) exist in a device plane; (2) a marking end (650), (665) of each of the two frame elements (640), (655), respectively, wherein each marking end (650), (665) comprises a marking tip (685), (690), respectively, extending from each of the two marking ends (650) and (665) of the frame elements (640), (655), respectively, to form a pair of marking tips, wherein each marking tip (a) has a length (692); and (b) extends from each of the two frame elements (640), (655), respectively in a direction perpendicular to the device plane, and wherein (c) the pair of marking tips ((685), (690)) are positioned parallel to one another at all times and are in physical contact with one another across the entirety of their length when the MT-ASD (600) is established at its minimum marking distance position, and further wherein marking tip(s) (685), (690), or both are coated with, covered by, or are made of a non-metal colorant application material (695); or marking end(s) (650), (665), or both, and marking tip(s) (685), (690), or both are coated with, covered by, or are made of a non-metal colorant application material (695), wherein the non-metal colorant application material is capable of maintaining an effective amount of a colorant (710) such that when the colorant (710) is present on one or both of marking tip(s) (685) and (690), the MT-ASD (600) applies visible markings to human skin when the MT-ASD (600) is in use. (ASPECT 130.)

In aspects, the disclosure provides the MT-ASD (600) of aspect 130, wherein the length of the device is no more than about 115 mm. (ASPECT 131.)

In aspects, the disclosure provides the MT-ASD (600) of one or both of aspect 130 and aspect 131, wherein the maximum width of the device is no more than about 65 mm when the MT-ASD is set to its lowest possible marking distance. (ASPECT 132.)

In aspects, the disclosure provides the MT-ASD (600) of one or more of aspects 130-132, wherein the height of the device is no more than about 5 mm. (ASPECT 133.)

In aspects, the disclosure provides the MT-ASD (600) of one or more of aspects 130-133, wherein the maximum marking distance is a distance of no more than about 40 mm. (ASPECT 134.)

In aspects, the disclosure provides the MT-ASD (600) of one or more of aspects 130-134, wherein the minimum marking distance is zero mm. (ASPECT 135.)

In aspects, the disclosure provides the MT-ASD (600) of one or more of aspects 130-135, wherein the angle at which each of the marking tips (685), (690) extends from each of the two respective frame elements (640), (655) is no more than 90 degrees. (ASPECT 136.)

In aspects, the disclosure provides the MT-ASD (600) of one or more of aspects 130-136, wherein each of the marking tips (685), (690) extends at least substantially in the same, single direction, wherein the direction is a direction perpendicular to the device plane. (ASPECT 137.)

In aspects, the disclosure provides the MT-ASD (600) of one or more of aspects 130-137, wherein the non-metal material is sufficiently flexible, soft, non-pointed, or combination(s) thereof so as to not detectably or significantly damage mammalian skin when the device is used to stretch the skin, apply a visible marking to the skin, or both. (ASPECT 138.)

In aspects, the disclosure provides the MT-ASD of any one or more of aspects 130-138, wherein the colorant is a non-permanent ink or dye. (ASPECT 139.)

In aspects, the disclosure provides the MT-ASD (600) of any one or more of aspects 130-139, wherein the MT-ASD is designed to apply one or more, such as two or more markings in a subnasal upper lip lift procedure. (ASPECT 140.)

In aspects, the disclosure provides the MT-ASD (600) of any one or more of aspects 130-140, wherein the MT-ASD is designed to mark paramedial (philtral) lines for reference in a subnasal upper lip lift procedure. (ASPECT 141.)

In aspects, the disclosure provides the MT-ASD (600) of any one or more of aspects 130-141, wherein the device is used to mark paramedial (philtral) lines for reference in a subnasal upper lip lift procedure while applying a detectable or significant stretch to the upper lip to which the paramedial (philtral) lines are applied. (ASPECT 142.)

In aspects, the disclosure provides a method of performing a subnasal upper lip lift procedure, the method comprising the use of the MT-ASD (600) described in any one or more of aspects 130-142. (ASPECT 143.)

In aspects, the disclosure provides a method of aesthetically modifying the face of a human subject, the method comprising use of the marking system of one or more of aspects 1-48; incorporation of one or more of the procedure(s) or method(s) of aspects 49-129; use of the marking device of one or more of aspects 130-142; or a combination of any or all thereof. (ASPECT 144.)

In aspects, the disclosure provides a method of performing an upper lip lift procedure, e.g., a subnasal upper lip lift procedure, e.g., a subnasal and corner lip lift aesthetic modification procedure, the method comprising use of the marking system of any one or more of aspects 1-48; incorporation of one or more of the procedure(s) or method(s) of aspects 49-129; use of the marking device of one or more of aspects 130-142; or a combination of any or all thereof. (ASPECT 145.)

In aspects, the disclosure provides a method of performing a deep plane release upper lip aesthetic modification procedure, the method comprising use of the marking system of any one or more of aspects 1-48; incorporation of one or more of the procedure(s) or method(s) of aspects 49-129; use of the marking device of one or more of aspects 130-142; or a combination of any or all thereof. (ASPECT 146.)

In aspects, the disclosure provides a method of performing a corner lip lift procedure, the method comprising use of the marking system of any one or more of aspects 1-48; incorporation of one or more of the procedure(s) or method(s) of aspects 49-129; use of the marking device of one or more of aspects 130-142; or a combination of any or all thereof. (ASPECT 147.)

In aspects, the disclosure provides a method of performing a nasal base suspension procedure, the method comprising use of the marking system of any one or more of aspects 1-48; incorporation of one or more of the procedure(s) or method(s) of aspects 49-129; use of the marking device of one or more of aspects 130-142; or a combination of any or all thereof. (ASPECT 148.)

In aspects, the disclosure provides a method of performing a muscle suspension procedure, the method comprising use of the marking system of any one or more of aspects 1-48; incorporation of one or more of the procedure(s) or method(s) of aspects 49-129; use of the marking device of one or more of aspects 130-142; or a combination of any or all thereof. (ASPECT 149.)

### Automated System(s) and Related Method(s)

According to aspects, the technology(ies) herein provide automated system(s) (e.g., computer system(s), device(s), or application(s)) for (1) characterizing a visual image, e.g., the image of a human face, wherein, e.g., providing for a characterization of an image by a computer system/application that carries our function(s)/engine(s) that comprise(s) the identification of one or more feature(s) of the image, such as, e.g., two or more specific anatomical locations associated with, e.g., the human face, and (2) applying pre-established rule(s) associated with or directed to target relationship(s) between such anatomical locations to establish guidance for the performance of surgical procedure(s) aimed at obtaining the target relationships between such anatomical locations. In aspects, the characterization of the image comprises the identification of at least about 2, ≥˜3, ≥˜4, ≥˜5, ≥˜6, ≥˜7, ≥˜8, ≥˜9, or ≥˜10 different features, locations, or combination(s) thereof, of an image, e.g., an image of a human face or select(ed) region(s) of a face. In aspects, the automated system(s) is programmed/adapted or trained to indicate(s) the location(s) for, generate(s) marking(s) or facilitate(s) marking generation (e.g., by providing pointers or temporary marking(s)), or both indicate(s) the location(s) for and generate(s) marking(s) on the skin of which, when used to guide related surgical procedure(s), provide a detectably or significantly increased likelihood of the procedure attaining a target aesthetic outcome (such as those described herein), provide detectably or significantly improved reproducibility across a number of surgical procedure(s) in achieving a target aesthetic outcome (such as those described herein), or both. In aspects, the system works with robotic controls or the like, which make physical adjustment(s) or application(s) to the skin of a patient based on the execution of function(s) carried out by a programmed computer or by an artificial intelligence engine/model that is trained to carry out such functions.

In aspects, the disclosure provides the procedure(s) and method(s) of any one or more of aspects 62-129, wherein the procedure(s) or method(s) comprise(s) the application of one or more automated system(s) comprising application of artificial intelligence to an image of the subject's face to establish one or more feature(s) of one or more marking(s) of marking system(s) used in the method. (ASPECT 150.)

In aspects, the disclosure provides the procedure(s) and method(s) of any one or more of aspects 62-129, and 150, wherein the procedure(s) or method(s) comprise use of preoperative photography to aid in the assessment of any requirement for asymmetric excision to be utilized in the upper lip lift procedure and optionally one or more ancillary procedure(s) performed in association with the upper lip lift procedure. (ASPECT 151.)

In aspects, the disclosure provides marking system(s), procedure(s), and method(s), as appropriate, of one or more aspects of marking system(s), procedure(s), and method(s) provided herein, wherein computer-aided technology is applied such that one or more element(s) of marking system(s), one or more element(s) of procedure(s), or one or more element(s) of method(s), or any combination of any or all thereof, is/are performed in an at least partially computer-driven, automated manner. (ASPECT 152.)

In aspects, the disclosure provides marking system(s), procedure(s), and method(s), as appropriate, of one or more aspects of marking system(s), procedure(s), and method(s) provided herein, wherein (a) marking system(s) resulting in target aesthetic effect(s) when applied in procedure(s) and method(s) described herein, (b) procedure(s) and method(s) performed to achieve target aesthetic effect(s) described herein, or both (a) and (b) are established/performed utilizing artificial intelligence technology. (ASPECT 153.)

In aspects, the disclosure provides marking system(s), procedure(s), and method(s), as appropriate, of one or more aspects of marking system(s), procedure(s), and method(s) provided herein, wherein marking system(s) resulting in target aesthetic effect(s), or procedure(s) and method(s) performed to achieve target aesthetic effect(s), are established utilizing landmark recognition technology, morphing technology, or both, wherein the use of landmark recognition technology, morphing technology, or both detectably or significantly improve(s) the predictability of result(s)/outcome(s) of aesthetic modification procedures comprising their incorporation. (ASPECT 153.)

In aspects, the disclosure provides an automated system for generating a plurality of measurements associated with at least 7 locations associated with the upper lip of a human face, the plurality of measurements being measurements which, when established by application of an upper lip lift aesthetic modification procedure to the human face, provide an aesthetic appearance predicted by the automated system, the automated system comprising (1) processing and memory functionality; (2) landmark detection functionality; (3) visual guide(s) generation, application, & measurement functionality(ies); (4) image morphing functionality; (5) artificial intelligence-based calculation capability; and (6) data capture and retrieval functionality, wherein the automated system is designed to analyze an original image of at least the upper lip of a human face; apply landmark detection functionality to identify at least 7 specific locations on the image associated with dimensions of the upper lip of the human face; apply measurement functionality to the identified locations to obtain measurement(s) associated with the original image; morph the original image according to one or more input(s), rule(s), or setting(s) to establish a morphed image representing a target aesthetic appearance; and present to a user of the automated system data associated with modification(s) at one or more of the at least 7 specific location(s) required to be applied in the upper lip lift aesthetic modification procedure in order to obtain the target aesthetic appearance predicted by the morphed image. (ASPECT 154.)

In aspects, the disclosure provides the automated system of aspect 154, wherein the at least 7 specific locations comprise: (1) the middle of the columella and philtral dimple, at the lowest point of the trough of Cupid's bow and extending from the nasal base to the upper vermillion border (location 1, identified as “marker 1” of marking system(s) herein); (2) 2 lateral locations, one on either side of the columella at the peaks of Cupid's bow and at the points where the medial crural footplates diverge into the nose at the junction(s) with the alar sill, each location extending from the nasal base to the upper vermillion border (locations 2A and 2B, identified as “marker 2A” and “marker 2B” of marking system(s) herein); (3) 2 lateral locations, one on either side of the upper lip and each extending from the internal angle of the alar recurvature radially downward and following the natural curvature and natural tension lines, radial angulation of the pores of the upper lip, or any combination thereof, each location extending from the nasal base to the upper vermillion border (locations 4A and 4B, identified as “marker 4A” and “marker 4B” of marking system(s) herein); (4) 2 lateral locations, one in between each of the locations identified as location(s) (2) and (3) above on both sides of the upper lip, each location extending from the nasal base to the upper vermillion border (locations 3A and 3B, identified as “marker 3A” and “marker 3B” of marking system(s) herein. (ASPECT 155.)

In aspects, the disclosure provides the automated system of one or both of aspect 154 and 155, wherein the at least 7 specific locations further comprise locations positioned lateral to each of the positions identified as position (3) in aspect 147 above, on either side of the upper lip for a total of 2 locations, wherein each location extends downward from the nasal base and follows each of the relaxed skin tension lines (RSTLs) (location 5, identified as “marker 5A” and “marker 5B” of marking system(s) herein). (ASPECT 156.)

In aspects, the disclosure provides the automated system of one or more of aspects 154-156, wherein the at least 7 specific locations further comprise (1) locations extending laterally on either side of the upper lip from the points on the upper vermillion border of locations 4A and 4B above on either side of the upper lip (for a total of two locations) to a point at or near the nasolabial fold line(s) of the relaxed skin tension line globe (locations H-A and H-B, identified as “marker H-A” and “marker H-B” herein); (2) locations extending laterally on either side of the upper lip from a point located between the nasal sill and the upper vermillion border along the locations identified as locations 4A and 4B above (for a total of two locations) (locations LE-A and LE-B (identified as “marker LE-A” and “marker LE-B” herein); or one, two, three or all four of locations H-A, H-B, LE-A, and LE-B. (ASPECT 157.)

In aspects, the disclosure provides the automated system of one or more of aspects 154-157, wherein the automated system generates and identifies specific points along each of at least locations 1, 2A, 2B, 3A, 3B, 4A, and 4B, wherein each of the specific points divides the respective location into two segments, wherein one segment at each location represents a final lip height (“FH”, such as segment 2A-FH, 2B-FH, etc.) and the other segment at leach location represents an excision height at such location (“EX”, such as segment 2A-EX, 2B-EX, etc.), and wherein the automated system generates and identifies the specific points along each of the locations such that upon excision of the upper lip according to the excision height at each identified location in an upper lip lift aesthetic modification procedure, the final lip height of the upper lip at each identified location yields a slope of the upper vermillion border of between about 1 mm and about 3 mm, such as about 2 mm, between points 2A and 3A; between points 3A and 4A; between points 2B and 3B; between points 3B and 4B; or between combinations of some or all of such pairs of points. (ASPECT 158.)

In aspects, the disclosure provides the automated system of one or more of aspects 154-158, wherein the automated system generates and identifies specific points along each of at least locations 1, 2A, 2B, 3A, 3B, 4A, and 4B, wherein each of the specific points divides the respective location into two segments, wherein one segment at each location represents a final lip height (“FH”, such as segment 2A-FH, 2B-FH, etc.) and the other segment at leach location represents an excision height at such location (“EX”, such as segment 2A-EX, 2B-EX, etc.), and wherein the automated system generates and identifies the specific points along each of the locations such that upon excision of the upper lip according to the excision height at each identified location in an upper lip lift aesthetic modification procedure, the final lip height of the upper lip at each identified location yields an incisor display of between about 1 mm and about 4 mm, such as, e.g., about 2 mm to about 3 mm. (ASPECT 159.)

In aspects, the disclosure provides the automated system of any one or more of aspects 154-159, wherein the predicted aesthetic appearance yielded by defined characteristic(s) of location(s) generated or compiled by the automated system are used to morph the original image and to present to the system user the predicted aesthetic appearance if such characteristic(s) of such location(s) were applied in an aesthetic modification procedure. (ASPECT 160.)

In aspects, the disclosure provides the automated system of any one or more of aspects 154-160, wherein the characteristic(s) of location(s) generated or compiled by the automated system yielding the predicted aesthetic appearance can be saved or transmitted by the automated system. (ASPECT 161.)

In aspects, the disclosure provides the automated system of aspect 161, wherein the characteristic(s) of location(s) generated or compiled by the automated system yielding the predicted aesthetic appearance are saved as a feature set, the characteristic(s) of which used in an aesthetic modification procedure, such as an upper lip lift procedure, to achieve the predicted aesthetic appearance. (ASPECT 162.)

In aspects, the disclosure provides the automated system of any one or more of aspects 154-162, wherein the automated system makes recommendation(s) for intervention(s) to be applied as an ancillary procedure to the upper lip lift procedure. (ASPECT 163.)

In aspects, the disclosure provides the automated system of aspect 163, wherein the ancillary procedure is a corner lip lift procedure. (ASPECT 164.)

In aspects, the disclosure provides the automated system of any one or more of aspects 154-164, wherein the automated system is a mobile application. (ASPECT 165.)

In aspects, the disclosure provides the automated system of aspect 165, wherein the mobile application operates on a smartphone or tablet. (ASPECT 166.)

In aspects, the disclosure provides the automated system of any one or more of aspects 154-166, wherein the automated system functions on both Android and iOS platforms, or functions on platform(s) comprising one, some, most, generally all, substantially all, or all feature(s) and function(s) of Android and iOS platform(s); or functions on platform(s) developed from, derived from, or comprising, some, most, generally all, substantially all, or all feature(s) and function(s) of Android platform(s), iOS platform(s), or both, existing at the time of this disclosure. (ASPECT 167.)

In aspects, the disclosure provides the automated system of any one or more of aspects 154-167, wherein the automated system comprises use of mobile user interface framework(s) providing a single codebase enabling use across a plurality of platform(s); coding language(s) capable of supporting numerical and scientific computing; web server gateway interface(s) addressing HTTP request(s) and backend service(s) of the automated system(s); machine learning library(ies) to train an artificial intelligence model capable of detecting and modifying face landmark(s); and cloud-based web service(s). (ASPECT 168.)

In aspects, the disclosure provides the automated system of any one or more of aspects 154-168, wherein the automated system comprises use of one or more of Flutter, Python, Flask, PyTorch, and AWS; comprises use of technology(ies) providing one, some, most, generally all, substantially all, essentially all, or all of the feature(s) and function(s) of one, some, most, generally all, substantially all, essentially all, or all of Flutter, Python, Flask, PyTorch, and AWS; or comprises use of one or mor technology(ies) built upon, developed from, derived from, or otherwise related to one, some, most, generally all, substantially all, essentially all, or all of Flutter, Python, Flask, PyTorch, and AWS. (ASPECT 169.)

In aspects, the disclosure provides the automated system of any one or more of aspects 167-169, wherein the automated system is provided by a mobile application provided on a smartphone or tablet. (ASPECT 170.)

In aspects, the disclosure provides the automated system of any one or more of aspects 154-170, wherein the predicted aesthetic appearance presented as the morphed image is generated by the automated system based upon a first set of user inputs and, after the automated system user reviews the presented morphed image representing the predicted target aesthetic appearance, input parameter(s) of the system is/are adjusted and a new morphed image is generated by the system, representing an updated predicted result/outcome, wherein the process of iterating the morphed image representing the target appearance is iterated a plurality of times, e.g., 2 or more times, e.g., 5, 10, 15, or 20 or more times until parameters yielding target effect(s) are achieved. (ASPECT 171.)

In aspects, the disclosure provides the automated system of aspect 171, wherein the plurality of iterations are captured by machine-learning process(es)/programming/mechanism(s) present in the automated system and suitable target aesthetic appearance(s), e.g., target aesthetic effect(s), are generated with detectably or significantly fewer iterations over time. (ASPECT 172.)

In aspects, the disclosure provides the automated system(s) of any one or more of aspects 154-172, wherein the automated system(s) are used in any procedure(s) and method(s) described in this exemplary aspects section of this disclosure. (ASPECT 173.) In aspects, the disclosure provides a method of using automated system(s) of any one or more of aspect(s) 154-172 in an aesthetic modification procedure, such as an upper lip lift, e.g., a deep plane release upper lip lift procedure. (ASPECT 174.)

In aspects, the disclosure provides a method of generating marking system(s) according to any one or more of aspects 1-48, wherein the marking system(s) are generated virtually using any one or more of the automated system(s) described in aspects 154-172, and wherein the marker(s) as described in aspects 1-48 correspond to location(s) within such system(s), the corresponding location(s) having any one or more of the attribute(s) of their corresponding marker(s) described in aspect 1-48. (ASPECT 175.)

### SPECIFIC EXEMPLARY EMBODIMENTS

### Exemplary Embodiment 1

In certain facets, the disclosure herein provides technology/devices and related methods for generating predicted outcome(s) of surgical interventions, e.g., surgical aesthetic modification procedure(s), e.g., face lift(s), e.g., upper lip lift modification procedure(s).

According to certain aspects, the invention herein provides method(s) of generating predicted outcome imagery of a surgical aesthetic modification procedure. In aspects, such a method comprises providing a user with a computerized device. In aspects, such a computerized device can be, e.g., any automated system(s) described herein, such as, e.g., a laptop, smart device such as a smart phone or tablet, etc.

In aspects, such a computerized device comprises an interface that facilitates the collection of user input, such as, e.g., user input(s) described herein. In aspects, such an interface facilitates the transmission of the user input(s) to a computer component. In aspects, a computer component can be, e.g., part of a cloud computing facility. In aspects, a computer component can be housed at a medical facility. In aspects, a computer component can be located in any location reachable by the computerized device interface.

In aspects, the computer component comprises a computer component processor component that performs function(s) on the input(s). In aspects, the computer component returns output to the user through the interface. In aspects, a computer component processor component performs processing function(s) alone or in cooperation with a computer component processor component.

In aspects, the computerized device comprises a user interface processing component. In aspects, a user interface processing component performs one or more functions locally on the computerized device, alone or in cooperation with a computer component processor component.

In aspects, a computerized device comprises a memory component. In aspects, a memory component can store data and one or more system instructions to be carried out by the user interface processing component, the computer component processor component, or both.

According to aspects, a method of generating predicted outcome imagery of a surgical aesthetic modification procedure comprises prompting the computerized device user to provide user-identifying information through the interface of the computerized device. In aspects, via the user interface processing component, the computer component processor component, or both, user-identifying information is collected via the user interface processing component, the computer component processor component, or both. In aspects, such data is stored in the computerized device memory component.

According to aspects, a method of generating predicted outcome imagery of a surgical aesthetic modification procedure comprises prompting the computerized device user to provide, via the computerized device interface, a computer component with one or more image data files. In aspects, an image data file corresponds to a photographic image of at least a portion of the computerized device user's face, wherein such a user may be the subject of a potential surgical aesthetic modification procedure. In aspects, the method comprises prompting the user to provide one or more images for each of two or more distinct areas of the face through the interface and facilitating the association of the user's image data file input with at least two different areas of the face.

In certain aspects, a method of generating predicted outcome imagery of a surgical aesthetic modification procedure comprises subjecting one or more image data files provided by a computerized device user to analysis by a facial feature recognition artificial intelligence module (FFRAI), including, e.g., landmark recognition technology(ies), to identify and locate a plurality of mapped facial features in the photographic image. In aspects such facial features are associated with the image data file. In certain aspects, the FFRAI comprises at least one facial feature recognition neural network (FFRNN) that is trained to recognize facial feature(s), such as a plurality of facial features.

In aspects, a method of generating predicted outcome imagery of a surgical aesthetic modification procedure comprises, e.g., through the operation of the user interface processing component, computer component processor component, or both, applying a face image morphing function. In aspects, a face image morphing function modifies one or more of the image data files according to preprogrammed instruction(s). In aspects, a face image morphing function modifies one or more of the image data files according to artificial intelligence model(s). In aspects, a face image morphing function modifies one or more of the image data files according to preprogrammed instruction(s) or one or more artificial intelligence models in a manner resulting in one or more initial morphed facial image files. In aspects, each morphed facial image file is expected to correspond with the outcome of performing the surgical aesthetic modification procedure according to the preprogrammed instruction(s) or the artificial intelligence model(s) resulting in the morphed image(s). According to aspects, the image morphing function is carried out in part by a face imaging morphing artificial intelligence model (FIMAIM) that is trained to generate morphed facial images based on the expected parameters of a surgical aesthetic modification procedure. According to aspects, the FIMAIM comprises at least one facial image morphing neural network (FIMNN), and the method comprises training the FIMNN based on user evaluation, medical expert evaluation, or a combination thereof.

In some aspects, a method of generating predicted outcome imagery of a surgical aesthetic modification procedure comprises permitting the user of a computerized device to cause the user interface processing component of the device, the computer component processor component of the device, or both, to modulate one or more parameters associated with one or more selected facial feature elements from among the mapped facial features to generate one or more secondarily morphed facial image files. Such modulated elements can be, e.g., the adjustment of lengths associated with marking(s)/location(s) described herein, such as one or more of marking 1, 2A, 2B, 3A, 3B, 4A, 4B, or others, or, e.g., segment(s) thereof. In certain aspects, at least one parameter of the one or more parameters associated with a plurality of facial feature elements is modulated to generate at least one secondarily morphed image file. In certain aspects, at least one user evaluation of at least one secondarily morphed image file is collected. In aspects, the method comprises evaluating the secondarily morphed image files by a morphing function evaluation artificial intelligence model (MFEAI), which optionally comprises one or more morphing function evaluation neural networks (MFENN(s)).

In some aspects, a method of generating predicted outcome imagery of a surgical aesthetic modification procedure comprises permitting the user to evaluate each of the initial morphed facial image files and each of the secondarily morphed facial image file(s) through the computerized device interface.

In some aspects, a method of generating predicted outcome imagery of a surgical aesthetic modification procedure comprises storing information regarding the user's evaluation in the memory component of the computerized device.

According to certain aspects, a method of generating predicted outcome imagery of a surgical aesthetic modification procedure comprises performing a side-by-side comparison of at least two images selected from among an unmodified photographic image of the user's face provided by the user input, an initially morphed image file image, and one or more secondarily morphed image file images. In aspects, such a method comprises performing a side-by-side comparison of at least two images selected from among an unmodified photographic image of the user's face provided by the user input, an initially morphed image file image, and one or more secondarily morphed image file images.

According to certain aspects, a method of generating predicted outcome imagery of a surgical aesthetic modification procedure comprises providing the user with the option of selecting among a plurality of surgical aesthetic modification procedures or a combination of two or more plastic surgery procedures, wherein each surgical aesthetic modification procedure among the plurality of surgical aesthetic modification procedures corresponds to a surgical aesthetic modification procedure performed within a discrete portion of the face. In aspects, such a discrete portion of the upper face is characterizable as the upper lip, e.g., a region generally between the relaxed skin tension lines, and below the nasal base down to the upper vermillion border. In aspects, the method optionally comprises subjecting the image input provided by the user to a plurality of facial feature recognition artificial intelligence models, wherein each facial feature recognition artificial intelligence model of the plurality of facial feature recognition artificial intelligence models is trained to recognize one or more different facial features associated with a distinct part of the face (such as, e.g., the upper lip and regions of the face relatively near the upper lip).

According to certain aspects, a method of generating predicted outcome imagery of a surgical aesthetic modification procedure comprises providing the computerized device user with the option of selecting among a plurality of surgical aesthetic modification procedures or, e.g., the option of selecting from a combination of two or more surgical aesthetic modification procedures, wherein each surgical aesthetic modification procedure among the plurality of surgical aesthetic modification procedures corresponds to a surgical aesthetic modification procedure performed within a discrete portion of the face such as, e.g., the upper lip. In aspects, such method(s) of generating predicted outcome imagery optionally comprises subjecting the image input provided by the user to a plurality of facial feature recognition artificial intelligence models. In aspects, each facial feature recognition artificial intelligence model of the plurality of facial feature recognition artificial intelligence models is trained to recognize one or more different facial features associated with a distinct part of the face.

According to certain aspects, a method of generating predicted outcome imagery of a surgical aesthetic modification procedure comprises the user interface processing component, the computer component processor component, or both, carrying out one or more functions to evaluate whether the user's image data file input is sufficient to perform an effective evaluation and prompting the user to provide additional or alternative image input if the one or more functions determine the image input is insufficient to provide an effective evaluation. In aspects, one or more such functions are performed at least in part by the operation of one or more artificial intelligence models that are trained in evaluating the sufficiency of user image input.

### Exemplary Embodiment 2

According to alternative embodiments provided herein are method(s) of directing surgical aesthetic modification procedure(s). In certain aspects, the method comprises any of the step(s) of the method provided in exemplary embodiment 1In aspects, through the operation of the computer component processor component, the method comprises using user evaluation data to generate procedure-related output. In aspects, such output provides a medical practitioner with information that assists the medical practitioner in conducting related surgical aesthetic modification procedure(s).

In certain aspects, the technology herein provides a method of directing a surgical aesthetic modification procedure wherein the surgical aesthetic modification procedure-related output comprises facial image data corresponding to the user's face and provides marking(s) for guiding a plastic surgeon in applying (performing) the surgical aesthetic modification procedure. In certain aspects, the method comprises the computer component of computerized system(s) executing one or more functions that evaluate the user evaluation and computerized system(s) providing a system-driven evaluation of the user evaluation to the medical practitioner. In aspects, the system-driven evaluation of the user evaluation comprises a proposed modification of the user evaluation. According to particular aspects, the operation of the computer component processor component facilitates a review of the user input, the system-driven evaluation, or both by the medical practitioner by relaying output and one or more prompts or one or more directions to a computer device used by the medical practitioner that is in communication with the computer component.

In certain aspects, a method of directing a surgical aesthetic modification procedure wherein the surgical aesthetic modification procedure-related output comprises augmented reality output, such as a display of marking(s) for guiding a plastic surgeon in applying the surgical aesthetic modification procedure in an augmented reality output.

According to some aspects, a method of directing a surgical aesthetic modification procedure further comprises the medical practitioner carrying out the surgical aesthetic modification procedure on the user based at least in part on the procedure-related output. In certain aspects, the method further comprises conducting a post-surgical aesthetic modification procedure evaluation and using the post-procedure evaluation to modify one or more AI models or other functions carried out by the processor component.

### Exemplary Embodiment 3

According to certain aspects, the technology herein provides computer system(s) for assisting surgical procedure(s), e.g., aesthetic modification procedures, e.g., face lifts, e.g., upper lip lift procedures. In aspects, computer system(s) facilitate the communication between the patient and the practitioner in advance of such procedure(s). In aspects, patient input, practitioner input, or both, are used as inputs for analytical program(s) (e.g., to adjust parameters of method(s) described herein which are also programmed into the applicable computer/device) or are used as inputs for an artificial intelligence model or used to training an artificial intelligence model. In aspects, computer system(s) provided herein detectably or significantly improve the communication between patient and practitioner; detectably or significantly improve the predictability of the outcome of such procedures; detectably or significantly improve the reproducibility of such procedures across a plurality of such procedures; or any combination of any or all thereof.

This disclosure provides, i.a., new, inventive, and useful computer systems for, e.g., estimating the outcome of a plastic surgery method or facilitating aesthetic modification surgery patient and medical practitioner consultation comprising (1) a user interface device (UID) comprising a user interface processing component, a memory component, a data transmission component, and an interface component, wherein the UID interface component facilitates the collection of user input comprising user-identifying information and one or more image data files which are processed by the UID user interface processing component and stored in the UID memory component, and (2) a computer component (a primary computing component) that in operation is in communication with the UID and comprises a computer component processor component and a computer component memory component, wherein the computer component processor component comprises artificial intelligence models (AIM(s)) that are specifically trained to recognize facial features, to modify facial images to reflect the likely outcome of a surgical aesthetic modification procedure, or both, and (3) a medical practitioner user interface device (MPID) that comprises a medical practitioner interface device processor component, a medical practitioner device memory component, a medical practitioner device data transmission component, and a medical practitioner interface device interface component, wherein the medical practitioner device interface facilitates the display of one or more image data files which are processed by the computer component processor component and which comprise procedure-related output comprising information that can direct the medical practitioner in performing one or more surgical aesthetic modification procedures.

Devices that can be used as UIDs or MPIDs are described herein and include smartphones, tablets, and laptops/personal computing devices. The “computer component” or primary computing component can be any suitable device or system that performs most of the processing steps, maintains most of the memory, or both, with respect to the overall capability of the system. In some aspects, the computer component comprises or is a cloud-based computing system, such as AWS, Azure, or a Google computing system. Although described separately here, in aspects, processing functionality is carried out in part on a local device level (an MPID, UID, or both) and in part at a primary computing component level. For example, some parts of a function can be carried out by engine(s)/AIM(s) that operate at the local device level, the computing component level, or in part at both levels. In some aspects, some engines/AIMs are performed by a user/medical professional device, and other engines/AIMs are implemented at the computer component level.

The computer component processor component executes/operates artificial intelligence models (AIM(s)) that are specifically trained to recognize a plurality of facial features, to modify facial images to reflect the likely outcome of a surgical aesthetic modification procedure, or to perform both types of functions. Typically, the AIM(s) of a system include at least one facial feature image recognition AIM (in other parts of the disclosure referred to as landmark identification/marking systems). In aspects, systems include at least two different facial feature image recognition AIMs, wherein each can identify facial features in part(s) of the face. In aspects, a computer component processor component is configured to operate a plurality of artificial intelligence models, wherein at least one of the plurality of artificial intelligence models is trained to recognize facial feature(s) in user image data file input.

In an aspect, a system comprises at least two distinct facial feature recognition artificial intelligence models, wherein each of the at least two distinct facial feature recognition artificial intelligence models is trained to recognize one or more facial features in different portions of the face. For example, a system may be configured to allow a user to analyze the outcome of two or more different types of plastic surgery, which may be alternatively applied or concurrently performed (e.g., a subnasal lip lift and a corner lip lift). In such aspects, the system can include distinct AIMs that are configured to (e.g., trained to) recognize facial features/landmarks in two distinct parts of the face.

In aspects, a system comprises at least one artificial intelligence model that generates a morphed version of one or more of the image data files to establish one or more morphed image data files in response to user input comprising one or more proposed changes in a parameter (characteristic or measurement) associated with a facial feature (e.g., the length of a line of the face). Such an AIM can take input data and, based on its training, prepare a morphed image based on the expected outcome of a modification. The modification can be a baseline modification or one or more further modifications that arise from user changes in one or more parameters of the method (e.g., changing the length of one or more facial lines).

In aspects, a system further comprises at least one artificial intelligence model that evaluates the sufficiency of the user image data file input. Such an AIM can be trained to determine when a set of image data file inputs is effective for achieving a desirable level of performance in terms of, e.g., identifying, locating, or characterizing/measuring facial feature elements. Such systems can comprise instructions when such a level of sufficient data is not achieved, such as a looping function/prompting function that motivates or forces a user to add additional or alternative input data.

In aspects, systems can further comprise one or more artificial intelligence models that evaluate the efficacy of one or more system prompts, one or more system user instructions, one or more system outputs, or a combination thereof. These AIMs, which, like other AIMs described herein, can be NNs, can help facilitate and improve user interactions resulting in more effective collection and evaluation of data.

### DETAILED DESCRIPTION OF THE INVENTION

For convenience, both combinations of elements/steps and individual elements/steps may be described in this section of this disclosure. Despite the inclusion of passages focused on specific elements/steps, any aspect, facet, embodiment, or other description of particular step(s) or element(s) can be applied to any general description of the system(s)/method(s) of the disclosure, or any other recited element(s)/step(s) thereof, which are provided in any part of this disclosure.

As used herein, the word “exemplary” means “serving as an example, instance, or illustration.” The following detailed description is merely exemplary in nature and is not intended to limit specific application(s) and use(s). Any embodiment described herein as “exemplary” is not necessarily to be construed as preferred or advantageous over other embodiments.

Throughout this disclosure, elements exemplified in the figure(s) provided herewith may be indicated by their label reference identifier (label ID number). For example, reference to radial marker 1 may be followed by “(006)” herein, indicating that the element is labeled as number “6” in the provided Figures. Leading zeroes of label ID numbers provided in this disclosure are not provided in the labeling of figures. Accordingly, for example, “006” herein references label ID number “6” in the figures; “010A” references label ID number “10A” in the figures, and so on. Typically, 3-digit identifiers provided in this disclosure, e.g., “(640)”, correspond directly to the same 3-digit identifiers provided in the figures.

Provided herein are marking system(s) comprising a plurality of markers for use as a guide in the performance of aesthetic modification procedure(s), such as, e.g., an upper lip lift procedure, e.g., a subnasal upper lip lift, e.g., a deep plane subnasal and corner lip lift procedure, e.g., such procedure(s) using a bull's horn technique (bull's horn subnasal incision technique). Herein, an “aesthetic modification procedure” is a surgical procedure performed with the intent of modifying the aesthetic appearance of the subject, such as the aesthetic appearance of, e.g., feature(s) of, the subject's face. Herein, use of the term “plastic surgery” or “plastic surgery procedure,” and the like should be interpreted as an aesthetic modification procedure. Herein, “aesthetic modification procedure” may also be interchanged with “surgical aesthetic modification procedure.” In aspects, a surgical aesthetic modification procedure is a modification technique performed within a discrete portion of the face, e.g., the upper lip. In aspects, a discrete portion of the face is between and including the relaxed skin tension lines and between the nose and to and including the upper vermillion border. In aspects, the disclosure provides upper lip lift procedure(s), e.g., subnasal upper lip lift procedures, e.g., deep plan subnasal and corner lip lift procedure(s) with targeted design feature(s)/aesthetic effect(s) which provide at least generally standardized, at least substantially standardized, at least essentially standardized, predictable, consistent, or otherwise reliable outcome(s) by way of the incorporation of carefully designed/established marking system(s). In aspects, the disclosure provides aesthetic modification procedure(s) comprising the use of, e.g., the application of, such marking system(s). In aspects, the disclosure provides methods of using the marking system(s) in aesthetic modification procedure(s), such as, e.g., procedure(s) that aesthetically modify the upper lip. Further, in aspects, the disclosure provides method(s) of creating a visual guide for use in aesthetic modification procedure(s), e.g., method(s) of facial marking to establish a system of markers suitable for guiding aesthetic modification procedure(s) such as, e.g., an upper lip lift procedure, e.g., a subnasal upper lip lift procedure (e.g., with or without concomitant corner lip lift(s)) comprising application of a bull's horn incision technique.

According to certain aspects, the disclosure provides marking system(s) and method(s) of their creation, application, and use(s), comprising the evaluation of facial feature(s) in order to establish characteristic(s) of the marking system(s). Thus, in aspects, the disclosure provides method(s) of evaluation and characterization of a human subject (method(s) of assessment of human subject(s)) comprising the development of marking system(s) for use in achieving targeted aesthetic modification(s) resulting from procedure(s) incorporating use of the marking system(s).

According to certain aspects, the disclosure herein provides marking system(s) and related calculation(s) or other assessment(s) for use in upper lip lift procedure(s), such as a deep plane upper lip lift procedure. In aspects, such procedure(s) detectably or significantly improve(s) lip shape, lip contour, lip volume, or both. In aspects, use of marking system(s) and related calculation(s) or other assessment(s) associated with the establishment of such marking system(s) avoid exaggerated, fake, or otherwise characterizable as negative appearance outcomes which can be associated with lip lift procedure(s) which lack use of such system(s), calculation(s), or assessment(s). In aspects, the marking system(s) provided herein are characterizable as design marking(s). In aspects, such marking system(s) are established prior to the start of an aesthetic modification procedure. In aspects, such marking system(s) comprise a series of markers, e.g., radial marker(s)/marking(s), which enable a practitioner, patient, or both, to design what an upper lip will look like as a result of the performance of an upper lip lift procedure comprising the use of the marking system(s). In aspects, the marking system(s) disclosed herein allow for detectable or significant, e.g., improved, more refined, or combination(s) thereof, control of lip shape, lip slope, and lip display resulting from the performance of an upper lip lift procedure comprising use of the marking system(s). In aspects, marking system(s) comprise demarcation(s) indicating differentiation between the subfacial and subnasal lip. In aspects, marking system(s) aid in identifying whether the subject patient would benefit from the performance of a corner lip lift in addition to an upper lip lift (e.g., deep plane upper lip lift).

Further, regarding the differentiation between the subfacial and subnasal lip, in aspects, marking system(s) providing such differentiation between the subfacial and subnasal lip is used to guide decision(s) related to lip aging and the effect(s) thereof and the effect of surgery(ies), injectable(s), or both, on area(s) of the upper lip. In aspects, the differentiation between the subfacial and subnasal lip as indicated by marking system(s) provided herein differentiate two distinct areas: a first, more lateral area identified as the subfacial lip and lateral mouth, hanging from the anterior cheek and face, which will experience a greater degree of aging than a second, more medial area identified as the central subnasal lip, which is more densely attached to the pyriform and supported by the front teeth, and which will experience a greater sensitivity or effect of or to a lip lift procedure. In aspects, radial marker 4 (e.g., radial markers 4A (012A) and 4B (012B) described herein provides the differentiation between the subfacial and subnasal lip described here.

According to certain aspects, the disclosure provides marking system(s), described in detail herein, for use in aesthetic modification procedures, such as upper lip lift procedures, e.g., subnasal upper lip lifts (e.g., with or without concomitant corner lip lift(s)) and, e.g., deep plane release upper lip lift modification procedures.

In certain specific aspects, the disclosure provides method(s) of aesthetically modifying the face of a human subject, the method(s) comprising performing aesthetic modification procedure(s) on the face of the human subject wherein the aesthetic modification procedure(s) include(s), use(s), or otherwise incorporate(s) (1) marking system(s) comprising of the characteristic(s) of marking system(s) provided herein; (2) method(s) of incorporating marking system(s) comprising one or more of the characteristic(s) of marking system(s) provided herein; (3) method(s) comprising the development/establishment of marking system(s) comprising one or more characteristics of the marking system(s) described herein; or (4) any combination of (1)-(3). Marking system(s) and such related method(s) are described herein.

In certain specific aspects, the disclosure provides method(s) of performing an upper lip lift procedure on a human subject, the method(s) comprising performing the upper lip lift procedure on the human subject wherein the procedure includes, uses, or otherwise incorporates (1) marking system(s) comprising one or more of the characteristic(s) of marking system(s) provided herein; (2) method(s) of incorporating marking system(s) comprising one or more of the characteristic(s) of marking system(s) provided herein; (3) method(s) comprising the development/establishment of marking system(s) comprising one or more characteristics of the marking system(s) described herein; or (4) any combination of (1)-(3). As stated above, marking system(s) and such related method(s) are described herein.

In certain specific aspects, the disclosure provides method(s) of performing an upper lip lift procedure, e.g., a subnasal upper lip lift procedure (e.g., with or without concomitant corner lip lift(s)) on a human subject, the method(s) comprising performing the upper lip lift procedure on the human subject wherein the procedure include(s), use(s), or otherwise incorporate(s) (1) marking system(s) comprising one or more of the characteristic(s) of marking system(s) provided herein; (2) method(s) of incorporating marking system(s) comprising one or more of the characteristic(s) of marking system(s) provided herein; (3) method(s) comprising the development/establishment of marking system(s) comprising one or more characteristics of the marking system(s) described herein; or (4) any combination of (1)-(3). As stated above, marking system(s) and such related method(s) are described herein.

In certain specific aspects, the disclosure provides method(s) of performing a deep plane release upper lip lift procedure on a human subject, the method(s) comprising performing the deep plane release upper lip lift procedure on the human subject wherein the procedure include(s), use(s), or otherwise incorporate(s) (1) marking system(s) comprising one or more of the characteristic(s) of marking system(s) provided herein; (2) method(s) of incorporating marking system(s) comprising one or more of the characteristic(s) of marking system(s) provided herein; (3) method(s) comprising the development/establishment of marking system(s) comprising one or more characteristics of the marking system(s) described herein; or (4) any combination of (1)-(3). As stated above, marking system(s) and such related method(s) are described herein.

In certain specific aspects, the disclosure provides method(s) of performing a corner lip lift procedure on a human subject, the method(s) comprising performing the corner lip lift procedure on the human subject wherein the procedure includes, uses, or otherwise incorporates (1) marking system(s) comprising one or more of the characteristic(s) of marking system(s) provided herein; (2) method(s) of incorporating marking system(s) comprising one or more of the characteristic(s) of marking system(s) provided herein; (3) method(s) comprising the development/establishment of marking system(s) comprising one or more characteristics of the marking system(s) described herein; or (4) any combination of (1)-(3). As stated above, marking system(s) and such related method(s) are described herein.

In certain specific aspects, the disclosure provides method(s) of performing a nasal base suspension procedure on a human subject, the method(s) comprising performing the nasal base suspension procedure on the human subject wherein the procedure includes, uses or otherwise incorporates (1) marking system(s) comprising one or more of the characteristic(s) of marking system(s) provided herein; (2) method(s) of incorporating marking system(s) comprising one or more of the characteristic(s) of marking system(s) provided herein; (3) method(s) comprising the development/establishment of marking system(s) comprising one or more characteristics of the marking system(s) described herein; or (4) any combination of (1)-(3). As stated above, marking system(s) and such related method(s) are described herein.

In certain specific aspects, the disclosure provides method(s) of performing a muscle suspension procedure on a human subject, the method(s) comprising performing the muscle suspension procedure on the human subject wherein the procedure include(s), use(s), or otherwise incorporate(s) (1) marking system(s) comprising one or more of the characteristic(s) of marking system(s) provided herein; (2) method(s) of incorporating marking system(s) comprising one or more of the characteristic(s) of marking system(s) provided herein; (3) method(s) comprising the development/establishment of marking system(s) comprising one or more characteristics of the marking system(s) described herein; or (4) any combination of (1)-(3). As stated above, marking system(s) and such related method(s) are described herein.

In certain specific aspects, the disclosure provides method(s) of performing an upper lip lift procedure, a subnasal upper lip lift procedure, a deep plane release upper lip lift procedure, a corner lip lift procedure, a nasal base suspension procedure, a muscle suspension procedure, or a combination of any or all thereof on a human subject, the method(s) comprising performing the procedure(s) on the human subject wherein the procedure(s) include(s), use(s), or otherwise incorporate(s) (1) marking system(s) comprising one or more of the characteristic(s) of marking system(s) provided herein; (2) method(s) of incorporating marking system(s) comprising one or more of the characteristic(s) of marking system(s) provided herein; (3) method(s) comprising the development/establishment of marking system(s) comprising one or more characteristics of the marking system(s) described herein; or (4) any combination of (1)-(3). As stated above, marking system(s) and such related method(s) are described herein.

In aspects, the disclosure herein provides method(s) of achieving target aesthetic design feature(s) of the upper lip of a human subject, such as, e.g., a target vermillion slope or other design feature(s) (e.g., target aesthetic effect(s)) described herein, wherein the method(s) comprise use of, application of, or incorporation of any of the method(s) described above.

In aspects, the disclosure herein provides method(s) of addressing age-related changes in the upper lip of a human subject, such age-related changes in the upper lip being described elsewhere herein, wherein the method(s) comprise use of, application of, or incorporation of any of the method(s) described above.

In aspects, the disclosure herein provides method(s) of achieving target aesthetic design feature(s) of the upper lip, e.g., a target vermillion slope or other design feature(s) (e.g., target aesthetic effect(s)) described herein; addressing specific age-related changes in the upper lip in a human subject; or both, wherein the method(s) comprise use of, application of, or incorporation of any of the method(s) described above.

In aspects, the disclosure provides method(s) for addressing (e.g., detectably or significantly reducing or eliminating) difference(s), e.g., asymmetry(ies), in one or more feature(s) of the upper lip, such as asymmetry(ies) associated with the nasal base, upper lip height, vermillion slope, philtrum, etc. In aspects, such asymmetry(ies) can be naturally occurring or as a result of prior aesthetic intervention(s), e.g., application of lip fillers, surgical procedure(s), or both. In aspects, such method(s) comprise the use of, application of, or incorporation of any of the method(s) described above.

In aspects, the disclosure provides method(s) of achieving target aesthetic design feature(s) such as a target vermillion slope, addressing age-related change(s) in the upper lip, or both, in a human subject wherein the method(s) further allow for addressing (e.g., detectably or significantly reducing or eliminating) difference(s), e.g., asymmetry(ies) across the upper lip, wherein the method(s) comprise use of, application of, or incorporation of any of the method(s) described above.

In aspects, method(s) provided by the disclosure comprise the use of, application of, or otherwise incorporating marking system(s) described herein. In aspects, method(s) provided by the disclosure comprise use of, application of, or otherwise incorporating marking system(s) described herein, wherein the marking system(s) comprise any feature(s) of marking system(s) provided herein.

### Evaluation & Characterization—Pre-Procedure: Establishing Marking System(s)

According to certain aspects, the disclosure provides marking system(s), e.g., facial marking system(s), method(s) of their establishment, and method(s) of their use in and application to aesthetic modification procedure(s), wherein characteristic(s) of marking system(s) are established based upon an evaluation and characterization of one or more facial feature(s) or characteristic(s) of a human subject undergoing an aesthetic modification procedure.

As is described in more detail elsewhere herein, establishment of marking system(s) of this disclosure can be partially or completely automated. In aspects, pre-procedure evaluation is conducted only by human(s). In alternative aspects, pre-procedure evaluation is conducted by a combination of human(s) and technology, such as technology comprising artificial intelligence function(s). In still further aspects, pre-procedure evaluation is conducted only by technology (technology alone), such as by technology comprising artificial intelligence function(s).

In aspects, the evaluation and characterization of facial feature(s) or characteristic(s) identifies specific feature(s)/characteristic(s) of the face (e.g., of the upper lip) which require(s) modification in order to attain one or more design features, such design feature(s) being identified as element(s) of a target design or target aesthetic appearance as described elsewhere herein. In aspects, an exemplary element of a target design can be, e.g., a preferred upper vermillion slope. In aspects, the evaluation and characterization of facial feature(s) or characteristic(s) identifies genetically predetermined or, e.g., age-related change(s), e.g., age-related change(s) of the upper lip and difference(s) in the same across the span of the upper lip.

In certain aspects, the evaluation and characterization of facial feature(s) is performed in advance of, in conjunction with, or at least in part in advance of, and in conjunction with, the establishment of marking system(s) described herein. In aspects, the evaluation and characterization of facial feature(s) is performed in preparation for an upper lip aesthetic modification procedure, such as, e.g., an upper lip lift, e.g., a subnasal upper lip lift, e.g., a subnasal upper lip lift comprising use of a bull's horn incision technique, such as a deep plane release upper lip aesthetic modification procedure comprising use of a bull's horn incision technique. In aspects, the evaluation and characterization of facial feature(s) further comprises evaluating whether the subject may benefit from a procedure distinct from a subnasal upper lip lift. In aspects, the evaluation and characterization of facial feature(s) further comprises evaluating the subject for their ability to benefit from an ancillary procedure such as, e.g., a concomitant corner lip lift, nasal base suspension, muscle suspension, neurotoxin injection(s) into depressor(s), volumization of a triangle inferior to the commissure, facelift, or a combination of any or all thereof, in order to attain a target aesthetic appearance.

According to certain aspects, the evaluation and characterization of facial feature(s) or characteristic(s) informs parameter(s) or design of marking system(s). In aspects, the evaluation and characterization of facial feature(s) or characteristic(s) establishes one or more parameters related to an upper lip lift procedure and, optionally, a concomitant corner lip lift, nasal base suspension, muscle suspension, neurotoxin injection(s) into depressor(s), volumization of a triangle inferior to the commissure, facelift, or a combination of any or all thereof.

In aspects, step(s) of evaluation and characterization of facial feature(s) or characteristic(s) provided herein can be performed by a human. In aspects, the evaluation and characterization step(s) described herein can be performed by a human aided by other tool(s) or system(s), such as computer- or software-related tool(s) or system(s). In aspects, step(s) of evaluation and characterization of facial feature(s) or characteristic(s) provided herein can be performed by a human aided by robotic tool(s) or system(s). In aspects, step(s) of evaluation and characterization of facial feature(s) or characteristic(s) provided herein can be performed by a tool(s) or system(s) incorporating the use of artificial intelligence. In aspects, step(s) of evaluation and characterization of facial feature(s) or characteristic(s) provided herein can be performed at least mostly without, at least generally without, at least substantially without, at least essentially without, essentially without, or without human involvement. In aspects, evaluation and characterization of facial feature(s) or characteristic(s) provided herein comprise(s) use of preoperative photography. In aspects, preoperative photography detectably or significantly improves upon the assessment of (detectably or significantly increases the sensitivity in identifying) a requirement for asymmetric excision to be utilized in the upper lip lift procedure, ancillary procedure(s) performed in association with the upper lip lift procedure, or both. In aspects, the disclosure provides method(s) of facial assessment in preparation for aesthetic modification procedure(s), such as, e.g., upper lip lift procedure(s), e.g., subnasal upper lip lift procedures or, e.g., deep plane release upper lip modification procedure(s) (e.g., with or without concomitant corner lip lift(s)), wherein the method(s) comprise(s) the application of automated, non-human, or computer-based system(s) comprising software-driven analysis to, e.g., application of artificial intelligence of, a scan of, an image of, a live presentation of, or any combination thereof, the subject's face to establish feature(s) of marking(s) of the guide/marking system used in the method(s).

According to aspects, step(s) of evaluation and characterization of facial feature(s) or characteristic(s) provided herein comprise viewing the profile view of the subject. In aspects, considering the profile view of the subject allows for consideration of slope and projection as step(s) used in establishing target aesthetic effect(s), e.g., a target aesthetic appearance, and to identify surgical requirement(s) required to attain the target aesthetic effect(s)/target aesthetic appearance.

### Target Design

In aspects, the disclosure provides marking system(s) which serve(s) as a guide in the performance of aesthetic modification procedure(s), wherein one or more element(s) of the marking system(s) (e.g., one or more marker(s)/indicator(s) of the marking system(s)) are designed according to a targeted aesthetic outcome or effect(s), e.g., to achieve a target aesthetic appearance resulting from the procedure(s) incorporating application or use of the marking system(s). According to particular aspects, a target design can be, e.g., presented as a morphed image within automated system(s) described elsewhere herein.

In aspects, a target aesthetic appearance can be any target aesthetic appearance deemed desirable, such as identified by subject(s) or practitioner(s) as desired outcome(s) of aesthetic modification procedure(s). In certain aspects, a target aesthetic appearance can comprise, e.g., one or more targeted aesthetic feature(s) or characteristic(s) of feature(s) such as vermillion slope, tooth show, and facial balance/symmetry. In certain specific aspects, a target aesthetic appearance can comprise, e.g., one or more targeted aesthetic feature(s) or characteristic(s) of feature(s) such as, e.g., natural facial balance or more specifically natural balance of the upper lip; optimized muscle function; a proportionate lip height-to-lip width; lip proportion to one or more surrounding structures, e.g., the nose, chin, etc.; lip proportion to the remainder of the entirety of the face; amount of tooth (incisor) display; easy or appropriate mouth closure; slope of the upper vermillion border (020) from medial (center) to lateral (side); amount of (level or degree of) exposure of the red vermillion along the length of the upper lip; volume of the red vermillion along the length of the lip; alleviated angular depression or buried vermillion at corner(s) of the mouth; continuity of the internal arc of the upper lip along the wet-dry border; symmetry at the bow peaks of Cupid's bow; amount of anterior (forward) projection on lateral (side/profile) view; vertical slope on lateral (side/profile) view; or other such feature(s) or characteristic(s) identified in the art.

### Procedure Candidates

In aspects, the marking system(s) and method(s)/application(s) of their use, e.g., marking system(s) herein for use in upper lip lift procedure(s), e.g., deep plane upper lip lift procedures (e.g., using the bull's horn incision technique) are suitable for use in aesthetic modification procedure(s) for a wide variety of candidate subjects, including subject(s) for whom one or more procedure(s) (such as an upper lip lift, deep plane upper lip lift, or, e.g., such lifts without the marking system(s) herein) would not otherwise be appropriate. In aspects, suitable candidate(s) on or with which marking system(s) and method(s)/application(s) of their use provided by the disclosure are suitable include, e.g., subjects at or under the age of 50 years; subjects older than 50 years; subjects from any number of ethnicities; subjects having typical skin thickness; subjects having skin which is detectably or significantly thinner than the average population of people of the same age and ethnicity; subjects having skin which is detectable or significantly thicker than the average population of people of the same age and ethnicity; subjects having smooth, youthful skin; subjects having wrinkled skin; subjects with distinct facial features such as particularly shaped noses, e.g., upturned noses; and in aspects other characteristic(s) known in the art which would otherwise exclude patients from being candidates for particular procedure(s). In aspects, marking system(s) and method(s)/application(s) of their use, or, e.g., procedure(s) incorporating marking system(s) provided herein provide detectably or significantly more predictable outcomes than comparable technique(s) practiced in the art, e.g., upper lip lift procedure(s) or corner lift procedure(s) lacking system(s) and method(s) herein, provide detectably or significantly reduced scarring compared to comparable technique(s) practiced in the art, e.g., upper lip lift procedure(s) or corner lift procedures lacking system(s) and method(s) herein, or both. Thus, in providing detectably or significantly more control over, e.g., tooth show/incisor display, scarring, nasal base position, nasal base widening, lip shape, etc., system(s) and method(s) provided herein are applicable to a broad range of candidate(s). In aspects, system(s) and method(s) described herein are applicable to subject(s) characterized as having a “mean” or “snarling” appearance, as incorporating within system(s) and method(s) described herein are mechanisms (e.g., calculations, considerations, or algorithms) which can detectably or significantly change the character of one's appearance (e.g., upper lip) such that a “nicer” or more “friendly” appearance is provided.

According to aspects, a candidate is a user of automated system(s) described herein. In aspects, user(s) of automated system(s) described herein can be candidate(s) for procedure(s) described herein, such as, e.g., upper lip lift procedure(s), corner lift procedure(s), or both.

### Upper Lip Lift Procedure

In aspects, the disclosure herein provides marking system(s) suitable for use in an aesthetic modification procedure, e.g., an upper lip lift surgical procedure. In aspects, the marking system(s) herein are suitable for use in any upper lip lift procedure. In aspects, the marking system(s) provided herein are suitable for use in an upper lip lift procedure utilizing a bull's horn surgical incision technique. In aspects, marking system(s) provided herein are suitable for use in an upper lip lift procedure utilizing a bull's horn surgical technique, such a technique recognized by those skilled in the art, including one or more variations of such a technique comprising maintenance/use of the bull's horn-shaped incision/excision characteristic of the technique.

### Deep Plane Release Upper Lip Lift

In certain specific aspects, the disclosure herein provides marking system(s) suitable for use in an aesthetic modification procedure, e.g., an upper lip lift surgical procedure, wherein the upper lip lift procedure is a subnasal upper lip lift procedure, e.g., a deep plane release upper lip aesthetic modification procedure (e.g., with or without concomitant corner lip lift(s)). In aspects, the disclosure herein provides a subnasal and corner lip lift procedure, e.g., a deep plane release subnasal and corner lip lift procedure comprising specific design element(s) provided by marking system(s) described herein. In aspects, a deep plane release upper lip aesthetic modification procedure is similar to a centrally vectored advancement flap, wherein lateral portions are pulled superior-medially rather than purely in a vertical vector. This technique is described in detail by the present inventor, Dr. Benjamin Talei, in Facial Plast Surg Clin N Am 27 (2019) 385-398 (supra). In aspects, however, such deep plane release upper lip lift aesthetic modification procedure(s) described herein comprise, in aspects, incorporation of concomitant corner lip lift(s). Further, in aspects, such deep plane release upper lip lift aesthetic modification procedure(s) described herein comprise use of specific design system(s), e.g., marking system(s), e.g., marking systems established using specific design principle(s) (as described herein), to provide detectably or significantly increased predictability of results(s) compared to prior procedures or comparable procedure(s) known in the art, and at least generally, at least substantially, or at least essentially standardized outcomes.

According to certain aspects, upper lip lift procedure(s) referenced herein are characterizable as deep plane lip lift(s), and the marking system(s) herein yield a deep plane lip lift design. In aspects, such a deep plane lip lift design uses a rotational advancement of lip skin toward the nose. In aspects, a rotational advancement of lip skin toward the nose is hinged around the apex of the incision at the lateral ala. In aspects, radial marking(s) of marking system(s) disclosed herein indicate certain location(s) where excision will result in a more effective vertical lift than other location(s). E.g., in aspects, the marking system(s) provided herein can comprise one or more medial marking(s) and one or more lateral marking(s), wherein medial marking(s) will experience a detectably or significantly more effective vertical lift than lateral radial marking(s), which have a more diagonal vector. In aspects, such marking(s) aid in the rotational advancement described here.

### Marking System(s)

Described in detail herein are marking system(s) which, when the feature(s) and characteristic(s) of which are applied in aesthetic modification procedure(s), result in a predicted aesthetic appearance. As provided by this disclosure, marker(s) of marking system(s) herein are associated with specific location(s) of a human face, e.g., specific location(s) of the upper lip. In certain aspects, the marker(s) described herein can be literal, physical mark(s) on the skin. In other aspects, marker(s) can represent location(s) on or of the human face, e.g., the location(s) described herein associated with each of the marker(s) of the marking system(s) described herein. As such, in aspects, this disclosure identifies specific location(s), and specific characteristic(s) at and of such specific location(s), wherein modification(s) made at such specific location(s) (as described herein related to the use of marking system(s) disclosed herein) result in the modification of the aesthetic appearance of the human face. In certain aspects, marker(s) can indicate point(s), or characteristic(s) between point(s), such as, e.g., length(s) or distance(s) between point(s), positioning between point(s), and the like. In certain aspects, marker(s) can also or alternatively indicate incision point(s), incision path(s), or both.

According to aspects, marking system(s) provided herein may not necessarily be physical marking(s) made on human skin; rather, in aspects, such marking(s) can be virtual, as generated by automated system(s). In aspects, such marking(s) exist only as data. In aspects, one, some most, generally all, substantially all, essentially all, or all characteristic(s) of such marking(s), e.g., their location(s), total length(s), segment length(s), and the like as described herein exist only as data, wherein such data can be applied to an original image of, e.g., a human face, wherein the application of such data to the original image can result in the generation of a new, morphed image, reflecting the results of such application to the human face wherein feature(s)/landmark(s) of the morphed image reflect that of the marking data.

Accordingly, disclosure related to a length and positioning of marker(s), including segment(s) thereof, should be interpreted as also or alternatively disclosing the length of the path associated with such a location identifiable by such a marker in virtual form (e.g., a virtual marker identifies a location, and the location can be identified by the language used to describe associated marking(s) of marking system(s) herein.)

According to aspects, one, some, most, generally all, substantially all, essentially all, or all marking(s) of marking system(s) provided herein are generated by computer or are generated in a computer-aided manner, e.g., with the use of computer-aided technology.

In aspects, the disclosure provides marking system(s), procedure(s), and method(s) wherein the marking system(s) results in target aesthetic effect(s) when applied in procedure(s) and method(s) described herein; the procedure(s) and method(s) performed to achieve target aesthetic effect(s) described herein; or both are established, performed, or both established and performed utilizing artificial intelligence technology. According to certain aspects, the use of landmark recognition technology, morphing technology, or both detectably or significantly improve(s) the predictability of result(s)/outcome(s) of aesthetic modification procedures comprising their incorporation compared to the predictability of result(s)/outcome(s) of the same aesthetic modification procedure(s) lacking their use. In aspects, this is because the use of such automated system(s) and method(s) can comprise presenting or the ability to present an image representing the predicted outcome(s) of such procedure(s).

In aspects, the disclosure described herein provides marking system(s) for use on a human subject in the preparation for, execution of, or both, an aesthetic modification procedure. In aspects, marking system(s) provided herein are made on the face of a subject, e.g., a human subject, in preparation for one or more aesthetic modification procedure(s). In aspects, marking system(s) are made virtually, e.g., are applied to digital image(s) of, e.g., the face of a subject within automated system(s) described herein. In aspects, marking(s) of virtual marking system(s) of automated system(s) disclosed herein are visible indicator(s) placed upon an image, and which a viewer of the image can see/visually identify. In aspects, marking(s) of virtual marking system(s) of automated system(s) described herein are not visible indicators, yet are digitally present as data that can be applied to image(s) according to automated system use. In certain aspects, the digital presence of data representing marking(s) of virtual marking system(s) is visible as recognizable morphing of an original image according to characteristic(s)/attribute(s) of the marker(s). In aspects, marking system(s) are used on the face or image of a face of a human subject in the preparation for, execution of, or both, such aesthetic modification procedure(s). In aspects, the marking system(s) are used in an upper lip lift procedure, e.g., a subnasal upper lip lift procedure, e.g., a deep plane release upper lip lift procedure (e.g., with or without concomitant corner lip lift(s)). In aspects, the upper lip lift procedure comprises the use of a bull's horn surgical technique. In aspects, marking system(s) comprise one or more components (e.g., marking(s)) suitable for use in one or more procedures performed concomitant to an upper lip lift procedure, such as, e.g., a corner lip lift, nasal base suspension, muscle suspension, or combination of any or all thereof.

In the context of disclosure regarding “use” of marking system(s) described herein, phrases such as (but not necessarily limited to) “incorporation,” “application,” or “use” (and the like) mean the marking system(s) being a functional component of procedure(s) and method(s).

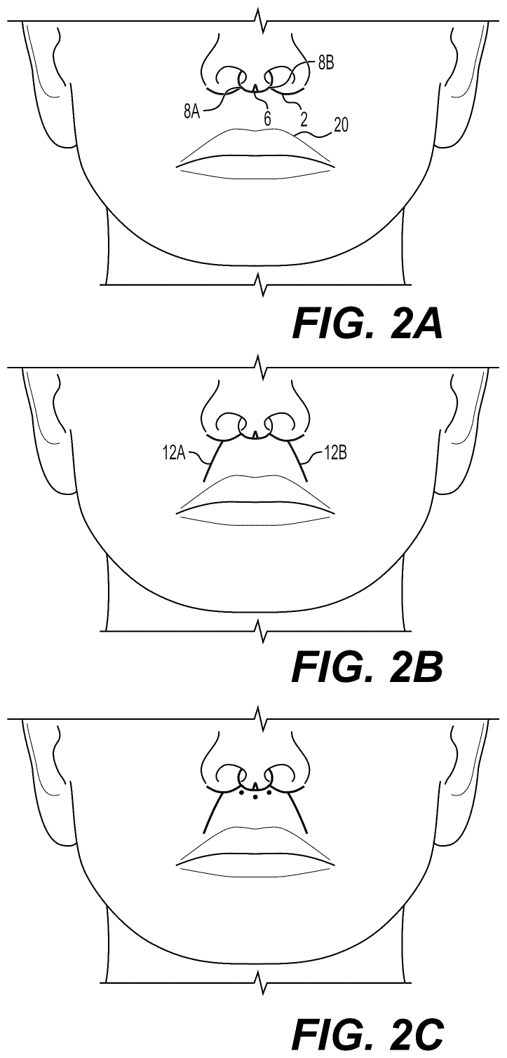

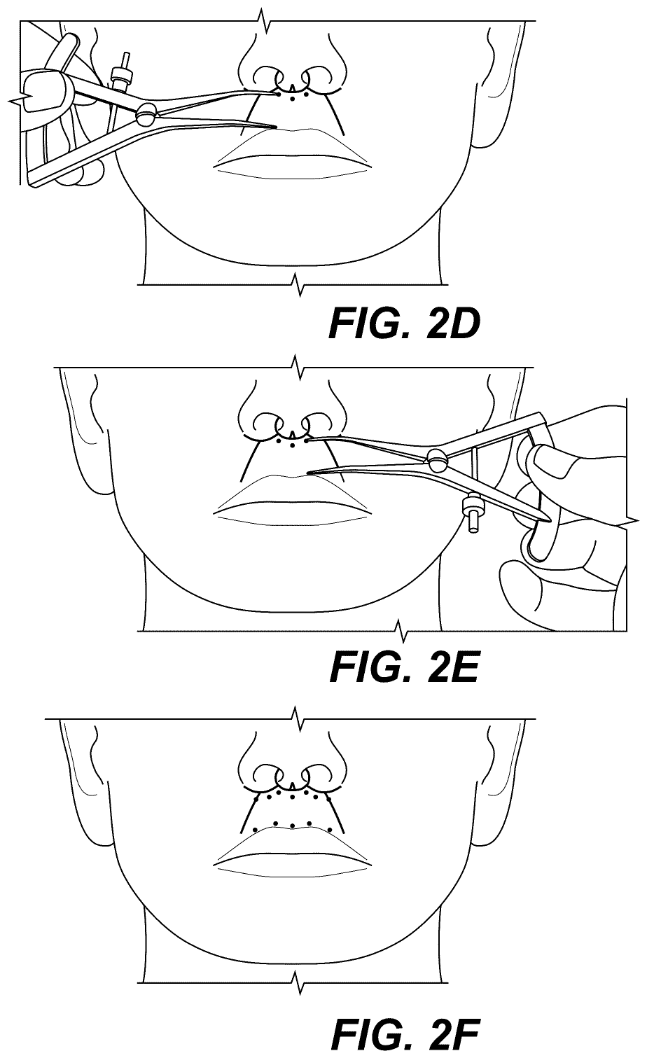

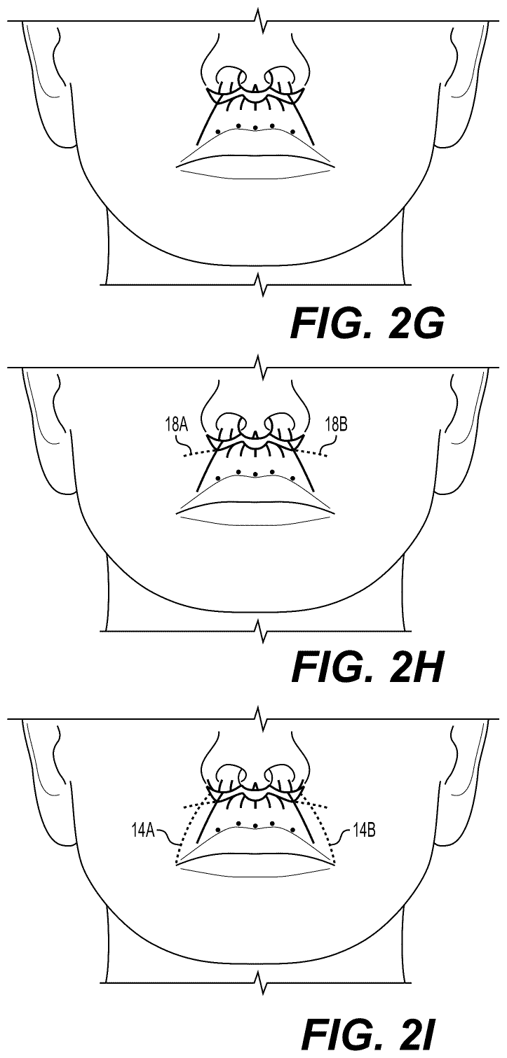

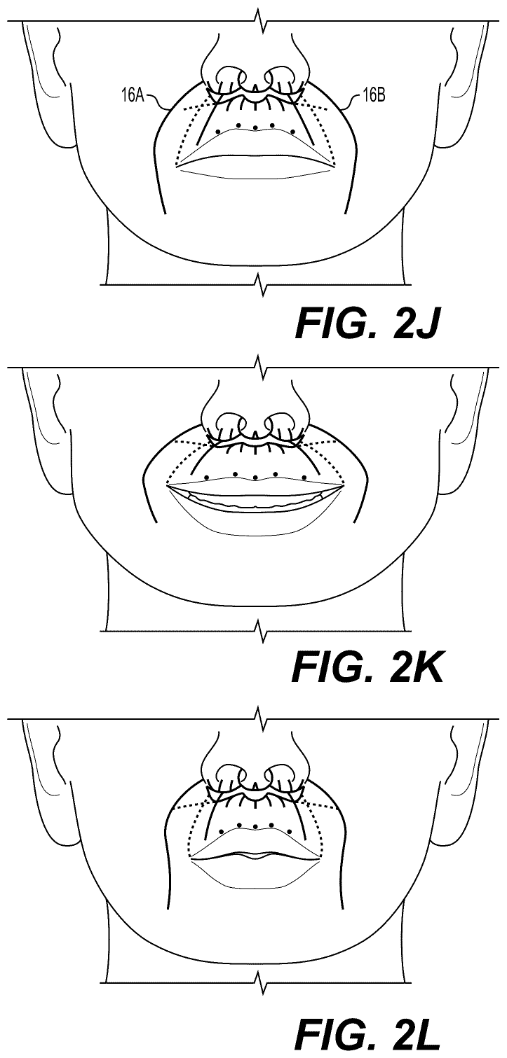

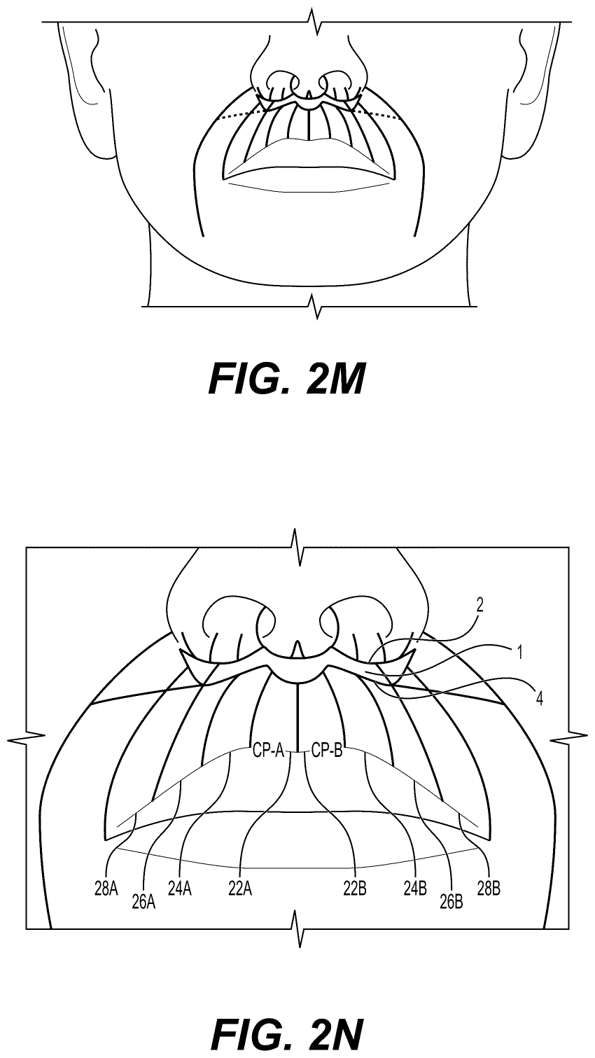

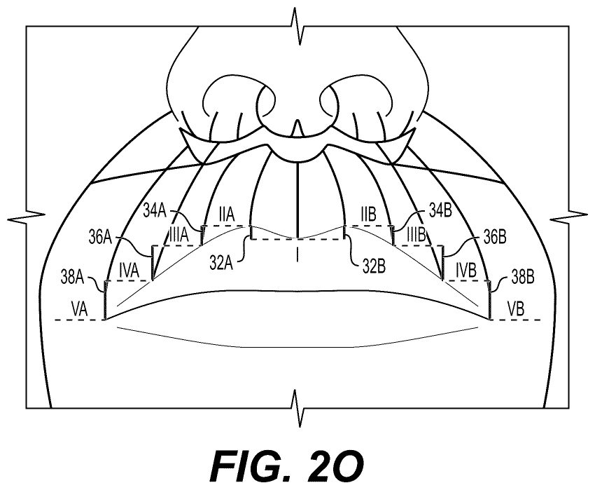

In aspects, the marking system(s) provided herein provide mathematical guidance for attaining a target aesthetic design result. In aspects, the mathematical guidance of the marking system further indicates when or if an ancillary procedure, such as, e.g., a corner lip lift procedure, should be added/included to a first or primary procedure, such as an upper lip lift, in order to achieve a target aesthetic effect such as a target slope of the upper vermillion border (020) (the upper vermillion border being identified in the figure(s) provided herewith; see, e.g., FIG. 2A.)

In aspects, marking system(s) provided herein provide mathematical relationship(s) between two or more marker(s) of the marking system(s) which, when marking system(s) are used/incorporated in method(s) such as method(s) comprising performance of aesthetic modification procedure(s), the marking system(s) yield target effective effect(s) with significant predictability and consistency, e.g., predictability, consistency, or both, compared to at least generally the same or similar, at least substantially the same or similar, at least essentially the same or similar, essentially the same or similar, or the same procedure(s)/method(s) performed on subject(s) or populations of subject(s) which present with at least generally similar, at least substantially similar, at least essentially similar, essentially the same, or the same targeted aesthetic effect performed without such system(s).

In aspects, these mathematical relationships can be programmed and used in automated methods, such as, e.g., automated methods described herein. In aspects, these mathematical relationships can be programmed and used in automated methods, such as, e.g., morphing software, wherein such software is provided an image of a subject and software program(s) utilize the provided mathematical relationships, without user input or in conjunction with one or more user-provided parameters, to provide images to the subject or practitioner exemplifying a predicted aesthetic result if such relationship(s) and parameter(s) are applied to guide aesthetic modification procedure(s). In aspects, results from such program(s) can be modified or edited by a system user to adjust results, e.g., adjust the calculations, to achieve desired aesthetic effect(s).

In certain aspects, marking system(s) comprise a single marking described herein. In certain aspects, marking system(s) comprise at least 2, such as for example, ≥˜3, ≥˜4, ≥˜5, ≥˜6, ≥˜7, ≥˜8, ≥˜9, ≥˜10, ≥˜11, ≥˜12, ≥˜13, ≥˜14, or ≥˜15 markings. In aspects, marking(s) are only suitable for use in/applicable to upper lip lift, e.g., subnasal upper lip lift, procedure(s). In aspects, marking(s) are used in/applicable to/suitable for use in corner lip lift procedure(s), nasal base suspension procedure(s), or muscle suspension procedure(s). In aspects, marking(s) are suitable for use in both an upper lip lift procedure and one or more ancillary/concomitantly performed procedure(s), e.g., a corner lip lift procedure, a nasal base suspension procedure, a muscle suspension procedure, or combination of any or all thereof. In aspects, one or more marking(s) of a marking system are applicable to (suitable for use in or beneficially used in) a first procedure but not applicable to (suitable for use in or beneficially used in) at least a second concomitantly performed procedure(s) wherein the marking(s) are present for the conduct of the at least second concomitantly performed procedure(s). In aspects, marker(s) or at a minimum one or more element(s) of marker(s), e.g., point(s) along marker(s), position(s) of marker(s), length(s) of marker(s), etc. are applicable to (suitable for use in or beneficially used in) both a first procedure and an at least second procedure. For the purposes of exemplification, in aspects, a first procedure in this paragraph can be, e.g., an upper lip lift procedure, e.g., a subnasal upper lip lift procedure, e.g., a deep plane release upper lip lift procedure or a corner lip lift procedure, and an at least second procedure in this paragraph can be, e.g., a corner lip lift procedure, nasal base suspension procedure, muscle suspension procedure, or a combination of any or all thereof.

In aspects, marking system(s) provided herein are guide(s), e.g., comprise marking(s) serving as guide(s) for use before, during, or both before and during an aesthetic modification procedure, e.g., an upper lip lift procedure, e.g., a subnasal upper lip lift procedure (e.g., (e.g., with or without concomitant corner lip lift(s)) and provide a “map” for guiding a practitioner during the procedure toward a predetermined, target aesthetic effect/result. In aspects, the marking system(s) establish a target aesthetic result, e.g., lip shape, resulting from the aesthetic modification procedure (e.g., subnasal upper lip lift procedure performed concomitantly with a corner lip lift procedure).

In aspects, marking system(s) provided herein establish a natural balance of the upper lip when incorporated into an upper lip aesthetic modification procedure. In aspects, the marking system(s) provided herein, when incorporated into/used in/applied to aesthetic modification procedure(s), yield an optimized muscle function resulting from the procedure(s). In aspects, the marking system(s) provided herein indicate when an ancillary procedure, such as, e.g., a corner lip lift procedure, should be added/included to a first or primary procedure, such as an upper lip lift. In aspects, the mathematical guidance of the marking system indicates when an ancillary procedure, such as, e.g., a corner lip lift procedure, should be added/included to a first or primary procedure, such as an upper lip lift, in order to achieve a target aesthetic effect, such as a target slope of the upper vermillion border (020).

According to aspects, the disclosure herein provides marking system(s) and analysis(es) leading to the development thereof, wherein the complexity of upper lip lift design is detectably or significantly reduced by the application of predetermined standard(s) directed to specific characteristic(s) of upper lip, nasal, or upper lip and nasal feature(s) known to yield aesthetically pleasing result(s), e.g., consistent upper vermillion border (020) slope. In aspects, such predetermined standard(s) are provided as characteristic(s) of marker(s) of marking system(s) herein, such as length(s) of marker(s), positioning of marker(s), differences in length(s) or positioning of marker(s), differences in portions of marker(s), relationship(s) between marker(s) or portion(s) thereof (e.g., ratios of length(s) of marker(s) or ratio(s) of certain portion(s)/segment(s) of marker(s), etc. as described herein. Upper lip lift design requires balancing many considerations to yield a target outcome. In aspects, marking system(s) and method(s) of their use/application provided herein, and, e.g., procedure(s) comprising the system(s) and method(s) provided herein detectably or significantly simplify such considerations by providing a mechanism for guiding practitioner(s) to establish condition(s), e.g., parameter(s) according to which significantly more predictable outcome(s) are achieved.

According to certain aspects, marking(s) and marking system(s) described here can comprise any one or more of the feature(s) or characteristic(s) described by any one or more of FIGS. 2A-2O, FIGS. 3A and 3B, FIGS. 4-7, or any combination(s) of any or all thereof.

### Group A (e.g., “Left”) Marking(s) & Group B (e.g., “Right”) Marking(s)

In aspects, the marking system(s) provided herein comprise marker(s) which individually may be located on a single side of the midline (e.g., on a single lateral side of radial marker 1 (006). In aspects, such marker(s) can be present in pairs, wherein the first marker of a pair is positioned on a first lateral side of the midline (e.g., put simply, on one side of the subject's upper lip, e.g., left or right side of the midline of the subject's upper lip) which the second marker of a pair is positioned on the opposing lateral side of the midline (e.g., on the opposite side of the subject's upper lip, e.g., the left or right side of the midline of the subject's upper lip opposite its pair.) In certain aspects, marking system(s) provided by the disclosure comprise one or more markers, e.g., 2 or more, 3 or more, or 4 or more markers positioned on a single side of a centrally positioned first marker (e.g., radial marking 1 (006)). In aspects, marker(s) positioned on a single side of the midline of the upper lip of a subject, e.g., on a first side of radial marking 1 (006) (lateral to radial marking 1 (006) in a single direction), are referred to herein for ease of reference as “Group A” markings.

For example, Group A marking(s) may be markings positioned on the left side of a subject's upper lip. In certain aspects, Group A marking(s) may each have a corresponding Group B marking on the opposing side of the subject's upper lip, such as, e.g., a corresponding marking positioned on the right side of a subject's lip.

In aspects, marker(s) positioned on a single side of the midline of the upper lip of a subject, e.g., on a second side of radial marking 1 (006) (lateral to radial marking 1 (006) in a single direction), and opposite that of a first group of markings (Group A markings), are identifiable as a second group of markings, referred to herein for ease of reference as “Group B” markings.

For example, Group B marking(s) may be markings positioned on the right side of a subject's upper lip. In certain aspects, a Group B marking(s) may each have a corresponding Group A marking on the opposing side of the subject's upper lip, such as, e.g., a corresponding marking positioned on the right side of a subject's lip.

In aspects, a Group A-Group B marking pair (“A-B pair”) are at least generally, at least substantially, at least essentially, essentially, or are, mirror image(s) of one another in terms of position/positioning on the upper lip, e.g., in their identification of facial feature(s) on opposite sides of a midline. However, one member of such an A-B marking pair may have characteristic(s) which are detectably or significantly different from its counterpart in order to address an asymmetry, such as, e.g., it may comprise an excision height segment length, a final lip height segment length, etc., which is detectably or significantly different from its counterpart.

### Upper Excision Boundary Marker(s)

In aspects, the marking system(s) provided herein comprise a marking along the nasal base. In aspects, the marking along the nasal base indicates an upper excision boundary (002), e.g., the uppermost boundary of a surgical excision performed as at least a part of a procedure incorporating the marking system(s). In aspects, the upper excision boundary (002) is the upper excision boundary of a bull's horn-shaped excision made as part of an upper lip lift procedure, e.g., subnasal upper lip lift procedure, e.g., deep plane release upper lip lift modification procedure (e.g., with or without concomitant corner lip lift(s)). Figures, e.g., 2A, 2N, 3A and 3B illustrate exemplary positions of an upper excision boundary marker/marking, indicated as element (002) of the figures. As with any one or more other marker(s) described herein, such a marker can be, e.g., a virtual marker, e.g., identified by landmark detection technology(ies) of automated system(s) provided herein.

In aspects, an upper excision boundary marking (002) comprises the uppermost point (e.g., incision point of each of at least one, typically a plurality of, radial marking(s) of the marking system(s) (described elsewhere herein).

In aspects, the upper excision boundary marking (002) is an at least substantially horizontal marking. In aspects, the upper excision boundary marking (002) follows horizontally along the subnasal, perialar crease. According to certain aspects, the upper excision boundary marking (002) identifies the uppermost portion of the upper lip to be excised in an upper lip lift procedure comprising use of the marking system(s). In aspects, the upper excision boundary marking (002) does not extend beyond the natural ending of the subnasal, perialar crease of the subject.

In aspects, an upper excision boundary marking (002) comprises two peaks. In aspects, the two peaks are positioned on either side of the midline at the divergence of the medial crural footplate and the nasal sill.

In aspects, an upper excision boundary/boundary marking (002), in conjunction with a lower excision boundary/boundary marking (004), defines a portion of the upper lip to be excised in an upper lip lift procedure comprising use of the marking system(s).

### Lower Excision Boundary Marker(s)

In aspects, marking system(s) comprise a lower excision boundary marker (004). In aspects, the lower excision boundary marker (004) is highly adjustable from patient to patient and is made according to specific requirements of the procedure needed to attain a target aesthetic effect. In aspects, the lower excision boundary marker (004) comprises a collection of second, lower incision point markers (with the first, upper incision point markers being encompassed by the upper excision boundary marker (002)) and is a mid-line marking between the nasal sill and the upper vermillion border (020). As is described here, in aspects, the lower excision boundary marker (004) indicates the lower incision point of each radial marking(s). A lower excision boundary marker (004), in aspects, is established by connecting two or more lower incision point markers, each such lower incision point marker established based upon target aesthetic effect(s). In aspects, a lower excision boundary marker (004) is not completely established until at least some, most, generally all, or all lower incision point markers are established, until some, most, generally all, or all upper vermillion border (020) points are established, or some, most, generally all, or all of both lower incision point markers and upper vermillion border (020) points are established. Figures, e.g., 2N and 3, illustrate an exemplary position of a lower excision boundary marker (004), illustrated as element (004) of the figures.

In aspects, a lower excision boundary marker (004) is substantially horizontal. In aspects, a lower excision boundary marker (004) is positioned below the upper excision boundary marker (002), with the distance between the upper excision boundary marker (002) and the lower excision boundary marker (004) defining a portion of the upper lip to be excised in an upper lip lift procedure incorporating the marking system(s). In aspects, the distance between any point along the lower excision boundary marker (004) and the upper excision boundary marker (002) defines (represents) the excision height at such location.

In aspects, a lower excision boundary marker (004) is positioned above the upper vermillion border (020) with the distance between the upper excision boundary marker (002) and the upper vermillion border (020) defining the final lip height, e.g., remaining lip height after the excision of a portion of the lip located between the upper (002) and lower excision boundary (004) markers.

In certain aspects, the lower excision boundary marker (004) meets the upper excision boundary marker (002) at the lateral ends of each marker. In aspects, there is at least one detectable or significant distance between the lower (004) and upper (002) excision boundary marker(s) across the substantially horizontal span of the two markers.

In aspects, the respective point(s) establishing the lower excision boundary marker (004) corresponding to location(s)/position(s) of radial marking line(s) are each located such that use of/application of the marking system in the aesthetic procedure(s) yields target aesthetic result(s). In aspects, the individual positioning of respective point(s) according to target aesthetic result(s) provides a mechanism for customizing target aesthetic result(s) by providing for the modification of the excision height across the span of the upper (002) and lower excision boundary (004) marker(s). Modification of the excision height across the span of the upper (002) and lower excision boundary (004) marker(s) is accomplished by modifying the position of a plurality of points along the lower excision boundary marker (004) at location(s)/position(s) of radial marking lines(s), establishing the shape of the portion of the upper lip to be excised and establishing the final lip height at the location(s)/position(s) of specific radial marking line(s).

In aspects, the lower excision boundary marker (004) comprises point(s) which fall(s) along the course of each of at least 1, ≥˜2, ≥˜3, ≥˜4, ≥˜5, ≥˜6, or ≥˜7 radial marker(s). In aspects, as described above, the location of each such point on the upper lip, between the upper excision boundary marker (002) and the upper vermillion border (020), is established according to a target aesthetic effect (e.g., the final lip height desired/targeted at such location to achieve a target aesthetic effect). In aspects, such point(s), and thus the lower excision boundary marker (004) itself, represent(s) interim point(s) along the course of the radial marking(s) as the marking(s) span(s) from the upper excision boundary marker (002) to the upper vermillion border (020). In aspects, such a span of each radial marker is specifically marked/identified on the skin of the subject. In aspects, such a span may be broken (e.g., by the line not being indicated within the portion of the upper lip between the upper (002) and lower excision boundary (004) markers indicating the portion of the upper lip to be excised); however, the trajectory of the marker(s) is clearly indicated despite any such existing gap.

### Radial Markers

In aspects, the disclosure provides a marking system comprising at least one, e.g., ≥˜2, ≥˜3, ≥˜4, ≥˜5, ≥˜6, ≥˜7, ≥˜8, ≥˜9, ≥˜10, ≥˜11, ≥˜12, ≥˜13, ≥˜14, or, e.g., ≥˜15 radially spaced markings (“radial markers” or “radial markings”). Typically, marking system(s) provided herein comprise at least about 7, such as ≥˜8, ≥˜9, ≥˜10, ≥˜11, ≥˜12, ≥˜13, ≥˜14, or ≥˜15 radial markers. In certain aspects, the marking system comprises at least 7 radially spaced markings. Use of the term “radial” marker(s) or “radially spaced” marker(s) herein can, in aspects, reflect the positioning of one or more such marker(s) relative to a center point, wherein the center point is, e.g., a central location of a human face. For example, a single radial marker can, e.g., be positioned outward from such a center point, e.g., on one side or the other of the center point. In another example, two or more markers can, e.g., be positioned outward from such a center point on either side of the center point (e.g., one or more radial markers (s) on one side of the center point and one or more markers on the other side of the center point). In yet another example, two or more markers can, e.g., be positioned outward from such a center point on the same side of the center point, but, for example, at different distances from such a point, with one being closer to the center point and another being further from the center point.

In aspects, marking system(s) comprise radially spaced markings identified as “primary radial marking(s)” or “p-radial marking(s)”/“p-radial marker(s).” In aspects, marking system(s) comprise both p-radial marker(s) and additional radial markers in between two or more neighboring p-radial markers identified herein as “intermediary radial marking(s)” or “i-radial marking(s).” This is described further elsewhere herein.

In aspects, radial marker(s) are at least generally vertical (e.g., ranging from vertical to more diagonal; but not characterizable as generally or substantially horizontal). In aspects, radial marker(s) span the distance between the nasal base and the upper vermillion. In aspects, radial marker(s) have a first, upper point located along an upper excision boundary marker (002) and a second, lower point located along the upper vermillion border (020). In aspects, the span of the marker is fully visible/indicated on the skin of the subject. In aspects, the span is not completely visually indicated, e.g., a break in the span of the marker is present; however, the path of the marker is clearly comprehensible given an indication of the marker being present on either side of any such gap(s). For example, in aspects, radial marker(s) have an at least first point indicated along the upper excision boundary marker (002) and indicated at/along the nasal base or nasal sill as indicated by an upper excision boundary (002) and an at least second point indicated along the upper vermillion border (020), with the trajectory of the radial marker(s) gradually radially rotating laterally toward the angle of the nasolabial fold(s) (016A) and (016B) (respectively), following the lip's natural relaxed tension lines, pores, or both. In aspects, at least first and second points are fully visually connected. In aspects, the visual connection between such at least first and second points is interrupted by a portion of the upper lip identified as the portion to be excised, such a segment of the radial marker(s) within such a portion of the upper lip to be excised representing the excise height at the location of such radial marker. In aspects, one or more radial marker(s) may be continuous while one or more radial marker(s) may be interrupted.

In aspects, radial marker(s) of marking system(s) provided herein each identify independent target locations within the upper lip for modification of the upper lip in an aesthetic modification procedure (e.g., an upper lip lift, e.g., a subnasal upper lip lift, e.g., a deep plane release upper lip lift procedure(s) (e.g., with or without concomitant corner lip lift(s)). In aspects, independent modifications made at the independent target locations provide one or more target aesthetic effect(s).

In aspects, each radial marker/marking comprises at least 3 distinct points within its length, wherein a first distinct point within each marks a first upper incision point along the nasal base (together serving to form an upper excision boundary marker (002)); a second distinct point within each marks a second lower incision point and helps to establish the lower excision boundary marker (004); and a third point within each located on the upper vermillion border (020). In aspects, a first upper incision point along the nasal base may mark the end of the radial marker. In aspects, the upper excision boundary marker (002) may mark the end of one or more radial marker(s). In aspects, radial marker(s) may detectably extend beyond an upper incision point along the nasal base. In aspects, radial marker(s) may extend above the upper excision boundary marker (002).

As alluded to above, in aspects, radial marking(s) are positioned between the nasal base, e.g., an upper excision boundary (002) along the nasal base, and the upper vermillion border (020). In aspects radial marking(s), e.g., at least 7 radial marking(s), progress from at least substantially vertical at the midline point of the Cupid's bow trough with gradual rotation laterally toward the angle of the nasolabial fold(s) (016A) and (016B) (respectively). In aspects, as the set of radial marking(s) progresses laterally, radial marking(s) follow(s) the upper lip's natural relaxed tension lines, pores, or both.

### Radial Marking 1

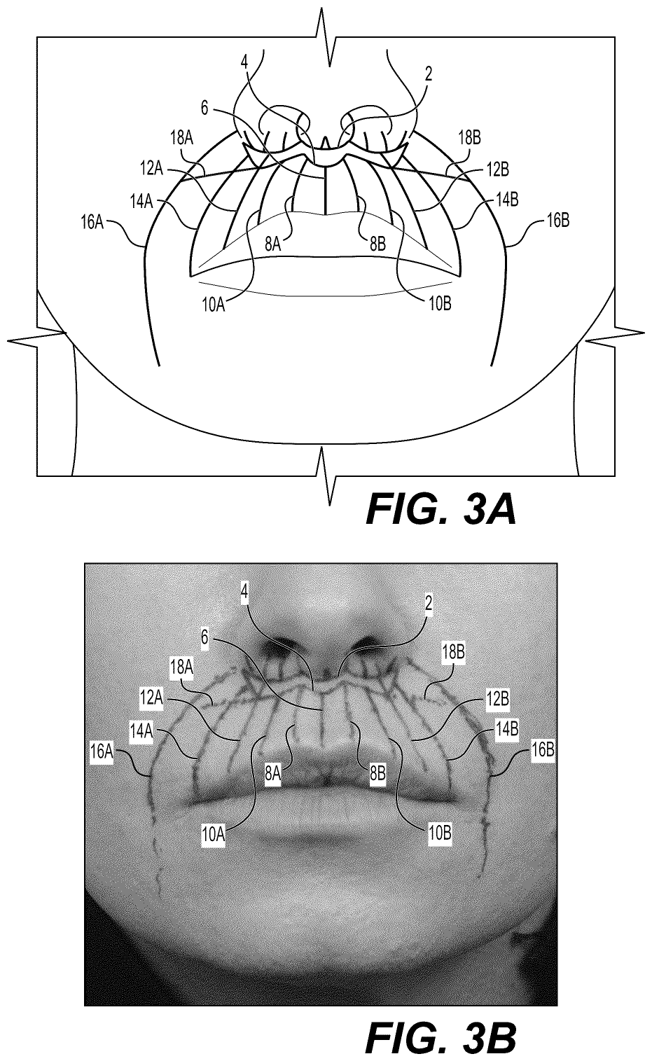

In aspects, marking system(s) provided herein comprise a radial marking, e.g., a first radial marking, for ease of reference herein identified as “radial marking 1” (006), positioned in the middle of the columella and philtral dimple. In aspects, radial marking 1 (006) is positioned at the lowest point of the trough of Cupid's bow. In aspects, radial marking 1 (006) is the most centrally located of any radial marker(s)/marking(s) present in marking system(s) provided herein. FIGS. 3A and 3B illustrate an exemplary position of radial marking 1, element (006) of the figure. In aspects, radial marking 1 (006) is identified herein as a primary radial marker, e.g., a p-radial marker.

According to certain aspects, radial marking 1 (006) is an at least generally, at least substantially, at least essentially, essentially, or is a vertical marking. In aspects, as radial marker(s) progress laterally from radial marking 1 (006), they detectably or significantly rotate laterally toward the angle of the nasolabial fold(s) (016A) and (016B). FIG. 2A shows the position of one end of radial marking 1 (006). Shown as a pointed “v,” such a positioning mark can be, e.g., in another aspect, indicated as a straight line or other indicator. See also FIGS. 3A and 3B, indicating radial marker (006).

In certain aspects, radial marking 1 (006) comprises a total length, extending from the nasal base, e.g., the upper excision boundary (002), to the upper vermillion border (020). In aspects, the total length is represented by the combination of an excision height segment and a final lip height segment. In aspects, a first portion of radial marking 1 (006) comprises an excision height segment bound by the upper excision boundary marker (002) and the lower excision boundary marker (004). In aspects, the excision height segment identifies the portion of the upper lip along radial marking 1 (006) to be excised during the procedure to which the marking system is applied, e.g., an upper lip lift procedure. In aspects, a second portion of radial marking 1 (006) comprises a final lip height segment bound by the lower excision boundary marker (004) and the upper vermillion border (020). In aspects, the final lip height segment identifies the portion of the upper lip along radial marking 1 (006) which will remain post-excision.

### Radial Marking 1: Total Length

In aspects, the total length of radial marking 1 (006), and, similarly, in aspects one, some, most, or in aspects all other radial marking(s) described herein, is the distance between its point along the upper excision boundary marker (002) and its point along the upper vermillion border (020). In aspects, radial marking 1 (006), and similarly, in aspects, one, some, most, or in aspects all other radial marking(s) described herein, is/are not continuous. In aspects, there is a gap along the course of radial marking(s) representing the excision height at such a position along its length. In aspects, as has been described, and which applies to the discussion of all other marking(s) disclosed herein, the marking(s) are virtual and are element(s) of automated system(s) described herein.

In aspects, radial marking 1 (006) can have any total length (“1-T”) suitable and effective for achieving targeted aesthetic effect(s).

In aspects, radial marking 1 (006) has a total length (“1-T”) of between about 8 mm and about 20 mm, such as, e.g., ˜8 mm-˜19 mm, ˜8 mm-˜18 mm, ˜8 mm-˜17 mm, ˜8 mm-˜16 mm, ˜8 mm-˜15 mm, ˜8 mm-˜14 mm, ˜8 mm-˜13 mm, ˜8 mm-˜12 mm, ˜8 mm-˜11 mm, or, e.g., ˜8 mm-˜10 mm.

In aspects, radial marking 1 (006) has a total length of, e.g., ˜9 mm-˜20 mm, ˜10 mm-˜20 mm, ˜11 mm-˜20 mm, ˜12 mm-˜20 mm, ˜13 mm-˜20 mm, ˜14 mm-˜20 mm, or ˜15 mm-˜20 mm.

In aspects, radial marking 1 (006) has a total length of ˜8 mm-˜19 mm, ˜9 mm-˜18 mm, ˜10 mm-˜17 mm, ˜10 mm-˜16 mm, or, e.g., ˜11 mm-˜15 mm, such as, e.g., ˜11 mm, ˜12 mm, ˜13 mm, ˜14 mm, or, e.g., ˜15 mm.

### Radial Marking 1: Excision Height Segment Length

In aspects, radial marking 1 (006) can have any excision height segment length (“1-EX”) suitable and effective for achieving targeted aesthetic effect(s).

In aspects, radial marking 1 (006) comprises an excision height segment length (“1-EX”) of about 0 mm to about 10 mm, such as, e.g., ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, or, e.g., ˜0 mm-˜2 mm.

In aspects, radial marking 1 (006) comprises an excision height segment length of about 1 mm-˜10 mm, 2 mm-˜10 mm, 3 mm-˜10 mm, 4 mm-˜10 mm, 5 mm-˜10 mm, 6 mm-˜10 mm, 7 mm-˜10 mm, 8 mm-˜10 mm, or, e.g., 9 mm-˜10 mm.

In aspects, radial marking 1 (006) comprises an excision height segment length of about 0 mm-˜8 mm, ˜0 mm-˜6 mm, ˜1 mm-˜8 mm, ˜1 mm-˜6 mm, ˜1 mm-˜5 mm, ˜1 mm-˜4 mm, ˜2 mm-˜6 mm, ˜2 mm-˜5 mm, ˜2 mm-˜4 mm, or, e.g., ˜1 mm-˜2 mm.

### Radial Marking 1: Final Lip Height Segment Lenth

In aspects, radial marking 1 (006) can have any final lip height segment length (“1-FH”) suitable and effective for achieving targeted aesthetic effect(s).

In aspects, radial marking 1 (006) comprises a final lip height segment length (“1-FH”) of between about 1 mm-˜18 mm, such as, e.g., ˜1 mm-˜17 mm, ˜1 mm-˜16 mm, ˜1 mm-˜15 mm, ˜1 mm-˜14 mm, ˜1 mm-˜13 mm, ˜1 mm-˜12 mm, ˜1 mm-˜11 mm, ˜1 mm-˜10 mm, ˜1 mm-˜9 mm, or, e.g., ˜1 mm-˜8 mm.

In aspects, radial marking 1 (006) comprises a final lip height segment length of ˜2 mm-˜18 mm, ˜3 mm-˜18 mm, ˜4 mm-˜18 mm, ˜5 mm-˜18 mm, ˜6 mm-˜18 mm, ˜7 mm-˜18 mm, ˜8 mm-˜18 mm, ˜9 mm-˜18 mm, or, e.g., ˜10 mm-˜18 mm.

In aspects, radial marking 1 (006) comprises a final lip height segment of ˜2 mm-˜17 mm, ˜3 mm-˜16 mm, ˜4 mm-˜15 mm, ˜5 mm-˜15 mm, ˜6 mm-˜15 mm, ˜7 mm-˜15 mm, ˜8 mm-˜15 mm, ˜9 mm-˜15 mm, ˜10 mm-˜14 mm, or, e.g., ˜11 mm-˜13 mm.

In aspects, the excision height segment length is between about 8% and about 20% of the length of final lip height segment, such as, e.g., ˜8%-˜18%, ˜8%-˜16%, ˜8%-˜14%, ˜8%-˜12%, or ˜8%-˜10% of the final lip height segment length, as in, e.g., ˜10%-˜20%, ˜12%-˜20%, ˜14%-˜20%, ˜16%-˜20% or ˜18%-˜20% of the final lip height segment length, for example ˜9%-˜19%, ˜10%-˜18%, ˜11%-˜17%, ˜12%-˜16%, or, e.g., ˜13%-˜15% of the final lip height segment length.

### Radial Markings 2A/2B

In aspects, marking system(s) provided herein comprise 2 radial markings, e.g., a set of paramedian markings, for ease of reference herein identified as “radial marking 2A” (or radial marker 2A or radial indicator 2A) (008A) and “radial marking 2B,” (or radial marker 2B or radial indicator 2B) (008B) positioned lateral to and on either side of the columella. In aspects, radial marking lines 2A (008A) and 2B (008B) are located at the peaks of Cupid's bow. In aspects, radial marking lines 2A (008A) and 2B (008B) are located at the point where the medial crural footplates diverge into the nose at the junction with the alar sill. In aspects, radial marking(s) 2A (008A) and 2B (008B) are characterizable as paramedian lines or philtral lines due to their anatomical positioning. FIGS. 3A and 3B illustrate an exemplary position of radial markings 2A (008A) and 2B (008B), identified as element (008) in the figure, e.g., one of radial marking 2A (008A) or 2B (008B), wherein the other is the corresponding marker on the opposite side of the face. In aspects, radial marking(s) 2A (008A), 2B (008B), or both is/are identified herein as primary radial marker(s), e.g., p-radial marker(s). FIG. 2A shows the position of at least one end of radial markers 2A (008A) and 2B (008B), respectively. See the location of radial marking(s) 2A (008A) and 2B (008B) also in, e.g., FIGS. 3A and 3B.

In certain aspects, only one of the radial marking lines 2A (008A) and 2B (008B) may be present in a marking system. Typically, a marking system will comprise both radial marking 2A (008A) and radial marking 2B (008B).

According to certain aspects, radial marking(s) 2A (008A) and 2B (008B) are each at least generally, at least substantially, at least essentially, or essentially vertical marking(s). In aspects, as radial marker(s) 2A (008A) and 2B (008B) are positioned laterally to radial marking 1 (006), each of radial marker 2A (008A) and 2B (008B) may detectably or significantly rotate laterally toward the angle of the nasolabial fold(s) (016A) and (016B), respectively. In aspects, the lateral rotation of radial marker(s) 2A (008A) and 2B (008B) is detectably or significantly greater than that of radial marker 1 (006) but detectably or significantly less than that of one or more of radial markers 3A (010A), 3B (010B), 4A (012A), 4B (012B), 5A (014A), 5B (014B), including combination(s) of any or all thereof.

In certain aspects, radial markings 2A (008A), 2B (008B), or both comprise(s) a total length, extending from the nasal base, e.g., upper excision boundary (002), to the upper vermillion border (020). In aspects, the total length is represented by the combination of an excision height segment and a final lip height segment. In aspects, a first portion of radial marking(s) 2A (008A), 2B (008B), or both comprise(s) an excision height segment bound by the upper excision boundary marker (002) and the lower excision boundary marker (004). In aspects, the excision height segment identifies the portion of the upper lip along radial marking 1 (006) to be excised during the procedure to which the marking system is applied, e.g., an upper lip lift procedure. In aspects, a second portion of radial marking(s) 2A (008A), 2B (008B), or both comprise(s) a final lip height segment bound by the lower excision boundary marker (004) and the upper vermillion border (020). In aspects, the final lip height segment identifies the portion of the upper lip along radial marking(s) 2A (008A), 2B (008B), or both, which will remain post-excision.

### Radial Markings 2A/2B: Total Length

In aspects, radial marking 2A (008A), radial marking 2B (008B), or both can have any total length (“2A-T” and “2B-T”, respectively) suitable and effective for achieving targeted aesthetic effect(s).

In aspects, radial marking 2A (008A) has a total length (“2A-T”) of between about 8 mm and about 20 mm, such as, e.g., ˜8 mm-˜19 mm, ˜8 mm-˜18 mm, ˜8 mm-˜17 mm, ˜8 mm-˜16 mm, ˜8 mm-˜15 mm, ˜8 mm-˜14 mm, ˜8 mm-˜13 mm, ˜8 mm-˜12 mm, ˜8 mm-˜11 mm, or, e.g., ˜8 mm-˜10 mm.

In aspects, radial marking 2A (008A) has a total length of, e.g., ˜9 mm-˜20 mm, ˜10 mm-˜20 mm, ˜11 mm-˜20 mm, ˜12 mm-˜20 mm, ˜13 mm-˜20 mm, ˜14 mm-˜20 mm, or ˜15 mm-˜20 mm.

In aspects, radial marking 2A (008A) has a total length of ˜8 mm-˜19 mm, ˜9 mm-˜18 mm, ˜10 mm-˜17 mm, ˜10 mm-˜16 mm, or, e.g., ˜11 mm-˜15 mm, such as, e.g., ˜11 mm, ˜12 mm, ˜13 mm, ˜14 mm, or, e.g., ˜15 mm.

In aspects, radial marking 2B (008B) has a total length (“2B-T”) of between about 8 mm and about 20 mm, such as, e.g., ˜8 mm-˜19 mm, ˜8 mm-˜18 mm, ˜8 mm-˜17 mm, ˜8 mm-˜16 mm, ˜8 mm-˜15 mm, ˜8 mm-˜14 mm, ˜8 mm-˜13 mm, ˜8 mm-˜12 mm, ˜8 mm-˜11 mm, or, e.g., ˜8 mm-˜10 mm.

In aspects, radial marking 2B (008B) has a total length of, e.g., ˜9 mm-˜20 mm, ˜10 mm-˜20 mm, ˜11 mm-˜20 mm, ˜12 mm-˜20 mm, ˜13 mm-˜20 mm, ˜14 mm-˜20 mm, or ˜15 mm-˜20 mm.

In aspects, radial marking 2B (008B) has a total length of ˜8 mm-˜19 mm, ˜9 mm-˜18 mm, ˜10 mm-˜17 mm, ˜10 mm-˜16 mm, or, e.g., ˜11 mm-˜15 mm, such as, e.g., ˜11 mm, ˜12 mm, ˜13 mm, ˜14 mm, or, e.g., ˜15 mm.

### Radial Markings 2A/2B: Excision Height Segment Length

In aspects, radial marking 2A (008A), radial marking 2B (008B), or both can have any excision height segment length (“2A-EX” and “2B-EX”, respectively) suitable and effective for achieving targeted aesthetic effect(s).

In aspects, radial marking 2A (008A) comprises an excision height segment length (“2A-EX”) of about 0 mm to about 10 mm, such as, e.g., ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, or, e.g., ˜0 mm-˜2 mm.

In aspects, radial marking 2A (008A) comprises an excision height segment length of about 1 mm-˜10 mm, 2 mm-˜10 mm, 3 mm-˜10 mm, 4 mm-˜10 mm, 5 mm-˜10 mm, 6 mm-˜10 mm, 7 mm-˜10 mm, 8 mm-˜10 mm, or, e.g., 9 mm-˜10 mm.

In aspects, radial marking 2A (008A) comprises an excision height segment length of about 0 mm-˜8 mm, ˜0 mm-˜6 mm, ˜1 mm-˜8 mm, ˜1 mm-˜6 mm, ˜1 mm-˜5 mm, ˜1 mm-˜4 mm, ˜2 mm-˜6 mm, ˜2 mm-˜5 mm, ˜2 mm-˜4 mm, or, e.g., ˜1 mm-˜2 mm.

In aspects, radial marking 2B (008B) comprises an excision height segment length (“2B-EX”) of about 0 mm to about 10 mm, such as, e.g., ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, or, e.g., ˜0 mm-˜2 mm.

In aspects, radial marking 2B (008B) comprises an excision height segment length of about 1 mm-˜10 mm, 2 mm-˜10 mm, 3 mm-˜10 mm, 4 mm-˜10 mm, 5 mm-˜10 mm, 6 mm-˜10 mm, 7 mm-˜10 mm, 8 mm-˜10 mm, or, e.g., 9 mm-˜10 mm.

In aspects, radial marking 2B (008B) comprises an excision height segment length of about 0 mm-˜8 mm, ˜0 mm-˜6 mm, ˜1 mm-˜8 mm, ˜1 mm-˜6 mm, ˜1 mm-˜5 mm, ˜1 mm-˜4 mm, ˜2 mm-˜6 mm, ˜2 mm-˜5 mm, ˜2 mm-˜4 mm, or, e.g., ˜1 mm-˜2 mm.

### Radial Markings 2A/2B: Final Lip Height Segment Length

In aspects, radial marking 2A (008A), radial marking 2B (008B), or both can have any final lip height segment length (“2A-FH” and “2B-FH”, respectively) suitable and effective for achieving targeted aesthetic effect(s).

In aspects, radial marking 2A (008A) comprises a final lip height segment length (“2A-FH”) of between about 1 mm-˜18 mm, such as, e.g., ˜1 mm-˜17 mm, ˜1 mm-˜16 mm, ˜1 mm-˜15 mm, ˜1 mm-˜14 mm, ˜1 mm-˜13 mm, ˜1 mm-˜12 mm, ˜1 mm-˜11 mm, ˜1 mm-˜10 mm, ˜1 mm-˜9 mm, or, e.g., ˜1 mm-˜8 mm.

In aspects, radial marking 2A (008A) comprises a final lip height segment length of ˜2 mm-˜18 mm, ˜3 mm-˜18 mm, ˜4 mm-˜18 mm, ˜5 mm-˜18 mm, ˜6 mm-˜18 mm, ˜7 mm-˜18 mm, ˜8 mm-˜18 mm, ˜9 mm-˜18 mm, or, e.g., ˜10 mm-˜18 mm.

In aspects, radial marking 2A (008A) comprises a final lip height segment of ˜2 mm-˜17 mm, ˜3 mm-˜16 mm, ˜4 mm-˜15 mm, ˜5 mm-˜15 mm, ˜6 mm-˜15 mm, ˜7 mm-˜15 mm, ˜8 mm-˜15 mm, ˜9 mm-˜15 mm, ˜10 mm-˜14 mm, or, e.g., ˜11 mm-˜13 mm.

In aspects, radial marking 2B (008B) comprises a final lip height segment length (“2B-FH”) of between about 1 mm-˜18 mm, such as, e.g., ˜1 mm-˜17 mm, ˜1 mm-˜16 mm, ˜1 mm-˜15 mm, ˜1 mm-˜14 mm, ˜1 mm-˜13 mm, ˜1 mm-˜12 mm, ˜1 mm-˜11 mm, ˜1 mm-˜10 mm, ˜1 mm-˜9 mm, or, e.g., ˜1 mm-˜8 mm.

In aspects, radial marking 2B (008B) comprises a final lip height segment length of ˜2 mm-˜18 mm, ˜3 mm-˜18 mm, ˜4 mm-˜18 mm, ˜5 mm-˜18 mm, ˜6 mm-˜18 mm, ˜7 mm-˜18 mm, ˜8 mm-˜18 mm, ˜9 mm-˜18 mm, or, e.g., ˜10 mm-˜18 mm.

In aspects, radial marking 2B (008B) comprises a final lip height segment of ˜2 mm-˜17 mm, ˜3 mm-˜16 mm, ˜4 mm-˜15 mm, ˜5 mm-˜15 mm, ˜6 mm-˜15 mm, ˜7 mm-˜15 mm, ˜8 mm-˜15 mm, ˜9 mm-˜15 mm, ˜10 mm-˜14 mm, or, e.g., ˜11 mm-˜13 mm.

In aspects, the excision height segment length of radial marking 2A, radial marking 2B, or both radial markings 2A and 2B is/are between about 8% and about 20% of the length of final lip height segment of the same radial marking, such as, e.g., ˜8%-˜18%, ˜8%-˜16%, ˜8%-˜14%, ˜8%-˜12%, or ˜8%-˜10% of the final lip height segment length of the same radial marking, as in, e.g., ˜10%-˜20%, ˜12%-˜20%, ˜14%-˜20%, ˜16%-˜20% or ˜18%-˜20% of the final lip height segment length, for example ˜9%-˜19%, ˜10%-˜18%, ˜11%-˜17%, ˜12%-˜16%, or, e.g., ˜13%-˜15% of the final lip height segment length of the same radial marking.

In aspects, the final lip height segment length of radial marking 2A (008A) is at least generally, at least substantially, at least essentially, is essentially, or is the same as the final lip height segment length of radial marking 2B, and vice-versa. In aspects, the final lip height segment of radial marking 2A (008A), radial marking 2B (008B), or both, is/are at least generally, at least substantially, at least essentially, is/are essentially, or is/are the same as the final lip height segment of radial marking 1. In aspects, the final lip height segment of two or more of radial markings 2A (008A), 2B (008B), and radial marking 1 (006) are within about 20%, or within ˜15%, ˜10%, ˜9%, ˜8%, ˜7%, ˜6%, ˜5%, ˜4%, ˜3%, ˜2%, ˜1%, or within ˜0.5% or ˜0.1% of the final lip height segment of one or more another of radial markings 2A (008A, 2B (008B, and radial marking 1 (006).

### Radial Markings 3A/3B

In aspects, marking system(s) provided herein comprise 2 radial markings, for ease of reference herein identified as “radial marking 3A” (010A) and “radial marking 3B” (010B), positioned lateral to each of radial markings 2A (008A) and 2B (008B), respectively. In aspects, radial markings 3A (010A) and 3B (010B) are positioned intermedially, e.g., centrally, between radial markings 2A (008A) and 4A (012A) and between radial markings 2B (008B) and 4B (012B), respectively. FIGS. 3A and 3B illustrate an exemplary position of radial markings 3A (010A) and 3B (010B), identified as elements (010A) and (010B), respectively. In aspects, radial marking(s) 3A (010A), 3B (010B), or both is/are identified herein as primary radial marker(s), e.g., p-radial marker(s). In certain aspect(s) of method(s) herein describing the formation of such marking system(s) described here, a portion or all of each of radial marking(s) 3A (010A), 3B (010B), or both is/are not made until a portion or all of each of radial markings 2 (e.g., 2A (008A), 2B (008B) or both) and 4 (e.g., 4A (012A), 4B (012B), or both) are established.

In certain aspects, only one of the radial marking lines 3A (010A) and 3B (010B) are present in a marking system. Typically, a marking system will comprise both radial marking 3A (010A) and radial marking 3B (010B).

According to certain aspects, radial marking(s) 3A (010A) and 3B (010B) are detectably or significantly non-vertical. In aspects, radial marker(s) 3A (010A) and 3B (010B) are positioned laterally to radial marking 1 (006) and radial marking(s) 2A (008A) and 2B (008B), and each of radial marker 3A (010A) and 3B (010B) may detectably or significantly rotate laterally toward the angle of the nasolabial fold(s) (016A) and (016B), respectively. In aspects, the lateral rotation of radial marker(s) 3A (010A) and 3B (010B) is detectably or significantly greater than that of radial marker 1 (006), radial marker 2A (008A), radial marker 2B (008B), or a combination thereof, but detectably or significantly less than that of one or more of radial markers 4A (012A), 4B (012B), 5A (014A), 5B (014B), including combination(s) of any or all thereof.

In certain aspects, radial markings 3A (010A), 3B (010B), or both comprise(s) a total length, extending from the nasal base (e.g., as indicated by the upper excision boundary (002) to the upper vermillion border (020). In aspects, the total length is represented by the combination of an excision height segment and a final lip height segment. In aspects, a first portion of radial marking(s) 3A (010A), 3B (010B), or both comprise(s) an excision height segment bound by the upper excision boundary marker (002) and the lower excision boundary marker (004). In aspects, the excision height segment identifies the portion of the upper lip along radial marking 3A (010A) or radial marking 3B (010B) to be excised during the procedure to which the marking system is applied, e.g., an upper lip lift procedure. In aspects, a second portion of radial marking(s) 3A (010A), 3B (010B), or both comprise(s) a final lip height segment bound by the lower excision boundary marker (004) and the upper vermillion border (020). In aspects, the final lip height segment identifies the portion of the upper lip along radial marking(s) 3A (010A), 3B (010B), or both which will remain post-excision.

### Radial Markings 3A/3B: Total Length

In aspects, radial marking 3A (010A), radial marking 3B (010B), or both can have any final length (“3A-T” and “3B-T”, respectively) suitable and effective for achieving targeted aesthetic effect(s).

In aspects, radial marking 3A (010A) has a total length (“3A-T”) of between about 8 mm and about 24 mm, such as, e.g., ˜8 mm-˜23 mm, ˜8 mm-˜22 mm, ˜8 mm-˜21 mm, ˜8 mm-˜20 mm, ˜8 mm-˜19 mm, ˜8 mm-˜18 mm, ˜8 mm-˜17 mm, ˜8 mm-˜16 mm, ˜8 mm-˜15 mm, ˜8 mm-˜14 mm, ˜8 mm-˜13 mm, ˜8 mm-˜12 mm, ˜8 mm-˜11 mm, or, e.g., ˜8 mm-˜10 mm.

In aspects, radial marking 3A (010A) has a total length of, e.g., ˜9 mm-˜24 mm, ˜10 mm-˜24 mm, ˜11 mm-˜24 mm, ˜12 mm-˜24 mm, ˜13 mm-˜24 mm, ˜14 mm-˜24 mm, ˜15 mm-˜24 mm, ˜16 mm-˜24 mm, ˜17 mm-˜24 mm, or ˜18 mm-˜24 mm.

In aspects, radial marking 3A (010A) has a total length of ˜8 mm-˜23 mm, ˜9 mm-˜22 mm, ˜10 mm-˜21 mm, ˜11 mm-˜20 mm, ˜11 mm-˜18 mm, ˜11 mm-˜16 mm, or, e.g., ˜11 mm-˜15 mm, such as, e.g., ˜11 mm, ˜12 mm, ˜13 mm, ˜14 mm, ˜15 mm, ˜16 mm, ˜17 mm, or, e.g., ˜18 mm.

In aspects, radial marking 3B (010B) has a total length (“3B-T”) of between about 8 mm and about 24 mm, such as, e.g., ˜8 mm-˜23 mm, ˜8 mm-˜22 mm, ˜8 mm-˜21 mm, ˜8 mm-˜20 mm, ˜8 mm-˜19 mm, ˜8 mm-˜18 mm, ˜8 mm-˜17 mm, ˜8 mm-˜16 mm, ˜8 mm-˜15 mm, ˜8 mm-˜14 mm, ˜8 mm-˜13 mm, ˜8 mm-˜12 mm, ˜8 mm-˜11 mm, or, e.g., ˜8 mm-˜10 mm.

In aspects, radial marking 3B (010B) has a total length of, e.g., ˜9 mm-˜24 mm, ˜10 mm-˜24 mm, ˜11 mm-˜24 mm, ˜12 mm-˜24 mm, ˜13 mm-˜24 mm, ˜14 mm-˜24 mm, ˜15 mm-˜24 mm, ˜16 mm-˜24 mm, ˜17 mm-˜24 mm, or ˜18 mm-˜24 mm.

In aspects, radial marking 3B (010B) has a total length of ˜8 mm-˜23 mm, ˜9 mm-˜22 mm, ˜10 mm-˜21 mm, ˜11 mm-˜20 mm, ˜11 mm-˜18 mm, ˜11 mm-˜16 mm, or, e.g., ˜11 mm-˜15 mm, such as, e.g., ˜11 mm, ˜12 mm, ˜13 mm, ˜14 mm, ˜15 mm, ˜16 mm, ˜18 mm, or, e.g., ˜18 mm.

### Radial Markings 3A/3B: Excision Height Segment Length

In aspects, radial marking 3A (010A), radial marking 3B (010B), or both can have any excision height segment length (“3A-EX” and “3B-EX”, respectively) suitable and effective for achieving targeted aesthetic effect(s).

In aspects, radial marking 3A (010A) comprises an excision height segment length (“3A-EX”) of about 0 mm to about 10 mm, such as, e.g., ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, or ˜0 mm-˜2 mm.

In aspects, radial marking 3A (010A) comprises an excision height segment length of about 1 mm-˜10 mm, 2 mm-˜10 mm, 3 mm-˜10 mm, 4 mm-˜10 mm, 5 mm-˜10 mm, 6 mm-˜10 mm, 7 mm-˜10 mm, 8 mm-˜10 mm, or 9 mm-˜10 mm.

In aspects, radial marking 3A (010A) comprises an excision height segment length of about 0 mm-˜8 mm, ˜0 mm-˜6 mm, ˜1 mm-˜8 mm, ˜1 mm-˜6 mm, ˜1 mm-˜5 mm, ˜1 mm-˜4 mm, ˜2 mm-˜6 mm, ˜2 mm-˜5 mm, ˜2 mm-˜4 mm, or ˜1 mm-˜2 mm.

In aspects, radial marking 3B (010B) comprises an excision height segment length (“3B-EX”) of about 0 mm to about 10 mm, such as, e.g., ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, or ˜0 mm-˜2 mm.

In aspects, radial marking 3B (010B) comprises an excision height segment length of about 1 mm-˜10 mm, 2 mm-˜10 mm, 3 mm-˜10 mm, 4 mm-˜10 mm, 5 mm-˜10 mm, 6 mm-˜10 mm, 7 mm-˜10 mm, 8 mm-˜10 mm, or 9 mm-˜10 mm.

In aspects, radial marking 3B (010B) comprises an excision height segment length of about 0 mm-˜8 mm, ˜0 mm-˜6 mm, ˜1 mm-˜8 mm, ˜1 mm-˜6 mm, ˜1 mm-˜5 mm, ˜1 mm-˜4 mm, ˜2 mm-˜6 mm, ˜2 mm-˜5 mm, ˜2 mm-˜4 mm, or ˜1 mm-˜2 mm.

### Radial Markings 3A/3B: Final Lip Height Segment Length

In aspects, radial marking 3A (010A), radial marking 3B (010B), or both can have any final lip height segment length (“3A-FH” and “3B-FH”, respectively) suitable and effective for achieving targeted aesthetic effect(s).

In aspects, radial marking 3A (010A) comprises a final lip height segment length (“3A-FH”) of between about 1 mm-˜19 mm, such as, e.g., ˜1 mm-˜18 mm, ˜1 mm-˜17 mm, ˜1 mm-˜16 mm, ˜1 mm-˜15 mm, ˜1 mm-˜14 mm, ˜1 mm-˜13 mm, ˜1 mm-˜12 mm, ˜1 mm-˜11 mm, ˜1 mm-˜10 mm, ˜1 mm-˜9 mm, or, e.g., ˜1 mm-˜8 mm.

In aspects, radial marking 3A (010A) comprises a final lip height segment length of ˜2 mm-˜19 mm, ˜3 mm-˜19 mm, ˜4 mm-˜19 mm, ˜5 mm-˜19 mm, ˜6 mm-˜19 mm, ˜7 mm-˜19 mm, ˜8 mm-˜19 mm, ˜9 mm-˜19 mm, ˜10 mm-˜19 mm, ˜11 mm-˜19 mm, or, e.g., ˜12 mm-˜19 mm.

In aspects, radial marking 3A (010A) comprises a final lip height segment of ˜2 mm-˜18 mm, ˜3 mm-˜17 mm, ˜4 mm-˜16 mm, ˜5 mm-˜15 mm, ˜6 mm-˜15 mm, ˜7 mm-˜15 mm, ˜8 mm-˜15 mm, ˜9 mm-˜15 mm, ˜10 mm-˜14 mm, or, e.g., ˜12 mm-˜16 mm.

In aspects, radial marking 3B (OOB) comprises a final lip height segment length (“3B-FH”) of between about 1 mm-˜19 mm, such as, e.g., ˜1 mm-˜18 mm, ˜1 mm-˜17 mm, ˜1 mm-˜16 mm, ˜1 mm-˜15 mm, ˜1 mm-˜14 mm, ˜1 mm-˜13 mm, ˜1 mm-˜12 mm, ˜1 mm-˜11 mm, ˜1 mm-˜10 mm, ˜1 mm-˜9 mm, or, e.g., ˜1 mm-˜8 mm.

In aspects, radial marking 3B (OOB) comprises a final lip height segment length of ˜2 mm-˜19 mm, ˜3 mm-˜19 mm, ˜4 mm-˜19 mm, ˜5 mm-˜19 mm, ˜6 mm-˜19 mm, ˜7 mm-˜19 mm, ˜8 mm-˜19 mm, ˜9 mm-˜19 mm, ˜10 mm-˜19 mm, ˜11 mm-˜19 mm, or, e.g., ˜12 mm-˜19 mm.

In aspects, radial marking 3B (OOB) comprises a final lip height segment of ˜2 mm-˜18 mm, ˜3 mm-˜17 mm, ˜4 mm-˜16 mm, ˜5 mm-˜15 mm, ˜6 mm-˜15 mm, ˜7 mm-˜15 mm, ˜8 mm-˜15 mm, ˜9 mm-˜15 mm, ˜10 mm-˜14 mm, or, e.g., ˜12 mm-˜16 mm.

In aspects, the excision height segment length of radial marking 3A (010A), radial marking 3B (OOB), or both radial markings 3A (010A) and 3B (OOB) is/are between about 8% and about 20% of the length of final lip height segment of the same radial marking, such as, e.g., ˜8%-˜18%, ˜8%-˜16%, ˜8%-˜14%, ˜8%-˜12%, or ˜8%-˜10% of the final lip height segment length of the same radial marking, as in, e.g., ˜10%-˜20%, ˜12%-˜20%, ˜14%-˜20%, ˜16%-˜20% or ˜18%-˜20% of the final lip height segment length, for example ˜9%-˜19%, ˜10%-˜18%, ˜11%-˜17%, ˜12%-˜16%, or, e.g., ˜13%-˜15% of the final lip height segment length of the same radial marking.

### Radial Markings 4A/4B

In aspects, marking system(s) provided herein comprise 2 radial markings, e.g., a set of radial markings, for ease of reference herein identified as “radial marking 4A” (012A) and “radial marking 4B” (012B), positioned lateral to each of radial markings 3A (010A) and 3B (010B), respectively. In aspects, radial markings 4A (012A) and 4B (012B) each (respectively) begin at the internal angle of the alar recurvature and extend radially downward. In aspects, radial markings 4A (012A) and 4B (012B) each (respectively) follow the natural curvature of the upper lip. In aspects, radial markings 4A (012A) and 4B (012B) each (respectively) follow the natural tension line(s) of the upper lip. In aspects, radial markings 4A (012A) and 4B (012B) each respectively follow the radial angulation of the pores of the upper lip. In aspects, radial markings 4A (012A) and 4B (012B) each respectively follow the natural curvature of the upper lip, following the natural tension line(s) and the radial angulation of the pores of the upper lip. FIGS. 3A and 3B illustrate an exemplary position of radial markings 4A (012A) and 4B (012B), identified as elements (012A) and (012B) in the figure, e.g., one of radial marking 4A (012A) or 4B (012B) wherein the other is the corresponding marker on the opposite side of the face. In aspects, radial marking(s) 4A (012A), 4B (012B), or both is/are identified herein as primary radial marker(s), e.g., p-radial marker(s). Radial markings 4A (012A) and 4B (012B) are illustrated in, e.g., FIG. 2A of the figures provided herewith. See also, e.g., FIGS. 3A and 3B.

In aspects, radial markings 4A (012A) and 4B (012B) each respectively mark a border between a first region of the upper lip identified as the subnasal lip as it transitions to a second region of the upper lip identified as the subfacial lip and lateral mouth. This is discussed further elsewhere herein. In aspects, radial markings 4A (012A) and 4B (012B) delineate where the effect of the lifting of an upper lip lift procedure, e.g., a deep plane upper lip lift procedure, on the lateral lip will detectably or significantly taper.

In certain aspects, only one of the radial marking lines 4A (012A) and 4B (012B) may be present in a marking system. Typically, a marking system will comprise both radial marking 4A (012A) and radial marking 4B (012B).

According to certain aspects, radial marking(s) 4A (012A) and 4B (012B) are detectably or significantly non-vertical. In aspects, radial marker(s) 4A (012A) and 4B (012B) are positioned laterally to radial markings 1, 2A (008A), 2B (008B), 3A (010A), and 3B (010B) (respectively), and each of radial marker 4A (012A) and 4B (012B) may detectably or significantly rotate laterally toward the angle of the nasolabial fold(s) (016A) and (016B), respectively. In aspects, the lateral rotation of radial marker(s) 4A (012A) and 4B (012B) is detectably or significantly greater than that of radial marker 1 (006), radial marker 2A (008A), radial marker 2B (008B), radial marker 3A (010A), radial marker 3B (010B), or a combination thereof, but detectably or significantly less than that of one or more of radial markers 5A (014A) or 5B (014B) or both.

In certain aspects, radial markings 4A (012A), 4B (012B), or both comprise(s) a total length, extending from the nasal base, e.g., from the upper excision boundary (002) to the upper vermillion border (020). In aspects, the total length is represented by the combination of an excision height segment and a final lip height segment. In aspects, a first portion of radial marking(s) 4A (012A), 4B (012B), or both comprise(s) an excision height segment bound by the upper excision boundary marker (002) and the lower excision boundary marker (004). In aspects, the excision height segment identifies the portion of the upper lip along radial marking 4A (012A) or radial marking 4B (012B) to be excised during the procedure to which the marking system is applied, e.g., an upper lip lift procedure. In aspects, a second portion of radial marking(s) 4A (012A), 4B (012B), or both comprise(s) a final lip height segment bound by the lower excision boundary marker (004) and the upper vermillion border (020). In aspects, the final lip height segment identifies the portion of the upper lip along radial marking(s) 4A (012A), 4B (012B), or both, which will remain post-excision.

### Radial Markings 4A/4B: Total Length

In aspects, radial marking 4A (012A), radial marking 4B (012B), or both can have any total length (“4A-T” and “4B-T”, respectively) suitable and effective for achieving targeted aesthetic effect(s).

In aspects, radial marking 4A (012A) has a total length (“4A-T”) of between about 10 mm and about 26 mm, such as, e.g., ˜10 mm-˜25 mm, ˜10 mm-˜24 mm, ˜10 mm-˜23 mm, ˜10 mm-˜22 mm, ˜10 mm-˜21 mm, ˜10 mm-˜20 mm, ˜10 mm-˜19 mm, ˜10 mm-˜18 mm, ˜10 mm-˜17 mm, ˜10 mm-˜16 mm, ˜10 mm-˜15 mm, ˜10 mm-˜14 mm, ˜10 mm-˜13 mm, or, e.g., ˜10 mm-˜12 mm.

In aspects, radial marking 4A (012A) has a total length of, e.g., ˜11 mm-˜26 mm, ˜12 mm-˜26 mm, ˜13 mm-˜26 mm, ˜14 mm-˜26 mm, ˜15 mm-˜26 mm, ˜16 mm-˜26 mm, ˜17 mm-˜26 mm, ˜18 mm-˜26 mm, ˜19 mm-˜26 mm, or ˜20 mm-˜26 mm.

In aspects, radial marking 4A (012A) has a total length of ˜11 mm-˜25 mm, ˜12 mm-˜24 mm, ˜13 mm-˜22 mm, ˜14 mm-˜21 mm, ˜15 mm-˜20 mm, or, e.g., ˜15 mm-˜19 mm, such as, e.g., ˜12 mm, ˜13 mm, ˜14 mm, ˜15 mm, ˜16 mm, ˜17 mm, ˜18 mm, or, e.g., ˜19 mm or ˜20 mm.

In aspects, radial marking 4B (012B) has a total length (“4B-T”) of between about 10 mm and about 26 mm, such as, e.g., ˜10 mm-˜25 mm, ˜10 mm-˜24 mm, ˜10 mm-˜23 mm, ˜10 mm-˜22 mm, ˜10 mm-˜21 mm, ˜10 mm-˜20 mm, ˜10 mm-˜19 mm, ˜10 mm-˜18 mm, ˜10 mm-˜17 mm, ˜10 mm-˜16 mm, ˜10 mm-˜15 mm, ˜10 mm-˜14 mm, ˜10 mm-˜13 mm, or, e.g., ˜10 mm-˜12 mm.

In aspects, radial marking 4B (012B) has a total length of, e.g., ˜11 mm-˜26 mm, ˜12 mm-˜26 mm, ˜13 mm-˜26 mm, ˜14 mm-˜26 mm, ˜15 mm-˜26 mm, ˜16 mm-˜26 mm, ˜17 mm-˜26 mm, ˜18 mm-˜26 mm, ˜19 mm-˜26 mm, or ˜20 mm-˜26 mm.

In aspects, radial marking 4B (012B) has a total length of ˜11 mm-˜25 mm, ˜12 mm-˜24 mm, ˜13 mm-˜22 mm, ˜14 mm-˜21 mm, ˜15 mm-˜20 mm, or, e.g., ˜15 mm-19 mm, such as, e.g., ˜12 mm, ˜13 mm, ˜14 mm, ˜15 mm, ˜16 mm, ˜17 mm, ˜18 mm, or, e.g., ˜19 mm or ˜20 mm.

### Radial Markings 4A/4B: Excision Height Segment Length

In aspects, radial marking 4A (012A), radial marking 4B (012B), or both can have any excision height segment length (“4A-EX” and “4B-EX”, respectively) suitable and effective for achieving targeted aesthetic effect(s).

In aspects, radial marking 4A (012A) comprises an excision height segment length (“4A-EX”) of about 0 mm to about 10 mm, such as, e.g., ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, or ˜0 mm-˜2 mm.

In aspects, radial marking 4A (012A) comprises an excision height segment length of about 1 mm-˜10 mm, 2 mm-˜10 mm, 3 mm-˜10 mm, 4 mm-˜10 mm, 5 mm-˜10 mm, 6 mm-˜10 mm, 7 mm-˜10 mm, 8 mm-˜10 mm, or 9 mm-˜10 mm.

In aspects, radial marking 4A (012A) comprises an excision height segment length of about 0 mm-˜8 mm, ˜0 mm-˜6 mm, ˜1 mm-˜8 mm, ˜1 mm-˜6 mm, ˜1 mm-˜5 mm, ˜1 mm-˜4 mm, ˜2 mm-˜6 mm, ˜2 mm-˜5 mm, ˜2 mm-˜4 mm, or ˜1 mm-˜2 mm.

In aspects, radial marking 4B (012B) comprises an excision height segment length (“4B-EX”) of about 0 mm to about 10 mm, such as, e.g., ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, or ˜0 mm-˜2 mm.

In aspects, radial marking 4B (012B) comprises an excision height segment length of about 1 mm-˜10 mm, 2 mm-˜10 mm, 3 mm-˜10 mm, 4 mm-˜10 mm, 5 mm-˜10 mm, 6 mm-˜10 mm, 7 mm-˜10 mm, 8 mm-˜10 mm, or 9 mm-˜10 mm.

In aspects, radial marking 4B (012B) comprises an excision height segment length of about 0 mm-˜8 mm, ˜0 mm-˜6 mm, ˜1 mm-˜8 mm, ˜1 mm-˜6 mm, ˜1 mm-˜5 mm, ˜1 mm-˜4 mm, ˜2 mm-˜6 mm, ˜2 mm-˜5 mm, ˜2 mm-˜4 mm, or ˜1 mm-˜2 mm.

### Radial Markings 4A/4B: Final Lip Height Segment Length

In aspects, radial marking 4A (012A), radial marking 4B (012B), or both can have any final lip height segment length (“4A-FH” and “4B-FH”, respectively) suitable and effective for achieving targeted aesthetic effect(s).

In aspects, radial marking 4A (012A) comprises a final lip height segment length (“4A-FH”) of between about 1 mm-˜19 mm, such as, e.g., ˜1 mm-˜18 mm, ˜1 mm-˜17 mm, ˜1 mm-˜16 mm, ˜1 mm-˜15 mm, ˜1 mm-˜14 mm, ˜1 mm-˜13 mm, ˜1 mm-˜12 mm, ˜1 mm-˜11 mm, ˜1 mm-˜10 mm, ˜1 mm-˜9 mm, or, e.g., ˜1 mm-˜8 mm.

In aspects, radial marking 4A (012A) comprises a final lip height segment length of ˜2 mm-˜19 mm, ˜3 mm-˜19 mm, ˜4 mm-˜19 mm, ˜5 mm-˜19 mm, ˜6 mm-˜19 mm, ˜7 mm-˜19 mm, ˜8 mm-˜19 mm, ˜9 mm-˜19 mm, ˜10 mm-˜19 mm, ˜11 mm-˜19 mm, or, e.g., ˜12 mm-˜19 mm.

In aspects, radial marking 4A (012A) comprises a final lip height segment of ˜2 mm-˜18 mm, ˜3 mm-˜17 mm, ˜4 mm-˜16 mm, ˜5 mm-˜15 mm, ˜6 mm-˜15 mm, ˜7 mm-˜15 mm, ˜8 mm-˜15 mm, ˜9 mm-˜15 mm, ˜10 mm-˜14 mm, or, e.g., ˜12 mm-˜18, e.g., ˜14 mm-˜18 mm.

In aspects, radial marking 4B (012B) comprises a final lip height segment length (“4B-FH”) of between about 1 mm-˜19 mm, such as, e.g., ˜1 mm-˜18 mm, ˜1 mm-˜17 mm, ˜1 mm-˜16 mm, ˜1 mm-˜15 mm, ˜1 mm-˜14 mm, ˜1 mm-˜13 mm, ˜1 mm-˜12 mm, ˜1 mm-˜11 mm, ˜1 mm-˜10 mm, ˜1 mm-˜9 mm, or, e.g., ˜1 mm-˜8 mm.

In aspects, radial marking 4B (012B) comprises a final lip height segment length of ˜2 mm-˜19 mm, ˜3 mm-˜19 mm, ˜4 mm-˜19 mm, ˜5 mm-˜19 mm, ˜6 mm-˜19 mm, ˜7 mm-˜19 mm, ˜8 mm-˜19 mm, ˜9 mm-˜19 mm, ˜10 mm-˜19 mm, ˜11 mm-˜19 mm, or, e.g., ˜12 mm-˜19 mm.

In aspects, radial marking 4B (012B) comprises a final lip height segment of ˜2 mm-˜18 mm, ˜3 mm-˜17 mm, ˜4 mm-˜16 mm, ˜5 mm-˜15 mm, ˜6 mm-˜15 mm, ˜7 mm-˜15 mm, ˜8 mm-˜15 mm, ˜9 mm-˜15 mm, ˜10 mm-˜14 mm, or, e.g., ˜12 mm-˜18, e.g., ˜14 mm-˜18 mm.

In aspects, the excision height segment length of radial marking 4A (012A), radial marking 4B (012B), or both radial markings 4A (012A) and 4B (012B) is/are between about 8% and about 20% of the length of final lip height segment of the same radial marking, such as, e.g., ˜8%-˜18%, ˜8%-˜16%, ˜8%-˜14%, ˜8%-˜12%, or ˜8%-˜10% of the final lip height segment length of the same radial marking, as in, e.g., ˜10%-˜20%, ˜12%-˜20%, ˜14%-˜20%, ˜16%-˜20% or ˜18%-˜20% of the final lip height segment length, for example ˜9%-˜19%, ˜10%-˜18%, ˜11%-˜17%, ˜12%-˜16%, or, e.g., ˜13%-˜15% of the final lip height segment length of the same radial marking.

### Radial Markings 5A/5B

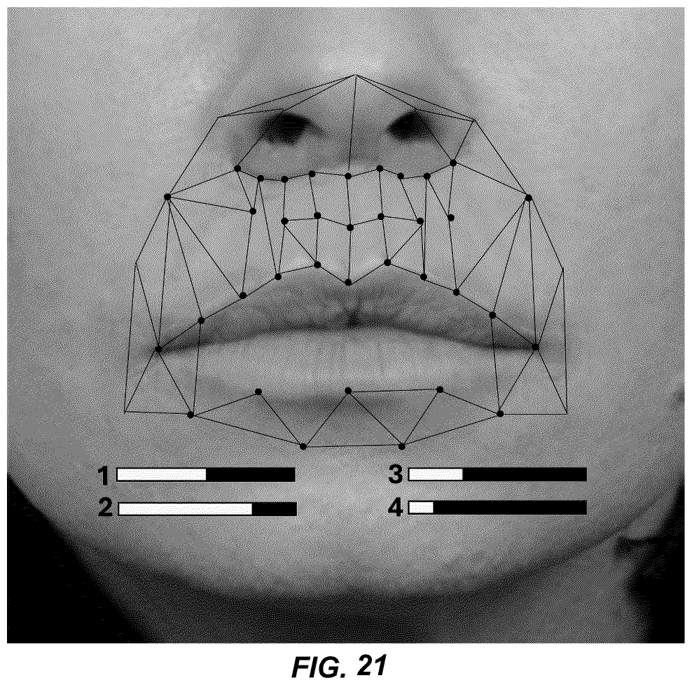

In aspects, marking system(s) provided herein comprise 2 radial markings, e.g., a set of outer radial markings (e.g., most lateral marking(s) of the radial marking(s) of marking system(s) provided herein), for ease of reference herein identified as “radial marking 5A” (014A) and “radial marking 5B” (014B), positioned lateral to each of radial markings 4A (012A) and 4B (012B), respectively. In aspects, radial markings 5A (014A) and 5B (014B) each (respectively) follow each of the relaxed skin tension lines (RSTLs). In aspects, radial markings 5A (014A) and 5B (014B), respectively, extend downward from the apex of the lateral end of the upper excision boundary marker (002) to be made at the nasal base. FIGS. 3A and 3B illustrate an exemplary position of radial markings 5A (014A) and 5B (014B). Note that the figure further identifies the nasolabial fold(s) (016A) and (016B). In aspects, radial marking(s) 5A (014A), 5B (014B), or both is/are identified herein as primary radial marker(s), e.g., p-radial marker(s). Radial markers 5A (014A) and 5B (014B) are provided in, e.g., FIG. 21 of the figure(s) provided herewith. See also, e.g., Figure(s) 3A and 3B.

In certain aspects, only one of the radial marking lines 5A (014A) and 5B (014B) may be present in a marking system. Typically, a marking system will comprise both radial marking 5A (014A) and radial marking 5B (014B).

According to certain aspects, radial marking(s) 5A (014A) and 5B (014B) are detectably or significantly non-vertical or comprise at least one portion that is detectably or significantly non-vertical. In aspects, radial marking(s) 5A (014A) and 5B (014B) comprise at least a portion of their length, which is at least generally, is at least substantially, is at least essentially, is essentially, or is vertical. In aspects, radial marking(s) 5A (014A) and 5B (014B) comprise at least a portion of their length which is at least generally, is at least substantially, is at least essentially, is essentially, or is vertical while also comprising a portion of their length which is detectably or significantly non-vertical. In aspects, radial marker(s) 5A (014A) and 5B (014B) are positioned laterally to radial markings 1, 2A (008A), 2B (008B), 3A (010A), 3B (010B), 4A (012A), and 4B (012B) (respectively), and each of radial marker 5A (014A) and 5B (014B), or at least a portion thereof, respectively, may detectably or significantly rotate laterally toward the angle of the nasolabial fold(s) (016A) and (016B), respectively. See, e.g., FIG. 2J wherein nasolabial fold(s) (016A) and (016B) are identified. See also, e.g., FIGS. 3A and 3B. In aspects, the lateral rotation of radial marker(s) 5A (014A) and 5B (014B) is detectably or significantly greater than that of radial marker 1 (006), radial marker 2A (008A), radial marker 2B (008B), radial marker 3A (010A), radial marker 3B (010B), radial marker 4A (012A), radial marker 4B (012B), or a combination thereof. In aspects, a portion of the upper lip height segment of radial marker 5A (014A), 5B (014B), or both is at least generally, is at least substantially, is at least essentially, is essentially, or is vertical. In aspects, a portion of radial segment 5A (014A), 5B (014B), or both spanning a portion of the upper lip to be excised (excised height segment) is detectably or significantly non-vertical.

In certain aspects, radial markings 5A (014A), 5B (014B), or both comprise(s) a total length, extending from the nasal base, e.g., an upper excision boundary (002) along the nasal base, to the upper vermillion border (020). In aspects, the total length is represented by the combination of an excision height segment and a final lip height segment. In aspects, a first portion of radial marking(s) 5A (014A), 5B (014B), or both comprise(s) an excision height segment bound by the upper excision boundary marker (002) and the lower excision boundary marker (004). In aspects, the excision height segment identifies the portion of the upper lip along radial marking 5A (014A) or radial marking 5B (014B) to be excised during the procedure to which the marking system is applied, e.g., an upper lip lift procedure. In aspects, a second portion of radial marking(s) 5A (014A), 5B (014B), or both comprise(s) a final lip height segment bound by the lower excision boundary marker (004) and the upper vermillion border (020). In aspects, the final lip height segment identifies the portion of the upper lip along radial marking(s) 5A (014A), 5B (014B), or both, which will remain post-excision.

According to certain aspects, at least a portion, part, or point of/along radial marking(s) 5A (014A), 5B (014B), or both, guide the performance of one or more steps of an upper lip lift procedure. In aspects, as is described elsewhere herein, at least a portion, part, or point of radial marking(s) 5A (014A), 5B (014B), or both guide the performance of one or more ancillary or concomitantly performed aesthetic modification procedure (e.g., performed in addition to an upper lip lift procedure, e.g., subnasal upper lip lift procedure), such as, e.g., a corner lip lift procedure.

### Radial Markings 5A/5B: Total Length

In aspects, radial marking 5A (014A), radial marking 5B (014B), or both can have any total length (“5A-T” and “5B-T”, respectively) suitable and effective for achieving targeted aesthetic effect(s).

In aspects, radial marking 5A (014A) has a total length (“5A-T”) of between about 10 mm and about 26 mm, such as, e.g., ˜10 mm-˜25 mm, ˜10 mm-˜24 mm, ˜10 mm-˜23 mm, ˜10 mm-˜22 mm, ˜10 mm-˜21 mm, ˜10 mm-˜20 mm, ˜10 mm-˜19 mm, ˜10 mm-˜18 mm, ˜10 mm-˜17 mm, ˜10 mm-˜16 mm, ˜10 mm-˜15 mm, ˜10 mm-˜14 mm, ˜10 mm-˜13 mm, or, e.g., ˜10 mm-˜12 mm.

In aspects, radial marking 5A (014A) has a total length of, e.g., ˜11 mm-˜26 mm, ˜12 mm-˜26 mm, ˜13 mm-˜26 mm, ˜14 mm-˜26 mm, ˜15 mm-˜26 mm, ˜16 mm-˜26 mm, ˜17 mm-˜26 mm, ˜18 mm-˜26 mm, ˜19 mm-˜26 mm, or ˜20 mm-˜26 mm.

In aspects, radial marking 5A (014A) has a total length of ˜11 mm-˜25 mm, ˜12 mm-˜24 mm, ˜13 mm-˜22 mm, ˜14 mm-˜21 mm, ˜15 mm-˜20 mm, or, e.g., ˜15 mm-˜19 mm, such as, e.g., ˜12 mm, ˜13 mm, ˜14 mm, ˜15 mm, ˜16 mm, ˜17 mm, ˜18 mm, or, e.g., ˜19 mm or ˜20 mm.

In aspects, radial marking 5B (014B) has a total length (“5B-T”) of between about 10 mm and about 26 mm, such as, e.g., ˜10 mm-˜25 mm, ˜10 mm-˜24 mm, ˜10 mm-˜23 mm, ˜10 mm-˜22 mm, ˜10 mm-˜21 mm, ˜10 mm-˜20 mm, ˜10 mm-˜19 mm, ˜10 mm-˜18 mm, ˜10 mm-˜17 mm, ˜10 mm-˜16 mm, 10 mm-˜15 mm, ˜10 mm-˜14 mm, ˜10 mm-˜13 mm, or, e.g., ˜10 mm-˜12 mm.

In aspects, radial marking 5B (014B) has a total length of, e.g., ˜11 mm-˜26 mm, ˜12 mm-˜26 mm, ˜13 mm-˜26 mm, ˜14 mm-˜26 mm, ˜15 mm-˜26 mm, ˜16 mm-˜26 mm, ˜17 mm-˜26 mm, ˜18 mm-˜26 mm, ˜19 mm-˜26 mm, or ˜20 mm-˜26 mm.

In aspects, radial marking 5B (014B) has a total length of ˜11 mm-˜25 mm, ˜12 mm-˜24 mm, ˜13 mm-˜22 mm, ˜14 mm-˜21 mm, ˜15 mm-˜20 mm, or, e.g., ˜15 mm-˜19 mm, such as, e.g., ˜12 mm, ˜13 mm, ˜14 mm, ˜15 mm, ˜16 mm, ˜17 mm, ˜18 mm, or, e.g., ˜19 mm or ˜20 mm.

### Radial Markings 5A/5B: Excision Height Segment Length

In aspects, radial marking 5A (014A), radial marking 5B (014B), or both can have any excision height segment length (“5A-EX” and “5B-EX”, respectively) suitable and effective for achieving targeted aesthetic effect(s).

In aspects, radial marking 5A (014A) comprises an excision height segment length (“5A-EX”) of about 0 mm to about 10 mm, such as, e.g., ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, or ˜0 mm-˜2 mm.

In aspects, radial marking 5A (014A) comprises an excision height segment length of about 1 mm-˜10 mm, 2 mm-˜10 mm, 3 mm-˜10 mm, 4 mm-˜10 mm, 5 mm-˜10 mm, 6 mm-˜10 mm, 7 mm-˜10 mm, 8 mm-˜10 mm, or 9 mm-˜10 mm.

In aspects, radial marking 5A (014A) comprises an excision height segment length of about 0 mm-˜8 mm, ˜0 mm-˜6 mm, ˜1 mm-˜8 mm, ˜1 mm-˜6 mm, ˜1 mm-˜5 mm, ˜1 mm-˜4 mm, ˜2 mm-˜6 mm, ˜2 mm-˜5 mm, ˜2 mm-˜4 mm, or ˜1 mm-˜2 mm.

In aspects, radial marking 5B (014B) comprises an excision height segment length (“5B-EX”) of about 0 mm to about 10 mm, such as, e.g., ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, or ˜0 mm-˜2 mm.

In aspects, radial marking 5B (014B) comprises an excision height segment length of about 1 mm-˜10 mm, 2 mm-˜10 mm, 3 mm-˜10 mm, 4 mm-˜10 mm, 5 mm-˜10 mm, 6 mm-˜10 mm, 7 mm-˜10 mm, 8 mm-˜10 mm, or 9 mm-˜10 mm.

In aspects, radial marking 5B (014B) comprises an excision height segment length of about 0 mm-˜8 mm, ˜0 mm-˜6 mm, ˜1 mm-˜8 mm, ˜1 mm-˜6 mm, ˜1 mm-˜5 mm, ˜1 mm-˜4 mm, ˜2 mm-˜6 mm, ˜2 mm-˜5 mm, ˜2 mm-˜4 mm, or ˜1 mm-˜2 mm.

### Radial Markings 5A/5B: Final Lip Height Segment Length

In aspects, radial marking 5A (014A), radial marking 5B (014B), or both can have any final lip height length (“5A-FH” and “5B-FH”, respectively) suitable and effective for achieving targeted aesthetic effect(s).

In aspects, radial marking 5A (014A) comprises a final lip height segment length (“5A-FH”) of between about 1 mm-˜22 mm, such as, e.g., ˜1 mm-˜21 mm, ˜1 mm-˜20 mm, ˜1 mm-˜19 mm, ˜1 mm-˜18 mm, ˜1 mm-˜17 mm, ˜1 mm-˜16 mm, ˜1 mm-˜15 mm, ˜1 mm-˜14 mm, ˜1 mm-˜13 mm, ˜1 mm-˜12 mm, ˜1 mm-˜11 mm, ˜1 mm-˜10 mm, ˜1 mm-˜9 mm, or, e.g., ˜1 mm-˜8 mm.

In aspects, radial marking 5A (014A) comprises a final lip height segment length of ˜2 mm-˜22 mm, ˜3 mm-˜22 mm, ˜4 mm-˜22 mm, ˜5 mm-˜22 mm, ˜6 mm-˜22 mm, ˜7 mm-˜22 mm, ˜8 mm-˜22 mm, ˜9 mm-˜22 mm, ˜10 mm-˜22 mm, ˜11 mm-˜22 mm, ˜12 mm-˜22 mm, ˜13 mm-˜22 mm, ˜14 mm-˜22 mm, ˜15 mm-˜22 mm, or, e.g., ˜16 mm-˜22 mm.

In aspects, radial marking 5A (014A) comprises a final lip height segment of ˜2 mm-˜21 mm, ˜3 mm-˜20 mm, ˜4 mm-˜19 mm, ˜5 mm-˜18 mm, ˜6 mm-˜17 mm, ˜7 mm-˜16 mm, or ˜8 mm-˜15 mm, e.g., ˜12 mm-˜20 mm, ˜12 mm-˜19 mm, or, e.g., ˜13 mm-˜19, e.g., ˜16 mm-˜18 mm.

In aspects, radial marking 5B (014B) comprises a final lip height segment length (“5B-FH”) of between about 1 mm-˜22 mm, such as, e.g., ˜1 mm-˜21 mm, ˜1 mm-˜20 mm, ˜1 mm-˜19 mm, ˜1 mm-˜18 mm, ˜1 mm-˜17 mm, ˜1 mm-˜16 mm, ˜1 mm-˜15 mm, ˜1 mm-˜14 mm, ˜1 mm-˜13 mm, ˜1 mm-˜12 mm, ˜1 mm-˜11 mm, ˜1 mm-˜10 mm, ˜1 mm-˜9 mm, or, e.g., ˜1 mm-˜8 mm.

In aspects, radial marking 5B (014B) comprises a final lip height segment length of ˜2 mm-˜22 mm, ˜3 mm-˜22 mm, ˜4 mm-˜22 mm, ˜5 mm-˜22 mm, ˜6 mm-˜22 mm, ˜7 mm-˜22 mm, ˜8 mm-˜22 mm, ˜9 mm-˜22 mm, ˜10 mm-˜22 mm, ˜11 mm-˜22 mm, ˜12 mm-˜22 mm, ˜13 mm-˜22 mm, ˜14 mm-˜22 mm, ˜15 mm-˜22 mm, or, e.g., ˜16 mm-˜22 mm.

In aspects, radial marking 5B (014B) comprises a final lip height segment of ˜2 mm-˜21 mm, ˜3 mm-˜20 mm, ˜4 mm-˜19 mm, ˜5 mm-˜18 mm, ˜6 mm-˜17 mm, ˜7 mm-˜16 mm, or ˜8 mm-˜15 mm, e.g., ˜12 mm-˜20 mm, ˜12 mm-˜19 mm, or, e.g., ˜13 mm-˜19, e.g., ˜16 mm-˜18 mm.

In aspects, the excision height segment length of radial marking 5A (014A), radial marking 5B (014B), or both radial markings 5A (014A) and 5B (014B) is/are between about 8% and about 20% of the length of final lip height segment of the same radial marking, such as, e.g., ˜8%-˜18%, ˜8%-˜16%, ˜8%-˜14%, ˜8%-˜12%, or ˜8%-˜10% of the final lip height segment length of the same radial marking, as in, e.g., ˜10%-˜20%, ˜12%-˜20%, ˜14%-˜20%, ˜16%-˜20% or ˜18%-˜20% of the final lip height segment length, for example ˜9%-˜19%, ˜10%-˜18%, ˜11%-˜17%, ˜12%-˜16%, or, e.g., ˜13%-˜15% of the final lip height segment length of the same radial marking.

### Intermediary Marking(s)

In aspects, the marking system(s) provided herein comprise one or more additional markings, referred to herein as “intermediary marking(s).” In aspects, a primary radial marking is one or more of radial markings 1, 2A (008A), 2B (008B), 3A (010A), 3B (010B), 4A (012A), 4B (012B), 5A (014A), and 5B (014B) (“p-radial marking(s)”), as described elsewhere herein, and an intermediary marking is an intermediary radial marking (“i-radial marking”) positioned in between two neighboring primary radial markings, e.g. in between radial markings 1 and 2A (008A); in between radial markings 1 and 2B (008B); in between radial markings 2A (008A) and 3A (010A); in between radial markings 2B (008B) and 3B (010B); in between radial markings 3A (010A) and 4A (012A); in between radial markings 3B (010B) and 4B (012B); in between radial markings 4A (012A) and 5A (014A); or in between radial markings 4B (012B) and 5B (014B). In aspects, i-radial marking(s) further divide the space between two p-radial markings as described above. In aspects, an i-radial marking present between two neighboring p-radial markings detectably or significantly increase(s) the level of control that the marking system(s) provides to a practitioner for attaining targeted aesthetic design feature(s) resulting from the aesthetic modification procedure(s), e.g., upper lip lift procedure(s), e.g., deep plane release upper lip lift procedure(s) comprising the application or use of marking system(s) provided herein.

In certain aspects, the space between radial markings 1 and 2A (008A), 1 and 2B (008B), 2A (008A) and 3A (010A), 2B (008B) and 3B (010B), 3A (010A) and 4A (012A), 3B (010B) and 4B (012B), 4A (012A) and 5A (014A), 4B (012B) and 5B (014B), or any two or more thereof is/are divided in half with an additional radial marking line (an i-radial marker). In aspects, the space between radial markings 1 and 2A (008A), 1 and 2B (008B), 2A (008A) and 3A (010A), 2B (008B) and 3B (010B), 3A (010A) and 4A (012A), 3B (010B) and 4B (012B), 4A (012A) and 5A (014A), 4B (012B) and 5B (014B), or any two or more thereof is/are divided in half with an additional radial marking line (an i-radial marker) and the target slope of the upper vermillion border (020) established by the marking system comprises an about 1 mm to an about 2 mm, e.g., an about 1.5 mm or, e.g., an about 1 mm, height difference between the point(s) of any new i-radial marking line along the upper vermillion border (020) and any immediately neighboring one or two points along the upper vermillion border (020) associated with p-radial markings 2A (008A), 3A (010A), 4A (012A), 5A (014A), 2B (008B), 3B (010B), 4B (012B), or 5B (014B).

In certain aspects a single i-radial marking is present (e.g., an i-radial marking on one lateral side of radial marker 1 (006) such that the i-radial marker is not present as an A-B pair (A-B pairs being described elsewhere herein.) In aspects, an i-radial marker is present as an A-B pair, with one marker of the A-B pair of i-radial markers being positioned on one lateral side of radial marker 1 (006) and another marker of the A-B pair of i-radial markers being position on another lateral side of radial marker 1 (006).

According to certain aspects, i-radial marking(s) are detectably or significantly non-vertical or comprise at least one portion which is detectably or significantly non-vertical. In aspects, i-radial marking(s) comprise at least a portion of their length, which is at least generally, is at least substantially, is at least essentially, is essentially, or is vertical. In aspects, i-radial marking(s) comprise at least a portion of their length which is at least generally, is at least substantially, is at least essentially, is essentially, or is vertical while also comprising a portion of their length which is detectably or significantly non-vertical. In aspects, i-radial marker(s) is/are positioned lateral to one or more of radial markings 1, 2A (008A), 2B (008B), 3A (010A), 3B (010B), 4A (012A), or 4B (012B), and the i-radial marker, or at least a portion thereof, may detectably or significantly rotate laterally toward the angle of the nasolabial fold(s) (016A) and (016B), respectively. In aspects, the lateral rotation of an i-radial marker(s) is detectably or significantly greater than that of one or more of radial marker 1 (006), radial marker 2A (008A), radial marker 2B (008B), radial marker 3A (010A), radial marker 3B (010B), radial marker 4A (012A), and radial marker 4B (012B). In aspects, a portion of the upper lip height segment of an i-radial marker is at least generally, is at least substantially, is at least essentially, is essentially, or is vertical. In aspects, a portion of an i-radial segment spanning a portion of the upper lip to be excised (excised height segment) is detectably or significantly non-vertical.

In certain aspects, i-radial marking(s) comprise(s) a total length, extending from the nasal base, e.g., an upper excision boundary marker (002) following the nasal base, to the upper vermillion border (020). In aspects, the total length is represented by the combination of an excision height segment and a final lip height segment. In aspects, a first portion of i-radial marking(s) comprise(s) an excision height segment bound by the upper excision boundary marker (002) and the lower excision boundary marker (004). In aspects, the excision height segment identifies the portion of the upper lip along the i-radial marking(s) to be excised during the procedure to which the marking system is applied, e.g., an upper lip lift procedure. In aspects, a second portion of i-radial marking(s) comprise(s) a final lip height segment bound by the lower excision boundary marker (004) and the upper vermillion border (020). In aspects, the final lip height segment identifies the portion of the upper lip along the i-radial marking(s) which will remain post-excision.

In aspects, i-radial marking(s) can have any total length (“iRM-T”) suitable and effective for achieving targeted aesthetic effect(s). In aspects, a total length can be any total length presented for any one or more p-radial marker(s) provided herein or falling within range(s) of total length(s) of p-radial marker(s) presented herein.

In aspects, i-radial marking(s) can have any excision height segment length (“iRM-EX”) suitable and effective for achieving targeted aesthetic effect(s). In aspects, an excision height segment length can be any excision height segment length presented for any one or more p-radial marker(s) provided herein or falling within range(s) of excision height segment length(s) of p-radial marker(s) presented herein.

In aspects, i-radial marking(s) can have any final upper lip height length (“iRM-FH”) suitable and effective for achieving targeted aesthetic effect(s). In aspects, a final upper lip height length can be any final upper lip height length presented for any one or more p-radial marker(s) provided herein or falling within range(s) of final upper lip height length(s) of p-radial marker(s) presented herein.

In certain aspects, i-radial marking(s) can have a total length which falls between the total length(s) of each neighboring p-radial marking(s). In certain aspects, i-radial marking(s) can have an excision height segment length which falls between the excision height segment length(s) of each neighboring p-radial marking(s). In certain aspects, i-radial marking(s) can have a final upper lip height length which falls between the final upper lip height length(s) of each neighboring p-radial marking(s).

### Subnasal Lip/Subfacial Lip & Lateral Mouth Demarcation

In aspects, marking system(s) provided herein comprise at least one, such as, e.g., at least 2, marking(s)/marker(s) which delineate at least a first defined region of the upper lip from at least a second defined region of the upper lip.

In aspects, marking system(s) provided herein comprise at least one, such as, e.g., at least 2, marking(s)/marker(s) which mark a boundary of a first defined region of the upper lip identifiable as the “subnasal lip.” In aspects, the marking system(s) provided herein comprise at least one, such as, e.g., at least 2, marking(s)/marker(s) which mark a boundary of a second defined region of the upper lip identifiable as the “subfacial lip and lateral mouth.” In aspects, marking system(s) provided herein comprise at least one, such as, e.g., at least 2, marking(s)/marker(s) which mark a boundary between a first defined region of the upper lip, identifiable as the subnasal lip and a second defined region of the upper lip identifiable as the subfacial lip and lateral mouth. In aspects, the marker(s) delineate the change in physical characteristic(s) of the upper lip which characterize the two regions, e.g., the subnasal lip and the subfacial lip and lateral mouth. In aspects, the marking system(s) provided herein comprise 2 marker(s) which delineate the two regions, wherein the first marker is positioned lateral to the mouth on one side, the second marker is positioned lateral to the mouth on the opposing side. In aspects, the marking system(s) provided herein comprise 2 marker(s) which delineate the two regions, wherein the first marker is positioned lateral to the mouth on one side, the second marker is positioned lateral to the mouth on the opposing side, and the region in between the two markers is identified as the subnasal lip. In aspects, marking system(s) provided herein comprise 2 marker(s) which delineate the two regions, wherein the first marker is positioned lateral to the mouth on one side, the second marker is positioned lateral to the mouth on the opposing side, and the region(s) lateral to each marker on both sides of the mouth is/are identified as the subfacial lip and lateral mouth.

In aspects, the at least first and second defined regions described above, e.g., the subnasal lip and the subfacial lip and lateral mouth of the upper lip, indicated by marking system(s) detectably or significantly physically differ from one another in one or more structurally identifiable ways. In aspects, the at least first and second defined regions described above, e.g., the subnasal lip and the subfacial lip and lateral mouth of the upper lip, indicated by marking system(s) detectably or significantly physically differ from one another in one or more structurally identifiable ways which result in each of the regions differing from the other in their sensitivity to the effect(s) of aesthetic modification procedure(s), e.g., their sensitivity to the lift effect of upper lip lift procedure(s).

In certain aspects, a first defined region of the upper lip indicated or demarcated by the marking system, e.g., a region identified as the subnasal lip, is more densely attached to the pyriform than the at least second defined region of the upper lip indicated or demarcated by the marking system, e.g., a region identified as the subfacial lip and lateral mouth. In aspects, the first defined region, e.g., the subnasal lip, is supported by the front teeth.

In aspects, a second defined region of the upper lip indicated or demarcated by the marking system, e.g., a region identified as the subfacial lip and lateral mouth, hangs from the anterior cheek and face.

According to certain aspects, marking system(s) provided herein comprise at least one, such as, e.g., at least 2, marking(s)/marker(s) which mark the boundary between first and second defined regions of the upper lip, e.g., the subnasal lip and the subfacial lip and lateral mouth. In aspects, the marking system(s) provide 2 such markers. In aspects, the markers are bilateral radial markings 4A (012A) and 4B (012B), wherein each of markings 4A (012A) and 4B (012B) respectively mark the border of the first region of the upper lip identified as the subnasal lip as it transitions to the second region of the upper lip identified as the subfacial lip and lateral mouth. In aspects, the subnasal lip region and the subfacial lip and lateral mouth regions are demarcated by the 2 bilateral radial markings 4A (012A) and 4B (012B), the subnasal lip region being located between radial markings 4A (012A) and 4B (012B) and the subfacial lip and lateral mouth regions being located lateral to each of 4A (012A) and 4B (012B), respectively.

In aspects, as stated above, differ from one another in their sensitivity to the effects of one or more element(s) of an aesthetic modification procedure, such as, e.g., the lift effect(s) of an upper lip lift procedure. In aspects, the marking(s), e.g., radial markings 4A (012A) and 4B (012B), identify and therefore determine the lateral extent of the lift effect on the upper lip caused by the upper lip lift. That is, in aspects, an amount of lift performed within a first defined region, e.g., subnasal lip, may yield a greater degree of lift than the same amount of lift performed within a second defined region. Such a difference, in aspects, results from the structural difference(s) between the two regions.

In certain aspects, the demarcation of the two defined regions described in this section is relevant to both a primary aesthetic modification procedure, e.g., an upper lip lift, and one or more ancillary or concomitant procedure(s), such as a corner lip lift procedure. In aspects, two markers, e.g., bilateral radial markings 4A (012A) and 4B (012B), demarcate a region (e.g., subnasal lip) lateral to which the corner lift procedure is initiated (as applicable to the side(s) of the subject's mouth undergoing the corner lip procedure). In aspects, two markers, e.g., bilateral radial markings 4A (012A) and 4B (012B), demarcate a region (e.g., subnasal lip) medial to which the corner lift procedure is initiated (as applicable to the side(s) of the subject's mouth undergoing the corner lip procedure) to shorten the final lip height (segment of each marking between the second lower incision point/lower excision boundary along the lower excision boundary marker (004) and a third point located along the upper vermillion border (020)) of radial marking(s) 4A (012A) and 4B (012B) (as applicable to the side(s) of the subject's mouth undergoing the corner lip procedure).

### Type I & Type II Marking(s)

In aspects, marking system(s) provided by the disclosure further comprise at least one marking not characterizable as a radial marking (e.g., as a radial marking as described herein) or an excision boundary marker, such as those described herein. In aspects, marking system(s) provided by the disclosure comprise marking(s) suitable for use in an aesthetic modification procedure, such as an upper lip lift procedure, or a procedure performed separately from or concomitant to an upper lip lift procedure. In aspects, the marking system(s) provided by the disclosure comprise marking(s) suitable for use in an ancillary procedure. In aspects, marking system(s) provided by the disclosure comprise marking(s) suitable for use in an ancillary procedure performed concomitant to an upper lip lift procedure, such as, e.g., a deep plane release upper lip lift, wherein the ancillary procedure is one or more of a corner lift, a nasal base suspension, a muscle suspension, or a combination of any or all thereof.

In aspects, the marking system(s) provided by the disclosure comprise one or more marking(s) for facilitating a corner lip lift procedure. In aspects, a corner lip lift procedure marking comprises an at least substantially elliptical or fusiform shape. In aspects, a corner lip lift procedure marking ends laterally at the oral commissure. In aspects, a corner lip lift procedure marking comprises an at least substantially elliptical or fusiform shape and ends laterally at the oral commissure, referred to herein as a “Type I” marking ((042A) or (042B)). In aspects, a Type I marking ((042A) or (042B)) is a guide in a corner lip lift procedure directed to the exposure and lifting of the lateral vermillion. In aspects, a Type I marking ((042A) or (042B)) is a guide in a corner lip lift procedure (e.g., a deep plane corner lip lift procedure) directed to revealing an increased amount of lateral vermillion.

In aspects, the marking system(s) provided by the disclosure comprise a corner lip lift procedure marking comprising an enclosed shape. In aspects, a corner lip lift procedure marking extends past the commissure. In aspects, a corner lip lift procedure marking comprises a detectably or significantly upward inflection. In certain aspects, a corner lip lift procedure marking comprises an enclosed shape which extends past the commissure and comprises a detectably or significantly upward inflection, referred to herein as a “Type II” marking ((044A) or (044B)). In aspects, a Type II marking ((044A) or (044B)) is a guide in a corner lip lift procedure (e.g., a deep plane corner lip lift procedure) directed to lengthening the vermillion, treating angular depression around the mouth, lifting the commissure, or neutralizing down-pointing rhytids extending inferior to the chelion, or, in aspects, the procedure is directed to combination(s) of lengthening the vermillion, treating angular depression around the mouth, lifting the commissure, and neutralizing down-pointing rhytids extending inferior to the chelion.

FIG. 5 herein provides an exemplary embodiment of a Type I marking. As provided, a Type I marking is shown on both sides of the mouth (labeled (042A) and (042B), respectively). FIG. 6 herein provides an exemplary embodiment of a Type II marking. As provided, a Type II marking is shown on both sides of the mouth (labeled (044A) and (044B), respectively). The diagonal lines in each of FIGS. 5 and 6 mark the transition from the subnasal to the subfacial lip, described elsewhere herein, wherein the subfacial lip lacks significant support and accordingly is where the vermillion roll tapers off (described as radial markers (012A) and (012B), respectively, elsewhere herein). In FIG. 5, a Type I marker (042A) or (042B) is provided utilizing an elliptical excision to replace philtral skin with red vermillion. In FIG. 6, a Type II marker (044A) or (044B) is provided wherein there is an inflection past the commissure which affects a change to the lower lateral lip angulation and insertion, and the Type II marker (044A) or (044B) is used to guide the lengthening and appearance of the upper lip. FIG. 6 further illustrates the horizontal markers H-A (046A) and H-B (046B), such markers being elements of Type II markers, and which are described elsewhere herein.

In certain aspects, marking systems do not comprise a Type I marker ((042A) or (042B)), a Type II marker ((044A) or (044B)), or lack both a Type I marker ((042A) or (042B)) and a Type II marker ((044A) or (044B)).

### Horizontal Reference Point Markings

In some aspects, marking system(s) provided herein comprise marker(s) indicating a reference point for use in performing a procedure ancillary to an upper lip lift procedure, such as, e.g., a concomitantly performed corner lip lift procedure. In aspects, marking system(s) comprise marker(s) indicating a reference point for use in performing a corner lift incision, wherein the position of the indicator is established based on the positioning of one or more radial marking(s). In aspects, the position of such indicator(s) is/are established based upon the position of point(s) along the upper vermillion border (020) of two or more radial markings (s). In aspects, the position of such indicators is based upon the point(s) on the upper vermillion border (020) of radial marking line(s) 4A (012A), 4B (012B), or both, as applicable to the side(s) of the subject's mouth undergoing the corner lip procedure.

In aspects, the point(s) on the upper vermillion border (020) of radial marking lines 4A (012A), 4B (012B), or both, as applicable to the side(s) of the subject's mouth undergoing the corner lip procedure, is/are used as a starting point for an at least generally, at least substantially, at least essentially, essentially, or completely horizontal marking line, e.g., an at least generally straight, at least substantially straight, at least essentially straight, essentially straight, or straight horizontal marking line drawn laterally from such point(s). In aspects, the marking(s) establish a reference point, e.g., an upper reference point e.g., indicating the “upper limb” of a corner lip incision, for use in performing a corner lift incision. These marking(s) are referred to herein as “horizontal marking A” (“H-A”) (046A) and “horizontal marking B” (“H-B”) (046B), respectively. In aspects, such marking(s) are a component of a Type II marking (044A) or (044B), respectively, described above. In aspects, additional radial marking(s) can assist in the performance of a corner lip lift procedure. In aspects, radial marker(s) 5A (014A), 5B (014B), or both, can be used to determine the height of an excision for which a Type II marker (044A) or (044B) is a guide (e.g., a Type II lift) by maintaining the final lip height segment length of radial markers 5A (014A), 5B (014B), or both at least generally the same, at least substantially the same, at least essentially the same, essentially the same, or the same as the final lip height segment(s) of radial markers 4A (012A), 4B (012B), or both. In certain aspects, where marking H-A (046A), marking H-B (046B), or both indicate the upper limb of a corner lift incision, the lower limb follows the red/white border of the vermillion. In aspects, horizontal marking(s) H-A (046A), H-B (046B), or both end before reaching the nasolabial fold line(s) (016A) and (016B), respectively, of the relaxed skin tension line globe. H-A (046A) and H-B (046B) are labeled in FIG. 6 provided herein.

### Extension Markings

In aspects, marking system(s) provided herein comprise one or more marker(s) indicating a reference point(s) for use in performing procedure(s) ancillary to an upper lip lift procedure, such as, e.g., a concomitantly performed corner lip lift procedure. In aspects, marking system(s) comprise one or more marker(s) indicating a reference point for use in performing a corner lift incision, wherein the position of the indicator is established based on the positioning of one or more radial marking(s). In aspects, marking system(s) comprise one or more marker(s) indicating reference point(s) for use in performing a corner lift procedure, wherein the marker(s) is/are (an) at least generally horizontal, at least substantially horizontal, at least essentially horizontal, essentially horizontal, or horizontal lateral line extension(s) beginning at the point where radial marker(s) 4A (012A), 4B (012B), or both, respectively, intersect with the lower excision boundary marker (004) (at a lower incision point marker of radial marker(s) 4A (012A), 4B (012B), or both, respectively) and are directed laterally therefrom. In aspects, such markers are referred to herein as “lateral extension marker A” (“LE-A”) (018A) and “lateral extension marker B” (“LE-B”) (018B). FIGS. 2H and 3A (010A) illustrate exemplary embodiment(s) of marker(s) LE-A (018A) and LE-B (018B). See, e.g., FIG. 2H wherein LE-A (018A) and LE-B (018B) are identified. See also, e.g., Figure(s) 3A and 3B.

### Marking System and Lip Shape, Slope

In aspects, length(s) of the segment(s) of each of one or more radial marking(s) representing the final height of the upper lip at each respective radial marking, the length of segment(s) of each of one or more radial marking(s) representing the excision height at each respective radial marking, or a combination thereof, establish(es) the lip shape resulting from an upper lip lift procedure (e.g., with or without concomitant corner lip lift(s)) comprising the application of the marking system(s) comprising such radial marking(s) provided herein. In aspects, the disclosure provides marking system(s) for guiding aesthetic modification procedure(s) wherein the marking system is established according to target aesthetic effect(s) resulting from the procedure(s) and wherein the application and incorporation of the marking system(s) in the procedure(s) predictably yield(s) the target aesthetic effect(s).

Herein, use of the word “predictably”, for example as used in the phrase above, “ . . . wherein the application and incorporation of the marking system(s) in the procedure(s) predictably yield(s) the target aesthetic effect(s),” should be interpreted to mean that procedure(s) yield the target effect(s) which marking system(s) is/are each designed to achieve at least about 50%, ≥˜55%, ≥˜60%, ≥˜65%, ≥˜70%, ≥˜75%, ≥˜80%, ≥˜85%, ≥˜90%, ≥˜95%, or, ˜100% of the time. In aspects, “predictably” as used here means that, e.g., about 55 out of every 100 ( 55/100) procedures comprising use of the marking system(s), e.g., about 55/100, ˜ 60/100, ˜ 65/100, ˜ 70/100, ˜ 75/100, ˜ 80/100, ˜ 85/100, ˜ 90/100, ˜ 95/100, or, e.g., 100/100 procedures comprising use of the marking system(s) achieve the target aesthetic effect(s) which the marking system(s) are designed to achieve without requiring a secondary procedure to amend results, e.g., without requiring revision.

In aspects, a target aesthetic shape of the upper lip, one or more curves of the upper vermillion, or both, establish or otherwise define, by the positioning of the lower excision boundary marker (004), a distance between the lower excision boundary marker (004) and the point located on the upper vermillion border (020) along of the radial marker(s), e.g., each of the at least 7 radial markings. In aspects, the distance between the lower excision boundary marker (004) and the upper vermillion border (020), e.g., the established distance between second and third points along each of the radial marking(s) (e.g., each of the at least 7 radial marking(s)) define a final lip height of the upper lip along each of the marking(s).

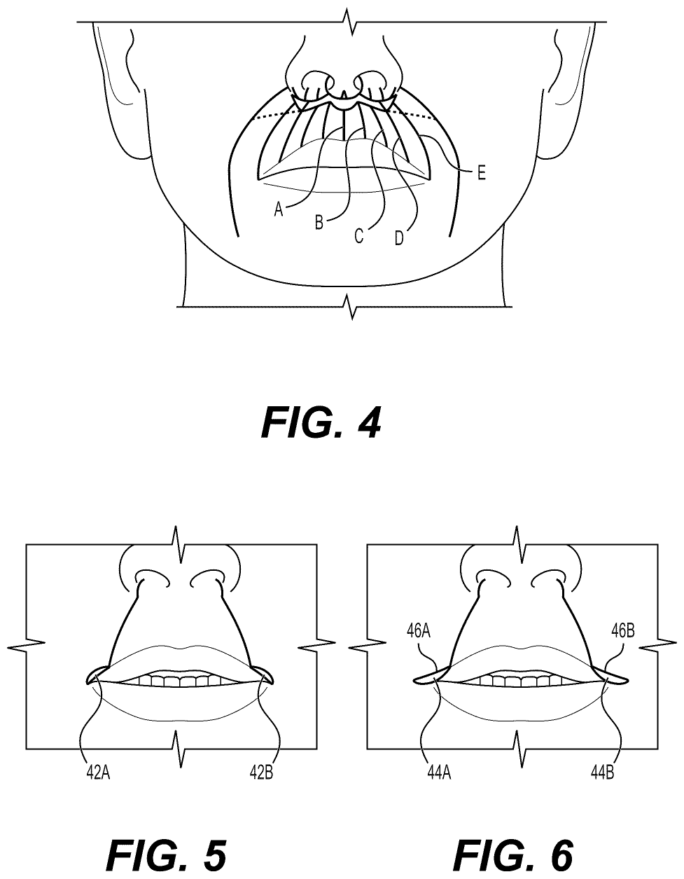

According to certain aspects, a final upper lip height indicated by any one or more of the radial marking(s) is at least about 8 mm, such as, e.g., ≥˜9 mm, ≥˜10 mm, ≥˜11 mm, ≥˜12 mm, ≥˜13 mm, ≥˜14 mm, ≥˜15 mm, or ≥˜16 mm. In aspects, a final upper lip height indicated by any one or more of the radial marking(s) is at least about 11 mm. FIG. 4 provided herein exemplifies measurements of the final lip height segment of each of radial markings 1 and radial markings 2, 3, 4, and 5 belonging to a single group (e.g., on one side of radial marking 1 (006) such that they are identifiable as a group of markers; e.g., Group A markers or Group B markers (such as radial marking(s) 2B (008B), 3B (010B), 4B (012B), and 5B (014B).) Therein, exemplified final upper lip heights are, e.g., length A for radial marker 1 (006), e.g., about 12 mm, length B for radial marker 2 (e.g., 2B (008B)), also, e.g., about 12 mm, length C for radial marker 3 (e.g., 3B (010B)), e.g., about 14 mm, length D for radial marker 4 (e.g., 4B (012B)), e.g., about 16 mm, and length E for radial marker 5 (e.g., 5B (014B)), e.g., about 17 mm.

In certain aspects, final lip height indicated by any two or more of the radial marking(s), e.g., the segment(s) of two or more radial marker(s) representing final lip height, can vary across radial markers by between about 0% and about 70%, such as, e.g., by ˜0%-˜65%, ˜0%-˜60%, ˜0%-˜55%, ˜0%-˜50%, ˜0%-˜45%, ˜0%-˜40%, ˜0%-˜35%, ˜0%-˜30%, ˜0%-˜25%, ˜0%-˜20%, ˜0%-˜15%, ˜0%-˜10%, or ˜0%-˜5%.

In certain aspects, final lip height indicated by any two or more of the radial marking(s), e.g., the segment(s) of two or more radial marker(s) representing final lip height, can vary across radial markers by between about 5% and about 70%, such as, e.g., ˜10%-˜70%, ˜15%-˜70%, ˜20%-˜70%, ˜25%-˜70%, ˜30%-˜70%, ˜35%-˜70%, ˜40%-˜70%, ˜45%-˜70%, ˜50%-˜70%, ˜55%-˜70%, ˜60%-˜70%, or ˜65%-˜70%.

In certain aspects, final lip height indicated by any two or more radial marking(s), e.g., the segment(s) of two or more radial marker(s) representing final lip height can vary across radial markers by about 0%, by ˜5%, by ˜10%, by ˜15%, by ˜20%, by ˜25%, by ˜30%, by ˜35%, by ˜40%, by ˜45%, by ˜50%, by ˜55%, by ˜60%, by ˜65%, or, e.g., by ˜70% or more. In aspects, marking system(s) comprise two or more radial marking(s) each having a segment representing the final lip height wherein the longest final lip height segment of the markings is no more than about 0%, 5%, 15%, 20%, 25%, 30%, 35%, 40%, 45%, 50%, 55%, 60%, 65%, or 70% longer than the shortest lip final lip height segment of the markings.

In aspects, the final upper lip height segment(s) is/are established, by the positioning of one or more marker(s) of marking system(s), to attain one or more target aesthetic effects, such as, e.g., overall facial balance, incisor display, final lip height, etc. In aspects, the final lip height segment of radial marking(s) for one or more radial marking(s) is established while the lip is in repose and is not detectably or significantly stretched. In aspects, the final lip height segment in/of radial marking(s) 2A (008A), 2B (008B), 3A (010A), 3B (010B), 4A (012A), 4B (012B), or combination(s) thereof is/are placed while the lip is in repose and is not held under detectable or significant tension. In aspects, the final lip height segment of radial marking(s), 2A (008A), 2B (008B), 3A (010A), 3B (010B), 4A (012A), 4B (012B), or combination(s) thereof is/are established while the lip is in repose and not detectably or significantly stretched while the final height segment of radial marking 1 (006) is established while the lip is held under gentle tension.

According to certain aspects, target aesthetic result(s) include a target shape of the upper vermillion. According to certain aspects, target aesthetic result(s) include a target slope of the upper vermillion border. In aspects, length(s) of segments(s) of one or more radial marking(s) representing the final height of the upper lip, the length(s) of segment(s) representing excision height, or a combination thereof establishes the slope of the upper vermillion (herein, “upper vermillion,” unless otherwise indicated or contradicted, typically can be read as “upper vermillion border,” and vice-versa) resulting from the aesthetic modification procedure(s)/method(s) incorporating the use of radial markings(s), such as method(s) of performing subnasal upper lip lift(s), e.g., deep plane release upper lip lift procedure(s) (e.g., with or without concomitant corner lip lift(s)). In aspects, marking system(s) and associated mathematical calculation(s) and assessment(s) disclosed herein establish target upper vermillion border slope(s) resulting from the performance of upper lip lift procedures(s) comprising use of the marking system(s) and associated mathematical calculation(s) and assessment(s).

In certain aspects, the length(s) of segment(s) of one or more radial marking(s) representing the final height of the upper lip, the length(s) of the segment(s) of one or more radial marking(s) representing the excision height, or a combination thereof, along each radial marking establish(es) the slope of the upper vermillion border (020) resulting from the application of/incorporation of the radial marking(s) in method(s) of performing subnasal lip lift procedure(s), wherein the slope of the upper vermillion border (020) is the vertical descent of the upper vermillion border (020) from the Cupid's peak following laterally toward the oral commissure when viewing the human subject face-on. FIG. 2N shows Cupid's peak on the left labeled as CP-A, and on the right labeled as CP-B.

In aspects, a target upper vermillion border (020) slope is established by the positioning of an upper excision boundary marker (002), lower excision boundary marker (004), and radial marking(s) of marking system(s) provided herein. In aspects, a target upper vermillion border (020) slope is established by at least a single central marking (e.g., radial marking 1 (006)), 3 pairs of radial markings (e.g., radial markers 2A (008A) and 2B (008B), radial markers 3A (010A) and 3B (010B), and radial markers 4A (012A) and 4B (012B)). In aspects, a target upper vermillion border (020) slope is established by the use of a marking system comprising lateral reminder marking(s) used for placement of a lateral deep suture.

According to aspects, a target vermillion slope can be any slope identified as aesthetically appropriate to the subject's accompanying facial features. In certain aspects, a target vermillion slope is characterized as a height difference along the upper vermillion border (020) between points associated with adjacent radial marker(s) of between about 1 mm and about 5 mm, e.g., ˜1 mm-˜4 mm, ˜1 mm-˜3 mm, or ˜1 mm-˜4 mm, such as ˜2 mm-˜5 mm, ˜3 mm-˜5 mm, or ˜4 mm-˜5 mm, as in, for example, ˜2 mm-˜4 mm, or, e.g., ˜2 mm. That is, in aspects, a target vermillion slope is characterized as points along the upper vermillion border (020) between, e.g., radial markers 2A (008A) and 3A (010A); 3A (010A) and 4A (012A); 2B (008B) and 3B (010B); 3B (010B) and 4B (012B); and, e.g., in aspects further 4A (012A) and 5A (014A) and between 4B (012B) and 5B (014B) having a difference in height of between about 1 mm and about 5 mm, e.g., ˜1 mm-˜4 mm, ˜1 mm-˜3 mm, or ˜1 mm-˜4 mm, such as ˜2 mm-˜5 mm, ˜3 mm-˜5 mm, or ˜4 mm-˜5 mm, as in, for example, ˜2 mm-˜4 mm, or, e.g., ˜2 mm. FIGS. 3A and 3B provided herein exemplify such a slope. Specifically, in aspects, a height difference of, for example, about 1 mm to about 3 mm, e.g., about 2 mm, between the points along the upper vermillion border (020) of two neighboring radial markers within radial markers 2A (008A), 3A (010A), 4A (012A), 2B (008B), 3B (010B), and 4B (012B) provides a post-excision slope of the upper vermillion border (020). FIGS. 3A and 3B, provided herein, exemplify this concept. FIG. 4 illustrates a final lip height segment of radial marker 2B (008B) of 12 mm, a final lip height segment of radial marker 3B (010B) of 14 mm, and a final lip height segment of radial marker 4B (012B) of 16 mm provides such a target 2 mm height difference and yields such an aesthetically pleasing, target vermillion slope.

See, for example, FIG. 2N.

The area defined between an upper excision boundary marker (002) and a lower excision boundary marker (004) establishes the area to be excised (001), e.g., a bullhorn-shaped area to be excised.

In aspects, the marking system(s) provided herein support the establishment of the height to be excised at a location on one side of the central marking 1, which is different from the height to be excised at the corresponding location on the opposite side of the central marking 1. That is, corresponding location(s) of an area to be excised (001), on either side of central marking 1, as created by marking system(s) provided herein, may not comprise uniform excision height(s). More specifically, the disclosure herein provides for targeted upper vermillion slope, tooth show, or other target feature(s) described herein which is/are established by the identification of an area to be excised (001), wherein the area to be excised comprises one or more locations identified by one or more of radial marker(s) excision segment(s) 2A-EX, 3A-EX, 4A-EX, or 5A-EX, which is/are different from one or more of corresponding marker(s) excision segment(s) 2B-EX, 3B-EX, 4B-EX, or 5B-EX, respectively.

Marking system(s) provided herein support the establishment of the area to be excised (001). As described elsewhere herein, excision height segments (“-EX” segments) of radial marker(s) are established by considering the target upper vermillion slope achieved when the height difference at the upper vermillion border of adjacent radial marker(s) (points along the upper vermillion border where two adjacent radial marker(s) meet the upper vermillion border) have a height difference of between about 1 mm and about 3 mm, such as, e.g., about 2 mm.

In aspects, non-uniformity in excision height across corresponding location(s) (e.g., right and left sides of center) of an area to be excised (001) as described here provided by the system(s) and method(s) herein provides for an improvement in consistency and predictability of aesthetic outcome(s) of procedures utilizing marking system(s) described herein.

FIG. 2N illustrates an area to be excised (001). Such an area to be excised is expected to result in specific slope(s) as identified therein: slope (022A) between radial marker 1 and radial marker 2A; slope (022B) between radial marker 1 and radial marker 2B; slope (0224A) between radial marker 2A and radial marker 3A; slope (0224B) between radial marker 3B and radial marker 3B; slope (0226A) between radial marker 3A and radial marker 4A; slope (0226B) between radial marker 3B and radial marker 4B; slope (0228A) between radial marker 4A and radial marker 5A; and slope (0228B) between radial marker (0228B) between radial marker 4B and radial marker 5B.

In aspects, slopes (022A) and (022B) resulting from the marking system(s) provided herein, or resulting from method(s) comprising use of the marking system(s) provided herein, are at least generally, at least substantially, at least essentially, are essentially, or are the same.

In aspects, slopes (024A) and (024B) resulting from the marking system(s) provided herein, or resulting from method(s) comprising use of the marking system(s) provided herein, are at least generally, at least substantially, at least essentially, are essentially, or are the same.

In aspects, slopes (026A) and (026B) resulting from the marking system(s) provided herein, or resulting from method(s) comprising use of the marking system(s) provided herein, are at least generally, at least substantially, at least essentially, are essentially, or are the same.

In aspects, slopes (028A) and (028B) resulting from the marking system(s) provided herein, or resulting from method(s) comprising use of the marking system(s) provided herein, are at least generally, at least substantially, at least essentially, are essentially, or are the same.

In aspects, slopes (022A) and (024A) resulting from the marking system(s) provided herein, or resulting from method(s) comprising use of the marking system(s) provided herein, are at least generally, at least substantially, at least essentially, are essentially, or are the same. In aspects, slopes (022B) and (024B) resulting from the marking system(s) provided herein, or resulting from method(s) comprising use of the marking system(s) provided herein, are at least generally, at least substantially, at least essentially, are essentially, or are the same.

In aspects, slopes (024A) and (026A) resulting from the marking system(s) provided herein, or resulting from method(s) comprising use of the marking system(s) provided herein, are at least generally, at least substantially, at least essentially, are essentially, or are the same. In aspects, slopes (024B) and (026B) resulting from the marking system(s) provided herein, or resulting from method(s) comprising use of the marking system(s) provided herein, are at least generally, at least substantially, at least essentially, are essentially, or are the same.

In aspects, slopes (026A) and (028A) resulting from the marking system(s) provided herein, or resulting from method(s) comprising use of the marking system(s) provided herein, are at least generally, at least substantially, at least essentially, are essentially, or are the same. In aspects, slopes (026B) and (028B) resulting from the marking system(s) provided herein, or resulting from method(s) comprising use of the marking system(s) provided herein, are at least generally, at least substantially, at least essentially, are essentially, or are the same.

In aspects, any 2 or more, 3 or more, 4 or more, 5 or more, 6 or more, 7 or more, or e.g., all 8 of slopes (022A), (022B), (024A), (024B), (026A), (026B), (028A), and (028B) are at least generally, at least substantially, at least essentially, are essentially, or are the same. In aspects, such slope(s) can provide a rise (or drop, as applicable) between adjacent radial marker/marking points along the upper vermillion border (e.g., between adjacent points where each of radial markers 1, 2A, 2B, 3A, 3B, 4A, 4B, 5A, and 5B meet the upper vermillion border) of between about 1 mm and about 3 mm, such as, e.g., about 1.1 mm, ˜1.2 mm, ˜1.3 mm, ˜1.4 mm, ˜1.5 mm, ˜1.6 mm, ˜1.7 mm, ˜1.8 mm, ˜1.9 mm, ˜2 mm, ˜2.1 mm, ˜2.2 mm, ˜2.3 mm, ˜2.4 mm, ˜2.5 mm, ˜2.6 mm, ˜2.7 mm, ˜2.8 mm, ˜2.9 mm, or, e.g., ˜3 mm, such as for example ˜1 mm-˜2.8 mm, ˜1 mm-˜2.6 mm, ˜1 mm-˜2.4 mm, ˜1 mm-˜2.2 mm, ˜1 mm-˜2 mm, or ˜1.2 mm-˜3 mm, ˜1.4 mm-˜3 mm, ˜1.6 mm-˜3 mm, ˜1.8 mm-˜3 mm, or ˜2 mm-˜3 mm, such as, for example, ˜1.2 mm-˜2.8 mm, ˜1.4 mm-˜2.6 mm, ˜1.6 mm-˜2.4 mm, ˜1.8 mm-˜2.2 mm, or, e.g., about 2 mm.

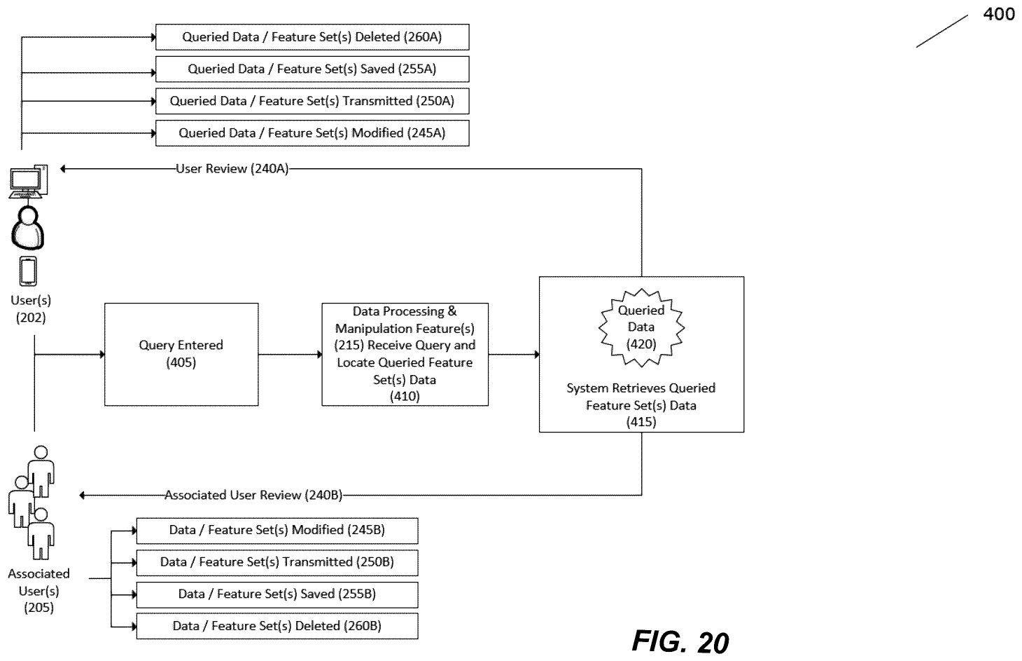

See, for example, FIG. 2O. FIG. 20 illustrates height differences (vertical distance between) between radial marking points along the upper vermillion border.

In FIG. 2O, dotted line I crosses the lower point of/lower trough of Cupid's bow (at the lower point of the philtral dimple), where radial marker 1 meets the upper vermillion border. Dotted lines IIA and IIB each extend laterally from the peak of Cupid's bow, where each of radial markers 2A and 2B (respectively) meet the upper vermillion border on each side of the upper lip. Height difference (vertical distance between) (032A) represents the height difference between (vertical distance between) where radial marker 1 and radial marker 2A meet the upper vermillion border; e.g., the height difference between (vertical distance between) dotted line I and dotted line IIA. Height (032B) represents the height difference between (vertical distance between) where radial marker 1 and radial marker 2B meet the upper vermillion border; e.g., the height difference between dotted line I and dotted line IIB.

Further, as shown in FIG. 2O, each of the dotted lines IIIA and IIIB extends laterally from the point at which each of the radial markers 3A and 3B (respectively) meet the upper vermillion border on each side of the upper lip. Height (034A) represents the height difference between (vertical distance between) where radial marker 2A and radial marker 3A meet the upper vermillion border; e.g., the height difference between (vertical distance between) dotted line IIA and dotted line IIIA. Height (034B) represents the height difference between (vertical distance between) where radial marker 2B and radial marker 3B meet the upper vermillion border; e.g., the height difference (vertical distance between) between dotted line IIB and dotted line IIIB.

Still further, as shown in FIG. 2O, each of the dotted lines IVA and IVB extends laterally from the point at which each of the radial markers 4A and 4B (respectively) meet the upper vermillion border on each side of the upper lip. Height (036A) represents the height difference between (vertical distance between) where radial marker 3A and radial marker 4A meet the upper vermillion border; e.g., the height difference between (vertical distance between) dotted line IIIA and dotted line IVA. Height (036B) represents the height difference between (vertical distance between) where radial marker 3B and radial marker 4B meet the upper vermillion border; e.g., the height difference between (vertical distance between) dotted line IIIB and dotted line IVB.

Yet further, as shown in FIG. 2O, each of the dotted lines VA and VB extends laterally from the point at which each of the radial markers 5A and 5B (respectively) meet the upper vermillion border on each side of the upper lip. Height (038A) represents the height difference between (vertical distance between) where radial marker 4A and radial marker 5A meet the upper vermillion border; e.g., the height difference between (vertical distance between) dotted line IVA and dotted line VA. Height (038B) represents the height difference between (vertical distance between) where radial marker 4B and radial marker 5B meet the upper vermillion border; e.g., the height difference between (vertical distance between) dotted line IVB and dotted line VB.

According to aspects, height differences (vertical distances) (032A) and (032B) are at least generally, at least substantially, at least essentially, are essentially, or are the same. According to aspects, height differences (vertical distances) (034A) and (034B) are at least generally, at least substantially, at least essentially, are essentially, or are the same. According to aspects, height differences (vertical distances) (036A) and (036B) are at least generally, at least substantially, at least essentially, are essentially, or are the same. According to aspects, height differences (vertical distances) (038A) and (038B) are at least generally, at least substantially, at least essentially, are essentially, or are the same.

According to aspects, height differences (vertical distances) (032A) and (034A) are at least generally, at least substantially, at least essentially, are essentially, or are the same. According to aspects, height differences (vertical distances) (034A) and (036A) are at least generally, at least substantially, at least essentially, are essentially, or are the same. According to aspects, height differences (vertical distances) (036A) and (038A) are at least generally, at least substantially, at least essentially, are essentially, or are the same.

According to aspects, height differences (vertical distances) (032B) and (034B) are at least generally, at least substantially, at least essentially, are essentially, or are the same. According to aspects, height differences (vertical distances) (034B) and (036B) are at least generally, at least substantially, at least essentially, are essentially, or are the same. According to aspects, height differences (vertical distances) (036B) and (038B) are at least generally, at least substantially, at least essentially, are essentially, or are the same.

In aspects, at least 2 of height differences (vertical distances), such as at least 3, at least 4, at least 5, at least 6, at least 7, or all 8 of height differences (vertical distances (032A), (032B), (034A), (034B), (036A), (036B), (038A), and (038B) are at least generally, at least substantially, at least essentially, are essentially, or are the same. In aspects, at least 2 of height differences (vertical distances), such as at least 3, at least 4, at least 5, at least 6, at least 7, or all 8 of height differences (vertical distances (032A), (032B), (034A), (034B), (036A), (036B), (038A), and (038B) are between about 1 mm and about 3 mm, such as, e.g., about 1.1 mm, ˜1.2 mm, ˜1.3 mm, ˜1.4 mm, ˜1.5 mm, ˜1.6 mm, ˜1.7 mm, ˜1.8 mm, ˜1.9 mm, ˜2 mm, ˜2.1 mm, ˜2.2 mm, ˜2.3 mm, ˜2.4 mm, ˜2.5 mm, ˜2.6 mm, ˜2.7 mm, ˜2.8 mm, ˜2.9 mm, or, e.g., ˜3 mm, such as for example ˜1 mm-˜2.8 mm, ˜1 mm-˜2.6 mm, ˜1 mm-˜2.4 mm, ˜1 mm-˜2.2 mm, ˜1 mm-˜2 mm, or ˜1.2 mm-˜3 mm, ˜1.4 mm-˜3 mm, ˜1.6 mm-˜3 mm, ˜1.8 mm-˜3 mm, or ˜2 mm-˜3 mm, such as, for example, ˜1.2 mm-˜2.8 mm, ˜1.4 mm-˜2.6 mm, ˜1.6 mm-˜2.4 mm, ˜1.8 mm-˜2.2 mm, or, e.g., about 2 mm.

### Mathematical Guidance

According to certain aspects, the disclosure herein provides (1) the application of mathematical guidance in the establishment of marking system(s) for use in aesthetic modification procedure(s), e.g., upper lip lift procedure(s), e.g., subnasal upper lip lift procedure(s), e.g., deep plane release upper lip lift procedure(s); (2) marking system(s) which themselves provide mathematical guidance (indication) for determining when a procedure ancillary to a subnasal upper lip lift procedure, e.g., a corner lip lift procedure, should be performed in order to achieve target aesthetic effect(s) such as a target slope of the upper vermillion border (020).

Herein, the term mathematical guidance and similar terms can be understood as referring to specifically identified mathematical relationships between two or more locations, e.g., two or more points, on a surface, e.g., a body, e.g., a human body, e.g., a human face (e.g., any of the relationships described herein with respect to face marking, analyses(is), modification, etc.). Mathematical guidance can refer to, e.g., mathematical principles defining the relationships between two or more points which, when factored into the establishment of marking system(s) which are then used in aesthetic modification procedure(s) improve the predictability of the outcome of such procedure(s), improve the reproducibility of the procedure(s), improve patient satisfaction with the procedure(s), or any combination of any or all thereof.

In aspects, the mathematical guidance of the marking system indicates when an ancillary procedure, such as, e.g., a corner lip lift procedure, should be added/included to a first or primary procedure, such as an upper lip lift, in order to achieve a target aesthetic effect, such as a target slope of the upper vermillion border (020).

According to certain aspects, the disclosure provides a system of markings for use as a guide in aesthetic modification procedure(s) to attain a target aesthetic appearance, such as a target slope of the upper vermillion border (020). In aspects, characteristic(s), e.g., excision height, final lip height, or both, of/along at least one marking, e.g., at least one radial marker, is established upon consideration of the targeted aesthetic appearance, such as final lip height and upper vermillion slope. In aspects, at least one marking of the system of markings is/are made by considering the mathematical relationship(s) between marking(s) having a different vertical/diagonal orientation(s). In aspects, the targeted aesthetic appearance, e.g., slope of the upper vermillion, is determined by the modification of one or more marking lines; however, the impact of such modification is dependent upon the degree of vertical orientation (e.g., vertical orientation vs. diagonal orientation) of the modified marking line. In aspects, due to the natural inclination of the upper lip to undergo a more exaggerated rise centrally compared to laterally, and due to the outward rotation of marking lines of a marking system from central to lateral as is described elsewhere herein, in aspects, excision at locations indicated by a marking system along more vertically oriented marking lines provide detectably or significantly more vertical lift than excision along marking line(s) having more diagonal orientation. This is demonstrated by the consideration of Pythagoras' theorem a²+b²=c², wherein “c” is the diagonal hypotenuse and “a” is a vertically oriented segment. Consideration of this mathematical relationship at one or more marking lines, e.g., marking lines 4A (012A), 4B (012B), or both, in aspects, results in a marking system design wherein the application of/incorporation of the marking system to/in an aesthetic modification procedure such as an upper lip lift, e.g., subnasal upper lip lift, e.g., deep plane release upper lip lift (e.g., with or without concomitant corner lip lift(s)) results in a target aesthetic effect such as a targeted slope of the upper vermillion. In aspects, the targeted slope is defined by the height difference along the upper vermillion border (020) of the points associated with radial markings 2A (008A) and 3A (010A); 2B (008B) and 3B (010B); 3A (010A) and 4A (012A); and 3B (010B) and 4B (012B) each being between about 1 mm and about 3 mm, such as, e.g., about 2 mm (wherein the height difference between each set of neighboring radial markers is at least generally, at least substantially, at least essentially, is essentially, or is the same as that between other sets of neighboring radial markers described here).

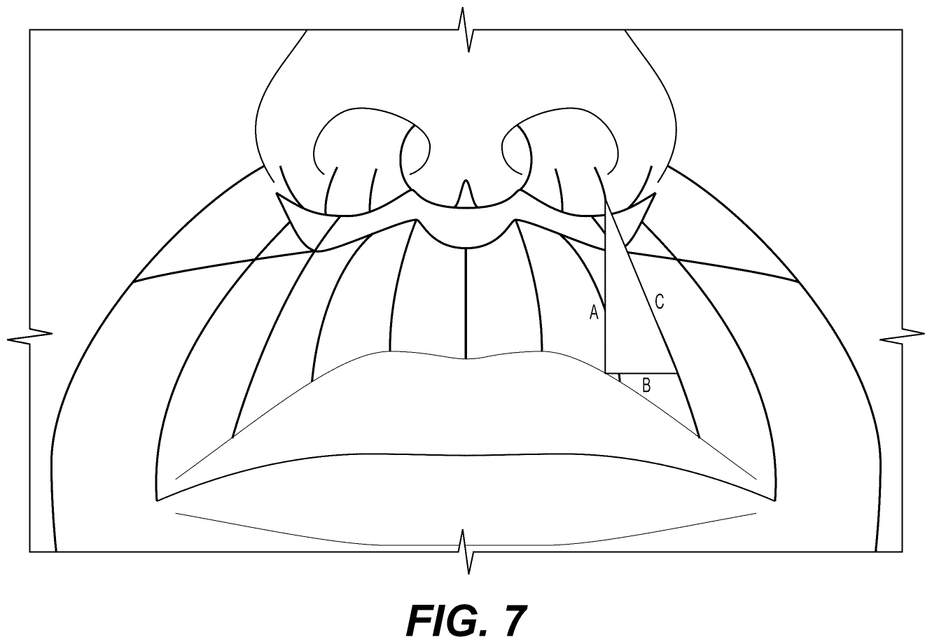

According to certain aspects, the marking system identifies a central portion of the upper lip wherein the lift applied to this portion of the lip is at least generally, at least substantially, at least essentially, is essentially, or is vertical. In aspects, such a central portion of the upper lip is the portion or region of the upper lip between radial markers 4A (012A) and 4B (012B). In aspects, as described above, radial markers 4A (012A) and 4B (012B) identify a transition from the subnasal lip and the lateral lip. In aspects, the marking system identifies a lateral portion of the upper lip wherein the lift applied to this portion of the lip is at least generally, at least substantially, at least essentially, is essentially, or is diagonal. In aspects, such a lateral portion of the upper lip is the portion or region of the upper lip lateral to radial markers 4A (012A) and 4B (012B). In aspects, at upper lip locations identified by radial markers 4A (012A) and 4B (012B), the design of the marking system, the use of the marking system, or both, comprises consideration of Pythagoras' theorem, a²+b²=c² as it relates to radial markers 1, 4A (012A), and 4B (012B). At radial markers 4A (012A) and 4B (012B), a diagonal (hypotenuse) measurement/length “X” of radial marker(s) with a given gap (horizontal) between points along the upper vermillion border (020) of adjacent radial markers, e.g., a diagonal lift, will yield a vertical lift which is detectably or significantly less than if the same measurement/length “X” was performed at radial marker 1 (006). That is, for example, as is shown in FIG. 7, a 7-mm diagonal (hypotenuse) measurement, radial marker 4B (012B), with a 3-mm gap (horizontal) will result in a 6.3 mm vertical lift as opposed to the 7 mm vertical lift that would be achieved with a 7 mm excision at radial marking 1 (006). Accordingly, an, e.g., 2 mm height difference between neighboring points on the upper vermillion border (020) (e.g., between radial markers 2A (008A) and 3A (010A); 3A (010A) and 4A (012A); 2B (008B) and 3B (010B); and 3B (010B) and 4B (012B)) will result in a final slope of the upper vermillion border (020) that is steady with a lift that is detectably or significantly, e.g., gradually, diminishing as it approaches radial markers 3A (010A) and 4A (012A) and beyond in a first lateral direction and as it approaches radial markers 3B (010B) and 4B (012B) and beyond in a second lateral direction.

Therefore, according to aspects, the disclosure provides marking system(s) of the upper lip for use in upper lip modification procedure(s) wherein Pythagoras' theorem a²+b²=c² is applied to determine the necessary length of the segment or gap identified as excision height of one or more radial marking(s) given a determined lateral distance between two points along the upper vermillion of two radial markers to result in a height difference between two points of at least two radial markings along the upper vermillion border (020) of between about 1 mm and about 3 mm, such as about 2 mm. More specifically, in one exemplary aspect, the disclosure provides marking system(s) of the upper lip for use in upper lip modification procedure(s) wherein Pythagoras' theorem a²+b²=c² is applied to determine the necessary length of the segment or gap identified as excision height of one or more of radial markings 3A (010A), 4A (012A), 3B (010B), and 4B (012B) given a determined lateral distance between two points along the upper vermillion border (020) to result in a height difference between the points of 3A (010A) and 4A (012A) along the upper vermillion border (020), the height difference between the points of 3B (010B) and 4B (012B) along the upper vermillion border (020), or to result in a height difference between the points of 3A (010A) and 4A (012A) and 3B (010B) and 4B (012B), of between about 1 mm and about 3 mm such as, e.g., about 2 mm.

According to certain aspects, the disclosure provides marking system(s) for use on a human subject in conjunction with the performance of an aesthetic modification procedure, e.g., for use on a human subject's upper use for use in a method of performing an aesthetic modification procedure, e.g., an upper lip lift, e.g., subnasal upper lip lift, e.g., a deep plane release upper lip lift procedure (e.g., with or without concomitant corner lip lift(s)), comprising radial marking(s) wherein the length of the radial marking(s) of the marking system(s) establish(es) a height differential between the trough of the subject's Cupid's bow and the peaks of the subject's Cupid's bow of between about 1 mm and about 5 mm, e.g., ˜1 mm-˜4 mm, ˜1-˜3 mm, or ˜1 mm-˜2 mm, such as ˜2 mm-˜5 mm, ˜3 mm-˜5 mm, or ˜4 mm-˜5 mm, such as ˜2 mm-˜4 mm, or, e.g., ˜2 mm.

According to certain aspects, the disclosure provides marking system(s) for use on a human subject in conjunction with the performance of an aesthetic modification procedure, e.g., for use on a human subject's upper use for use in a method of performing an aesthetic modification procedure, e.g., an upper lip lift, e.g., subnasal upper lip lift, e.g., a deep plane release upper lip lift procedure (e.g., with or without concomitant corner lip lift(s)), comprising radial marking(s) wherein the length of the radial marking(s) of the marking system(s) establish(es) a height differential between dip of the columella beneath the nose of the subject and the points where the medial crural footplates diverge into the nose at the junction with the alar sill of the subject is between about 1 mm and about 5 mm, e.g., ˜1 mm-˜4 mm, ˜1-˜3 mm, or ˜1 mm-˜2 mm, such as ˜2 mm-˜5 mm, ˜3 mm-˜5 mm, or ˜4 mm-˜5 mm, such as ˜2 mm-˜4 mm, or, e.g., ˜2 mm.

According to certain aspects, the disclosure provides marking system(s) for use on a human subject in conjunction with the performance of an aesthetic modification procedure, e.g., for use on a human subject's upper use for use in a method of performing an aesthetic modification procedure, e.g., an upper lip lift, e.g., subnasal upper lip lift, e.g., a deep plane release upper lip lift procedure (e.g., with or without concomitant corner lip lift(s)), comprising radial marking(s) wherein the length of the radial marking(s) of the marking system(s) establish(es) a height differential between the trough of the Cupid's bow and the peaks of Cupid's bow and the height differential between the dip of the columella beneath the nose and the points where the medial crural footplates diverge into the nose at the junction with the alar sill are each about 1 mm to about 5 mm, e.g., ˜1 mm-˜4 mm, ˜1-˜3 mm, or ˜1 mm-˜2 mm, such as ˜2 mm-˜5 mm, ˜3 mm-˜5 mm, or ˜4 mm-˜5 mm, such as ˜2 mm-˜4 mm, or, e.g., 2 mm.

### Excision Height & Shape

In aspects as described above, radial marker(s) of marking system(s) provided herein comprise an excision height segment, defined by the upper excision boundary marker (002) and the lower excision boundary marker (004). In aspects, the upper excision boundary marker (002) and the lower excision boundary marker (004), e.g., the distance along each of the radial marking(s), e.g., the distance along each of the at least 7 radial markings, between the upper excision boundary marker (002) and the lower excision boundary marker (004) define the excision height and identify the shape of a portion of the upper lip to be excised in an aesthetic modification procedure, e.g., an upper lip lift, e.g., a subnasal upper lip lift, e.g., a deep plane release upper lip lift procedure (e.g., with or without concomitant corner lip lift(s)).

According to the certain aspects, marking system(s) provided herein yield a portion of the upper lip to be excised which is at least generally, at least substantially, at least essentially, essentially, or which is shaped like “bull's horns,” e.g., whereby the marking system(s) provided herein lend themselves to use in procedure(s) encompassing use of a bull's horn surgical incision design well known in the art.

In aspects, the length(s) of segment(s) of each radial marking of a marking system identifying the final height of the upper lip detectably or significantly establish the excision height of a bull's horn-shaped excision at the location of each of the radial marking(s). According to certain aspects, marking system(s) provided herein are used to mark the face of a subject undergoing an aesthetic modification procedure, such as an upper lip lift, e.g., subnasal upper lip lift, e.g., deep plane release subnasal and corner lip lift procedure wherein, in aspects, the nasal base, upper lip, or both of the subject possess(es) one or more asymmetries. In aspects, one or more radial markings of Group A (e.g., one or more Group A markings), or at a minimum one or segments of one or more radial markings of Group A, is detectably or significantly different that one or more of the corresponding radial marking(s) of Group B (e.g., one or more corresponding Group B markings). In aspects, one or more radial markings of Group A are detectably or significantly different than its corresponding Group B marking so as to facilitate the variation in excision height; that is, such markings are intentionally different so as to vary the excision design of the excision, e.g., bull's horn-shaped excision, from side-to-side of the subject's upper lip in order to detectably or significantly improve, correct, or otherwise adjust an identifiable asymmetry.

According to certain aspects, the excision height remains constant across the length of the portion of the upper lip to be excised. According to certain aspects, the excision height changes across the length of the portion of the upper lip to be excised. In aspects, the excision height changes across the length of the portion of the upper lip to be excised by about 0% to about 1000% between the locations of two neighboring radial markers (also referred to herein as radial markings), such as, e.g., ˜0%-˜900%, ˜0%-˜800%, ˜0%-˜600%, ˜0%-˜400%, ˜0%-˜200%, ˜0%-˜100%, ˜0%-˜80%, ˜0%-˜60%, ˜0%-˜40% or, e.g., ˜0%-˜20%, such as, for example, ˜10%-˜1000%, ˜20%-˜1000%, ˜40%-˜1000%, ˜60%-˜1000%, ˜80%-˜1000%, ˜100%-˜1000%, ˜200%-˜1000%, ˜400%-˜1000%, ˜600%-˜1000%, or, e.g., ˜800%-˜1000%, as in, for example, between ˜10%-˜800%, ˜15%-˜600%, ˜20%-˜400%, ˜10%-˜200%, or, e.g., ˜15%-˜100%, such as, e.g., about 5%, ˜10%, ˜20%, ˜30%, ˜40%, ˜50%, ˜60%, ˜70%, ˜80%, ˜90%, or, e.g., ˜100% between the locations of two neighboring radial markers.

In aspects, the excision height ranges from between about 0 mm and about 15 mm across the length of the portion of the upper lip to be excised, such as, e.g., about 0 mm to about 14 mm, ˜0 mm-˜13 mm, ˜0 mm-˜12 mm, ˜0 mm-˜11 mm, ˜0 mm-˜10 mm, ˜0 mm-˜9 mm, ˜0 mm-˜8 mm, ˜0 mm-˜7 mm, ˜0 mm-˜6 mm, ˜0 mm-˜5 mm, or e.g., ˜0 mm-˜4 mm.

In aspects, the excision height ranges from between about 0 mm and about 15 mm across the length of the portion of the upper lip to be excised, such as, e.g., about 1 mm to about 15 mm, ˜2 mm-˜15 mm, ˜3 mm-˜15 mm, ˜4 mm-˜15 mm, ˜5 mm-˜15 mm, ˜6 mm-˜15 mm, ˜7 mm-˜15 mm, ˜8 mm-˜15 mm, ˜9 mm-˜15 mm, or, e.g., ˜10 mm-˜15 mm.

In aspects, incision height is established, by the positioning of one or more marker(s) of marking system(s), to attain one or more target aesthetic effects, such as, e.g., overall facial balance, incisor display, final lip height, etc. In aspects, the excision height segment or gap in/of radial marking(s) for one or more radial marking(s) is established while the lip is held under gentle tension. In aspects, the excision height segment or gap in/of radial marking 1 (006) is placed while the lip is held under gentle tension. In aspects, the excision height segment or gap of radial marking(s), e.g., radial marker (1) is established while the lip is held under gentle tension while the final height segment of radial marking(s) is/are established while the lip is in repose and is not detectably or significantly stretched. Herein, “gentle tension” or “gentle stretch” means the application of sufficient digital pressure to relieve any skin bunching or curling of the lip.

### Visual Indicators

In aspects, the marking system(s) provided herein comprise one or more marking(s) operating as visual indicator(s). In aspects, the visual indicator(s) identify or signal step(s) to be performed by a practitioner of a procedure performed by a method incorporating the use of the marking system(s). In aspects, an upper lip lift, e.g., subnasal upper lip lift, e.g., a deep plane release subnasal lip lift procedure (e.g., with or without concomitant corner lip lift(s)), is performed according to a method comprising application of/incorporation of/use of marking system(s) provided herein, wherein one or more radial marking(s) is/are a visual indicator(s) of one or more steps to be performed in the upper lip lift procedure with which the radial marking(s) are associated. In certain aspects, such visual indicator(s) remind a practitioner to place sutures as a step of a method incorporating the use of the marking system(s). In aspects, such visual indicator(s) remind practitioner(s) to place internal mattress sutures during an upper lip lift procedure incorporating the use of the marking system(s). According to certain specific aspects, radial marking(s) 5A (014A), 5B (014B), or both, can provide (among other thing(s)) visual indicator(s) reminding a practitioner to place lateral internal mattress sutures during an upper lip lift procedure comprising application of a marking system comprising radial marking(s) 5A (014A), 5B (014B), or both.

### Ratios

According to aspects, any component(s) described herein can be present in effective quantity(ies), wherein, e.g., a quantity is a length, a number of instances, etc. In aspects, any single component herein, such as, for example, a marking of a marking system or portion(s) thereof, can be present in a relationship with, such as, e.g., in a ratio with, any one or more other single components, e.g., marking of a marking system or portion(s) thereof. In aspects, any combination of component(s) provided herein can be present in a ratio with any other combination of component(s). In aspects, ratio(s) between such component(s) or combinations thereof can be established using any provided quantity(ies) for each disclosed herein, including, e.g., values within ranges of such amounts disclosed herein.

According to certain aspects, marker(s) of marking system(s) provided by the disclosure are characterizable by their relationship to other marker(s) of the marking system(s). In aspects, one or more segment(s) of marker(s) of marking system(s) provided by the disclosure are characterizable by their relationship to other segment(s) of marker(s) of marking system(s) provided by the disclosure. In certain aspects, the marking system(s) provided by the disclosure are characterizable as comprising relationship(s) between segment(s) of marker(s) and total length(s) of marker(s).

Discussion of ratio(s) here, in aspects, is directed to ratio(s) of marker(s) the characteristic(s) of which if established/applied in an aesthetic modification procedure, establish one or more upper vermillion border slope(s) between radial markers 5A and 4A (identified as (028A) in FIG. 2N); radial markers 4A and 3A (identified as (026A) in FIG. 2N); radial markers 3A and 2A (identified as (024A) in FIG. 2N); radial markers 2B and 3B (identified as (024B) in FIG. 2N); radial markers 3B and 4B (identified as (026B) in FIG. 2N); radial markers 4B and 5B (identified as (028B) in FIG. 2N), or combination(s) thereof of between about 1 and about 3, e.g., about 2, or also or alternatively a slope of the upper vermillion border between radial marker 1 and radial marker 2A that are at least substantially, at least essentially, are essentially, or are the same.

Discussion of ratio(s) here, in aspects, is directed to ratio(s) of marker(s) the characteristic(s) of which if established/applied in an aesthetic modification procedure, establish a height difference along the upper vermillion border between radial markers 5A and 4A (identified as (038A) in FIG. 2O); radial markers 4A and 3A (identified as (036A) in FIG. 2O); radial markers 3A and 2A (identified as (034A) in FIG. 2O); radial markers 2B and 3B (identified as (034B) in FIG. 2O); radial markers 3B and 4B (identified as (036B) in FIG. 2O); radial markers 4B and 5B (identified as (038B) in FIG. 2O), or combination(s) thereof of between about 1 mm and about 3 mm, e.g., about 2 mm.

Discussion of ratio(s) here, in aspects, is directed to ratio(s) of marker(s), the characteristic(s) of which, if established/applied in an aesthetic modification procedure, establish an incisor display of between about 1 mm and about 3 mm, e.g., about 2 mm.

In aspects, marking system(s) provided by the disclosure comprise one or more radial marking(s) comprising an excision segment (e.g., as indicated by the marker (or, e.g., location as described relevant to embodiments of the disclosure wherein the marking system is virtual, as established by automated system(s)) having an excision segment height length; a final lip height segment having a final lip height segment length; or both, wherein, in aspects, any single radial marking(s) having both an excision segment and a final lip height segment can comprise a ratio of the excision segment height length (“#-EX”) to final lip height segment length (“#-FH”) which is any ratio suitable and/or necessary for achieving target aesthetic effect(s). In aspects, such ratios(s) can be established using any values provided for excision height length(s) and final lip height segment length(s) provided herein. As alluded to above and described elsewhere herein, disclosure of ratios of marker(s) or segment(s) thereof to other marker(s) or segment(s) of other marker(s) should be interpreted as disclosure applying to such corresponding location(s) identified/identifiable by automated system(s) (and related method(s)) herein.

In aspects, marking system(s) provided by the disclosure comprise two or more radial marking(s) comprising an excision segment having an excision segment height length; a final lip height segment having a final lip height segment length; or both, wherein, in aspects, a first and second radial marking each comprise an excision segment and wherein the ratio of the excision segment height length of the first radial marking (“#1-EX”) to the excision segment height length of the second radial marking (“#2-EX”) is any ratio suitable and/or necessary for achieving target aesthetic effect(s). In aspects, such ratio(s) can be established using any values provided for excision height segment length(s) provided herein.

In aspects, marking system(s) provided by the disclosure comprise two or more radial marking(s) comprising an excision segment having an excision segment height length; a final lip height segment having a final lip height segment length; or both, wherein, in aspects, a first and second radial marking each comprise a final lip height segment and wherein the ratio of the final lip height segment length of the first radial marking (“#1-FH”) to the final lip height segment length of the second radial marking (“#2-FH”) is any ratio suitable and/or necessary for achieving target aesthetic effect(s). In aspects, such ratio(s) can be established using any values provided for the final lip height segment length(s) provided herein.

In aspects, marking system(s) provided by the disclosure comprise two or more radial marking(s) comprising an excision segment having an excision segment height length; a final lip height segment having a final lip height segment length; or both, wherein, in aspects, a first radial marking comprises an excision segment and second radial marking comprises a final lip height segment and wherein the ratio of the excision height segment length of the first radial marking (“#1-EX”) to the final lip height segment length of the second radial marking (“#2-FH”) is any ratio suitable and/or necessary for achieving target aesthetic effect(s). In aspects, such ratio(s) can be established using any values provided for excision segment height length(s) and final lip height segment length(s) provided herein.

In aspects, marking system(s) provided by the disclosure comprise two or more radial marking(s) each having a total length, wherein, in aspects, the ratio of the total length of the first radial marking (“#1-T”) to the total length of the second radial marking (“#2-T”) is any ratio suitable and/or necessary for achieving target aesthetic effect(s). In aspects, such ratio(s) can be established using any values provided for the total length(s) of marker(s) provided herein.

According to certain specific aspects, marking system(s) provided herein comprise a first radial marking (radial marker 1 (006)) comprising a segment indicating the final height (1-FH) of the upper lip at the position of radial marker 1 (006) and further comprise second and third radial markings (radial markers 2A (008A) and 2B (008B)) each comprising a segment indicating the final height of the upper lip at the position of radial marker(s) 2A (008A) and 2B (008B) respectively (2A-FH and 2B-FH, respectively) wherein 1-FH is at least generally the same (equal), at least substantially the same (equal), at least essentially the same (equal), essentially the same (equal), or which is the same/equal as/to that of 2A-FH, 2B-FH, or both. In certain such aspects, one or more of the excision height segments of radial marker 1 (1-EX), radial marker 2A (2A-EX), and radial marker 2B (2B-EX) are different from one or more of the others, such that the ratio of the lengths of 1-EX:2A-EX; 1-EX:2B-EX: or 2A-EX:2B-EX is not 1:1. Extending this example, in aspects, the final lip height of 2A (2A-FH) and 2B (2B-FH); 3A (3A-FH) and 3B (3B-FH), 4A (4A-FH) and 4B (4B-FH), 5A (5A-FH) and 5B (5B-FH), or two or more of such pairs of marker(s) can be the same or at least substantially, at least essentially, or essentially the same (e.g., can have a ratio of 1:1 or at least substantially, at least essentially, or essentially 1:1), while the excision heights (#-EH) of such pairs of markers may be different, e.g., wherein the ratios of excision heights of such pairs of markers is not 1:1.

In certain aspects, 1-FH varies from 2A-FH, 2B-FH, or both by no more than about 3 mm, such as, e.g., by no more than about 2.5 mm, no more than about 2 mm, no more than about 1.5 mm, no more than about 1 mm, no more than about 0.75 mm, no more than about 0.5 mm, or no more than, e.g., about 0.25 mm. In aspects, the final height segments of pairs of markers, e.g., 2A (2A-FH) and 2B (2B-FH); 3A (3A-FH) and 3B (3B-FH), 4A (4A-FH) and 4B (4B-FH), 5A (5A-FH) and 5B (5B-FH), or two or more of such pairs of marker(s) can be the same or at least substantially, at least essentially, or essentially the same (e.g., can have a ratio of 1:1 or at least substantially, at least essentially, or essentially 1:1), varying between them by no more than about 3 mm, such as, e.g., by no more than about 2.5 mm, no more than about 2 mm, no more than about 1.5 mm, no more than about 1 mm, no more than about 0.75 mm, no more than about 0.5 mm, or no more than, e.g., about 0.25 mm. In aspects, the excision heights (#-EH) of such pairs of markers may be different, e.g., wherein the ratios of excision heights of such pairs of markers is not 1:1, e.g., they may vary by more than about 0.5 mm, by more than about 0.75 mm, by more than about 1 mm, by more than about 1.5 mm, by more than about 2 mm, by more than about 2.5 mm, by more than about 3 mm, or more.

According to certain aspects, the marking system(s) provided herein have a first set of Group A marker(s) and a second set of Group B markers, Group A and Group B marker(s) being described elsewhere herein. In aspects, each marker in the set of Group A markers, and each marker in the set of Group B markers has a total length. In aspects, the sum of the total length of all Group A markers is detectably or significantly greater than the sum of the total length of all Group B markers. In aspects, the ratio of the sum of the total length of all Group A markers to the sum of the total length of all Group B markers is detectably or significantly greater than 1. In aspects, the sum of the total length of all Group A markers is detectably or significantly less than the sum of the total length of all Group B markers. In aspects, the ratio of the sum of the total length of all Group A markers to the sum of the total length of all Group B markers is detectably or significantly less than 1. In aspects, the sum of the total length of all Group A markers is at least generally, at least substantially, at least essentially, is essentially, or is equal to the sum of the total length of all Group B markers. In aspects, the ratio of the sum of the total length of all Group A markers to the sum of the total length of all Group B markers is about 1.

According to certain aspects, the marking system(s) provided herein have a first set of Group A marker(s) and a second set of Group B marker(s), Group A and Group B marker(s) being described elsewhere herein. In aspects, at least one marker in the set of Group A marker(s), and at least one marker in the set of Group B marker(s) has an excision height segment length. In aspects, the sum of the excision height segment length(s) of all Group A marker(s) is detectably or significantly greater than the sum of the excision height segment length(s) of all Group B marker(s). In aspects, the ratio of the sum of the excision height segment length(s) of all Group A marker(s) to the sum of the excision height segment length(s) of all Group B marker(s) is detectably or significantly greater than 1. In aspects, the sum of the excision height segment length(s) of all Group A marker(s) is detectably or significantly less than the sum of the excision height segment length(s) of all Group B marker(s). In aspects, the ratio of the sum of the excision height segment length(s) of all Group A markers to the sum of the excision height segment length(s) of all Group B marker(s) is detectably or significantly less than 1. In aspects, the sum of the excision height segment length(s) of all Group A marker(s) is at least generally, at least substantially, at least essentially, is essentially, or is equal to the sum of the excision height segment length(s) of all Group B marker(s). In aspects, the ratio of the sum of the excision height segment length(s) of all Group A marker(s) to the sum of the excision height segment length(s) of all Group B marker(s) is about 1.

According to certain aspects, the marking system(s) provided herein have a first set of Group A marker(s) and a second set of Group B marker(s), Group A and Group B marker(s) being described elsewhere herein. In aspects, Group A markers and Group B markers have a final upper lip height segment length. In aspects, the sum of the final upper lip height segment length(s) of all Group A marker(s) is detectably or significantly greater than the sum of the final upper lip height length(s) of all Group B marker(s). In aspects, the ratio of the sum of the final upper lip height segment length(s) of all Group A marker(s) to the sum of the final upper lip height segment length(s) of all Group B marker(s) is detectably or significantly greater than 1. In aspects, the sum of the final upper lip height segment length(s) of all Group A marker(s) is detectably or significantly less than the sum of the final upper lip height segment length(s) of all Group B marker(s). In aspects, the ratio of the sum of the final upper lip height segment length(s) of all Group A markers to the sum of the final upper lip height segment length(s) of all Group B marker(s) is detectably or significantly less than 1. In aspects, the sum of the final upper lip height segment length(s) of all Group A marker(s) is at least generally, at least substantially, at least essentially, is essentially, or is equal to the sum of the final upper lip height segment length(s) of all Group B marker(s). In aspects, the ratio of the sum of the final upper lip height segment length(s) of all Group A marker(s) to the sum of the final upper lip height segment length(s) of all Group B marker(s) is about 1.

According to certain aspects, the marking system(s) provided herein have a first set of Group A marker(s) and a second set of Group B marker(s), Group A and Group B marker(s) being described elsewhere herein. In aspects, each Group A marker and each Group B marker has a total length. In aspects, the total length of at least one Group A marker is detectably or significantly greater than the total length of its corresponding Group B marker. In aspects, the ratio of the total length of at least one Group A marker to the total length of its corresponding Group B marker is detectably or significantly greater than 1. In aspects, the total length of at least one Group A marker is detectably or significantly less than the total length of its corresponding Group B marker. In aspects, the ratio of the total length of a Group A marker to the total length of its corresponding Group B marker is detectably or significantly less than 1. In aspects, the total length of a Group A marker is at least generally, at least substantially, at least essentially, is essentially, or is equal to the total length of its corresponding Group B marker. In aspects, the ratio of the total length of a Group A marker to the total length of its corresponding Group B marker is about 1.

According to certain aspects, the marking system(s) provided herein have a first set of Group A marker(s) and a second set of Group B marker(s), Group A and Group B marker(s) being described elsewhere herein. In aspects, marking system(s) herein comprise at least one Group A marker and at least one corresponding Group B marker, wherein each of the A-B pair of markers comprises an excision height, e.g., excision height segment. In aspects, the excision height segment length of at least one Group A marker is detectably or significantly greater than the excision height segment length of its corresponding Group B marker. In aspects, the ratio of the excision height segment length of at least one Group A marker to the excision height segment length of its corresponding Group B marker is detectably or significantly greater than 1. In aspects, the excision height segment length of at least one Group A marker is detectably or significantly less than the excision height segment length of its corresponding Group B marker. In aspects, the ratio of the excision height segment length of a Group A marker to the excision height segment length of its corresponding Group B marker is detectably or significantly less than 1. In aspects, the excision height segment length of a Group A marker is at least generally, at least substantially, at least essentially, is essentially, or is equal to the excision height segment length of its corresponding Group B marker. In aspects, the ratio of the excision height segment length of a Group A marker to the excision height segment length of its corresponding Group B marker is about 1.

According to certain aspects, the marking system(s) provided herein have a first set of Group A marker(s) and a second set of Group B marker(s), Group A and Group B marker(s) being described elsewhere herein. In aspects, each Group A marker and each Group B marker has a final lip height. In aspects, the final lip height segment length of at least one Group A marker is detectably or significantly greater than the final lip height segment length of its corresponding Group B marker. In aspects, the ratio of the final lip height segment length of at least one Group A marker to the final lip height segment length of its corresponding Group B marker is detectably or significantly greater than 1. In aspects, the final lip height segment length of at least one Group A marker is detectably or significantly less than the final lip height segment length of its corresponding Group B marker. In aspects, the ratio of the final lip height segment length of a Group A marker to the final lip height segment length of its corresponding Group B marker is detectably or significantly less than 1. In aspects, the final lip height segment length of a Group A marker is at least generally, at least substantially, at least essentially, is essentially, or is equal to the final lip height segment length of its corresponding Group B marker. In aspects, the ratio of the final lip height segment length of a Group A marker to the final lip height segment length of its corresponding Group B marker is about 1.

According to some aspects, the disclosure herein provides marking system(s) comprising marker(s) finding application in procedure(s) which are not upper lip lift procedure(s), but which may be concomitantly performed with an upper lip lift procedure(s) (ancillary marking(s)). In aspects, the disclosure comprises such marking(s) wherein the marking system(s) is/are characterized by ratio(s) of one or more element(s) of such ancillary markings to one or more elements of marking(s) of the marking system(s) finding application in upper lip lift procedure(s).

In certain aspects, marking system(s) provided herein comprise(s) at least one marking, e.g., at least two markings positioned lateral to each of radial marking(s) 4A (012A) and 4B (012B) which are applicable to one or more ancillary procedure(s) performed concomitant to an upper lip lift procedure, e.g., corner lip lift procedure. In aspects, marking system(s) comprise at least one marking, e.g., at least two markings (radial marking 5A (014A), radial marking 5B (014B), or both) positioned lateral to each of radial marking(s) 4A (012A) and 4B (012B), respectively and which are applicable for incorporation into methods of performing a corner lip lift procedure. In aspects, at least two such markers are present, e.g., radial marking(s) 5A (014A) and 5B (014B). In aspects, the at least two marking(s) positioned lateral to radial marking(s) 4A (012A) and 4B (012B), respectively, e.g., radial marking(s) 5A (014A), 5B (014B), or both as applicable to the side(s) of the subject's mouth undergoing the corner lip lift procedure (such markings being described elsewhere herein) comprise a second lower incision point along their respective lengths (lower excision boundary marker(s) forming a lower excision boundary marker (004)). In aspects, the lower excision boundary marker (004) position establishes a segment of each of the respective applicable radial markings 4A (012A) and 5A (014A), 4B (012B) and 5B (014B), or any or all thereof representing the final lip height (4A-FH, 4B-FH, 5A-FH, 5B-FH, or any or all thereof) which is at least generally, at least substantially, at least essentially, is essentially, or is equal in length to that of one or more other of radial marking(s) of said group. That is, in aspects, at least 2, at least 3, or all of 4A-FH, 4B-FH, 5A-FH, and 5B-FH are at least generally, at least substantially, at least essentially, are essentially, or are equal in length.

In aspects, height(s) defined by, i.e., defined as the distance between (a) extension marking(s), e.g., lateral extension line(s) e.g., LE-A (018A), LE-B (018B), or both, where each of LE-A (018A) and LE-B (018B) extends laterally from the lower excision point(s) along radial markers 4A (012A) and 4B (012B) (respectively), and (b) the top limb(s) of corner lift marker(s) (e.g., horizontal reference point marking(s)), e.g., H-A (046A), H-B (046B), or both, respectively, is/are at least generally, at least substantially, at least essentially, are essentially, or is equal to one or both (each) of the final lip height(s) of radial marker(s) 4A (012A) and 4B (012B).

In aspects, the distance between H-A (046A) and LE-A (018A) is at least generally the same as, at least substantially the same as, at least essentially the same as, is essentially the same as, or is the same as the final lip height (4A-FH) defined by radial marker 4A (012A).

In aspects, the distance between H-B (046B) and LE-B (018B) is at least generally the same as, is at least substantially the same as, is at least essentially the same as, is essentially the same as, or is the same as the final lip height (4B-FH) defined by radial marker 4B (012B).

In aspects, the distance between H-A (046A) and LE-A (018A) is at least generally the same as, at least substantially the same as, at least essentially the same as, is essentially the same as, or is the same as, the distance between H-B (046B) and LE-B (018B).

In aspects, the final lip height (4A-FH) defined by radial marker 4A (012A)) is at least generally the same as, at least substantially the same as, at least essentially the same as, is essentially the same as, or is the same as, the final lip height (4B-FH) defined by radial marker 4B (012B).

In aspects, two or more of, three or more of, or all four of the distances identified as (a) the distance between H-A (046A) and LE-A; (b) the distance between H-B (046B) and LE-B (018B); (c) the length identified as 4A-FH; and (d) the length identified as 4B-FH, are at least generally the same, at least substantially the same, at least essentially the same, are essentially the same, or are the same. In aspects, two or more of, such as three or more of, or, all of such distances (lengths) are within, e.g., about 10%, ˜9%, ˜8%, ˜7%, ˜6%, ˜5%, ˜4%, ˜3%, ˜2%, ˜1%, ˜0.9%, ˜0.8%, ˜0.7%, ˜0.6%, ˜0.5%, ˜0.4%, ˜0.3%, ˜0.2%, or, e.g., ˜0.1%, of one another, such as within less than about 5%, <˜4%, <˜3%, <˜2%, or <˜1%, such as <˜0.5% of one another.

In certain aspects, marking system(s) provided herein comprise(s) at least one marking, e.g., at least two markings positioned lateral to each of radial marking(s) 4A (012A) and 4B (012B) which are applicable to one or more ancillary procedure(s) performed concomitant to an upper lip lift procedure, e.g., corner lip lift procedure. In aspects, the at least two marking(s) positioned lateral to radial marking(s) 4A (012A) and 4B (012B), respectively, e.g., radial marking(s) 5A (014A), 5B (014B), or both as applicable to the side(s) of the subject's mouth undergoing the corner lip lift procedure (such markings being described elsewhere herein) comprise a second lower incision point along their respective lengths (lower excision boundary marker(s) forming the single lower excision boundary marker (004)). In aspects, the lower excision boundary marker (004) position establishes a segment of each of the respective applicable radial markings 4A (012A) and 5A (014A), 4B (012B) and 5B (014B), or any or all thereof representing the excision height (4A-EX, 4B-EX, 5A-EX, 5B-EX, or any or all thereof) which is at least generally, at least substantially, at least essentially, is essentially, or is equal in length to that of one or more other of radial marking(s) of said group. That is, in aspects, at least 2, at least 3, or all of 4A-EX, 4B-EX, 5A-EX, and 5B-EX are at least generally, at least substantially, at least essentially, are essentially, or are equal in length. In aspects, the lower excision boundary marker (004) position establishes a segment of each of the respective applicable radial markings 4A (012A) and 5A (014A), 4B (012B) and 5B (014B), or any or all thereof representing the excision height (4A-EX, 4B-EX, 5A-EX, 5B-EX, or any or all thereof) which is detectably or significantly different in length to that of one or more other of radial marking(s) of said group. That is, in aspects, at least 2, at least 3, or all of 4A-EX, 4B-EX, 5A-EX, and 5B-EX are detectably or significantly different in length.

### Markings & Ancillary Procedure(s)

In aspects, the disclosure provides marking system(s), e.g., marking system(s) operating as a guide, useful in the conduct of, applicable to the performance of, suitable for incorporation into methods of performing, or any or all thereof, one or more aesthetic modification procedure(s). In aspects, an aesthetic modification procedure suitable for or finding benefit from the incorporation of marking system(s) herein is an upper lip lift procedure, e.g., a subnasal upper lip lift procedure, e.g., a subnasal upper lip lift procedure utilizing a bull's horn surgical technique, e.g., a deep plane release upper lip lift procedure or a deep plane release upper lip lift modification procedure utilizing a bull's horn surgical technique (e.g., with or without concomitant corner lip lift(s)). In certain aspects, an aesthetic modification procedure(s) suitable for or finding benefit from the incorporation of marking system(s) herein are one or more procedures ancillary to an upper lip lift procedure, e.g., an aesthetic modification procedure performed concomitant to a subnasal upper lip lift procedure. In aspects, such ancillary or concomitantly performed procedure(s) is/are corner lip lift(s), nasal base suspension(s), muscle suspension(s), or combination(s) of any or all thereof.

### Corner Lift

In aspects, marking(s), e.g., radial marking(s) of the marking system(s) provided herein, operate as a guide for one or more steps in the performance of a corner lip lift procedure incorporating such system(s). In aspects, one or more radial markings establish one or more locations applicable to the conduct of a corner lift procedure performed in addition to, e.g., ancillary to or concomitantly to, an upper lip lift, e.g., subnasal upper lip lift procedure, e.g., a deep plane release upper lip lift modification procedure. In aspects, radial marking(s) 5A (014A), 5B (014B), or both of marking system(s) described herein establish one or more location(s) applicable to the conduct of a corner lip lift procedure, e.g., a corner lip lift procedure performed on the subject in addition to (e.g., ancillary to or concomitant to) a subnasal upper lip lift procedure such as a deep plane release upper lip lift procedure.

According to certain aspects, marking system(s) comprise at least one marking not applicable to a primary procedure, e.g., upper lip lift procedure, e.g., a subnasal upper lip lift procedure, but which is applicable to a secondary procedure, e.g., an ancillary procedure or procedure performed concomitantly to the primary (e.g., subnasal upper lip lift) procedure, such as, e.g., a corner lip lift procedure. In certain aspects, marking system(s) comprise at least one independent marking used to facilitate a corner lip lift procedure accompanying a subnasal upper lip lift procedure.

In aspects, the at least one corner lip marking comprises an at least generally elliptical, at least substantially elliptical, at least essentially elliptical, essentially elliptical, or an elliptical shape. In aspects, the at least one corner lip marking comprises an at least generally fusiform, at least substantially fusiform, at least essentially fusiform, essentially fusiform, or a fusiform shape. In aspects, the at least one corner lip marking comprises an at least generally elliptical or fusiform, at least substantially elliptical or fusiform, at least essentially elliptical or fusiform, essentially elliptical or fusiform, or an elliptical or fusiform shape. In aspects, the at least one corner lip marking ends laterally at the oral commissure. In aspects, the at least one corner lip marking comprises an at least generally elliptical or fusiform, at least substantially elliptical or fusiform, at least essentially elliptical or fusiform, essentially elliptical or fusiform, or a fusiform shape ending laterally at the oral commissure. In aspects, marking(s) described in this paragraph are referred to herein as “Type I marking(s)” or “Type I marker(s)” ((042A) or (042B)) and are applicable to “Type I” corner lip lift procedure(s).

According to aspects, Type I marking(s) (042A) or (042B) guide a corner lip procedure when detectable or significant exposure of the lateral vermillion, detectable or significant lifting of the lateral vermillion, or both is/are the target aesthetic outcome(s) of the corner lip lift procedure.

In aspects, the at least one corner lip marking comprises an enclosed shape extending past the commissure. In aspects, the enclosed shape comprises a detectable or significant upward inflection. In aspects, the at least one corner lip marking comprises an enclosed shape extending past the commissure and comprising a detectable or significant upward inflection. In aspects, marking(s) described in this paragraph are referred to herein as “Type II marking(s)” or “Type II marker(s)” ((044A or (044B)) and are applicable to “Type II” corner lip lift procedures.

According to aspects, Type II marking(s) (044A) or (044B) guide a corner lip procedure when detectable or significant lifting of the commissure, detectable or significant neutralization of (improvement of or reduction in degree of) down-pointing rhytids extending inferior to the chelion, or when both detectable or significant lifting of the commissure and detectable or significant neutralization of (improvement of or reduction in degree of) down-pointing rhytids extending inferior to the chelion is/are target aesthetic outcome(s) of the corner lip lift procedure.

In aspects, point(s) on the upper vermillion border (020) of one or more radial marking lines of marking system(s) herein is/are used as a starting point for the establishment of marker(s) applicable to the performance of a corner lip lift procedure. In aspects, radial marking line(s) 4A (012A), 4B (012B), or both, as applicable to the side(s) of the subject's mouth undergoing the corner lip procedure, is/are used as a starting point for the establishment of one or more marker(s) applicable to the performance of a corner lip lift procedure. In aspects, one or more radial marking lines of marking system(s) herein, e.g., radial marking line(s) 4A (012A), 4B (012B), or both, is/are used as starting point(s) for the establishment of one or more marker(s) applicable to the performance of a corner lip lift procedure, wherein the marker(s) is/are at least generally horizontal, at least substantially horizontal, at least essentially horizontal, essentially horizontal, or is/are horizontal, e.g., at least generally straight, horizontal, at least substantially straight, horizontal, at least essentially straight, horizontal, essentially straight, horizontal, or is/are straight, horizontal marking line(s) drawn laterally from such point(s). These marking(s) are referred to as horizontal marking H-A (046A) and horizontal marking H-B (046B), respectively, discussed further elsewhere herein. In aspects, horizontal marking H-A (046A), horizontal marking H-B (046B), or both, establish a reference point, e.g., a reference point for establishing the upper limb of a corner lift, for use in performing a corner lift incision.

### Nasal Suspension

In aspects, one or more marking(s), e.g., one or more radial marking(s) of the marking system(s) provided herein, operate as a guide for one or more steps in the performance of a nasal base suspension procedure incorporating such system(s). In aspects, one or more radial markings establish one or more locations applicable to the conduct of a nasal base suspension procedure performed in addition to, e.g., ancillary to or concomitantly to, an upper lip lift procedure, e.g., subnasal upper lip lift procedure, e.g., a deep plane release upper lip lift modification procedure. In aspects, one or more of radial marking(s) 3A (010A), 3B (010B), 4A (012A), and 4B (012B) of marking system(s) described herein establish one or more location(s) applicable to the conduct of a nasal base suspension procedure, e.g., a nasal base suspension procedure performed on the subject in addition to (e.g., ancillary to or concomitant to) an upper lip lift procedure, e.g., subnasal upper lip lift procedure, such as a deep plane release upper lip lift procedure (e.g., with or without concomitant corner lip lift(s)).

According to certain aspects, marking system(s) comprise at least one marking not applicable to a primary procedure, e.g., an upper lip lift, e.g., a subnasal upper lip lift procedure, but which is applicable to a secondary procedure, e.g., an ancillary procedure or procedure performed concomitantly to the primary (e.g., subnasal upper lip lift) procedure, such as, e.g., a nasal base suspension procedure. In certain aspects, marking system(s) comprise at least one independent marking used to facilitate a nasal base suspension procedure accompanying a subnasal upper lip lift procedure (e.g., with or without concomitant corner lip lift(s)).

In aspects, a nasal base suspension procedure comprises passing at least one suture, e.g., typically 2 or sometimes 2 or more sutures, from inside the pyriform aperture out through the nasal labial muscle junction. In aspects, the nasal labial muscle junction is marked by radial marker(s) 4A (012A) and 4B (012B) on each respective side of a subject's mouth.

In aspects, a nasal base suspension procedure performed on a patient demonstrating hyperlaxity at locations indicated by radial markers 3A (010A), 3B (010B), or both, the dermis can be incorporated on a second pass of suture(s) as the suture(s) is/are reversed and passed back internally. Thus, in aspects, radial marker(s) 3A (010A), 4B (012B), or both identify the location wherein an assessment of hyperlaxity is appropriate to guide one or more action(s) taken in the nasal base suspension procedure.

### Muscle Suspension

In aspects, one or more marking(s), e.g., one or more radial marking(s) of the marking system(s) provided herein, operate as a guide for one or more steps in the performance of a muscle suspension procedure incorporating such system(s). In aspects, one or more radial markings establish one or more locations applicable to the conduct of a muscle suspension procedure performed in addition to, e.g., ancillary to or concomitantly to, an upper lip lift, e.g., subnasal upper lip lift procedure, e.g., a deep plane release upper lip lift modification procedure (e.g., with or without concomitant corner lip lift(s)). In aspects, one or more of radial marking(s) 1, 2A (008A), 2B (008B), 4A (012A), and 4B (012B) of marking system(s) described herein establish one or more location(s) applicable to the conduct of a muscle suspension procedure, e.g., a muscle suspension procedure performed on the subject in addition to (e.g., ancillary to or concomitant to) a subnasal upper lip lift procedure such as a deep plane release upper lip lift procedure (e.g., with or without concomitant corner lip lift(s)).

According to certain aspects, marking system(s) comprise at least one marking not applicable to a primary procedure, e.g., a muscle suspension procedure, but which is applicable to a secondary procedure, e.g., an ancillary procedure or procedure performed concomitantly to the primary (e.g., subnasal upper lip lift) procedure (e.g., with or without concomitant corner lip lift(s)), such as, e.g., a muscle suspension procedure. In certain aspects, marking system(s) comprise at least one independent marking used to facilitate a muscle suspension procedure accompanying a subnasal upper lip lift procedure (e.g., with or without concomitant corner lip lift(s)).

In aspects, a muscle suspension procedure is performed, comprising the incorporation of muscle plication. In aspects, suture(s), e.g., a plurality of suture(s), e.g., about 5 sutures, are used to perform the muscle plication by attaching to the pyriform ligament or periosteum in an e.g. mattress fashion at a plurality of locations indicated by a plurality of radial marker(s). In aspects, suture(s), e.g., a plurality of suture(s), e.g., about 5 sutures, are used to perform the muscle plication by attaching to the pyriform ligament or periosteum in an, e.g., mattress fashion at a plurality of locations indicated by radial marker 1 (006), 2A (008A), 2B (008B), 4A (012A), 4B (012B), or any combination of any or all thereof. Thus, in aspects, radial marker(s) 1 (006), 2A (008A), 2B (008B), 4A (012A), 4B (012B), or any/all thereof identify location(s) where one or more action(s) are to be taken during a muscle suspension procedure, e.g., where sutures are used to perform muscle plication. In aspects, such step(s) are only taken at locations identified by radial marker(s), e.g., at location(s) identified by radial markers 1, 2A (008A), 2B (008B), 4A (012A), 4B (012B), or any/all thereof.

In certain aspects, marking system(s) as described herein are physical marking(s) on the skin of a subject used as a guide during the performance of aesthetic modification procedure(s). However, also or alternatively, marking system(s) can be the parameter(s) which yield such marking(s), e.g., marking system(s) can be effectively virtual, wherein the rules for establishing such marking(s) are programmed and are applied programmatically and/or in an at least partially or completely automated fashion. In aspects, marking system(s) exist as a set of rules, algorithms, mathematical considerations, etc., wherein as a user defines one or more target effect(s), a virtual marking system is established which can then be used to guide a practitioner in the performance of an aesthetic modification procedure. In aspects, a translation of such a virtual marking system to a physical marking system is an element of the inventive system(s) and method(s) described herein. In aspects, such a translation can be a manual translation or an automated translation, such as, e.g., performed by automated technology(ies) such as robotics.

### Methods (General)

In aspects, the disclosure herein provides method(s) of achieving target aesthetic design feature(s) of the upper lip of a human subject, such as, e.g., a target vermillion slope or other design feature(s) (e.g., target aesthetic effect(s)) described herein. In aspects, the method(s) comprise (1) establishing a marking system having one or more characteristic(s) of marking system(s) provided herein; and (2) performing one or more aesthetic modification procedure(s) wherein the procedure(s) use, apply, reference during their conduct, or otherwise incorporate the marking system. According to aspects, marking system(s) herein represent a plurality of considerations required to achieve target effect(s). In aspects, such considerations are characterized as mathematical calculations or mathematical considerations or algorithms, as they are based upon the comparison of two or more elements or upon requirements for achieving specific end result(s), such as defined feature(s), such as slope, distance between anatomical location(s), relative positioning between anatomical location(s), etc.

In aspects, the disclosure herein provides method(s) of addressing age-related changes in the upper lip, wherein the method(s) comprise (1) establishing a marking system having one or more characteristic(s) of marking system(s) provided herein; and (2) performing one or more aesthetic modification procedure(s) wherein the procedure(s) use, apply, reference during their conduct, or otherwise incorporate the marking system.

In aspects, the disclosure herein provides method(s) of achieving target aesthetic design feature(s) of the upper lip, e.g., a target vermillion slope or other design feature(s) (e.g., target aesthetic effect(s)) described herein; addressing specific age-related changes in the upper lip in a human subject; or both, wherein the method(s) comprise (1) establishing a marking system having one or more characteristic(s) of marking system(s) provided herein; and (2) performing one or more aesthetic modification procedure(s) wherein the procedure(s) use, apply, reference during their conduct, or otherwise incorporate the marking system.

In aspects, the disclosure provides method(s) for addressing (e.g., detectably or significantly reducing or eliminating) difference(s), e.g., asymmetry(ies), in one or more feature(s) of the upper lip such as asymmetry(ies) associated with the nasal base, upper lip height, vermillion slope, philtrum, etc. (e.g., component(s) of the upper lip or marking system(s) referenced herein), wherein the method(s) comprise (1) establishing a marking system having one or more characteristic(s) of marking system(s) provided herein; and (2) performing one or more aesthetic modification procedure(s) wherein the procedure(s) use, apply, reference during their conduct, or otherwise incorporate the marking system. In aspects, such asymmetry(ies) can be naturally occurring or as a result of one or more prior aesthetic intervention(s), e.g., application of lip fillers, surgical procedure(s), or both.

In aspects, the disclosure provides method(s) of achieving target aesthetic design feature(s) such as a target vermillion slope, addressing age-related change(s) in the upper lip, or both, in a human subject wherein the method(s) further allow for addressing (e.g., detectably or significantly reducing or eliminating) difference(s), e.g., asymmetry(ies) across the upper lip, wherein the method(s) comprise (1) establishing a marking system having one or more characteristic(s) of marking system(s) provided herein; and (2) performing one or more aesthetic modification procedure(s) wherein the procedure(s) use, apply, reference during their conduct, or otherwise incorporate the marking system.

In aspects, the disclosure provides method(s) of aesthetically modifying the face of a human subject, wherein the method(s) comprise (1) establishing a marking system having one or more characteristic(s) of marking system(s) provided herein; and (2) performing one or more aesthetic modification procedure(s) wherein the procedure(s) use, apply, reference during their conduct, or otherwise incorporate the marking system.

In aspects, the disclosure provides method(s) of performing an upper lip lift procedure, e.g., subnasal upper lip lift procedure (e.g., with or without concomitant corner lip lift(s)), wherein the method(s) comprise (1) establishing a marking system having one or more characteristic(s) of marking system(s) provided herein directed to target aesthetic effect(s) to result from the procedure; and (2) performing an upper lip lift procedure, e.g., subnasal upper lip lift procedure (e.g., with or without concomitant corner lip lift(s)) wherein the procedure uses, applies, references during its conduct, or otherwise incorporates the marking system.

In aspects, the disclosure provides method(s) of performing a deep plane release upper lip aesthetic modification procedure, wherein the method(s) comprise (1) establishing a marking system having one or more characteristic(s) of marking system(s) provided herein directed to target aesthetic effect(s) to result from the procedure; and (2) performing a deep plane release upper lip aesthetic modification procedure wherein the procedure uses, applies, references during its conduct, or otherwise incorporates the marking system.

In aspects, the disclosure provides method(s) of performing a corner lip lift procedure, wherein the method(s) comprise (1) establishing a marking system having one or more characteristic(s) of marking system(s) provided herein directed to target aesthetic effect(s) to result from the procedure; and (2) performing a corner lip lift procedure wherein the procedure uses, applies, references during its conduct, or otherwise incorporates the marking system.

In aspects, the disclosure provides method(s) of performing a nasal base suspension procedure, wherein the method(s) comprise (1) establishing a marking system having one or more characteristic(s) of marking system(s) provided herein directed to target aesthetic effect(s) to result from the procedure; and (2) performing a nasal base suspension procedure wherein the procedure uses, applies, references during its conduct, or otherwise incorporates the marking system.

In aspects, the disclosure provides method(s) of performing a muscle suspension procedure, wherein the method(s) comprise (1) establishing a marking system having one or more characteristic(s) of marking system(s) provided herein directed to target aesthetic effect(s) to result from the procedure; and (2) performing a muscle suspension procedure wherein the procedure uses, applies, references during its conduct, or otherwise incorporates the marking system.

In aspects, the disclosure provides a method of establishing and further using, applying, referencing, or otherwise incorporating a marking system operating as a guide in aesthetic modification procedure(s), wherein the aesthetic modification procedure(s) is/are performed to attain predetermined target aesthetic effect(s) such as, e.g., to create a detectably or significantly improved balance in the appearance of the upper lip, to optimize muscle function, etc., wherein the marking system is any one or more of the marking system(s) described herein. In aspects, an aesthetic modification procedure is an upper lip lift procedure, such as, e.g., an upper lip lift procedure, e.g., subnasal upper lip lift procedure, e.g., a deep plane release upper lip lift procedure, and the use of, application of, reference to, or otherwise incorporation of the marking system indicate(s) whether/when an ancillary procedure, e.g., a corner lift procedure, nasal base suspension, muscle suspension, or combination thereof should be added/included in procedure to attain target aesthetic effect(s).

According to aspects, the method(s) provided herein comprise a step of evaluating the human subject for the presence of lip filler(s). In aspects, method(s) herein comprise a step for dissolving at least some, at least most, at least generally all, at least substantially all, at least essentially all, essentially all, or all lip filler(s) identified as present at least about 7 days (1 week), at least about 8 days, at least about 9 days, at least about 10 days, at least about 11 days, at least about 12 days, at least about 13 days, at least about 14 days (2 weeks), at least about 15 days, at least about 16 days, at least about 17 days, at least about 18 days, at least about 19 days, at least about 20 days, at least about 21 days (3 weeks), at least about 22 days, at least about 23 days, at least about 24 days, at least about 25 days, at least about 26 days, at least about 27 days, at least about 28 days (4 weeks), or more before an aesthetic modification procedure, e.g., subnasal upper lip lift procedure, e.g., with or without concomitant corner lip lift(s) (and or any other associated ancillary procedure(s) performed in association with a method).

In aspects, the marking system(s) provided herein are created/established prior to the start of any incision or excision performed as a part of procedure(s) incorporating the marking(s) of the marking system(s).

In certain aspects, step(s) of the method(s) described herein are performed by a human, e.g., a human medical practitioner. In aspects, the human performance of one or more step(s) of method(s) can be detectably or significantly aided by computer system(s), such as, e.g., robot assistance, by image analysis system(s) (e.g., in the evaluation of a subject as a step in establishing a marking system), mathematical analysis system(s), or combination(s) thereof. In aspects, the human performance of one or more step(s) of method(s) can be at least generally, at least substantially, at least essentially, essentially, or completely replaced by computer system(s), such as, e.g., robot assistance, by image analysis system(s) (e.g., in the evaluation of a subject as a step in establishing a marking system), mathematical analysis system(s), or combination(s) thereof. In aspects, steps(s) of the method(s) described herein utilize standard equipment known in the art, e.g., marking utensil(s), surgical instrument(s), equipment, and supplies, etc. In aspects, step(s) of the method(s) described herein utilize laser(s) and associated laser equipment, such as in the establishment of marking system(s) or for making incision(s)/excision(s). In aspects, one or more steps of the one or more methods described herein utilize marking utensil(s), surgical instrument(s), equipment, or supplies which are not typically used in the practice of similar aesthetic modification procedures. In aspects, one or more step(s) of methods) described herein utilize novel equipment provided herein, designed to address step(s) of method(s) described herein.

### Method of Facial Marking in Preparation for Performing Procedure(s)

In aspects, the disclosure provides method(s) of marking a human face with a marking system wherein the marking system indicates, communicates, or otherwise identifies one or more aesthetic modification(s) required to attain a target aesthetic effect. In aspects, the marking system resulting from the method is made in preparation for the performance of an aesthetic modification procedure, such as, e.g., an upper lip lift procedure, e.g., a subnasal upper lip lift procedure, e.g., a deep plane release upper lip lift modification procedure, and, optionally, one or more ancillary or concomitantly performed procedures (e.g., a corner lip lift, a nasal base suspension, a muscle suspension, or any combination of any or all thereof). In aspects, the marking system resulting from the method is used in, applied to, or otherwise is incorporated into the performance of (or, e.g., a method of performing) an aesthetic modification procedure (e.g., an upper lip lift procedure, e.g., a subnasal upper lip lift procedure, e.g., a deep plane release upper lip lift modification procedure), and, optionally, one or more ancillary or concomitantly performed procedures (e.g., a corner lip lift, a nasal base suspension, a muscle suspension, or any combination of any or all thereof). In aspects, the method of marking a human face with a marking system comprises any one or more of the step(s) provided herein.

In aspects, the disclosure provides method(s) of marking a human face with a marking system wherein the marking system establishes a visual guide. In aspects, the visual guide (marking system) comprises a set of markings, such as, e.g., at least about 3 markings, at least about 4 markings, at least about 5 markings, at least about 6 markings, at least about 7 markings, at least about 8 marking, at least about 9 markings, or, e.g., at least about 10 markings, or at least about 11 markings, such as, e.g., at least about 7 markings, on the face of a human subject and guides an upper lip lift procedure, e.g., subnasal upper lip lift procedure and, optionally, one or more ancillary procedure(s) (e.g., a subnasal and corner lip lift procedure).

FIGS. 2A-2O and FIGS. 3A and 3B provided herein exemplify the establishment of some, mostly all, generally all, substantially all, or all marking(s) which can be present in embodiment(s) of marking system(s) provided herein.

In aspects, the disclosure provides method(s) of marking a human face with a marking system wherein the method is performed with the patient maintained in an at least generally upright, at least substantially upright, at least essentially upright, essentially upright, or an upright position.

According to aspects, the disclosure provides method(s) of marking a human face comprising a step for assessing the vermillion body of the human subject for shape, previous scarring, volume, or any combination of any or all thereof prior to the application of any facial marking. In aspects, such an evaluation can be, as is described elsewhere herein, performed by a human. In aspects, such an evaluation can be, as is described elsewhere herein, performed by or at least in part aided by a computer or computer system. In aspects, such an evaluation can be, as is described elsewhere herein, performed by, or aided at least in part by tools incorporating use of artificial intelligence, facial recognition technology, or both.

In aspects, method(s) of marking a human face comprise(s) a step for marking the upper boundary of a portion of the upper lip to be excised in, e.g., an upper lip lift procedure, e.g., subnasal upper lip lift procedure, e.g., subnasal and corner lip lift procedure. In aspects, such an upper boundary follows horizontally along the subnasal, perialar crease. In aspects, the upper boundary of a portion of the upper lip to be excised comprises two peaks on either side of the midline at the divergence of the medial crural footplate and the nasal sill. In aspects, the marking for the upper boundary of a portion of the upper lip to be excised is, e.g., the upper boundary marker(s) described herein. In aspects, the upper boundary marker does not extend beyond the natural ending of the subnasal, perialar crease. In aspects, the upper boundary marker ends where the vector of the marking becomes vertical. In aspects, the upper boundary marker follows the border of the nasal skin and at least substantially avoids the mixture of nasal and labial skin via excision of nasal skin and transposition of lip skin into the nose during the upper lip lift procedure, e.g., subnasal upper lip lift procedure, e.g., subnasal and corner lip lift procedure. FIG. 2A, provided herein, illustrates an exemplary upper excision boundary marker (002), the marker being the 2-peaked, wave-like line following the subnasal, perialar crease. FIG. 2A includes, e.g., part(s) of radial markers 1 (006), 2A (008A), and 2B (008B). FIG. 2A further indicates the upper vermillion border (020). FIGS. 3A and 3B provide an exemplary upper boundary excision marker accompanied by several other markers of a marking system.

In aspects, method(s) of marking a human face comprise(s) a step for marking a central marking line. In aspects, the central marking line is an at least generally, at least substantially, at least essentially, essentially, or is a vertical line passing down the middle of the philtral trough. In aspects, the central marking line is, e.g., radial markers 1 (006) described herein. In aspects, the central marking line, e.g., radial marking line 1 (006), is made while holding the upper lip on a gentle stretch. In aspects, this marking does not initially, e.g. at this point in the method of marking a human face, span the entirety of the upper lip from the point of the upper excision boundary marker (002) to the upper vermillion border (020). FIG. 2A, provided herein, illustrates an indicator of the location of radial marking line 1 (radial marker 1) (006), indicated by the central hashmark along the upper excision boundary marker (002). A portion of radial marking line/radial marker 1(006) is shown in, e.g., FIG. 2G, with the portion of the marker shown as extending centrally superior (caudal) from the lower extension boundary marker, and in FIG. 2M, in a final form, extending centrally fully to the upper vermillion border (020). FIGS. 3A and 3B illustrate an exemplary radial marking line 1 (006) accompanied by several other markers of a marking system.

In aspects, method(s) of marking a human face comprise(s) a step for marking at least one, such as, e.g., 2 paramedian/paramedial line(s) each respectively positioned laterally to radial marking line 1 (006) on opposing (e.g., first and second) sides of radial marker 1 (006). In aspects, the at least 1, e.g., 2 paramedian line(s) is/are positioned along the subnasal perialar crease (e.g., the upper boundary marker), wherein each of the paramedian/paramedial line(s) is located on the first, second, or both philtral column(s), respectively. In aspects, the paramedian/paramedial line(s) are radial marking line(s) 2A (008A), 2B (008B), or both described elsewhere herein. In aspects, radial marking line(s) 2A (008A), 2B (008B), or both are made while gently stretching the lip. In aspects, radial marking line(s) 2A (008A), 2B (008B), or both are made using the assistance of an instrument such as a caliper. In aspects, radial marking line(s) 2A (008A), 2B (008B), or both (and, optionally, one or more additional line(s) of marking system(s) provided herein) are made with a right-angle caliper, such as a right-angle caliper described herein. In aspects, this/these marking(s) does/do not initially, e.g., at this point in the method of marking a human face, span the entirety of the upper lip from the point of the upper boundary marker to the upper vermillion border (020). FIG. 2A, provided herein, illustrates an indicator of the locations of radial marking lines 2A (008A) and 2B (008B) (radial markers 2A (008A) and 2B (008B)), indicated by the hashmark(s) to the left and right of the central mark (indicating the position of radial marker 1 (006)) along the upper excision boundary marker (002). A portion of radial marking line(s)/radial marker(s) 2A (008A) and 2B (008B) are shown in, e.g., FIG. 2G, with the portion of the markers shown as extending centrally superior (caudal) from the lower extension boundary marker, just lateral to the central marking line (portion of radial marker 1 (006)), and in FIG. 2M, in final form(s), each extending just lateral to the left and right of central marking line 1 (006) fully to the upper vermillion border (020). FIGS. 3A and 3B illustrate exemplary radial marking lines 2A (008A) and 2B (008B) accompanied by several other markers of a marking system.

In aspects, method(s) of marking a human face comprise(s) a step for marking at least one, such as, e.g., 2 lines positioned along the subnasal perialar crease (e.g., the upper boundary marker) demarcating the lateral extent of the vertical lift effect of a subnasal lip lift on the upper vermillion border (020). In aspects, such marking lines are placed lateral to each of the marking lines 2A (008A), 2B (008B), or both. In aspects, these laterally placed marking(s) are placed lateral to an additional set of markings, described elsewhere herein (e.g., radial markings 3A (010A), 3B (010B), or both). In aspects, these laterally placed markings are radial markings 4A (012A), 4B (012B), or both. In aspects, this/these marking(s) do not initially, e.g., at this point in the method of marking a human face, span the entirety of the upper lip from the point of the upper boundary marker to the upper vermillion border (020). In aspects, this/these marking(s) does/do not initially, e.g., at this point in the method of marking a human face, span the entirety of the upper lip from the point of the upper boundary marker to the upper vermillion border (020). Radial marking line(s)/radial marker(s) 4A (012A) and 4B (012B) are shown in, e.g., FIG. 2B, superiorly (caudally) from the lower excision boundary marker (004) to the upper vermillion border (020), and in FIG. 2M, in final form(s), as labeled. FIGS. 3A and 3B illustrate exemplary radial marking lines 4A (012A) and 4B (012B), accompanied by several other markers of a marking system.

In aspects, method(s) of marking a human face comprise(s) a step for marking at least one, such as, e.g., 2 lines positioned along the subnasal perialar crease (e.g., the upper boundary marker) from the nasal sill and extending to the upper vermillion border (020). In aspects, such lines are positioned between radial marking lines 2A (008A) and 4A (012A) and between radial marking lines 2B (008B) and 4B (012B), respectively. In aspects, such marking line(s) are radial marking line(s) 3A (010A), 3B (010B), or both. In aspects, this/these marking(s) does/do not initially, e.g., at this point in the method of marking a human face, span the entirety of the upper lip from the point of the upper boundary marker to the upper vermillion border (020). A portion of radial marking line(s)/radial marker(s) 3A (010A) and 3B (010B) are shown in, e.g., FIG. 2G, with the portion of the markers shown as extending superiorly (caudal) from the lower extension boundary marker, just lateral to each of radial marker(s) 2A (008A) and 2B (008B), and in FIG. 2M, as labeled. FIGS. 3A and 3B illustrate exemplary radial marking lines 3A (010A) and 3B (010B), accompanied by several other markers of a marking system.

In aspects, method(s) of marking a human face comprise(s) a step for determining or considering one or more aesthetic result(s) to be achieved by the performance of an aesthetic modification procedure comprising the use of a facial marking system established by performance of the method. In aspects, an aesthetic result can be, e.g., any target aesthetic effect, such as, e.g., a visual balance of two or more facial features including the size of the upper lip to the nose, the size of the upper lip to the chin, the size of the upper lip to the remainder of the face, or a combination thereof; a target incisor display; a target final lip height; a target final philtral height; the accent and definition of the Cupid's bow of the subject, the upper vermillion border (020) of the subject, or both; a target volume show of the subject's red vermillion; a target height differential along the upper vermillion border (020) between two or more of radial markings 2A (008A) and 3A (010A), 2B (008B) and 3B (010B), 3A (010A) and 4A (012A), 3B (010B) and 4B (012B), or a combination thereof; or any combination thereof.

In certain aspects, the evaluation of target aesthetic results, e.g., the determination or consideration of one or more aesthetic effect(s) is made or revisited after the placement of one or more of, e.g., 2 or more of, 3 or more of, 4 or more of 5 or more of, 6 or more of, 7 or more of, 8 or more of, or all of upper boundary marker, radial marker 1 (006), radial marker 2A (008A), radial marker 2B (008B), radial marker 4A (012A), radial marker 4B (012B), radial marker 3A (010A), and radial marker 3B (010B).

In aspects, the placement of, e.g., the positioning of one or more additional marker(s) is made based on the target aesthetic effect(s). In aspects, for example, the positioning of a lower excision boundary marker (004) is adjusted across its lateral span to accommodate for and achieve target aesthetic effect(s). In aspects, development of the lower excision boundary marker (004) is made point-by-point, e.g., by marking an incision point across the radial marker(s) required to yield a lower excision boundary marker (004), once connected, which yields a target aesthetic appearance. That is, in aspects, a lower excision boundary marker (004) is established by marking an incision point along each radial marker, establishing an excision height and a final lip height at the location of each radial marker, which, once excision is complete as part of an excision procedure, yields the target aesthetic effect(s). In aspects, the development of the lower excision boundary marker (004) comprises connecting the incision points indicated across each respective radial marking line. In aspects, method(s) of marking a human face described herein comprise determining the height of an excision to be made at a plurality of locations across a portion of the upper lip to be excised in an upper lip lift procedure, e.g., subnasal upper lip lift procedure, e.g., subnasal and corner lip lift procedure, required to achieve target aesthetic effect(s). In aspects, method(s) comprise marking the location between the nasal sill (e.g., indicated by the upper excision boundary marker (002)) and the upper vermillion border (020) along the projected course of each of two or more of, e.g., three or more of, four or more of, five or more of, six or more of, or all radial marking lines (e.g., radial marking lines 1, 2A (008A), 2B (008B), 3A (010A), 3B (010B), 4A (012A), and 4B (012B) or any combination of two, three, four, five, six or more thereof or all of radial marking lines 1, 2A (008A), 2B (008B), 3A (010A), 3B (010B), 4A (012A), and 4B (012B)) wherein the location indicates the lower excision boundary required to achieve the target aesthetic result(s)/effect(s). In aspects, this marker is the lower excision boundary marker (004) described elsewhere herein. In aspects, the lower excision boundary marker (004) establishes a length between the given location(s) and the point along the upper vermillion border (020) identified by the projected course of each of the radial marking lines 1, 2A (008A), 2B (008B), 3A (010A), 3B (010B), 4A (012A), and 4B (012B) which represents the target final lip height at the location of that radial marking(s). In aspects, the lower excision boundary marker (004) establishes an excision height at each radial marking, identified as the distance between the upper excision boundary marker (002) and the lower excision boundary marker (004) at the location of each radial marking line. FIG. 2C illustrates 3 incision points (provided as black dots) contributing to the lower boundary marker; from left to right (presuming here, that Group A markers are presented to the left as viewing the images and Group B markers are presented to the right as viewing the images), such incision points being along radial markers 2A (008A), 1 (006), and 2B (008B), respectively. FIG. 2F illustrates additional incision points contributing to the lower boundary marker, indicated as an upper set of black dots; from left to right, as described above, such incision points being positioned along radial markers 4A (012A), 3A (010A), 2A (008A), 1, 2B (008B), 3B (010B), and 4B (012B). FIG. 2G, for example, illustrates a completed lower excision boundary marker (004), as does, e.g., FIG. 2M.

FIGS. 3A and 3B illustrate an exemplary lower excision boundary marker (004) accompanied by several other markers of a marking system. FIGS. 2D and 2E illustrate the use of a marking tool used in embodiment(s) to establish the position of incision points (provided as black dots), e.g., incision points associated with radial marker 2A (008A) (FIG. 2D) and along radial marker 2B (008B) (FIG. 2E), wherein incision points along radial marker 2A (008A) and along radial marker 2B (008B) are used to help establish the lower excision boundary marker (004).

In aspects, the method(s) of marking a human face described herein comprise a step for extending one or more earlier/previously made marking(s). In aspects, method(s) of marking a human face described herein comprise(s) a step for completing a remainder of one or more of earlier/previously made radial marking(s) 1, 2A (008A), 2B (008B), 3A (010A), 3B (010B), 4A (012A), and 4B (012B) from the point of the lower excision boundary marker (004) to its corresponding point along the upper vermillion border (020). At this point in the method(s), marking lines may, in aspects, extend as an uninterrupted line from at or above the upper excision boundary marker (002) to the upper vermillion border (020). In aspects, at this point in the method(s), marking lines may comprise a gap representing the excision height (e.g., the excision height “segment” of a radial marking line; this “segment”, described elsewhere herein, can be present and measurable, having an excision height segment length even if such “segment” is not drawn), the excision height portion(s) or segment(s) of radial marking line(s) being described elsewhere herein. FIG. 2F indicates points along the upper vermillion border (020), provided as a lower set of black dots, for establishing the completed radial markings, as do, e.g. FIG. 2G, FIG. 2H, FIG. 21, FIG. 2J, FIG. 2K, and FIG. 2L, wherein in FIGS. 2G-2L, each radial marking is presented as partially complete. FIG. 2M presents an embodiment of the radial marking(s) having been completed. FIGS. 3A and 3B illustrate exemplary completed radial marking lines of a marking system.

According to certain aspects, if a target aesthetic effect is a detectable or significant improvement in the asymmetry of the philtral column height, marking(s) of the marking system, e.g., marking(s) of the guide, are made by (1) measuring the residual height of a side of the philtral column identified as comprising a desirable height, and (2) applying the same marking to the contralateral side without the placement of tension on the lip.

In some aspects, method(s) of marking a human face comprise, as part of evaluating the height of the excision across the portion of the upper lip to be excised in the upper lip lift procedure, e.g., subnasal upper lip lift procedure, e.g., subnasal and corner lip lift procedure required to achieve the target aesthetic result(s) (wherein one such target aesthetic result is a constant slope of the upper vermillion) comprises the application of a mathematical analysis. In certain aspects, evaluating the positioning of the lower excision boundary marker (004) and marking of the same along one or more radial marking lines comprise(s) consideration of the lateral spacing along the upper vermillion border (020) between two or more neighboring radial markings. In aspects, method(s) comprise the application of Pythagoras' theorem a²+b²=c² to determine the appropriate length of the excision height of one or more radial marking lines, given the lateral distance between two or more neighboring radial markings required to attain a constant target slope of the upper vermillion border (020). Use of Pythagoras' theorem and its application to determining length(s) of at least portion(s) of radial marking line(s) is described elsewhere herein. As is also described elsewhere herein a target slope is achieved by establishing a marking system wherein a height difference between the points of the at least two neighboring radial markings along the upper vermillion border (020) of between about 1 mm and about 5 mm, between about 1 mm and about 4 mm, between 1 mm and about 3 mm, or between about 1 mm and about 2 mm, such as, e.g., about 2 mm will result from the incorporation of the guiding marking system in the performance of the aesthetic modification procedure, e.g., upper lip lift procedure, e.g., subnasal upper lip lift procedure, e.g., subnasal and corner lip lift procedure. FIG. 7 herein illustrates this concept by superimposing a right triangle upon the upper lip, illustrating that the hypotenuse (“C”) of the triangle is longer than, e.g., side “A” of the triangle.

In aspects, method(s) of marking a human face comprise(s) marking at least 2 at least mostly horizontal, at least generally horizontal, at least substantially horizontal, at least essentially horizontal, essentially horizontal, or horizontal extension(s) of one or more radial marker(s). In aspects, method(s) of marking a human face comprise(s) marking at least 2 at least mostly horizontal, at least generally horizontal, at least substantially horizontal, at least essentially horizontal, essentially horizontal, or horizontal lateral line marking(s). In aspects, method(s) of marking a human face comprise(s) marking at least 2 at least mostly horizontal, at least generally horizontal, at least substantially horizontal, at least essentially horizontal, essentially horizontal, or horizontal lateral line marking(s) which are extension(s) of point(s) along radial marking line(s) 4A (012A), 4B (012B), or both. In aspects, method(s) of marking a human face comprise(s) marking at least 2 at least mostly horizontal, at least generally horizontal, at least substantially horizontal, at least essentially horizontal, essentially horizontal, or horizontal lateral line marking(s) which are extension(s) of the point along radial marking line(s) 4A (012A), 4B (012B), or both and which each extend toward radial marking line(s) 5A (014A) or 5B (014B), respectively as applicable. In aspects, these marker(s) are markings LE-A (018A) and LE-B (018B) as described elsewhere herein. In certain aspects, method(s) of marking a human face comprise(s) marking 1 or more, e.g., 2 at least generally horizontal, at least substantially horizontal, at least essentially horizontal, essentially horizontal, or horizontal line extensions beginning at the intersection of each of radial markings 4A (012A), 4B (012B), or both (as present) with the lower excision boundary marker (004) (e.g., at the lower incision point along each of radial markings 4A (012A), 4B (012B), or both (as present)), and extend(s) laterally therefrom. In aspects, such markings, LE-A (018A) and LE-B (018B) as described elsewhere herein, are useful in concomitantly performed procedure(s) such as, e.g., corner lip lift procedure(s). Exemplary markings LE-A (018A) and LE-B (018B) are illustrated in Figures provided herewith, such as, e.g., in FIGS. 2H and 3A.

In certain aspects, method(s) of marking a human face comprise(s) marking 2 radial markings positioned lateral to each of radial markings 4A (012A) and 4B (012B), respectively. In aspects, such markings follow the RSTL. In aspects, such markings extend downward from the apex of the upper boundary of the excision. In aspects, these marking(s) are radial marking 5A (014A), radial marking 5B (014B), or both. FIGS. 2I, 2M, and 3, as labeled, provide exemplary embodiments of radial markers 5A (014A) and 5B (014B).

In aspects, method(s) of marking a human face comprise(s) marking 2 at least mostly horizontal, at least generally horizontal, at least substantially horizontal, at least essentially horizontal, essentially horizontal, or horizontal extension(s) of one or more radial marker(s). In aspects, method(s) of marking a human face comprise(s) marking 2 at least mostly horizontal, at least generally horizontal, at least substantially horizontal, at least essentially horizontal, essentially horizontal, or horizontal lateral line marking(s). In aspects, method(s) of marking a human face comprise(s) marking 2 at least mostly horizontal, at least generally horizontal, at least substantially horizontal, at least essentially horizontal, essentially horizontal, or horizontal lateral line marking(s) which are extension(s) of one or more point(s) along radial marking line(s) 4A (012A), 4B (012B), or both. In aspects, method(s) of marking a human face comprise(s) marking 2 at least mostly horizontal, at least generally horizontal, at least substantially horizontal, at least essentially horizontal, essentially horizontal, or horizontal lateral line marking(s) which are extension(s) of the point along radial marking line(s) 4A (012A), 4B (012B), or both and which each extend toward radial marking line(s) 5A (014A) or 5B (014B), respectively as applicable. In aspects, these marker(s) are markings H-A (046A) and H-B (046B) as described elsewhere herein. In aspects, H-A (046A), H-B (046B), or both, are part of a Type II corner lift marking (044A) and (044B), respectively. In certain aspects, radial marking(s) 5A (014A), 5B (014B), or both, and markings H-A (046A), H-B (046B), or both, or combination(s) thereof, are made after the performance of a subnasal upper lip lift procedure is complete. In aspects, making marking(s) 5A (014A), 5B (014B), H-A (046A), H-B (046B), or combination(s) thereof after the performance of a subnasal upper lip lift procedure (comprising use of marking system(s) and method(s) of their generation described herein) ensures that the outcome of the subnasal upper lip lift procedure is assessed and considered, e.g., taken into account, when deciding if/how to perform an ancillary procedure, such as, e.g., an ancillary or concomitantly performed corner lip lift procedure.

According to certain aspects, method(s) of marking a human face with a marking system (wherein the marking system indicates, communicates, or otherwise identifies one or more aesthetic modification(s) required to attain a target aesthetic effect), when applied to an upper lip lift procedure, yield(s) an excision height disparity between the excision height along radial marking 2A (008A) (2A-EX) or 2B (008B) (2B-EX) marking and a 4A (012A) or 4B (012B) marking of no more than about 8 mm, such as, no more than about 7 mm, no more than about 6 mm, no more than about 5 mm, no more than about 4 mm, such as, e.g., about 2 mm to about 4 mm, about 2 mm to about 4 mm, or, e.g., about 3 mm to about 4 mm. In aspects, a disparity of more than about 4 mm can cause difficulty in redistributing the inferior relative to the superior flap.

According to certain aspects, method(s) of marking a human face with a marking system that indicates, communicates, or otherwise identifies one or more aesthetic modification(s) required to attain a height difference between the points of the at least two neighboring radial markings along the upper vermillion border (020) of between about 1 mm and about 5 mm, between about 1 mm and about 4 mm, between 1 mm and about 3 mm, or between about 1 mm and about 2 mm, such as, e.g., about 2 mm, yielding an at least generally constant, at least substantially constant, at least essentially constant, essentially constant, or constant slope of the upper vermillion border (020).

According to certain aspects, method(s) of marking a human face with a marking system that indicates, communicates, or otherwise identifies one or more aesthetic modification(s) required to attain a length of incisor display (“tooth show”) of between about 1 and about 5 mm, such as, e.g., ˜1 mm-˜4 mm, ˜1 mm-˜3 mm, or ˜1 mm-˜2 mm, such as, ˜2 mm-˜5 mm, ˜3 mm-˜5 mm, or ˜4 mm-˜5 mm, such as, e.g., ˜2 mm-˜4 mm, such as, e.g., about 1 mm, ˜2 mm, or ˜3 mm.

FIGS. 2J, 2K, and 2L illustrate further marking(s) which can be made in method(s) of marking a human face with a marking system provided herein, such as, e.g., marking RSTL line(s). FIG. 2J (straight face), FIG. 2K (smiling face), and FIG. 2L (puckered lips) present embodiments wherein RSTL lines/markings are included in a marking system. The RSTL globe boundary is indicated by the nasolabial folds (016A) and (016B) as provided in, e.g., FIGS. 2J and 3A.

### Method(s) of Evaluation and Characterization (Method of Assessment) to Achieve Target Aesthetic Appearance(s)

In aspects, the disclosure provides a method of assessing a human subject in preparation for aesthetic modification of the upper lip and accordingly characterizing surgical requirements for an upper lip lift procedure, e.g., subnasal upper lip lift procedure, e.g., subnasal and corner lip lift procedure. In aspects, such evaluation is conducted using the assistance of, or is conducted completely by, automated system(s) disclosed herein.

According to certain aspects, the disclosure provides method(s) evaluating the face of a human subject to identify target aesthetic effect(s) in preparation for aesthetic modification of the subject's upper lip by application of an upper lip lift procedure, e.g., subnasal upper lip lift procedure, e.g., subnasal and corner lip lift procedure addressed thereto and characterizing one or more surgical requirements required in the procedure such that the procedure results in the target aesthetic appearance, wherein the characterization of one or more surgical requirements required to attain the target aesthetic appearance is provided as a marking system used as a guide in the performance of the procedure. In aspects, such method(s) can be referred to as method(s) of assessment, wherein “assessment” refers to both “evaluation” and “characterization.” In aspects, the method(s) of assessment comprise(s) evaluating one or more features of the human subject's face. In aspects, the method(s) of assessment comprise(s) identifying a target aesthetic appearance. In aspects, the method(s) of assessment comprise(s) identifying one or more surgical modifications required to be made in an upper lip lift procedure, e.g., subnasal upper lip lift procedure, e.g., subnasal and corner lip lift procedure, to achieve the target aesthetic appearance. In aspects, the method(s) of assessment comprise(s) establishing a marking system having any one or more of the characteristic(s) described herein. In aspects, the method(s) of assessment comprise(s) (1) evaluating one or more features of the human subject's face; identifying a target aesthetic appearance; identifying one or more surgical modifications required to be made in an upper lip lift procedure, e.g., subnasal upper lip lift procedure, e.g., subnasal and corner lip lift procedure to achieve the target aesthetic appearance; and establishing a marking system having any one or more of the characteristic(s) described herein. In aspects, such method(s) of assessment are used in, applied to, or otherwise incorporated into method(s) of performing one or more aesthetic modification procedure(s) described herein, such as, e.g., an upper lip lift, e.g., a subnasal upper lip lift, e.g., a deep plane release subnasal upper lip lift, e.g., a subnasal or deep plane release subnasal upper lip lift accompanied by one or more ancillary procedure(s) such as corner lip lift, nasal base suspension, or muscle suspension procedure(s) (e.g., a subnasal and corner lip lift procedure).

According to certain aspects, method(s) of assessment provide a mathematical guide for maintaining the natural balance of the upper lip, for optimizing muscle function, indicating when to add a secondary (e.g., ancillary or e.g. concomitantly performed) procedure such as a corner lip lift, or any combination of any or all thereof. In aspects, the application of a mathematical guide detectably or significantly improves upon the consistency of procedural outcome(s) compared to procedure(s) performed without use of such mathematical analysis/consideration and or mathematically determined guide(s)/marking system(s). In aspects, the method(s) provided herein, e.g., the method(s) of assessment provided herein, represent a novel approach to lip lift design. In aspects, method(s) provided herein, e.g., method(s) of assessment provided herein, provide for a detectably or significantly improved lip balance; a detectable or significant improvement in facial harmony; a detectable or significant improvement in tooth show; a suitable, e.g., sufficient, adequate, or otherwise desirable (e.g., targeted) tooth show; a suitable, e.g., sufficient, adequate, or otherwise desirable (e.g., targeted) exposure of the lateral vermillion; or any combination of any or all thereof.

In aspects, the method(s) of assessment provide a detectable or significant ability to detectably or significantly control lip shape during and resulting from the subnasal lip lift procedure compared to such procedure(s) performed without one or more step(s) of the method(s) described here. As one example, method(s) of assessment provided by the disclosure result(s) in the establishment of guide(s), e.g., marking system(s) for use before, during, or both before and during a subnasal lip lift procedure and wherein the guide(s)/marking system(s) establish and control the shape of the upper vermillion resulting from the subnasal lip lift procedure. In a specific example, the guide(s) marking system(s) provided by the method establish a target slope of the upper vermillion resulting from the subnasal lip lift procedure wherein the slope of the upper vermillion border (020) is the vertical descent of the upper vermillion border (020) from the Cupid's peak following laterally toward the oral commissure when viewing the human subject face-on.

In aspects, method(s) of assessment provided herein provide detectable or significant control over the establishment of the slope of the upper vermillion border (020) resulting from a subnasal lip lift procedure, wherein the slope of the upper vermillion border (020) is the vertical descent of the upper vermillion border (020) from the Cupid's peak following laterally toward the oral commissure when viewing the human subject face-on.

In aspects, one or more step(s) of the method(s) of assessment provided herein can be performed by a human. In aspects, the method(s) of assessment provided herein can be performed by a human aided by one or more other tool(s) or system(s), such as one or more computer- or software-related tool(s) or system(s). In aspects, the method(s) of assessment provided herein can be performed by a human aided by one or more robotic tool(s) or system(s). In aspects, the method(s) of assessment provided herein can be performed by a tool(s) or system(s) incorporating the use of artificial intelligence. In aspects, one or more steps(s) of the method(s) of assessment provided herein can be performed at least mostly without, at least generally without, at least substantially without, at least essentially without, essentially without, or without human involvement. In aspects, the method(s) of assessment provided by the disclosure comprise(s) use of preoperative photography. In aspects, preoperative photography detectably or significantly improves upon the assessment of (detectably or significantly increases the sensitivity in identifying) a requirement for asymmetric excision to be utilized in the upper lip lift procedure, one or more ancillary procedure(s) performed in association with the upper lip lift procedure, or both. Accordingly, in aspects, the disclosure provides method(s) of assessment wherein the method(s) comprise(s) the application of one or more automated, non-human, or computer-based system(s) comprising software-driven analysis to, e.g., application of artificial intelligence of, a scan of, an image of, a live presentation of, or any combination thereof, the subject's face to establish one or more feature(s) of one or more marking(s) of the guide/marking system used in the method(s). Such automated system(s) and automated method(s) are further discussed elsewhere herein.

According to aspects, the method(s) of assessment provided herein comprise viewing the profile view of the subject. In aspects, considering the profile view of the subject allows for consideration of slope and projection as step(s) used in establishing target aesthetic effect(s), e.g., a target aesthetic appearance, and to identify one or more surgical requirement(s) required to attain the target aesthetic effect(s)/target aesthetic appearance.

In aspects, the method(s) of assessment described herein is/are applied in one or more surgical techniques wherein the alar sill and nasal base are maintained in an intact anatomic subunit and are not detectably or significantly crossed or violated.

In aspects, method(s) of assessment comprise(s) a step of marking the contour of the nasal base to establish a first generally horizontal indicator (“LEVEL 1 indicator”), wherein a third generally horizontal indicator (“LEVEL 3 indicator”) is formed by the upper vermillion border (020). In aspects, the “LEVEL 1 indicator” is the upper excision boundary marker (002) described elsewhere herein.

In aspects, method(s) of assessment comprise(s) step(s) for marking the anatomy of the upper lip of the human subject with at least 1, at least 2, at least 3, at least 4, at least 5, at least 6, at least 7, at least 8, or, e.g., at least 9, such as typically at least 7 radially-spaced markings (radial markings), wherein the at least 7 radial markings progress from at least substantially vertical at the midline point of the Cupid's bow trough with gradual radial rotation laterally towards the angle of the nasolabial fold(s) (016A) and (016B) (respectively), following the lip's natural relaxed tension lines, pores, or both. In aspects, these radial marking(s) are the radial marking(s) described herein, e.g., radial markers 1, 2A (008A), 2B (008B), 3A (010A), 3B (010B), 4A (012A), 4B (012B), 5A (014A), 5B (014B), or any combination of any or all thereof, wherein radial marker 1 (006) is at least generally vertical, at least substantially vertical, at least essentially vertical, is essentially vertical, or is vertical, and some, most, generally all, substantially all, or all of markings 2A (008A), 3A (010A), 4A (012A), and 5A (014A) gradually radially rotate, at least in part, laterally toward the angle of the nasolabial fold (016A) on one side of radial marker 1 (006), and some, most, generally all, substantially all, or all of markings 2B (008B), 3B (010B), 4B (012B), and 5B (014B) gradually radially rotate, at least in part, laterally toward the angle of the nasolabial fold (016B) on one side of radial marker 1 (006).

In aspects, method(s) of assessment comprise(s) a step for marking the anatomy of the upper lip with a first radial marking, radial marking 1 (006) described herein, positioned in the middle of the columella and philtral dimple, at the lowest point of the trough of Cupid's bow.

In aspects, method(s) of assessment comprise(s) a step for marking the anatomy of the upper lip with one or two radial markings, radial marking 2A (008A), radial marking 2B (008B), or both, positioned lateral to and on either side of radial marking 1 (006), respectively, each located lateral to the columella, at the peaks of Cupid's bow and at the point where the medial crural footplates diverge into the nose at the junction with the alar sill.

In aspects, method(s) of assessment comprise(s) a step for marking the anatomy of the upper lip with one or two radial markings, radial marking 3A (010A), radial marking 3B (010B), or both, positioned lateral to each of radial markings 2A (008A) and 2B (008B), respectively, such that the two radial markings 3A (010A) and 3B (010B) are positioned centrally between radial markings 2A (008A) and 4A (012A) and between radial markings 2B (008B) and 4B (012B), respectively.

In aspects, method(s) of assessment comprise(s) a step for marking the anatomy of the upper lip with one or two radial markings, radial marking 4A (012A), radial marking 4B (012B), or both, positioned lateral to each of radial markings 3A (010A) and 3B (010B), respectively, each following the natural tension lines, radial angulation of the pores of the upper lip, or both.

In aspects, method(s) of assessment comprise(s) a step for marking the anatomy of the upper lip with one or two radial markings, radial marking 5A (014A), radial marking 5B (014B), or both, established and positioned lateral to each of radial markings 4A (012A) and 4B (012B) and following each of the relaxed skin tension lines (RSTLs), extending downward from the apex of the lateral end of the upper excision to be made at the nasal base.

In certain aspects, radial markings 5A (014A) and 5B (014B) establish one or more locations applicable to the conduct of a supplemental procedure to the upper lip lift, e.g., subnasal upper lip lift procedure, e.g., an ancillary procedure or a concomitantly performed procedure described herein. In certain specific aspects, radial markings 5A (014A) and 5B (014B) establish one or more locations for reference in a corner lift procedure performed on the subject, in addition to the subnasal upper lip lift procedure.

According to aspects, the method(s) of evaluation provided herein comprise a step for evaluating for the need to supplement a subnasal upper lip lift procedure with one or more ancillary procedures to attain a desired aesthetic appearance. In aspects, method(s) of evaluation comprise making at least one independent marking which facilitates a corner lip lift procedure accompanying the subnasal upper lift procedure. In aspects, the at least one independent corner lip lift marking comprises either (1) an at least substantially elliptical or fusiform shape and ending laterally at the oral commissure (Type I marking (042A) or (042B) described elsewhere herein), or (2) an enclosed shape extending past the commissure and comprising an upward inflection (Type II marking ((044A) or (044B)) described elsewhere herein). In aspects, Type I marking(s) ((042A) or (042B)) are a guide in a corner lip procedure targeting exposure and lift of the lateral vermillion. In aspects, Type II marking(s) are a guide in a corner lip procedure targeting lift of the commissure, neutralization of down-pointing rhytids extending inferior to the chelion, or both lift the commissure and neutralization of down-pointing rhytids extending inferior to the chelion. In aspects, Type I markings ((042A) or (042B)), Type II markings ((044A) and (044B)), or both are made as a step in the method(s) if the evaluation for the need to supplement a subnasal upper lip lift procedure with a corner lip lift procedure is deemed necessary to achieve a desired target aesthetic appearance.

In aspects, method(s) of assessment comprise identifying point(s) on the upper vermillion border (020) of radial marking lines 4A (012A) and 4B (012B), as applicable to the side(s) of the subject's mouth undergoing the corner lip procedure, as a starting point and making an at least generally straight, at least substantially straight, at least essentially straight, essentially straight, or straight at least generally horizontal, at least substantially horizontal, at least essentially horizontal, essentially horizontal, or horizontal marking line drawn laterally from such point(s), e.g., horizontal marking H-A (046A) and horizontal marking H-B (046B), respectively, described elsewhere herein. In aspects, such marking(s) (e.g., horizontal marking H-A (046A), horizontal marking H-B (046B), or both) establish a reference point for use in performing an ancillary or concomitantly performed corner lift incision.

According to certain aspects, method(s) of assessment comprise identifying that an ancillary corner lift procedure in addition to a subnasal upper lip lift procedure is necessary to achieve target aesthetic effect(s)/target aesthetic appearance, and wherein the final upper lip height of radial markings 5A (014A), 5B (014B), or 5A (014A) and 5B (014B) (as applicable to the corner(s) of a/the mouth being lifted by a corner lift procedure) are established such that they are at least generally similar to/same as, at least substantially similar to/same as, at least essentially similar to/same as, are essentially similar to/same as, or are the same as the final upper lip height of corresponding radial markings 4A (012A), 4B (012B), or 4A (012A) and 4B (012B) (as applicable to the corner(s) of a/the mouth being lifted by a corner lift procedure).

In aspects, method(s) of assessment comprise(s) determining that an ancillary nasal base suspension procedure is necessary in addition to the subnasal upper lip lift procedure to achieve target aesthetic effect(s)/a target aesthetic appearance. In aspects, the method(s) comprise identifying radial markings 4A (012A), 4B (012B), or both as reference points for the placement of one or more suture(s), for the path of at least one of the one or more suture(s), or both. In aspects, method(s) of assessment comprise evaluating the degree of laxity of the lip at location(s) indicated by radial markings 3A (010A), 3B (010B), or both, and according to the outcome of that evaluation determining the placement of one or more sutures(s), determining the path of at least one of the one or more suture(s), or both. In aspects, method(s) of assessment comprise(s) (1) use of the location of radial markings 4A (012A), 4B (012B), or both; (2) the degree of laxity of the lip at location(s) indicated by radial markings 3A (010A), 3B (010B), or both; or (3) both (1) and (2) in a nasal base suspension procedure to determine placement of one or more suture(s), elect the path of at least one of the one or more suture(s), or both. In aspects, method(s) of assessment comprise determining that an ancillary nasal base suspension procedure is necessary in addition to the upper lip lift, e.g., subnasal upper lip lift procedure to achieve target aesthetic effect(s)/a target aesthetic appearance, wherein the method(s) comprise(s) use of the location of radial markings 1, 2A (008A), 2B (008B), 4A (012A), 4B (012B), or a combination thereof to establish the location of the placement of one or more sutures associated with the muscle suspension procedure, such as, e.g., 2 or more, 3 or more, 4 or more, 5 or more, 6 or more, 7 or more, 8 or more, 9 or more, or 10 or more sutures, e.g., about 5 sutures associated with the muscle suspension procedure.

In aspects, each of the at least 1, at least 2, at least 3, at least 4, at least 5, at least 6, at least 7, at least 8, or, e.g., at least 9, such as, e.g., each of the at least 7 radial markers/indicator(s) established in method(s) of assessment described herein have or otherwise mark a first discrete anatomical location along the nasal base (LEVEL 1 indicator or upper excision boundary marker (002)). In aspects, each of the at least 1, at least 2, at least 3, at least 4, at least 5, at least 6, at least 7, at least 8, or, e.g., at least 9, such as, e.g., each of the at least 7 radial markers/indicator(s) established in method(s) of assessment described herein have or otherwise mark a second discrete anatomical location along the upper vermillion border (020) (LEVEL 3 indicator or upper vermillion border (020)).

According to certain aspects, if each first and second discrete anatomical locations along the LEVEL 1/upper excision boundary marker (002) and LEVEL 3/upper vermillion border (020) indicator associated with each of the radial markings, e.g., with each of the at least 7 radial markings 1, 2A (008A), 2B (008B), 3A (010A), 3B (010B), 4A (012A), and 4B (012B), respectively were to be connected, each of the resulting 7 connecting lines would intersect with the LEVEL 1/upper excision boundary indicator/marker (002), LEVEL 3/upper vermillion border, and a second at least generally horizontal, at least substantially horizontal, at least essentially horizontal, essentially horizontal, or horizonal indicator (LEVEL 2 indicator, referred to as the lower excision boundary indicator/marker (004) described elsewhere herein) positioned between the LEVEL 1/upper excision boundary marker (002) and LEVEL 3/upper vermillion border (020) indicators.

In aspects, the distance between the LEVEL 1 (upper excision boundary marker (002)) and LEVEL 2 indicators (the distance between the upper excision boundary marker (002) and the lower excision boundary marker (004)) along each of the radial markings establishes an excision height at the location of the radial marking. In aspects, this can be described as the radial marker indicator number (e.g., “1” for radial marker 1 (006)) followed by a “EX” for excision height, such that it is presented herein as “1-EX.” Herein, portions of radial marker(s) referring to excision height along such marker(s) may be referred to as, e.g., “#-EX,” or more specifically, e.g., 1-EX, 2A-EX, 2B-EX, 3A-EX, 3B-EX, 4A-EX, 4B-EX, etc.

In aspects, the distance between the LEVEL 2 (lower excision boundary marker (004)) and LEVEL 3 indicators (upper vermillion border (020)) (the distance between the lower excision boundary marker (004) and the upper vermillion border (020)) along each of the radial markings establishes a final upper lip height at the location of the radial marking. In aspects, this can be described as the radial marker indicator number (e.g., “1” for radial marker 1) followed by a “FH” for final lip height, such that it is presented herein as “1-FH.” Herein, portions of radial marker(s) referring to final lip height along such marker(s) may be referred to as, e.g., “#-FH,” or more specifically, e.g., 1-FH, 2A-FH, 2B-FH, 3A-FH, 3B-FH, 4A-FH, 4B-FH, etc.

In aspects, lengths of lines and portions/segments thereof (e.g., excision height, final lip height, or combination(s) thereof) established by method(s) of assessment can be length(s) thereof described elsewhere herein. Further, ratios of/between total length(s) of radial marking lines, ratios of segment(s)/line length(s), etc., established by method(s) of assessment are also described elsewhere herein. In certain aspects, the final upper lip height indicated by any one or more of the radial markings is at least about 9 mm, such as, e.g., at least about 10 mm, at least about 11 mm, or, e.g., at least about 12 mm. In aspects, the final upper lip height indicated by any one or more of the radial markings is at least about 10 mm or at least about 11 mm.

In certain aspects, method(s) of assessment herein establish an upper (superior) end of radial markings 2A (008A), 2B (008B), or both, which identify(ies) the upper peak of the subnasal incision to be made in the upper lip lift, e.g., subnasal upper lip lift procedure (e.g., a subnasal upper lip lift procedure incorporating a bull's horn incision technique) (e.g., with or without concomitant corner lip lift(s)).

Returning now to the placement of the LEVEL 2 indicator, e.g., lower excision boundary marker (004), as a step-in method(s) of assessment provided herein, in aspects, method(s) of assessment provided herein comprise(s) a step for evaluating one or more anatomical features of the human subject to establish a target aesthetic appearance. In aspects, a step for evaluating one or more anatomical features of the human subject to establish a target aesthetic appearance is followed by a step for establishing the location along the length of each of the at least 7 connecting lines where the LEVEL 2 indicator (lower excision boundary marker (004)) is positioned to achieve the target aesthetic appearance. In aspects, once the location along the length of each of the at least 7 connecting lines where the LEVEL 2 indicator (lower excision boundary marker (004)) is positioned to achieve the target aesthetic appearance, the marker is then established/positioned there, such that when the space between the LEVEL 1 indicator (upper excision boundary marker (002)) and LEVEL 2 indicator (lower excision boundary marker (004)) is excised in the subnasal lip lift procedure, the result is an at least generally uniform, at least substantially uniform, at least essentially uniform, essentially uniform, or uniform difference in height between the discrete anatomical locations along the upper vermillion border (020) between each of radial markings 2A (008A) and 3A (010A); 2B (008B) and 3B (010B); 3A (010A) and 4A (012A); 3B (010B) and 4B (012B); or any combination thereof.

In aspects, the at least substantially uniform difference in height between the discrete anatomical locations along the upper vermillion border (020) of radial markings 2A (008A) and 3A (010A), 2B (008B) and 3B (010B), 3A (010A) and 4A (012A), 3B (010B) and 4B (012B), or combination thereof described in the preceding paragraph is between about 1 mm to about 5 mm or more typically between about 1 mm to about 3 mm, such as, e.g., about 1 mm, about 2 mm, or, e.g., about 3 mm, such as, e.g., about 2 mm. Such height difference(s) are further described elsewhere herein.

In aspects, method(s) of assessment described herein comprise(s) a step for considering the lateral spacing along the upper vermillion border (020) between two or more radial markings and, thereto applying Pythagoras' theorem a²+b²=c² to determine the appropriate excision height along radial marking(s) required to attain a constant about 1 mm to about 5 mm height difference, e.g., ˜1 mm-˜4 mm, ˜1 mm-˜3 mm, ˜1 mm-˜2 mm, ˜2 mm-˜5 mm, ˜2 mm-˜4 mm, ˜2 mm-˜3 mm, ˜2 mm-˜4 mm, or, e.g., about 2 mm height difference, between the discrete anatomical locations along the upper vermillion border (020) of two neighboring radial markings, e.g., the lateral spacing between radial markings 3A (010A), 4A (012A), 3B (010B), 4B (012B), or two or more thereof.

According to certain aspects, method(s) of assessment described herein comprise a step for adding one or more additional radial indicator(s) between any two or more of radial indicators 1 and 2A (008A), 1 and 2B (008B), 2A (008A) and 3A (010A), 2B (008B) and 3B (010B), 3A (010A) and 4A (012A), 3B (010B) and 4B (012B), or is/are added lateral to 4A (012A), 4B (012B), or both. In certain aspects, such one or more additional radial indicator(s) detectably or significantly increase the amount of control a practitioner of the method has available in establishing the at least substantially similar difference in height between the discrete anatomical locations along the upper vermillion border (020) of 2A (008A) and 3A (010A), 2B (008B) and 3B (010B), 3A (010A) and 4A (012A), 3B (010B) and 4B (012B), or any combination thereof. In aspects, such one or more additional radial indicator(s) detectably or significantly increase the sensitivity the established guide(s)/marking system(s) of method(s) provide for the establishment of an at least substantially similar difference in height between the discrete anatomical locations along the upper vermillion border (020) of 2A (008A) and 3A (010A), 2B (008B) and 3B (010B), 3A (010A) and 4A (012A), 3B (010B) and 4B (012B), or any combination thereof, such as the at least generally similar/uniform, at least substantially similar/uniform, at least essentially similar/uniform, essentially similar/uniform, or uniform difference(s) in height described elsewhere herein.

In aspects, additional radial indicator(s) established in method(s) are positioned between radial indicators 2A (008A) and 3A (010A), 2B (008B) and 3B (010B), 3A (010A) and 4A (012A), 3B (010B) and 4B (012B), or in any two or more such locations, and the difference in height between the discrete anatomical locations along the upper vermillion border (020) of the additional radial indicator(s) and any adjacent radial indicator(s) is about 5 mm, 4 mm, 3 mm, 2 mm, 1 mm, such as no more than about 3 mm, no more than about 2 mm, or, e.g., no more than about 1 mm.

According to certain aspects, method(s) of assessment provided herein establish marking system(s) comprising radial marker(s) on each lateral side of a midline of the upper lip, wherein the method(s) further establish excision height(s) within radial marker(s) on one lateral side of midline, e.g., radial marker 1 (006), which is detectably or significantly different than the excision height(s) within the corresponding radial marker(s) on the opposite lateral side of midline, e.g., radial marker 1 (006), but wherein the final lip height(s) within the radial marker(s) on the one lateral side of midline, e.g., radial marker 1 (006), is/are at least essentially the same as the final lip height(s) within the corresponding radial marker(s) on the opposite lateral side of midline, e.g., radial marker 1 (006).

In some aspects, method(s) of assessment provided herein comprise a step for evaluating the point(s) of termination along the upper vermillion of radial markings 4A (012A) and 4B (012B) and the shape of the subfacial cheek lip and cheek lateral thereto, respectively. In aspects, method(s) of assessment provided herein comprise determining whether the lateral lip slope of the upper vermillion requires adjustment by an ancillary procedure, such as, e.g., a corner lift procedure, to maintain the slope of the upper vermillion targeted between the points along the upper vermillion of 2A (008A) and 3A (010A), 2B (008B) and 3B (010B), 3A (010A) and 4A (012A), and 3B (010B) and 4B (012B), or combinations thereof lateral to the point(s) of termination along the upper vermillion of each of radial markings 4A (012A) and 4B (012B), respectively. In aspects, if such a lateral slope requires adjustment, the method(s) can comprise establishing or otherwise incorporating one or more marking(s) suitable for use in a corner lip lift procedure.

In aspects, the disclosure provides method(s) of assessment wherein one or more radial marking(s), e.g., radial marking(s) 5A (014A), 5B (014B), or both, or location(s) associated therewith, serve (at least in part) as visual indicator(s), e.g., visual indicator(s) of one or more step(s) to be taken in the upper lip lift procedure with which the radial markings are associated. In aspects, radial markings 5A (014A) and 5B serve to remind a practitioner to place lateral internal mattress sutures during the procedure.

In aspects, the disclosure provides method(s) of assessment wherein the method comprises identifying at least two radial markings which each, on opposite sides of the midline of the upper lip, demarcate a first defined region identifiable as the subnasal lip region from a second defined region identifiable as the subfacial lip and lateral mouth region. In aspects, the first defined region, e.g., the subnasal lip region, has a detectably or significantly greater sensitivity to a subnasal lip lift technique/procedure compared to the second defined regions, e.g., the subfacial lip and lateral mouth regions, identifiable as the subfacial lip and lateral mouth regions, demarcated by the same at least two markings. In aspects, subfacial lip and lateral mouth regions, e.g., regions lateral to each of the at least two radial markings, have a detectably or significantly decreased sensitivity to a subnasal lip lift technique/procedure. In aspects, a subnasal lip lift technique is detectably or significantly modified at one or more radial marking position(s) according to the differentiation of sensitivity to effect of the two regions and where each radial marking is located relative to the two regions. In aspects, such at least two radial markings demarcating the first and second defined regions, e.g., demarcating the subnasal lip and the subfacial lip and lateral mouth regions, are radial markings are 4A (012A) and 4B (012B).

In aspects, relatively central portions of the lip, e.g., portions directly under the nose, will experience a greater extent of lifting than the lateral portions; that is, the subnasal lip, e.g., the portion of the lip medial to each of radial markings 4A (012A) and 4B (012B), will be more relatively more sensitive to lifting. Relatively lateral portions of the lip, e.g., portions lateral to the width of the nose, hanging from the face at the oral commissure, will experience a lesser extent of lifting than the medial portion; that is, the subfacial lip and lateral mouth regions, e.g., the portions of the lip lateral to each of radial markings 4A (012A) and 4B (012B), will be relatively less sensitive to lifting. In aspects, the larger the discrepancy between the width of the nose and the width of the lip, the greater the degree of difference in lift will be present in central versus lateral lifting. In aspects, method(s) provide lateral marking(s) 4A (012A), 4B (012B), or both to delineate where the effect of lifting on the lateral lip will detectably or significantly taper off.

Similarly, in aspects, the method(s) of assessment provided by the disclosure provide two or more markings of a marking system which identify two or more regions of the upper lip differing in their sensitivity to the effect of a subnasal lift procedure to achieve a target slope of the upper vermillion border (020) (the slope of the upper vermillion border (020) being the vertical descent of the upper vermillion border (020) from the Cupid's peak following laterally toward the oral commissure when viewing the human subject face-on). In aspects, each of the at least two regions is independently considered and modified according to their sensitivity to the subnasal lift procedure. In aspects, the two or more regions comprise the subnasal lip and the subfacial lip, and the lateral mouth regions. In aspects, the two or more markings of the marking system are radial markings 4A (012A) and 4B (012B).

In aspects, radial marking(s) established in step(s) of method(s) of assessment provided herein are grouped according to the side of the midline to which they are positioned. For example, in aspects, for ease of reference, marking line(s) positioned to a first side of midline, e.g., to a first side of radial marking 1 (006), are identified as “Group A” markings, while marking line(s) positioned to the second side of midline, e.g., to the opposing side of radial marking 1 (006), are identified as “Group B” markings. Group A and Group B markings are further described elsewhere herein. According to certain aspects, method(s) of assessment provided herein determine that the nasal base, upper lip, or both, of the human subject possess(es) one or more asymmetry(ies). In aspects, method(s) herein establish one or more radial marking(s) on a first side of the first radial marking (e.g., one or more “Group A” marking(s)) comprising a different total length at that location which is different from the total marker length of the corresponding radial marking(s) located on a second side of the first radial marking (e.g., than that of the corresponding “Group B” marking(s)). In aspects, method(s) of assessment provide identification of an excision height which varies from side-to-side of the subject's upper lip in order to attain a final lip height at the two corresponding location(s) which is at least generally the same, at least substantially the same, at least essentially the same, is essentially the same, or which is the same. In aspects, such method(s) result in the detectable or significant reduction of or elimination of an asymmetry upon performance of a subnasal lift.

According to certain aspects, method(s) of assessment provided herein is/are performed prior to the commencement of any excision performed as part of the upper lip lift procedure to which the method(s) is/are being applied, in which the method(s) is/are being incorporated, or in which step(s) of the method are relevant.

In aspects, method(s) of assessment described herein establish marking system(s) comprising any one or more of the marking system characteristic(s) described herein.

### Computer-Aided System(s) and Method(s)

Disclosed herein are automated system(s)/device(s) and associated method(s), wherein such system(s) and method(s) establish one or more parameter(s) to be implemented in an aesthetic modification procedure to achieve a target result, where such parameters are typically associated with a facial feature. Such parameter(s) are typically characteristic(s) of specific location(s), such location(s) identified by the marker(s) of the location(s), such location(s) identified by the marker(s) of marking system(s) described herein.

Automated system(s) provided herein establish measurement(s) associated with such characteristic(s) of the, e.g., upper lip, as at location(s) defined herein by marker(s) associated therewith. Starting with, e.g., an image of a patient's original facial appearance, automated system(s) apply one or more stored input(s), one or more user input(s), or combination(s) thereof related to the measurement(s) of the upper lip at defined location(s) to the original image. System(s) present, as a morphed version of the original image, the patient's appearance predicted to result from the application of such input(s) in an aesthetic modification procedure. Methods further include the ability of users to modulate 2, 3, or 4, or more parameters associated with included facial features to generate alternative or further modified morphed images (sometimes called secondarily morphed images).

Automated system(s) provided herein can allow patient(s) to visualize what the outcome of an aesthetic modification procedure will be according to particular pre-defined measurements of specified facial feature(s)/location(s). System(s) allow for multiple iterations of input(s), such that predicted outcome(s) can be modified until such time a desired target aesthetic appearance is achieved.

In aspects, the disclosure provides system(s) and method(s) for modifying the appearance of a human face, e.g., the lip(s), e.g., the upper lip, of a human face, in (typically) two-dimensional image(s). Such system(s) and method(s) are designed for use in upper lip lift aesthetic modification procedures, such as, e.g., a deep plane release upper lip lift procedure(s). Further, automated system(s) and associated method(s) of their use can provide recommendation(s) and associated requirement(s) for one or more ancillary procedures (e.g., procedure(s) performed in addition to an upper lip lift procedure) such as, e.g., a corner lip lift, such as a Type I or Type II corner lip lift as described herein.

### Computer-Aided System(s)

According to aspects, the determination of one or more feature(s)/characteristic(s) of the marking system(s) described herein is automated.

Automated system(s) herein can, in aspects, apply artificial intelligence technology (artificial intelligence model(s)/system(s)) to an original image of a human face in order to recognize landmark(s)/location(s) (features and characteristics/parameters) of such face; apply change(s) to such an original image according to stored or received input(s) such that the characteristic(s) of the face at one or more location(s) is modified; and present a morphed image of the human face, wherein the morphed image of the human face predicts what the human face depicted in the original image would look like if the characteristic(s) of the input(s) were applied to the human face in an aesthetic modification procedure.

In aspects, one or more feature(s)/characteristic(s) of marking system(s) described herein which, when applied during or by the performance of an aesthetic modification procedure, result in a target visual appearance having one or more targeted visual characteristics (such as, e.g., target upper vermillion slope, target incisor display, and the like), is determined by automated process(es), e.g., as performed by automated system(s) (computer-aided system(s)).

In aspects, the disclosure provides automated system(s) designed to establish target effect(s), e.g., target aesthetic appearance(s), resulting from one or more aesthetic modification procedure(s), such as, e.g., an upper lip lift, a subnasal upper lip lift, a deep plane release upper lip lift, a corner lip lift, a nasal base suspension, a muscle suspension, or any combination of any or all thereof (e.g., subnasal and corner lip lift procedure). In aspects, automated system(s) are provided as a client-server application offering photographic manipulation capability(ies). In aspects, automated system(s) herein provide real-time image processing, real-time AI computation(s), or both real-time image processing and real-time AI computation(s).

In aspects, disclosed here are automated system(s) and components thereof, e.g., mobile application(s), for use in method(s) of establishing target effect(s), e.g., target aesthetic appearance(s), e.g., effect(s) and appearance(s) (e.g., of a human face, e.g., of the upper lip of a human face) resulting from one or more aesthetic modification procedure(s) described herein. Such method(s) are method(s) described herein or similar method(s) wherein such system(s), e.g., application(s), provide at least mostly, at least generally, at least substantially, at least essentially, essentially, or the same function(s).

Herein, the term “automated system(s)” may be also or alternatively be disclosed as, e.g., software, software application(s), automated program(s), mobile application(s), computer system(s), computer or otherwise automated application(s), or combination(s) of software-based system(s)/application(s), artificial intelligence-based system(s)/application(s), or both, with or without use of neural network(s) facilitating machine learning. Disclosure using any one such term should, uncontradicted, be interpreted as encompassing such disclosure using any other such term. Automated system(s) herein can be, e.g., system(s) with which a user interface(s) via any type of device, such as, e.g., a desktop computer, laptop computer, PDAs, mobile devices including smartphones, tablets, and the like. A feature of aspects of automated system(s) herein can be, e.g., that such system(s) are functional and visually appealing on a variety of device types, including a variety of screen sizes.

In aspects, the disclosure provides new automated system(s) (e.g., software application(s)), which may be implemented via the internet, mobile application(s), desktop application(s), and the like, which carry out the step(s) of method(s) described herein as functions. For example, in aspects the disclosure provides new automated system(s) that establish marking system(s) according to rule(s), feature(s), characteristic(s), or setting(s) for such marking system(s) described herein required to achieve target aesthetic appearance(s).

According to aspects disclosed herein are automated system(s), e.g., mobile application(s). According to aspects, disclosed herein are automated system(s), e.g., mobile application(s), providing the automated system user the ability to modify an image, e.g., a photograph. In aspects, the photograph is a photograph of a human face. In aspects, the photograph is of the face of the automated system user or of a patient/potential patient of the automated system user when, e.g., the automated system user is a medical professional. In aspects, the automated system(s), e.g., mobile application(s), described herein allow for the automated system user to experiment with possible changes to visual appearance as depicted in a photographic image of, e.g., a human face. In aspects, the automated system(s) are designed to allow the automated system user to experiment with modification(s) to the appearance of a human face with a specific focus on enhancing, reducing, or otherwise modifying the size, shape, positioning, or combination(s) thereof, of lip(s), e.g., the upper lip. In certain specific aspects, provided herein are automated system(s), e.g., mobile application(s), designed to provide user(s) thereof with an interactive tool allowing user(s) to modify an/their appearance in photograph(s), such as, e.g., modifying the appearance of an/their face in photographs(s), e.g., modifying the size, shape, positioning, or combination(s) thereof, of their lip(s), e.g., an/their upper lip.

According to aspects, automated system(s) provided herein typically operate using two-dimensional images and typically generate two-dimensional output images. However, it is contemplated that the concepts and principles described herein can be applied to three-dimensional inputs and three-dimensional outputs, and thus, such system(s) and associated method(s) are incorporated within the scope of this disclosure.

In aspects, the disclosure provides automated system(s) for generating a plurality of measurements associated with at least 7, such as ≥˜8, ≥˜9, ≥˜10, ≥˜11, ≥˜12, ≥˜13, ≥˜14, ≥˜15, ≥˜16, ≥˜17, ≥˜18, ≥˜19, ≥˜20, ≥˜21, ≥˜22, ≥˜23, ≥˜24, ≥˜25, ≥˜26, ≥˜27, ≥˜28, ≥˜29, ≥˜30, ≥˜31, ≥˜32, ≥˜33, ≥˜34, ≥˜35, ≥˜36, ≥˜37, ≥˜38, ≥˜39, ≥˜40, ≥˜41, ≥˜42, ≥˜43, ≥˜44, ≥˜45, ≥˜46, ≥˜47, ≥˜48, ≥˜49, or ≥˜50 (or even a significantly greater number of) locations associated with the upper lip or other portion of a human face (such as, e.g., about 7-about 15, e.g., 3-12, 2-20, 2-14, 2-8, 3-9, 2-6, 3-6, or 4-8 or 4-6 specific location(s) in certain aspects). In aspects, such location(s) are location(s) identified herein by marker(s), as described elsewhere.

In aspects, one such location is the middle of the columella and philtral dimple, at the lowest point of the trough of Cupid's bow and extending from the nasal base to the upper vermillion border. Herein this may be referred to as “location 1”, identified as “marker 1” of marking system(s) herein. In aspect, location 1 represents a centralized location and pair(s) of additional location(s) described here comprise one location on one side of location 1 and another location on the other side of location 1. (E.g., if the upper lip were to be divided by location 1 such that there is a “left” and a “right” side, pair(s) of additional location(s) described here can have an “A” position on one side of location one and a “B” position on the opposite side of location 1, as has been described in the context of “marker(s)” herein.)

In aspects, other such location(s) of the at least 7 location(s) described above include each of 2 lateral locations, one on either side of the upper lip, at the peaks of Cupid's bow, and at the points where the medial crural footplates diverge into the nose at the junction(s) with the alar sill. In aspects, each such location extends from the nasal base to the upper vermillion border. Herein, these locations may be referred to as “location 2A” and “location 2B,” identified as “marker 2A” and “marker 2B” of the marking system(s) herein.

In aspects, other such location(s) of the at least 3, 4, 5, 6, or 7 location(s) described above include each of 2 lateral locations, one on either side of the upper lip and each extending from the internal angle of the alar recurvature radially downward and following the natural curvature and natural tension lines, radial angulation of the pores of the upper lip, or any combination thereof, each location extending from the nasal base to the upper vermillion border. Herein, these locations may be referred to as “location 4A” and “location 4B,” identified as “marker 4A” and “marker 4B” of the marking system(s) herein.

In aspects, other such locations(s) of the, e.g., at least 3, 4, 5, 6, or 7 location(s) described above include each of 2 lateral locations, one in between each of locations 2A and 4A and locations 2B and 4B. These locations(s) each extend from the nasal base to the upper vermillion border. Herein, these locations may be referred to as “location 3A” and “location 3B,” identified as “marker 3A” and “marker 3B” of the marking system(s) herein. Location(s) 3A and 3B are identified as intermedial, positioned between locations 2A and 4A and 2B and 4B, respectively. In certain aspects of method(s) comprising their generation, such location(s) may be identified (and/or their marking(s) or marking characteristic(s) generated) after that/those of location(s) 2A, 2B, 4A, and 4B.

In aspects, other such location(s) of the, e.g., at least 3, 4, 5, 6, or 7 locations described above include each of 2 lateral locations, one on each side of the upper lip, wherein each location extends downward from the nasal base and follows each of the relaxed skin tension lines (RSTLs). Herein, these locations may be referred to as “location 5A” and “location 5B,” identified as “marker 5A” and “marker 5B” of the marking system(s) herein.

In aspects, other such location(s) of the, e.g., at least 3, 4, 5, 6, or 7 locations described above include locations extending laterally on either side of the upper lip from the points on the upper vermillion border of locations 4A and 4B, above (for a total of two locations) to a point at or near the nasolabial fold line(s) of the relaxed skin tension line globe. Herein, these locations may be referred to as “location H-A” and “location H-B,” identified as “marker H-A” and “marker H-B” herein.

In still further aspects, other such location(s) of the, e.g., at least 3, 4, 5, 6, or 7 locations described above include locations extending laterally on either side of the upper lip from a point located between the nasal sill and the upper vermillion border along the locations identified as locations 4A and 4B above (for a total of two locations). Herein, these locations may be referred to as “location LE-A” and “location LE-B,” identified as “marker LE-A” and “marker LE-B” herein.

According to particular aspects, the plurality of generated measurements established by automated system(s) herein are measurement(s) which, when established by application of an upper lip lift aesthetic modification procedure to the human face, provide an aesthetic appearance predicted by automated system(s) disclosed herein.

In aspects, automated system(s) of this disclosure comprise a plurality of feature(s) and function(s), which provide for the effect of establishing a guide for use in performing an aesthetic modification procedure to establish a result predicted by the system(s). In aspect(s), such feature(s) comprise, e.g., processing and memory functionality; landmark detection functionality; visual guide(s) generation, application, & measurement functionality(ies); image morphing functionality; artificial intelligence-based calculation capability; data capture and retrieval functionality; or combination(s) of any or all thereof.

In aspects, automated system(s) are designed to apply such feature(s) and function(s) to analyze an original image of at least the upper lip of a human face. Automated system(s) are further, in aspects, designed to apply landmark detection functionality to identify at a plurality of location(s) on the image, e.g., at least 2, 3, 4, 5, 6, or 7 specific locations, such as at least 8, at least 9, at least 10, at least 11, at least 12, at least 13, or, e.g., at least 14 specific locations on the image associated with dimensions of the upper lip of the human face. In aspects, automated system(s) apply measurement functionality to the identified locations to obtain measurement(s) at such location(s) associated with the original image.

In aspects, automated system(s) apply morphing technology(ies) to morph the original image according to one or more input(s), rule(s), or setting(s) to establish morphed image(s). A morphed image is a realistic yet modified version of a first image, having had one or more feature(s) or characteristic(s) of the first image modified according to one or more input(s). In aspects, the morphed image(s) represent(s) predicted aesthetic appearance(s) which would result from the performance of an aesthetic modification procedure(s) on the subject of an original image according to the characteristic(s) of measurement(s) at specified location(s) of the image.

In aspects, morphed image(s) generated by automated system(s) are presented to a user of the automated system(s) within the automated system(s), wherein the user is able to evaluate the predicted appearance and, if appropriate or otherwise desired, modify input(s) data and generate new morphed image(s) until a desired predicted appearance is achieved. In aspects, morphed images are generated using input data (stored or provided by user(s)) related to measurement(s) at one or more of the at least 3, 4, 5, 6, or 7 specific location(s) discussed herein. In aspects, characteristic(s) of data, e.g., measurement(s) related to one or more of location(s) 1, 2A, 2B, 3A, 3B, 4A, 4B, 5A, 5B, H-A, H-B, LE-A, and LE-B, yielding a predicted aesthetic appearance presented as a morphed image are required to be applied or otherwise established in the upper lip lift aesthetic modification procedure in order to obtain the predicted aesthetic appearance presented in the morphed image.

In certain aspects, automated system(s) herein generate and identify specific point(s) along each of at least locations 1, 2A, 2B, 3A, 3B, 4A, and 4B. In aspects, each such specific point divides the respective location into two segments.

One segment represents a final lip height, identified herein using the identifier “FH.” That is, for example, the automated system(s) generate and identify a specific point along (e.g.,) location 2A which divides location 2A into two segments; one segment indicating the final lip height at that location and identified as segment “2A-FH.” The term “segment” is used herein to reflect the language used herein to describe marking systems, as if the location represented by location (e.g.,) 2A were present as an actual marking line, and the line were divided into portion(s) or “segment(s).” However, the reader should understand that in embodiment(s) wherein such marker(s) are virtual, the term “segment” is used to similarly indicate a portion of the virtually identified location.

The other segment represents an excision height at such a location, identified herein using the identifier “EX.” That is, for example, the automated system(s) generate and identify a specific point along (e.g.,) location 2A which divides location 2A into two segments, wherein one segment indicates the final lip height at that location (2A-FH) and the other segment represents the portion of the upper lip at that location which, if excised, would yield the final lip height measurement of 2A-FH (2A-EX).

According to aspects, automated system(s) provided herein generate and identify specific points at each of the at least 7 locations 1, 2A, 2B, 3A, 3B, 4A, and 4B (and, e.g., optionally 5A and 5B) such that upon excision of the upper lip according to the excision height at each identified location (1-EX, 2A-EX, 2B-EX, 3A-EX, 3B-EX, 4A-EX, 4B-EX, and optionally 5A-EX and 5B-EX) in an upper lip lift aesthetic modification procedure, the final lip height of the upper lip at each identified location (1-FH, 2A-FH, 2B-FH, 3A-FH, 3B-FH, 4A-FH, 4B-FH, and optionally 5A-FH and 5B-FH) yield a slope of the upper vermillion border of between about 1 mm and about 3 mm, such as about 2 mm, between points along the upper vermillion border of 2A and 3A; between points along the upper vermillion border of 3A and 4A; between points along the upper vermillion border of 2B and 3B; between points along the upper vermillion border of 3B and 4B; optionally between points along the upper vermillion border of 4A and 5A and between points along the upper vermillion border of 4B and 5B; or between combinations of some or all of such pairs of points.

According to aspects, automated system(s) provided herein generate and identify specific points at each of the at least 3, 4, 5, 6, or 7 locations 1, 2A, 2B, 3A, 3B, 4A, and 4B (and, e.g., optionally 5A and 5B) such that upon excision of the upper lip according to the excision height at each identified location (1-EX, 2A-EX, 2B-EX, 3A-EX, 3B-EX, 4A-EX, 4B-EX, and optionally 5A-EX and 5B-EX) in an upper lip lift aesthetic modification procedure, the final lip height of the upper lip at each identified location (1-FH, 2A-FH, 2B-FH, 3A-FH, 3B-FH, 4A-FH, 4B-FH, and optionally 5A-FH and 5B-FH) yield an incisor display of between about 1 mm and about 5 mm, such as, e.g., about 1 mm, ˜2 mm, ˜3 mm, ˜4 mm, or about 5 mm, such as about 1-4 mm, or e.g., about 2-4 mm, or, e.g., about 1-3 mm.

According to aspects, characteristic(s) of location(s) generated or compiled by automated system(s) herein yielding the predicted aesthetic appearance can be saved by the automated system(s); transmitted by the automated system(s), or both. In aspects, transmission can be, e.g., to a medical professional to aid in the performance of an aesthetic modification procedure on the subject to attain the predicted aesthetic appearance. The compilation of such data may be referred to as a feature set herein.

Automated system(s) herein can further, in aspects, make one or more recommendation(s) for one or more intervention(s) to be applied as an ancillary procedure to the upper lip lift procedure. In aspects, the ancillary procedure is a corner lip lift procedure, such as a Type I or Type II corner lift procedure described herein. Automated system(s) can, in certain aspect(s), provide one or more measurement(s) associated with one or more location(s) described herein, which would result in a predicted aesthetic appearance if applied in a procedure such as a corner lip lift conducted alone or in conjunction with an upper lip lift procedure.

Automated system(s) provided herein can, in aspects, comprise feature(s) and function(s) providing iterative functionality(ies) wherein, for example, a first predicted aesthetic appearance is generated and presented as a morphed image based upon a first set of input(s) and, after system user(s) review the presented morphed image, one or more input parameter(s) of the system(s) is/are adjusted, resulting in the generation of a new morphed image. In aspects, the new morphed image represents an updated predicted result/outcome. In aspects, such a process of review, input modification, and re-presentation of morphed image(s) can be repeated (iterated) a plurality of times until parameters yielding target effect(s) are achieved. In aspects, such a process can be repeated any number of times required to achieve an acceptable predicted appearance (e.g., an appearance desired by the subject/patient.) For example, such a process may be iterated 2 or more, ˜3 or more, ˜4, ˜5, ˜6, ˜7, ˜8, ˜9, ˜10, ˜12, ˜14, ˜16, ˜18, ˜20, ˜22, ˜24, ˜26, ˜28, ˜30, ˜32, ˜34, ˜36, ˜38, ˜40, ˜42, ˜44, ˜46, ˜48, or even ˜50 or more times.

In certain aspects, iteration(s) are captured by machine-learning process(es)/programming/mechanism(s) present in the automated system(s). In aspect(s), machine-learning process(es)/programming/mechanism(s) present in the system(s) “teach” the system preference(s) with regard to desirable outcome(s). In aspects, as the system(s) capture more and more indication(s) of preference(s), suitable target aesthetic appearance(s), e.g., desirable (e.g., “target”) aesthetic appearance(s), are generated with detectably or significantly fewer iterations.

Any of the element(s) of description(s) of automated system(s) provided here and elsewhere within this disclosure can be applied to method(s) comprising such element(s). Such disclosure should be interpreted accordingly.

Specific aspects of exemplary feature(s) and function(s) of computer-aided system(s) (automated system(s)) provided by this disclosure are provided here.

### Automated System User(s)

In aspects, automated system(s), e.g., mobile application(s) provided herein, are designed for use by medical professional(s). In aspects, automated system(s), e.g., mobile application(s) provided herein are designed for use by the lay public, e.g., individual(s) who may be interested in undergoing an aesthetic modification procedure, such as, e.g., an upper lip lift.

User(s) of automated system(s), such as mobile application(s), provided herein, are typically associated with or establish, e.g., within such system(s), user-associated identification information such as, e.g., that described elsewhere herein (e.g., username, given name (first, last, middle, or combination(s) thereof), mailing address, email address, phone number, social security number, personal identification number (PIN), account number, password(s), gender, ethnicity, height, weight, age, and other types of identifying information and combination(s) thereof typically captured in user authentication method(s)/process(es), and preference data such as communication preference(s), application setting preference(s), and the like, which are exemplified herein).

User(s) of automated system(s), such as e.g., mobile application(s), disclosed here may also be associated with or establish, e.g., within such system(s), user-associated preference(s). In aspects, user-associated preference(s) can be, e.g., preference(s) associated with who can access the user account, sensitivity to morphing feature(s) of the application, aesthetic modification procedure preference(s), alert notification(s), and the like.

System(s) can comprise a plurality of user(s), wherein, e.g., certain user(s) are “associated user(s).” In aspects, associated user(s) are user(s) having access to some, most, generally all, or substantially all function(s) and feature(s) of system(s), or having access to some, most, generally all, or substantially all data associated with a user account, but for whom at least some access to function(s) and feature(s) or access to at least some data is limited. For example, a system user, an associated system user, or both may, in aspects, be able to participate in the generation of a target aesthetic appearance, however in aspects, an associated user may not be able to propose modification(s) to an image (e.g., participate in the morphing of image(s)), may have limited or no ability to transmit data, may have limited or no ability to transmit data, etc. In other aspects, associated user(s) may have access to one, some, generally all, substantially all, essentially, all or even all of the same function(s) and feature(s) of system(s) associated with an account, however, the associated user is not the account owner; for example, a user may be a prospective patient and an associated user may be, e.g., a medical professional (or vice versa: a user may be, e.g., a medical professional and an associated user may be, e.g., a patient). In certain aspects, system(s) only have one user.

Automated system(s) disclosed herein can comprise one or more control(s) designed to prevent user(s) and associated user(s) from generating conflicting instruction(s) within the system. For example, one or more action(s) taken, e.g., input(s) provided by an associate user, may be first presented to a user as a proposed action, at which time the user can accept or reject such proposal(s) (or vice versa). In another example, action(s) taken by, e.g., input(s) provided by, a user can be, e.g., identified as one set of instructions, leading to, for example, the generation of one proposed outcome, while action(s) take by an associated user can be, e.g., identified as a separate set of instruction, leading to, for example, the generation of another proposed outcome. In aspects, a plurality of proposed outcome(s), e.g., morphed images, can be maintained by system(s) and one, some, most, generally all, substantially all, essentially all, or all (each) presented to user(s), associated user(s), or both, at one or more stage(s) of method(s)/process(es) provided herein.

### Automated System Platform & Interface

In aspects, automated system(s), mobile application(s), described herein, can comprise any suitable operating system. According to aspects, operating system(s) provided herein are operable across a plurality of technological platforms, such as both Android and iOS platforms. In aspects, mobile application(s) comprise internal iOS software. In aspects, automated program(s), e.g., mobile application(s) described herein are characterizable as internal iOS application(s).

In aspects, automated system(s) utilize any suitable programming language. In aspects, automated system(s) provided herein utilize Swift programming language.

In aspects, automated system(s) comprise any suitable integrated development environment (IDE). In aspects, the IDE of automated system(s) described herein is Xcode. In aspects, a suitable IDE is an IDE comprising one, some, most, generally all, substantially all, essentially all, or all of the feature(s) and capability(ies) of Xcode.

### Automated System Memory & Processing Component(s)

In this regard, in one aspect, the disclosure provides new automated system(s), device(s), or both that comprise a memory component that maintains both instructions for carrying out such functions (e.g., programming regarding the relationship of lines, spacings, etc., described above with respect to the performance of the method(s) of the disclosure and for application thereof in, e.g., analyzing an individual's face, making facial predictions of the results of the surgery or other predictive modeling and the like, providing surgical guidance, training on performance of the technique, etc.), and optionally memory for capturing information specific to an individual's characteristic(s) (e.g., predicted facial modeling by application of the method, current facial state, etc.), and processor(s) for carrying out the programming instructions and optionally to further control the operation of computer devices such as display(s) (e.g., by illustrating such models, instructions, steps of the method, particular steps to be taken for a particular patient, etc.), or control of other device(s) (e.g., control of robotic surgical tools to perform some part(s) of the method(s) described herein).

In general, any suitable combination of computer system(s) or device(s) can be used for carrying out such functions. The inclusion of specific elements of method(s) described herein, in effect, results in providing a functional special-purpose computer for carrying out such one or more specific method(s). Thus, while the basic operating elements of such systems/devices can be done with a variety of computer systems and methods (e.g., systems using web services, such as Google services, Amazon Web Services (AWS), Microsoft Azure services, and similar cloud-based computer platforms and the like, or conventional computers such as WINDOWS-based computers, MAC OS based computers, or even handled platforms, the like), the programming of such computers (by conventional programming methods such as using PYTHON, SWIFT, RUBY programming tools, JAVA, C++, and the like), the incorporation of the various specific algorithms provided herein, as converted Into such programming, again provides concrete technical effects, functions, and specialized computing devices that can perform operations that cannot be achieved by computers lacking such specialized functions and memory components. According to aspects, PYTHON may be particularly suitable for use in system(s) provided herein, given its significant support in numerical and scientific computing, e.g., particularly with library(ies) suited for image processing, such as, e.g., AI, such as NumPy and OpenCV.

According to aspects, server-related element(s) of system(s) herein utilize one or more cloud-based computing service(s). In aspects, automated system(s) comprise a cloud infrastructure for deploying server-side component(s). In aspects, such cloud-based computing service(s) are scaled according to need. According to aspects, server-related element(s) are deployed on AWS. In aspects, such server-related element(s) of system(s) herein handle some, most, generally all, substantially all, essentially all, or all of machine learning task(s) associated with system(s) of this disclosure. For example, such task(s) can comprise, e.g., processing image data and performing required artificial intelligence operations to detect facial landmark(s) and further modifying them according to user-specified marker(s) values(s), and, further, providing such morphed image(s) to system(s) users. Task(s) can comprise, e.g., extracting face mesh (guide(s) and measurement(s) as described herein) data from one or more photographs (of, e.g., patient(s), e.g., system user(s)), and, e.g., generating new images with one or more morphed or otherwise altered lip feature(s) based on marker(s) input(s) described herein. In aspects, such functionality allows system(s) user(s) to visualize proposed aesthetic modification(s) directly from, e.g., mobile device(s).

According to aspects, automated system(s) herein comprise an application programming interface (API) to address request(s) between the user (front-end) and server(s). In aspects, such API(s) address task(s) such as, e.g., image capture or upload, sending parameter(s) for image manipulation (morphing), retrieving manipulated (morphed) images, and the like. In aspects, tools such as Flask can be suitable for developing such API(s). In aspects, Flask or tool(s) comprising one, some, most, generally all, substantially all, essentially all, or all feature(s), function(s), or both of Flask address HTTP request and server backend service(s).

### Visual Guide(s) & Measurement(s) Technology(ies)

In aspects, the disclosure provides automated system(s) to automate the evaluation and characterization process(es) associated with the establishment of marking system(s) described herein, and, e.g., other method(s) described herein (such as, e.g., performing one or more procedure(s) or evaluating the need therefore, comprising use of one or more feature(s), characteristic(s), or calculation(s) associated with marking system(s) provided herein). In aspects, the disclosure provides automated system(s) to automate the development of marking system(s) provided herein, which are suitable for acting upon in order to attain one or more target aesthetic effect(s).

In aspects, automated system(s), e.g., mobile application(s), provided by the disclosure automatically place one or more visual guide(s) upon a live image or, e.g., a photograph. In aspects, one or more visual guide(s) can be, e.g., marking(s)/marker(s). In aspects, such marking(s)/marker(s) can be, e.g., lines, dots, symbols, letters, numbers, or any other visually discernible indicator(s). In aspects, such marking(s) can be, e.g., lines, such as e.g. line(s) marking incision(s) to be made in an associated aesthetic modification procedure. In aspects, marking(s) can be, e.g., incision lines. In aspects, marking(s) can be radial marker(s), such as one or more radial marker(s) described herein. In aspects, marking(s) can be, e.g., at least generally, at least substantially, at least essentially, essentially, or completely diagonal lines; at least generally, at least substantially, at least essentially, essentially, or completely vertical lines; or, e.g., at least generally, at least substantially, at least essentially, essentially, or completely horizontal lines. In aspects, one or more marking(s) can radiate radially away from a given landmark as described herein. In aspects, visual guide(s), can be, e.g., one or more of an upper incision marker (upper excision boundary marker), lower incision marker (lower excision boundary marker), one or more radial marker(s) described herein, and, e.g., one or more marker(s) along the vermillion border of the upper lip (upper vermillion border). In aspects, visual guide(s) can be, e.g., one or more of an identification of the nasal labial fold or an indication of the endpoint of the crease of the nose.

In aspects, one, some, most, generally all, substantially all, essentially all, or all visual guide(s) established by automated program(s), e.g., mobile application(s), provided herein assist in the performance of or otherwise participate in (are used in or are used to aid in the performance of) one or more aesthetic modification procedure(s) such as, e.g., a lip lift procedure. In aspects, one, some, most, generally all, substantially all, essentially all, or all visual guide(s) established by mobile application(s) provided herein determine one or more measurements for one or more aesthetic modification procedure(s), such as, e.g., a lip lift procedure.

According to aspects, automated system(s), e.g., mobile application(s), provided by the disclosure automatically place one or more visual guide(s) upon a live image or, e.g., a photograph, which alone or together with an associated image, are stored or otherwise captured for use. In aspects, such visual guide(s) and data related thereto are stored or otherwise captured for use in, or are incorporated into, a feature set. Herein, a feature set is a compilation of data which, when applied to or used in a procedure, results in an outcome predicted by system(s), e.g., application(s) described herein.

In aspects, one or more visual guide(s) and data related thereto are available for referencing (for use in) or establishing parameter(s) of aesthetic modification procedure(s) such as upper lip lift procedure(s). In specific aspects, feature set(s) generated by system(s) and stored by storage capability(ies) (memory/memory component(s)) associated with such system(s), comprise data which, when applied to aesthetic modification procedure(s) such as, e.g., upper lip lift or specifically deep plane upper lip lift procedure(s), result in such procedure(s) yielding an outcome which is at least mostly the same as, as least substantially the same as, at least essentially the same as, is essentially the same as, or is the same as, e.g., is comparable to, predicted outcome(s) generated by morphing technology(ies) associated with the system(s).

According to aspects, automated system(s) provided herein can comprise any suitable visual guide and measurement technology(ies). In aspects, a suitable visual guide and measurement technology is a technology (e.g., framework) capable of drawing visual guide(s), applying a measurement to or taking a measurement of element(s) of an image (e.g., photograph), or combination(s) thereof. In one example, a suitable visual guide and measurement framework is an Apple framework. In aspects, the Apple framework is Apple's Core Graphics framework. In aspects, a suitable visual guide and measurement framework is a framework having one, some, most, generally all, substantially all, essentially all, or all of the feature(s) and functionality of Apple's Core Graphic framework.

In some aspects, visual guide and measurement technology(ies) of automated system(s) provided herein are capable of building one or more user interface component(s) for displaying visual guide(s) and measurement(s). In aspects, automated system(s) provided herein comprise any suitable visual guide and measurement technology(ies) capable of building one or more user interface component(s) for displaying visual guide(s) and measurement(s). In particular aspects, a suitable visual guide and measurement technology is an Apple framework. In aspects, the Apple framework is UIKit. In aspects, a suitable visual guide and measurement framework is a framework having one, some, most, generally all, substantially all, essentially all, or all of the feature(s) and functionality of Apple's UIKit framework.

According to certain aspects, automated system(s) provided herein comprise one or more visual guide(s) & measurement(s) technology(ies) comprising, or which are at least mostly, at least generally, at least essentially, are essentially, or are equivalent to, or in aspects which are superior to (in terms of breadth/scope/functionality/operational benefit) one or both of Apple's Core Graphics framework and UIKit framework.

The term “automated system” herein is used in view of the fact that one or more function(s) performed by the system are performed automatically in operation. The term, uncontradicted, is synonymous with the more general term system herein and, where appropriate, implicitly describes devices that perform the described system functions.

### Landmark Detection & Morphing Functionality

In aspects, automated system(s), e.g., mobile application(s) provided herein, utilize facial recognition technology(ies) to identify a plurality of facial features in facial image data (e.g., in image file(s)). In aspects, systems also identify one or more characteristics associated with each of the selected facial features.

According to aspects, an automated system(s) of this disclosure comprises an artificial intelligence model comprising a facial (face) landmark detection model/facial image recognition model. In aspects, such a model is a machine learning or other artificial intelligence model capable of accurately identifying facial landmark(s) in image(s). In aspects, such model(s) identify one, some, most, generally all, substantially all, essentially all, or all key facial landmarks, e.g., facial landmarks identified by each of, e.g., marker(s) 1, 2A, 2B, 3A, 3B, 4A, 4B, 5A, 5B, upper excision boundary, upper vermillion border, and other landmark(s) disclosed herein accurately in at least about 80% of instances of such challenge(s), such as, e.g., identifying such landmark(s) accurately in at least about 81%, at least ˜82%, at least ˜83%, at least ˜84%, at least ˜85%, at least ˜86%, at least ˜87%, at least ˜88%, at least ˜89%, at least ˜90, at least ˜91%, at least ˜92%, at least ˜93%, at least ˜94%, at least ˜95%, at least ˜96%, at least ˜97%, at least ˜98%, at least ˜99%, at least ˜99.2%, at least ˜99.5%, at least ˜99.7%, at least ˜99.9%, or, e.g., 100% of instances.

In aspects, models described here locate such landmark(s) described here accurately to within about 5 mm, within ˜4 mm, within ˜3 mm, within ˜2 mm, within ˜1 mm, within ˜0.8 mm, within ˜0.6 mm, within ˜0.5 mm, within ˜0.4 mm, within about 0.2 mm, or within about 0.1 mm, such as, e.g., within about 0.08 mm, ˜0.06 mm, ˜0.04 mm, or within ˜0.01 mm, even within ˜0.008 mm, ˜0.005 mm, ˜0.001 mm, or even less, in at least about 80% of instances, such as, in at least ˜85%, ˜90%, ˜95%, ˜96%, ˜97%, ˜98%, ˜99%, ˜99.5%, ˜99.6%, ˜99.7%, ˜99.8%, at least ˜99.9%, or in 100% of instances.

According to certain specific embodiments, the disclosure provides automated system(s) comprising programmed concepts provided herein, e.g., marking system(s) provided herein, concept(s) founding the establishment of marking system(s) provided herein, mathematical relationship(s) upon which various element(s) of marking system(s) are established, the combination of various decision point(s) contributing to procedure(s) herein (e.g., deep plane release surgical procedure(s) comprising subnasal and corner lip lift modification(s)), or combination(s) of some, most, generally all, or all thereof, wherein the concepts are associated with or otherwise accessible to morphing software.

Herein morphing software refers to software capable of combining a plurality of data sets related to images (picture(s)), such as, e.g., an image of a subject with data related to idealized or target parameter(s). In aspects, the programming of concept(s) and data described herein into morphing software results in the ability to increase the predictability of outcome(s) detectably or significantly; to provide subject(s), practitioner(s), or both with a preview of what an outcome of aesthetic modification procedure(s) will be prior to their performance; to guide practitioner(s) in the performance of procedure(s); or combination(s) thereof. In aspects, for example, image(s) of a subject can be made available to morphing software. Further, the morphing software can be programmed to comprise data related to system(s) and method(s) described herein, e.g., data related to excision height at particular location(s) of the upper lip; data related to final lip height at particular location(s) of the upper lip; data related to the relationship(s) between various segment(s)/portion(s) of radial marker(s) of marking system(s); data related to relationship(s) between various marker(s) position(s); etc., such as that/those described herein.

In aspects, the disclosure provides morphing system(s) (a system that can perform facial image morphing function(s)) comprising image(s) of subject(s) upon which aesthetic modification procedure(s) are to be performed, data related to system(s) and method(s) described herein (such as, e.g., data exemplified here), wherein the morphing system(s) combine such available input(s) and provide an output demonstrating an expected outcome given specific parameter(s) elected by morphing system operator(s). In aspects, parameter(s) elected by a morphing system operator(s) can comprise, e.g., parameter(s) related to one or more elected target effect(s) such as lip height, lip shape, lip slope, nose shape, nose-lip balance, lip-chin balance, nose-chin balance, nose-cheek balance, lip-cheek balance, etc. In aspects, parameter(s) elected by morphing system operator(s) can comprise, e.g., noting what type(s) of procedure(s) are to be performed, e.g., a subnasal upper lip lift, subfacial lip lifting, corner lip lift(s), nasal base suspension, muscle suspension, combination subnasal and corner lip lift procedure with or without nasal base suspension, muscle suspension, or both, etc. In aspects, parameter election can be facilitated by a system interface, e.g., by providing an operator with the ability to elect via the interface, e.g., by toggling on/off specific elements, by electing “yes” or “no” with regard to the incorporation of specific elements, by adjusting one or more, e.g., 2 or more, 3 or more, 4 or more, 5 or more, or, e.g., 6 or more (e.g., by a gradient adjustment mechanism such as, e.g., a slider button), etc. specific parameter(s). In aspects, a subject, practitioner, or both, can modify one or more parameter(s) based upon a provided output of the morphing system(s) provided here. In aspects, parameter(s) can comprise marking(s)/location(s) related to the total length of or, e.g., the excision height of, one or more 1 (006), 2A (008A), 2B (008B), 3A (010A), 3B (010B), 4A (012A), 4B (012B), 5A (014A), 5B (014B), H-A (046A), H-B (046-B), LE-A (018A), and LE-B (018B).

In aspects, a predicted aesthetic appearance is provided by morphing system(s) provided here based upon a first set of user inputs and, after a step of evaluating the output (e.g., consideration of the predicted outcome by the subject, practitioner, or both) one or more input parameter(s) is adjusted and the evaluation is re-performed by the morphing system(s), yielding an updated predicted result/outcome. This can, in aspects, be iterated a plurality of times, e.g., 2 or more times, e.g., 3, 4, 5, 6, 7, 8, 9, or 10 or more times, until parameters yielding target effect(s) are achieved. In aspects, iterations are captured by machine-learning process(es)/programming/mechanism(s) present in morphing software provided herein, such that the target aesthetic appearance, e.g., target aesthetic effect(s) are achieved with detectably or significantly fewer iterations over time, e.g., through use of the morphing software in such application(s).

In aspects, automated system(s) provided herein utilize one or more of facial recognition technology(ies), artificial intelligence technology(ies), and algorithmic calculation(s) to provide assistance in performing lip lift procedure(s), such as upper lip lift procedure(s), such as, e.g., deep plane lip lift procedure(s). In aspects, automated system(s) comprise the ability to receive one or more user input(s). In aspects, a user input can be any input that would modify a resulting appearance modification to a subject photograph or to an actual subject patient if implemented in an associated aesthetic modification procedure. In aspects, such an input can be, e.g., an excision height, incisive display, skin type, presence of fillers, and, e.g., a photograph (e.g., a photograph of a potential subject patient).

FIG. 21 provides an exemplary embodiment of the application of landmark recognition technology within automated system(s), e.g., within mobile application(s) provided herein. In aspects, landmark recognition technology is applied to an image, e.g., an original image (but which may also be applied to, e.g., a morphed image), wherein the landmark recognition technology identifies a plurality of facial landmark(s). In aspects, ≥1, ≥˜10, ≥˜25, ≥˜50, ≥˜75, ≥˜100, ≥˜125, ≥˜150, ≥˜170, ≥˜200, ≥˜225, ≥˜250, ≥˜275, ≥˜300, ≥˜325, ≥˜350, ≥˜375, ≥˜400, ≥˜425, ≥˜450, ≥˜475, or, e.g., ≥˜500 landmark(s) of a human face is identified by such technology(ies). In aspects, landmark recognition technology recognizes/identifies and establishes at least one location within an image of a human face representing 1-square inch (1 in²) of the actual human face comprising at least about 2 landmarks, such as ≥˜3, ≥˜4, ≥˜5, ≥˜6, ≥˜7, ≥˜8, ≥˜9, ≥˜10, ≥˜15, or ≥˜20 landmarks. In aspects, landmark recognition technology(ies) of automated system(s) herein recognize/identify and establish at least 1, such as at least about 2, ≥˜3, ≥˜4, ≥˜5, ≥˜6, ≥˜7, ≥˜8, ≥˜9, ≥˜10, ≥˜15, or, e.g., ≥˜20 landmarks along the upper vermillion border. In some aspects, landmark recognition technology(ies) of automated system(s) provided herein establish at least about 2, such as ≥˜5, ≥˜10, ≥˜15, ≥˜20, ≥˜25, ≥˜30, ≥˜35, ≥˜40, ≥˜45, ≥˜50, ≥˜55, ≥˜60, ≥˜65, ≥˜70, ≥˜75, ≥˜80, ≥˜85, ≥˜90, ≥˜95, ≥˜100, ≥˜110, ≥˜120, ≥˜130, ≥˜140, ≥˜150, ≥˜175, ≥˜200, ≥˜225, ≥˜250, ≥˜275, ≥˜300, ≥˜350, ≥˜400, ≥˜450, or even, in aspects, ≥˜500 landmarks between the left and right relaxed skin tension lines. In some aspects, landmark recognition technology(ies) of automated system(s) provided herein establish at least about 2, such as ≥˜5, ≥˜10, ≥˜15, ≥˜20, ≥˜25, ≥˜30, ≥˜35, ≥˜40, ≥˜45, ≥˜50, ≥˜55, ≥˜60, ≥˜65, ≥˜70, ≥˜75, ≥˜80, ≥˜85, ≥˜90, ≥˜95, ≥˜100, ≥˜110, ≥˜120, ≥˜130, ≥˜140, ≥˜150, ≥˜175, ≥˜200, ≥˜225, ≥˜250, ≥˜275, ≥˜300, ≥˜350, ≥˜400, ≥˜450, or even, in aspects, ≥˜500 landmarks between the nasal base and the upper vermillion border.

According to aspects, as shown in FIG. 21, landmark recognition technology(ies) can present such identified landmarks as points on an image. In aspects, landmark recognition technology(ies) of automated system(s) provided herein can connect such point(s). In aspects, distance(s) between points, orientation(s) (direction and distance, for example) between point(s), or both is/are captured by the automated system(s) and used in one or more morphing algorithm(s) herein. In aspects, landmark recognition technology(ies) of automated system(s) herein, e.g., mobile application(s) disclosed herein, present the results of the application of landmark recognition as a “face mesh,” e.g., a series of points connected by lines forming a network of connected locations, as shown in FIG. 21. According to aspects, one, some, most, generally all, substantially all, essentially all, or all of the facial features, locations, landmarks, etc. described herein are recognized and indicated by facial recognition technology(ies) and, in aspects, are presented as locations within a face mesh.

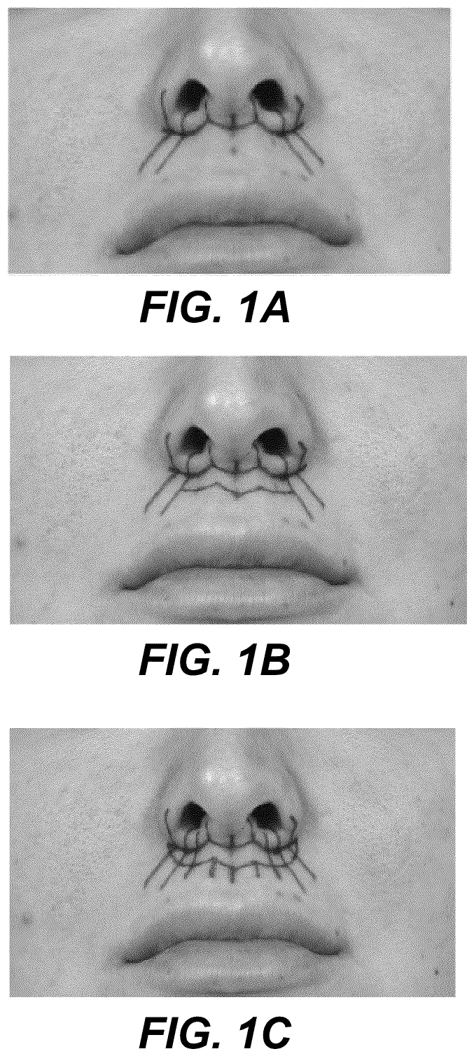

As provided in FIG. 1, and further discussed below, upon the identification of facial landmark(s) by facial recognition technology(ies) of automated system(s), a user can modify one or more input(s), e.g., at least 1 input, ≥2, ≥3, ≥4, ≥5, ≥6, ≥7, ≥8, ≥9, or, e.g., ≥10 inputs via an interface within which the results of the application of the landmark recognition technology to an image are presented to automated system user. For example, a screen within an automated system, e.g., a mobile application, in aspects, presents an image with landmark(s) identified, e.g., with a face mesh applied (as exemplified in FIG. 21), wherein the screen further comprises one or more selectable options that the automated system user can use to morph the image, modifying the position of location indicator(s) associated with one or more of the marking(s) of marking system(s) described herein. In aspects, an automated system user can modify the one or more attribute(s) of locations associated with one or more of, e.g., marker(s) (location(s)) 1, 2A, 2B, 3A, 3B, 4A, 4B, 5A, 5B, H-A, H-B, LE-A, LE-B, upper excision boundary, lower excision boundary, segment(s) of any marker(s) associated with those identified here or elsewhere herein, etc. For example, as shown in FIG. 21, four slider bars (identified as 1, 2, 3, and 4 in FIG. 21) are provided on a user interface of an automated system presenting an original image that has had landmark recognition technology applied. Each slider bar represents, e.g., location(s) associated with one or more of marker(s)/location(s), e.g., 1, 2A, 2B, 3A, 3B, 4A, 4B, 5A, 5B, H-A, H-B, LE-A, LE-B, upper excision boundary, or, e.g., lower excision boundary. In aspects, a user can adjust the slider on, e.g., slider bar 1, which may, for example, be associated with radial marker 1. In certain aspects, an adjustment may be, e.g., to shorten the location associated with radial marker 1—for example e.g., the final lip height at the location of radial marker 1. In aspects, such an adjustment is an exemplary input (input(s) described herein). In aspects, such an adjustment can be made, for example, by sliding the slider bar number 1 to the left. In aspects, in doing so, the image is morphed to provide what such an adjustment would look like in real life if applied during an aesthetic modification procedure, such as, e.g., by excising the upper lip an amount to result in the new final lip height input directed to radial marker 1. In aspects, a user may adjust any number of adjustable inputs presented; for example, input (e.g., illustrated as a slider bar) number 2 shown in FIG. 21 may allow for the adjustment of, e.g., radial marker(s) 2A, 2B, or both. Further, input number 3 in FIG. 21 may allow for the adjustment of, e.g., radial marker(s) 3A, 3B, or both. Still further, input number 4 in FIG. 21 may allow for the adjustment of, e.g., radial marker(s) 4A, 4B, or both. In aspects, as a user adjusts one, some, or all optional input(s), the presented image is morphed so as to predict what the outcome would be if such associated input(s) were established as a feature set and applied in an aesthetic modification procedure.

In aspects, a user can continue to adjust an image until a desired aesthetic appearance is reached or, e.g., until target feature characteristic(s) are attained. In aspects, automated system(s) can comprise stored rules which allow for the system(s) to automatically adjust input(s) to attain target feature characteristic(s). In aspects, target characteristic(s) can be any identifiable target characteristic; such as, e.g., typically one or more target aesthetic characteristic(s) described herein, such as a target slope of the upper vermillion border at one or more location(s), a target incisor show, or both.

In aspects, upon achieving a desired, or potentially suitable, target aesthetic appearance, the automated systems(s) can allow a user to save the morphed image for future retrieval and use.

In aspects, automated system(s), e.g., mobile application(s), provided herein can comprise landmark detection software, landmark detection library(ies), or both.

In aspects, landmark detection software, library(ies), or combination(s) thereof present in automated system(s), e.g., mobile application(s), of this disclosure automatically detect one or more visual landmark(s), e.g., facial landmark(s), on, within, or of a live image (e.g., a live camera image), a photograph, or both. In aspects, such detection feature(s) assist in placing one or more visual guide(s) and measurement(s) upon a live image or photograph, taking into consideration individual characteristic(s) of the live image or photograph. In aspects, the live image is a live image of a mammalian face, such as a human face. In aspects, automated system(s) described herein comprise landmark detection software, library(ies), or combination(s) thereof capable of automatically detecting one or more facial landmark(s). In aspects, the detection assists in placing visual guide(s) and measurement(s) accurately upon a subject patient's live image or photograph, considering the individual facial characteristics thereof.

According to aspects, automated system(s), e.g., mobile application(s), provided herein comprise morphing technology(ies). In aspects, morphing technology is the ability to apply one or more rule(s), input(s), setting(s), etc. to an image, e.g., a live image or, e.g., a photograph, to produce a modified image which take(s) into account the rule(s), input(s), setting(s) applied and which is different from the original image.

According to certain aspects, automated system(s), e.g., mobile application(s), disclosed herein comprise morphing technology(ies) which allow program or application user(s), e.g., physicians or potential patients, to visualize before and after effect(s) of a proposed aesthetic modification procedure, such as a lip lift.

In aspects, automated system(s), e.g., mobile application(s), comprise morphing technology which can apply data related to one or more desired feature(s) or characteristic(s) of visual appearance, e.g., facial feature(s) appearance, e.g., upper lip size, shape, or fullness, etc. to an image of a face (e.g., a mammalian, e.g., human face), wherein the image of the face fails to represent the desired feature(s) or characteristic(s) of visual appearance, and wherein the application of the data results in the generation of a new image which modifies the original image of the face to comprise the desired feature(s) or characteristic(s).

In aspects, morphing technology(ies) of automated system(s) herein evaluate or consider (take into consideration) one or more individual characteristic(s) of the original image, e.g., one or more facial characteristic(s) of an image of a face. For example, morphing technology(ies) of automated program(s) provided herein consider one or more facial characteristic(s). In aspects, such consideration aids in the generation of a resulting morphed image once the one or more rule(s), input(s), setting(s), etc. are applied to the image.

In aspects, data utilized to generate a morphed image, e.g., a morphed image representing a target appearance of a human face (such as, e.g., a target appearance of the upper lip) is captured or, e.g., is identified as a feature set. In aspects, morphed image(s) generated by morphing technology(ies) of automated system(s) provided herein can be identified as being a target desired outcome or appearance of an aesthetic modification procedure. In aspects, the captured data resulting in such morphed image(s) can be saved, transmitted, shared, or applied in aesthetic modification procedure(s) to realize the target desired outcome.

According to aspects, morphing technology(ies) provided in automated system(s), e.g., mobile application(s), provided by this disclosure comprise one or more intensity modulation setting(s) (e.g., morphing intensity settings) which provide a system (or, e.g., application) user with the ability to adjust the morphing intensity applied to an original image. In aspects, such morphing intensity adjustment capability provides user(s) with the ability to control the degree of morphing applied to an image. In aspects, the ability to control the degree of morphing results in the ability to apply variations, or levels, degrees, or otherwise modified version(s) of, one or more provided rule(s), input(s), or setting(s) to a given image, resulting in the ability to generate morphed image(s) which significantly vary from an/the original (e.g., applying high morphing intensity) or which only slightly or barely discernably vary from an/the original (e.g., applying low morphing intensity).

In aspects, morphing technology(ies) provided by (present as a feature of) automated system(s), e.g., mobile application(s) disclosed herein, are used to establish a feature set anticipated to result in a given visual appearance when applied in an aesthetic modification procedure. In aspects, application of the feature set established by the morphing technology(ies) in the aesthetic modification procedure results in the final aesthetic appearance resulting from the aesthetic modification procedure being at least generally the same as, at least mostly the same as, at least substantially the same as, at least essentially the same as, or the same, as that predicted by the morphing technology(ies) (e.g., as that provided in a generated in morphed image(s)). Such congruence can be established by, e.g., the comparison of one or more indicator(s), such as, e.g., final lip height at the position of one or more radial indicator(s) compared to the predicted final lip height at the position of such indicator(s), the comparison of the slope of the upper vermillion border at one or more location(s) compared to the predicted slope of the upper vermillion border at such location(s), etc. Such a comparison can be conducted by the patient, a medical professional participating in the conduct of the procedure or subsequent care of the patient, automated technology(ies), or by unrelated medical professional(s) appropriately trained in the field of aesthetic modification.

According to aspects, automated system(s) disclosed herein can comprise any suitable landmark detection system or technology(ies), e.g., any suitable landmark detection system platform.

In aspects, a suitable landmark detection technology of automated system(s) disclosed herein comprises a vision framework. In aspects, the vision framework can be any suitable vision framework providing face detection and landmark detection capabilities. In aspects, the vision framework is an Apple framework. In aspects, an Apple framework is Apple's Vision framework that is commonly built into Apple device(s), such device(s) being, in aspects, suitable for providing the automated system(s), e.g., mobile application(s), described herein. In aspects, a suitable vision framework is any vision framework having one, some, most, generally all, substantially all, essentially all, or all of the feature(s) or functionality of Apple's Vision framework.

In aspects, landmark detection technology(ies) of automated system(s) herein can comprise library(ies comprising one or more facial landmark detection algorithms. In aspects, such library(ies) can be any suitable library(ies) offering landmark detection algorithm(s). In aspects, the library(ies) can be open-source library(ies). In one example, a suitable library is OpenCV. In aspects, a suitable library, e.g., vision library, is any library, e.g., vision library having one, some, most, generally all, substantially all, essentially all, or all of the feature(s) or functionality of OpenCV; a library having at least generally the same, at least substantially the same, at least essentially the same, or the same depth and breadth of coverage as OpenCV in terms of vision/visual data; a library having at least generally the same, at least substantially the same, at least essentially the same, or the same algorithmic breadth, scope, capability, etc., as OpenCV, or combination(s) thereof.

In aspects, landmark detection technology(ies) of automated system(s) herein can comprise framework(s) providing landmark detection capability(ies), e.g., facial landmark detection capability(ies), which provide pre-trained model(s), support real-time processing, or which provide pre-trained model(s) and support real-time processing. In aspects, such framework(s) can be any suitable framework(s) providing pre-trained model(s), supporting real-time processing, or both. In aspects, a suitable framework is a framework developed by Google. In aspects, a suitable framework is Mediapipe. In aspects, a suitable framework is any framework having one, some, most, generally all, substantially all, essentially all, or all of the feature(s) or functionality of Mediapipe.

According to certain aspects, automated system(s) provided herein comprise one or more facial landmark detection technology(ies) comprising, or which are at least mostly, at least generally, at least essentially, are essentially, or are equivalent to, or in aspects which are superior to (in terms of breadth/scope/functionality/operational benefit) one, some, or all of Apple's Vision framework, Open CV vision library, and Mediapipe open-source framework.

According to aspects, automated system(s) disclosed herein comprise one or more morphing technology(ies) (image morphing technology(ies). In aspects, such technology(ies) provide one or more tools for image processing and morphing effect(s). In aspects, such technology(ies) suitable for use in automated system(s) disclosed herein can be any morphing technology(ies) providing tool(s) for image processing and morphing effect(s), In aspects, a suitable morphing functionality is provided by an Apple framework. In aspects, a suitable morphing functionality is provided by Apple's Core Image. In aspects, A suitable morphing functionality is a functionality/technology providing one, some, most, generally all, substantially all, essentially all, or all feature(s) and function(s) of Apple's Core Image framework.

In aspects, automated system(s) disclosed herein comprise one or more morphing technology(ies) capable of creating augmented reality experience(s) for visualizing one or more before and after effect(s) of an applied set of rules or actions, such as, e.g., before and after effects of a proposed lip lift procedure. In aspects, suitable morphing technology(ies) are any technology(ies) capable of creating augmented reality experience(s), e.g., experiences for visualizing before and after effect(s), e.g., effect(s) of the application of applied rule(s) or action(s). In aspects, a suitable morphing technology is an Apple framework such as, e.g., ARKit. In aspects, suitable morphing technology(ies) is/are technology(ies) comprising one, some, most, generally all, substantially all, essentially all, or all feature(s) and function(s) of ARKit. In aspects, a suitable morphing technology is an augmented reality experience platform suitable for use within Android device(s). In aspects, a suitable platform is a Google platform. In aspects, a suitable platform is ARCore. In aspects, suitable morphing functionality(ies)/technology(ies) are platform(s) comprising one, some, most, generally all, substantially all, essentially all, or all feature(s) and function(s) of ARCore.

In aspects, automated system(s) disclosed herein comprise one or more morphing technology(ies) capable of image processing and morphing effects, e.g., creating augmented reality experience(s), e.g., experiences for visualizing one or more before and after effect(s), such as, e.g., one or more of Core Image, ARKit, and ARCore, or one or more technology(ies) providing one, some, most, generally all, substantially all, essentially all, or all of the feature(s) and function(s) of one, two, or all of Core Image, ARKit, and ARCore.

According to aspects, automated system(s) provided herein can comprise facial landmark detection algorithms providing facial landmark detection and tracking capability(ies), landmark detection algorithms provided by libraries such as C++ library(ies), or, e.g., C++ library(ies) with Python binding(s), or both. In aspects, an exemplary library is dlib. In aspects, automated system(s) can comprise facial landmark detection algorithms such as those provided by dlib or other trained models providing at least mostly the same, generally the same, substantially the same, essentially the same, or at least the same (or better/more accurate) landmark, e.g., facial landmark, detection landmark algorithms as dlib. In aspects, an exemplary platform/toolkit is OpenFace. In aspects, automated system(s) can comprise facial landmark detection algorithms such as those provided by OpenFace or other toolkits providing at least mostly the same, generally the same, substantially the same, essentially the same, or at least the same (or better/more accurate) landmark, e.g., facial landmark, detection landmark algorithms as OpenFace. In aspects, automated system(s) can comprise facial landmark detection algorithms such as those provided by FaceSDK. In aspects, automated system(s) can comprise facial landmark detection algorithms such as those provided by FaceSDK or other toolkits providing at least mostly the same, generally the same, substantially the same, essentially the same, or at least the same (or better/more accurate) landmark, e.g., facial landmark, detection landmark algorithms as FaceSDK. According to aspects, one, some, most, generally all, substantially all, essentially all, or all frameworks used to provide facial landmark detection algorithms can support a plurality of programming languages, including, e.g., Swift.

### Artificial Intelligence-Based Calculations & Machine Learning Functionality

In aspects, automated system(s), e.g., mobile application(s) provided herein utilize artificial intelligence (AI) technology(ies).

In aspects, artificial intelligence model(s) of system(s) herein can be trained, updated/modified (e.g., fine-tuned), or both using one or more curated datasets. In aspects, the use of a curated dataset detectably or significantly improves the accuracy and performance of system(s) herein.

In aspects, automated system(s), e.g., mobile application(s) provide AI-based calculation(s) for aesthetic appearance modification measurement(s), e.g., facial modification measurement(s), e.g., calculation(s) for lip lift measurement(s).

In aspects, iterations are captured by machine-learning process(es)/programming/mechanism(s) present in morphing software provided herein, such that the target aesthetic appearance, e.g., target aesthetic effect(s) are achieved with detectably or significantly fewer iterations over time, e.g., through use of the morphing software in such application(s). In aspects, training or fine-tuning of system(s), e.g., application(s) herein, comprise a generative artificial intelligence component or generative adversarial network model. In aspects, AI model(s) provided herein can detect one or more facial landmark(s) identified herein by, e.g., radial markers 1, 2 (2A, 2B), 3 (3A, 3B), 4 (4A, 4B), 5 (5A, 5B), etc. markers of marking system(s) described herein. In aspects, a generative AI model of system(s) herein is a model that, given a photograph or image of a human face (e.g., of the user's face) and one or more required values for particular facial landmark(s), e.g., one or more required values for, e.g., one or more markers (1, 2A, 2B, 3A, 3B, 4A, 4B, 5A, 5B, etc.); slope of the upper vermillion border; and, e.g., height differential between where any two adjacent markers of 1, 2A, 3B, 3A, 4A, 4B, 5A, and 5B meet the upper vermillion border; as described herein, automatically generate(s) image(s) of a face having feature(s) that correspond(s) to the defined value(s).

In aspects, automated system(s), e.g., mobile application(s), herein utilize artificial intelligence (AI) algorithm(s). In aspects, AI algorithm(s) can comprise, e.g., one or more deep learning model(s). In aspects, deep learning model(s) can be trained on training data, such as, e.g., surgery-related data, e.g., aesthetic modification surgery data. In aspects, aesthetic modification surgery data can be related to any aesthetic modification surgical procedure, such as, e.g., a facial modification procedure, such as, e.g., a lip lift. Accordingly, in aspects, automated system(s), e.g., mobile application(s), herein utilize one or more AI algorithm(s), such as deep learning model(s), e.g., deep learning model(s) trained on lip lift data.

In aspects, lip lift data is any data related to lip size, shape, and volume; data related to lip length at a plurality of location(s); relative positioning of facial feature(s); relative size of facial features; result(s) data, such as result(s) from prior procedure(s) and parameter(s) applied achieving such results (such as, e.g., starting feature data, applied modification(s), such as, e.g., excision height(s)/length(s) at a plurality of location(s), excision shape, and the like); incisive display; skin type; presence or absence of filler(s), and the like.

In aspects, AI algorithms of automated system(s) are used to perform one or more calculations for lip lift measurements based on visual guide(s) provided by the automated program(s), e.g., mobile application(s). In aspects, visual guide placement(s) provided by the automated system(s) are used to calculate measurement(s) for modification(s) to be applied in an aesthetic modification procedure, such as, e.g., a lip lift procedure. In aspects, visual guide placement(s) provided by automated program(s) are used to calculate lip lift measurement(s). In aspects, the calculation(s) described here are performed by AI algorithm(s) of the automated system(s), e.g., mobile application(s) disclosed herein. In aspects, lip lift data used to train AI algorithm(s) disclosed herein comprise one or more feature(s)/characteristic(s), such as a plurality of features/characteristics, e.g. some, most, generally all, essentially all, or all features/characteristics of marking system(s) described herein (such as, e.g., feature(s)/characteristic(s) related to the positioning, length(s), ratio(s) between, etc. horizontal markers, corner lip lift marking(s), radial marker(s), target slope(s) of the vermillion border, target height differences between points of intersection of adjacent radial markers with the upper vermillion border, and other feature(s)/characteristic(s) described elsewhere herein). In aspects, AI algorithm(s) also or alternatively utilize data related to target excision height, incisive display, skin type, presence/absence of fillers to perform calculation(s).

In aspects, AI algorithm(s) perform calculation(s) to establish parameter(s) required to achieve a target outcome of an aesthetic modification procedure, e.g., upper lip lift procedure, wherein such a target outcome is, e.g., related to lip length, upper vermillion border slope, incisive display, or combination(s) thereof. According to certain aspects, AI algorithm(s) utilize one or more input(s) provided by automated system user(s), one or more input(s) present as existing data related to a preestablished a target outcome or feature thereof (e.g., one or more saved setting(s)), one or more input(s) established by a user selecting the one or more input(s) from a menu of saved option(s), or combination(s) thereof, to determine one or more change(s) required by an aesthetic modification procedure, such as a lip lift, to achieve a target outcome. In aspects, AI algorithm(s) utilize one or more input(s) (e.g., input(s) described here) to calculate modifications, such as changes in vertical height of the upper lip at one or more locations of the upper lip required to achieve a target outcome. In aspects, AI algorithms, e.g., deep learning models trained on lip lift data, are used to perform calculation(s) for lip lift measurement(s) based on visual guide placement(s) and utilize provided input(s) to calculate the vertical change required at one or more location(s) to achieve a target upper lip appearance.

In aspects, AI algorithm(s) evaluate(s) the amount of excess tissue (e.g., tissue characterizable as “sagging” tissue) to determine the necessary lift for achieving a target visual appearance. In aspects, excess tissue can be tissue of the subfacial lip. In aspects, excess tissue can be tissue lateral to radial marking 4 (e.g., radial marking 4A or 4B described herein). In aspects, AI algorithm(s) evaluate(s) the discrepancy between, e.g., the difference between, nasal width and labial width to determine intervention(s) required to achieve a target visual appearance. In aspects, AI algorithm(s) evaluate(s) the amount of excess tissue and the difference between nasal and labial width to calculate intervention(s) required to achieve a target aesthetic appearance, e.g., to achieve a target upper lip appearance.

In aspects, artificial intelligence capability(ies) described herein allow for system(s) to learn from one or more comparison(s), e.g., comparison(s) of a predicted outcome with an actual outcome (and the data associated with the predicted outcome relative to the actual outcome or data associated therewith) wherein such machine learning results in the detectable or significant improvement in predictability of final outcome(s) based on or incorporating one, some, most, generally all, substantially all, essentially all, or all element(s) of given feature set data.

In some aspects, predicted aesthetic appearance(s) (which can include, e.g., data associated therewith) provided by morphing system(s) provided here, which are based upon/generated according to a first set of user inputs, are evaluated by a system user (e.g., subject/patient, medical professional, or both), and one or more input parameter(s) is/are adjusted, whereafter morphing system(s) provide an updated predicted result/outcome. In aspects, just an image provision-review-editing cycle can be iterated a plurality of times, e.g., 2 or more times, e.g., 3, 4, 5, 6, 7, 8, 9, or 10 or more times until parameter(s) yielding target effect(s) are achieved. In aspects, such iterations are captured by machine learning. In aspects, such machine-learning detectably or significantly improves or reduces, in one or more subsequent system use(s), the number of provision-review-editing cycle(s) required to reach acceptable target outcome(s).

According to aspects, automated system(s) of this disclosure comprise one or more AI-based calculation technology(ies). In aspects, such technology(ies) can be any suitable technology(ies). In aspects, an AI-based calculation technology suitable for incorporation in automated system(s) disclosed herein enables integration of pre-trained deep learning models for such AI-based calculation(s). In aspects, suitable AI-based calculation technology(ies) is any AI-based calculation enabling integration of pre-trained deep learning models for such calculation(s). According to aspects, such a suitable technology is an Apple framework. In aspects, the Apple framework is Core ML. In aspects, suitable technology(ies) is/are technology(ies) which comprise one, some, most, generally all, substantially all, essentially all, or all of the feature(s) and function(s) of Apple's Core ML.

In aspects, automated system(s) of this disclosure comprise one or more AI-based calculation technology(ies) characterizable as machine learning framework(s) capable of training, deploying, or training and deploying customized deep learning model(s). In aspects, such framework(s) are specifically tailored for lip lift measurements. In aspect(s), automated system(s) comprise or utilize (e.g., are developed using) one or more machine learning library(ies) suitable for training AI model(s) that detect and modify facial landmark(s). According to aspects, suitable AI-based calculation technology(ies), e.g., machine learning frameworks capable of being used to train, deploy, or train and deploy custom deep learning models, e.g., models specifically tailored for lip lift measurements, can be any suitable technology(ies), such as, for example, TensorFlow, PyTorch, and the like. In aspects, suitable technology(ies) is/are technology(ies) which comprise one, some, most, generally all, substantially all, essentially all, or all of the feature(s) and function(s) of one or both of TensorFlow and PyTorch.

According to aspects, automated system(s) of this disclosure comprise one or more AI-based calculation technology(ies) enabling interoperability between a plurality of deep learning frameworks. In aspects, such a technology provides the ability to use one or more model(s) trained in a first framework, e.g., a framework described herein, such as, e.g., TensorFlow, PyTorch, etc., in a second framework. In aspects, such ability(ies) provide flexibility of design of AI framework(s) for specific calculation(s), such a calculation(s) directed to lip lift calculations. In aspects, AI-based calculation technology(ies) enabling interoperability between a plurality of deep learning frameworks can be any suitable framework providing, e.g., the ability to use model(s) trained in a first framework within a second framework. In aspects, a suitable technology is Open Neural Network Exchange (ONNX). In aspects, suitable technology(ies) is/are technology(ies) which comprise one, some, most, generally all, substantially all, essentially all, or all of the feature(s) and function(s) of ONNX.

According to aspects, automated system(s) of this disclosure comprise one or more AI-based calculation technology(ies). In aspects, such a technology can be any suitable technology, such as, e.g., Core ML, TensorFlow, PyTorch, ONNX, etc. In aspects, one or more AI-based calculation technology(ies) present in automated system(s) disclosed herein is one or more of Core ML, TensoFlow, PyTorch, and ONNX or comprises one, some, most, generally all, substantially all, essentially all, or all of the feature(s) and function(s) of one or more of Core ML, TensorFlow, PyTorch, ONNX, or combination(s) thereof.

According to aspects, automated system(s) provided herein comprise one or more artificial intelligence data sets for use in, e.g., training deep learning models for facial analysis task(s). In aspects, automated system(s) provided herein comprise any suitable artificial intelligence data sets capable of training deep learning models for facial analysis task(s), such as, e.g., CelebA. In aspects, automated system(s) herein comprise or utilize CelebA in or for training deep learning model(s) for facial analysis task(s) required by/for automated system(s) disclosed here. In aspects, automated system(s) herein comprise or utilize one or more data set(s) comprising at least mostly the same, at least generally the same, at least substantially the same, at least essentially the same, essentially the same, or the same, or greater scope (depth, breadth, and variety) of data as provided by CelebA.

In aspects, automated system(s) provided herein comprise one or more artificial intelligence data sets that can provide landmark annotations, e.g., facial, or specifically lip landmark annotations, for a plurality, e.g., tens, hundreds, thousands, or, e.g., tens of thousands of feature(s), e.g., faces, in real-world images. In aspects, such data sets can be used for training and evaluating facial landmark detection model(s). In aspects, annotations are ground truth annotations. In aspects, artificial intelligence data sets providing landmark annotation(s) can be any suitable AI data set(s) providing relevant landmark annotation(s) such as, e.g., the Annotated Facial Landmarks in the Wild (AFLW) data set, the 300 Faces In-the-Wild Challenge (300-W) dataset, the Lip4K dataset, and the like. In aspects, the deep learning model(s), artificial intelligence-based calculation(s) provided herein are trained using one or more of AFLW, 300-W, and Lip4K or one or more data set(s) comprising at least mostly the same, at least generally the same, at least substantially the same, at least essentially the same, essentially the same, or the same, or greater scope (depth, breadth, and variety) of data as provided by AFLW, 300-W, and Lip4K.

In aspects, automated system(s) provided herein comprise artificial intelligence algorithms comprising deep learning model(s) trained using one or more of CelebA, AFLW, 300-W, Lip4K, or one or more artificial intelligence data set(s) comprising at least mostly the same, at least generally the same, at least substantially the same, at least essentially the same, essentially the same, or the same, or greater scope (depth, breadth, and variety) of data as provided by CelebA, AFLW, 300-W, and Lip4K.

In aspects, automated system(s) of the present disclosure comprise artificial intelligence-based calculation(s) comprising deep learning model(s) which are at least as accurate and successful, or otherwise suitable as artificial intelligence-based calculation(s) comprising deep learning model(s) trained using one or more of CelebA, AFLW, 300 W, and Lip4K.

According to some aspects, automated system(s) provided in this disclosure comprise an ability to compare two or more images, such as, e.g., three or more, four or more, five or more, 10 or more, 20 or more, 50 or more, or e.g. even 100 or more images. In certain specific aspects, automated system(s) provided herein can compare, e.g., an original image to an idealized image. In aspects, automated system(s) provided herein can identify from such a comparison one or more input(s) requiring a change in order to create a morphed version of an original image having feature(s) which is/are closer to matching corresponding feature(s) of the idealized image. In aspects, automated system(s) provided herein can compare, e.g., a morphed image generated by the system(s) to an idealized image. In aspects, such a comparison can allow, e.g., a scoring system to be applied to rank or score how close to the feature(s) of the idealized image one or more corresponding feature(s) of the morphed image are. In aspects, automated system(s) provided herein can compare, e.g., an image reflecting the outcome of a conducted aesthetic modification procedure, e.g., an aesthetic modification procedure performed using automated system(s) described herein, to an idealized image, a morphed image (e.g., a morphed image predicting the outcome of the conducted aesthetic modification procedure, or both. In aspects, such comparison(s) can allow, e.g., a scoring system to be applied to rank or score how close to feature(s) of the image reflecting the final outcome of a procedure (or procedures) are to corresponding features of an idealized image, morphed image (predictive image), or both. In aspects, comparison(s) of images can facilitate, e.g., longitudinal tracking of, e.g., healing, promote early adverse event detection, or both. Comparison(s) described herein can be an element of both automated method(s), manual method(s), or both. Comparison(s) herein, when part of manual method(s), may be performed only by human(s). Comparison(s) herein, when part of automated method(s), may be performed by automated system(s) alone or in conjunction with human assistance (e.g., may comprise one or more elements of human evaluation.)

### Editing Visual Landmark(s)

In aspects, one, some, most, generally all, substantially all, essentially all, or all visual guide(s) generated by automated system(s), disclosed herein are modifiable by automated system user(s). In aspects, automated system(s) provided herein comprise the ability for one or more automated system user(s) to manually adjust one or more of the visual guide(s) generated by the automated system(s) via an interface of the automated program(s), e.g., mobile application(s).

According to aspects, automated system(s) of this disclosure comprise an artificial intelligence model comprising one or more generative model(s) capable of altering specific feature(s) of an image, e.g., facial feature(s), such as, e.g., lips, according to provided characteristic(s) of measurement(s) of marker(s) of marking system(s) provided herein. In aspects, once altered, such model(s) can, in aspects, generate a natural-looking face image which reflect(s) such characteristic(s). In aspects, a Generative Adversarial Network (GAN) is used to develop generative model(s) suitable for automated system(s) herein.

According to aspects, automated system(s) herein, e.g., mobile application(s) described herein, comprise one or more interactive element(s) or feature(s). In aspects, such interactive element(s) can be any feature of the system(s) with which a user interacts. In aspects, such interactive element(s) can be, e.g., data capture and data manipulation feature(s). In one aspect, an interactive element can allow a user to view one or more image(s), such as one or more manipulated (morphed) image(s). In one aspect, interactive element(s) can be element(s) that allow user(s) to capture an original image (e.g., a starting aesthetic appearance), such as allowing the user to take a photograph, retrieve a saved photograph, and the like. In aspects, interactive element(s) can be element(s) which allow user(s) to adjust one or more marking measurement(s), e.g., marking measurement(s) of one or more marker(s) described herein, e.g., measurement(s) associated with marker(s) 1, 2A, 2B, 3A, 3B, 4A, 4B, 5A, 5B, H-A, H-B, LE-A, LE-B, or any 2 or more, 3 or more, 4 or more, 5 or more, 6 or more, 7 or more, 8 or more, 9 or more, 10 or more, 11 or more, 12 or more, or, e.g., all 13 of such marker(s). In aspects, parameter(s) such as, e.g., measurement(s) associated with one or more marker(s) can be manipulated via interactive element(s) such as, e.g., slider(s), input field(s), or any suitable element for entering, changing, or saving data. This is described above, e.g., in the section entitled, “Landmark Detection & Morphing Functionality,” and is exemplified in, e.g., FIG. 21, providing an exemplary user interface with an automated system, e.g., a mobile application, comprising 4 exemplary adjustment features, illustrated as slider(s) (slider bar(s)), which can each be individually adjusted to adjust one or more measurement(s) associated with the location associated with each slider.

### Secondary Procedure Recommendation(s)

According to certain embodiments, automated system(s), e.g., mobile application(s), provided herein comprise the ability to assess collected data (e.g., data collected by landmark detection technology(ies)) and provide a determination to system user(s) regarding the possible benefit of performing an ancillary procedure, e.g., a procedure secondary to a primary aesthetic modification procedure, such as, e.g., a corner lift procedure conducted in combination with a primary lip lift procedure. In certain aspects, a target appearance is an appearance comprising adequate vermillion exposure across the entire length (or substantially entire length/essentially entire length or generally entire length) of the upper lip. Accordingly, for example, in aspects, treatment of (intervention directed to) the upper lip, lateral to radial marker(s) 4A (012A), 4B (012B), or both, may require a corner lip lift to expose, e.g., a buried vermillion or, e.g., to avoid a change (e.g., a drop) in slop of the vermillion border. According to certain aspects, automated system(s) of the present disclosure provide a recommendation for one or more additional procedure(s) to be performed concomitantly with an upper lip lift procedure, such as, e.g., a corner lift, based on the collection and assessment of data collected by the automated system(s).

In aspects, automated system(s) herein generate data related to requirement(s) to achieve a target outcome associated with a secondary procedure, such as, e.g., a corner lift. In aspects, such data can be addressed by system(s) herein in a manner at least mostly similar to, at least generally similar to, at least substantially similar to, at least essentially similar to, essentially similar to, or the same as data described herein associated with a primary surgical procedure such as an upper lip lift procedure. Thus, in aspects, system-generated data relating to or associated with a secondary procedure (such as a possible corner lip lift) can be utilized alone to present morphed image(s) (as described elsewhere), predicting an aesthetic appearance should the elements of such data be applied in a surgical procedure. Further, in aspects, data generated relating to or associated with a secondary procedure (such as a possible corner lip lift) can be utilized in combination with other data (e.g., system-generated data relating to or associated with a primary surgical procedure such as an upper lip lift procedure) to predict an aesthetic appearance should the elements of both/all data be applied in a surgical procedure.

In certain aspects, automated system(s), e.g., mobile application(s), disclosed herein comprise one or more algorithms, rule-based system(s), or both, that analyze one or more feature(s) of an image. In aspects, analysis of image feature(s) can comprise analysis of one or more facial landmarks; one or more measurement(s); specific facial feature criteria such as the amount of subfacial lip lateral to a marker (e.g., radial marker(s) 4A, 4B, or both) of marking system(s) provided herein; the discrepancy between nasal width and labial width; one or more criteria, elements, or feature(s) of marking system(s) described herein; or combination(s) thereof. In aspects, the analysis described here is used by the automated program(s), e.g., mobile application(s) to provide a recommendation for the performance of a corner lift as a part of an originally proposed aesthetic modification procedure, e.g., upper lip lift. In aspects, the recommendation is based upon one or more of the factor(s) contemplated by the algorithm(s), rule-based system(s), or both described here. In specific aspects, the recommendation considers the height of the LE-A or LE-B lines (described elsewhere herein). In aspects, the corner lift recommendation established by automated system(s), is determined, at least in part, by the position (height) of the LE-A, LE-B, or both the LE-A and LE-B lines.

According to aspects, corner lift recommendation(s) described herein, as provided by automated system(s), e.g., mobile application(s), e.g., as determined by algorithm(s) or rule-based system(s) described here, comprise generation and provision of a confidence score. In aspects, a confidence score is a statistical score referencing the real-life accuracy of measurement(s), e.g., measurement(s) related to location(s), e.g., one or more marking(s) of a corner lift procedure(s). In aspects, a confidence score can be presented in any manner suitable for relaying assurance level, such as, e.g., using scoring such as “low,” “medium,” “high,” scaled values (e.g., a value out of a scale of 1-5, 1-10, 1-100, and the like, or any suitable scale for communicating a rank of comparison of a proposed value to a true value.

In aspects, one, some, most, generally all, substantially all, essentially all or all proposal(s), e.g., feature set(s), corner lift recommendation(s), or other directional recommendation(s) or instruction(s) described as feature(s) of automated program(s), e.g., mobile application(s) disclosed herein can be provided with a confidence score. In aspects, a confidence score relates how predictable an actual final outcome incorporating such direction/recommendation is relative to the anticipated (predicted) final outcome. In aspects, a confidence score relates to a statistical score referring to the real-life accuracy of any such proposal(s), feature set(s), recommendation(s), or instruction(s) (e.g., comparing a measurement/location to a real-life measurement/location, comparing a predicted value to an actual outcome value, or the like). In aspects, a confidence score can be presented in any manner suitable for relaying assurance level, such as, e.g., using scoring such as “low,” “medium,” “high,” scaled values (e.g., a value out of a scale of 1-5, 1-10, 1-100, or any suitable scale, and the like.

According to aspects, automated system(s) provided herein comprise function(s) providing for the ability to make one or more recommendation(s) for one or more procedure(s) based on a comparison of one or more existing feature(s) of an image compared to known target or idealized feature(s). In aspects, such functionality is provided by a rule-based system provided as one or more custom algorithm(s) or rule-based system using any suitable coding language (such as, e.g., Swift) capable of analyzing relevant factor(s) and providing recommendation(s) for procedure(s), e.g., corner lift procedure(s), based on one or more specified criteria or measurement(s).

In certain aspects, one or more machine learning library(ies) are element(s) of automated system(s) provided herein, wherein one or more such machine learning library(ies) are used in the development of one or more custom machine learning model(s) for procedure recommendation(s), such as, e.g., corner lift recommendation(s), based on specified criteria and measurement(s). In aspects, such machine learning library(ies) can be any suitable library(ies), such as, e.g., skikit-learn (Python) or, e.g., Apple's Create ML framework. In aspects, one or more machine learning library(ies) can be any machine learning library such as, e.g., skikit-learn, Create ML, or any one or more library(ies) having one, some, most, generally all, substantially all, essentially all, or all of the function(s) and feature(s) of skikit-learn, Create ML, or both.

### Data Capture & Retrieval (Saved Patient Information)

In certain aspects, automated system(s), e.g., mobile application(s), described herein comprise or are associated with storage capability(ies). In aspects, storage capability(ies) of system(s), e.g., application(s), described herein can be local to the system or remote, e.g., as a cloud storage system, etc. In aspects, some, most, generally all, essentially all, or all data generated by system(s) provided herein are or can at least optionally be stored by one or more storage mechanism(s) associated with the system(s). In aspects, data are stored as feature set(s).

As discussed briefly elsewhere, in specific aspects, feature set(s) generated by system(s) and stored by storage capability(ies) associated with such system(s), comprise data which, when applied to aesthetic modification procedure(s) such as, e.g., upper lip lift or specifically deep plane upper lip lift procedures, result in such procedures yielding an outcome which is at least mostly the same as, as least substantially the same as, at least essentially the same as, is essentially the same as, or is the same as, e.g., is comparable to, predicted outcome(s) generated by morphing technology(ies) associated with the system(s). In aspects, such predicted outcome(s) generated by morphing technology(ies) are presented to system user(s) prior to the conduct of the aesthetic modification procedure(s). In aspects, final outcome(s), e.g., images of result(s) of an aesthetic modification procedure, can be captured and compared to predicted outcome(s). In aspects, system(s) can comprise artificial intelligence capability(ies) allowing for the system(s) to learn from such comparison(s), wherein such machine learning results in the detectable or significant improvement in predictability of final outcome(s) based on or incorporating one, some, most, generally all, substantially all, essentially all, or all element(s) of feature set data.

In certain aspects, automated system(s) provided herein can save a plurality of morphed images, e.g., ≥˜1, ≥˜2, ≥˜3, ≥˜4, ≥˜5, ≥˜6, ≥˜7, ≥˜8, ≥˜9, ≥˜10, ≥˜15, ≥˜20, ≥˜25, ≥˜30, or any number of morphed images created by the system(s) based on input(s). In aspects, automated system(s) can save one or more original image(s), e.g., a plurality of original image(s) of a human face, such as, e.g., a plurality of original image(s) of a human face from a plurality of angles (e.g., frontal view, left-side profile view, right-side profile view, etc.

According to aspects, user(s) of automated system(s), e.g., mobile application(s), such as, e.g., one or more medical professional user(s), are able to save, retrieve, modify, export/extract, apply data related to a predicted outcome, e.g. related to a target aesthetic appearance, quickly and easily within the automated system(s), e.g., mobile application(s). In aspects, stored data is efficiently retrievable by system(s) user(s). In aspects, stored data can be retrieved and used in future instances, e.g., future consultation(s) between a patient and physician, e.g., between a person contemplating an aesthetic modification procedure and a medical professional capable of performing such a procedure, or in the conduct of such procedure(s).

For example, in aspect(s), user(s) can save newly generated data from within the automated system(s), e.g., mobile application(s), described herein in less than about 1 minutes, such as, e.g., ≤˜1 minute, ≤˜55 seconds (secs), ≤˜50 secs, ≤˜45 secs, ≤˜40 secs, ≤˜35 secs, ≤˜30 seconds, ≤˜25 sec, ≤˜20 secs, ≤˜15 secs, ≤˜10 secs, ≤˜5 secs, ≤˜4 secs, ≤˜3 secs, ≤˜2 secs, ≤˜1 secs, ≤˜0.8 secs, ≤˜0.6 secs, ≤˜5 secs, ≤˜0.4 secs, ≤˜0.3 secs, ≤˜0.2 secs, or in ≤˜0.1 secs.

In aspect(s), user(s) can retrieve previously generated data from within the automated system(s), e.g., mobile application(s) described herein which has been saved by such system(s), e.g., application(s), in less than about 1 minutes, such as, e.g., ≤˜1 minute, ≤˜55 seconds (secs), ≤˜50 secs, ≤˜45 secs, ≤˜40 secs, ≤˜35 secs, ≤˜30 seconds, ≤˜25 sec, ≤˜20 secs, ≤˜15 secs, ≤˜10 secs, ≤˜5 secs, ≤˜4 secs, ≤˜3 secs, ≤˜2 secs, ≤˜1 secs, ≤˜0.8 secs, ≤˜0.6 secs, ≤˜5 secs, ≤˜0.4 secs, ≤˜0.3 secs, ≤˜0.2 secs, or in ≤˜0.1 secs.

In aspect(s), user(s) can modify (e.g., edit one or more feature(s) or characteristic(s) of) data generated by automated system(s), e.g., mobile application(s) described herein, such as newly generated data, previously generated data, or both, in less than about 1 minutes, such as, e.g., ≤˜1 minute, ≤˜55 seconds (secs), ≤˜50 secs, ≤˜45 secs, ≤˜40 secs, ≤˜35 secs, ≤˜30 seconds, ≤˜25 sec, ≤˜20 secs, ≤˜15 secs, ≤˜10 secs, ≤˜5 secs, ≤˜4 secs, ≤˜3 secs, ≤˜2 secs, ≤˜1 secs, ≤˜0.8 secs, ≤˜0.6 secs, ≤˜5 secs, 9-0.4 secs, ≤˜0.3 secs, ≤˜0.2 secs, or in ≤˜0.1 secs.

In aspect(s), user(s) can export data generated by automated system(s), e.g., mobile application(s) described herein, e.g., new data or data which has been saved by such system(s), e.g., application(s), in less than about 1 minutes, such as, e.g., 9-1 minute, ≤˜55 seconds (secs), ≤˜50 secs, ≤˜45 secs, ≤˜40 secs, ≤˜35 secs, ≤˜30 seconds, ≤˜25 sec, ≤˜20 secs, ≤˜15 secs, ≤˜10 secs, ≤˜5 secs, ≤˜4 secs, ≤˜3 secs, ≤˜2 secs, ≤˜1 secs, ≤˜0.8 secs, ≤˜0.6 secs, ≤˜5 secs, ≤˜0.4 secs, ≤˜0.3 secs, ≤˜0.2 secs, or in ≤˜0.1 secs. In aspects, such export can be in any format, such as, e.g., a format recognizable by a Windows operating system, a Mac operating system, a Linux operating system, or the like. In aspects, an export can be to a text document, a spreadsheet file, an email, etc. In aspects, the export can be of an image file.

In aspect(s), user(s) can apply data, e.g., feature set(s), generated by automated system(s), e.g., mobile application(s) described herein, such as apply newly generated data, previously generated data, or both, to an image such that the application of data results in the generation of a morphed image in less than about 1 minutes, such as, e.g., ≤˜1 minute, ≤˜55 seconds (secs), ≤˜50 secs, ≤˜45 secs, ≤˜40 secs, ≤˜35 secs, ≤˜30 seconds, ≤˜25 sec, ≤˜20 secs ≤˜15 secs, ≤˜10 secs, ≤˜5 secs, ≤˜4 secs, ≤˜3 secs, ≤˜2 secs, ≤˜1 secs, ≤˜0.8 secs, ≤˜0.6 secs, ≤˜5 secs, ≤˜0.4 secs, ≤˜0.3 secs, ≤˜0.2 secs, or in ≤˜0.1 secs.

According to aspects, automated system(s) provided herein comprise data saving functionality(ies) for local storage of data, e.g., patient information, within the automated system(s), e.g., mobile application(s). In aspects, automated system(s) provided herein comprise data saving functionality(ies) allowing for the ability to integrate with cloud storage services. In aspects, integration with cloud storage services allows for saved data, e.g., saved patient data or any data described herein, to be accessible from a plurality of devices (e.g., any device that can access the cloud storage service and by user(s) having sufficient authority to access such data). In aspects, a local storage framework can be any framework suitable for local storage of data, e.g., patient information, within the automated system(s), e.g., mobile application(s), such as, e.g., an Apple framework such as Core Data. In aspects, a local storage framework is a framework comprising one, some, most, generally all, substantially all, essentially all, or all of the feature(s) and function(s) of Core Data. In aspects, a cloud storage capability is any suitable functionality allowing for the integration with any service capable of storing data, e.g., patient data and allowing for access to such data across multiple devices. In aspects, suitable cloud storage service(s) is/are Firebase Cloud Storage, Amazon S3, and the like. In aspect, automated system(s) are integrated with one or both of Firebase Cloud Storage and Amazon S3 or one or more cloud storage services having one, some, most, generally all, substantially all, essentially all, or all of the feature(s) of one or both of Firebase Cloud Storage and Amazon S3.

According to aspects, automated system(s) disclosed here comprise one or more of a local data storage platform such as Core Data and is integrated with one or more cloud storage services such as Firebase Cloud Storage or Amazon S3 or comprises one or more local data storage frameworks and is integrated with one or more cloud storage services having one, some, most, generally all, substantially all, essentially all, or all of the features of Core Data, Firebase Cloud Storage, and Amazon S3.

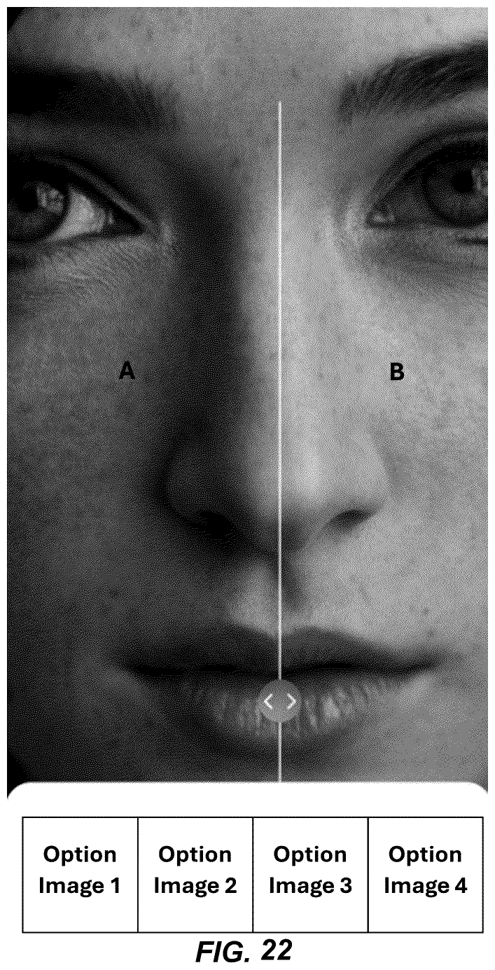

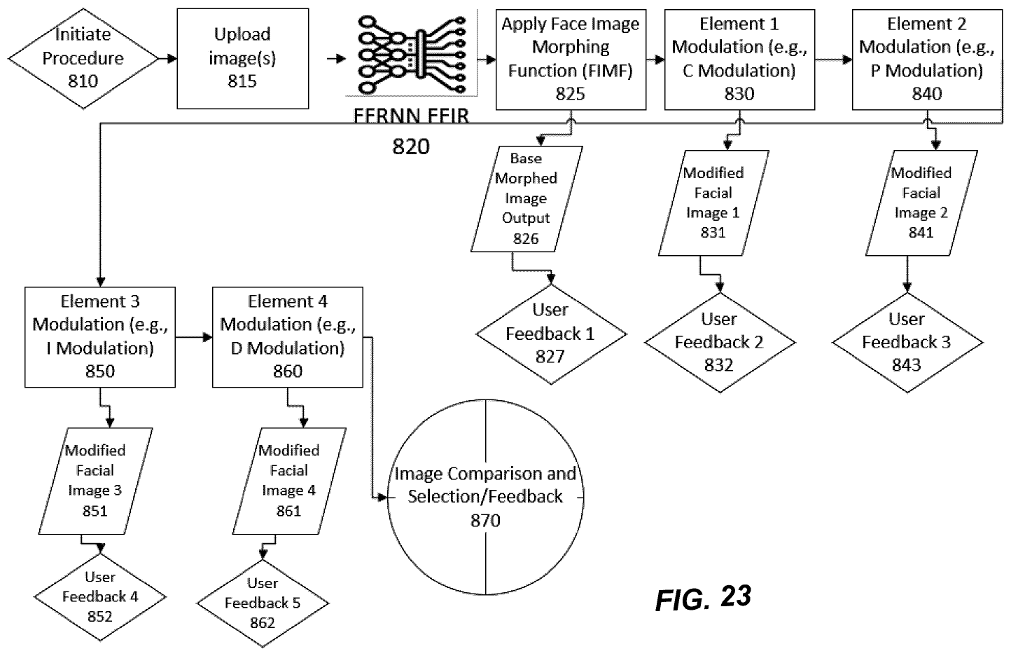

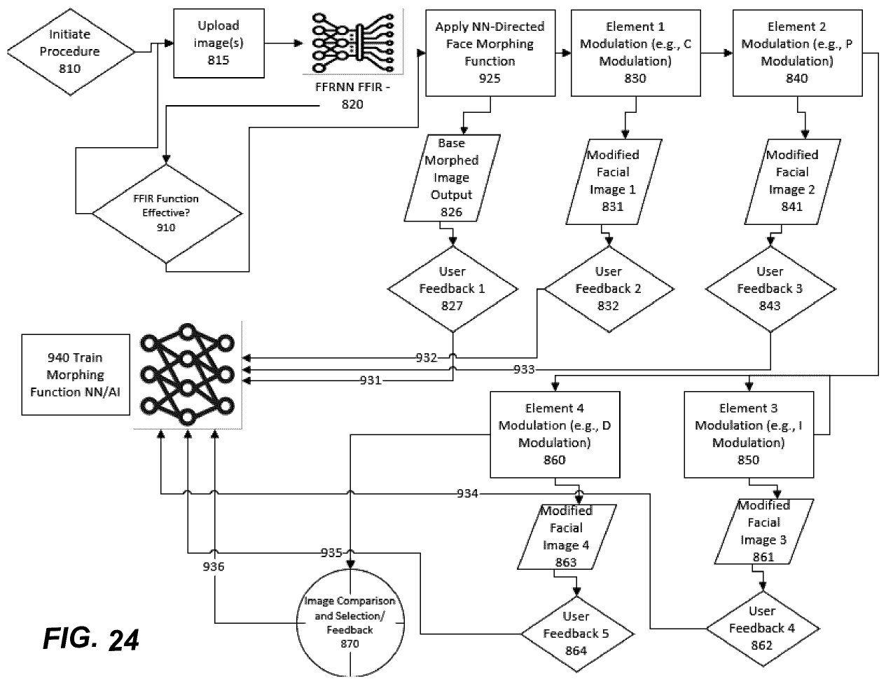

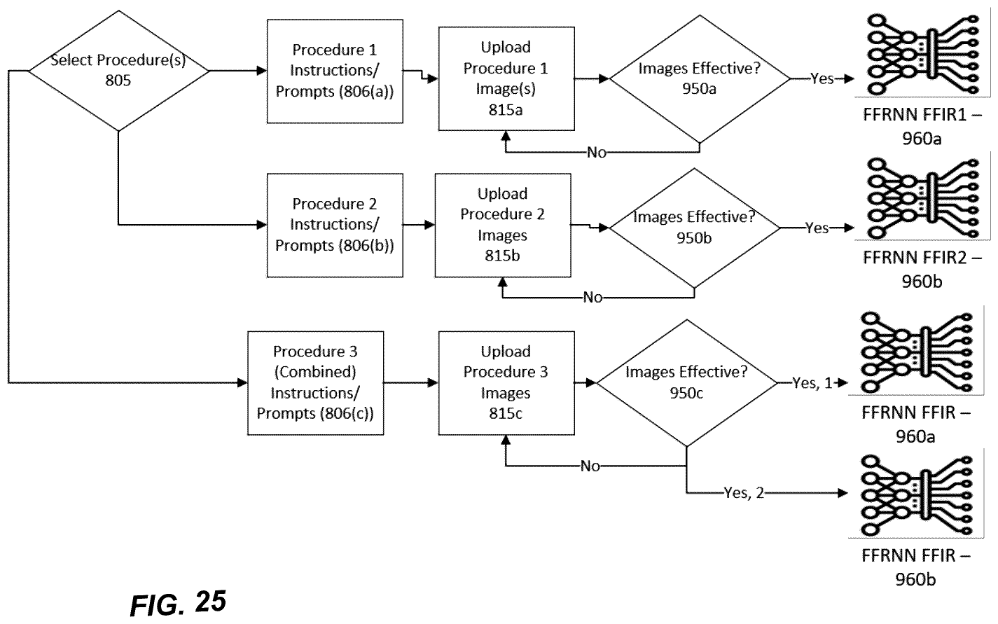

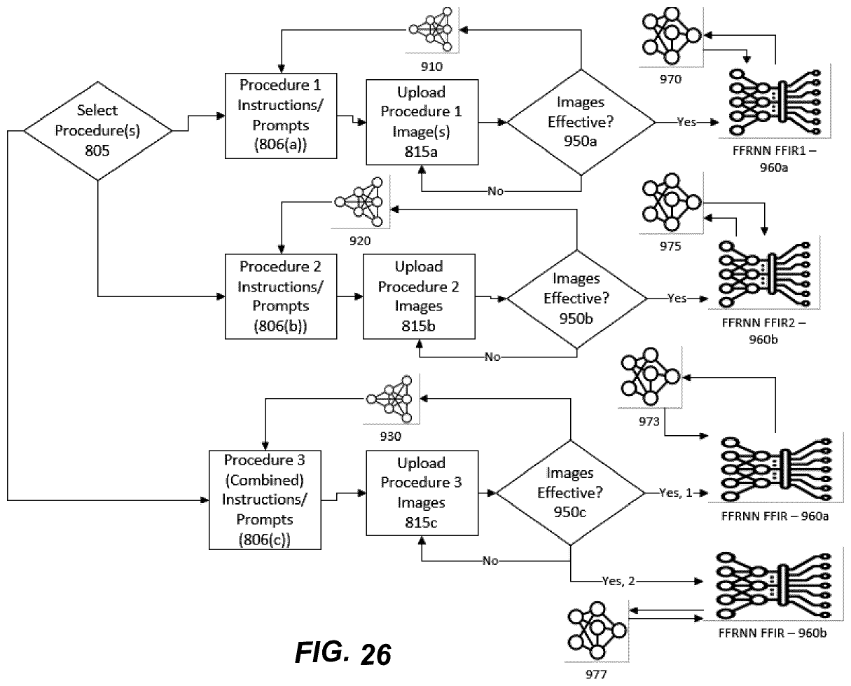

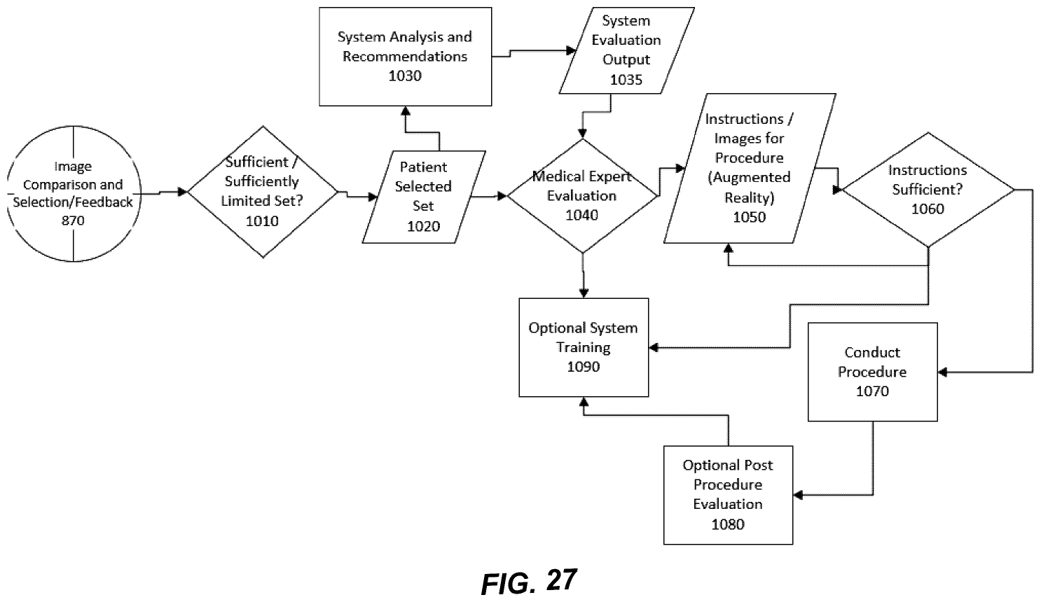

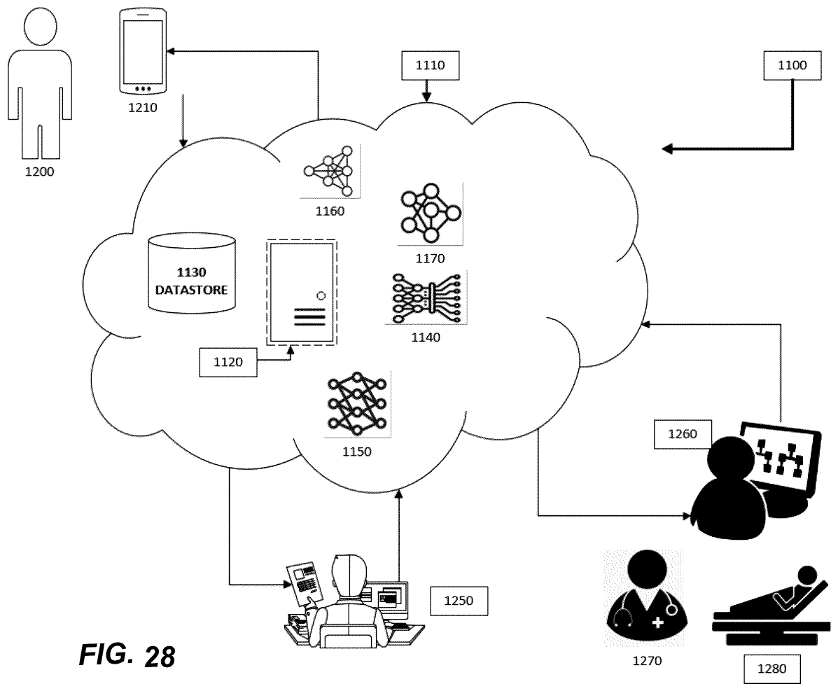

In certain aspects, retrieved images can be presented to a user via an interface within automated system(s), e.g., within mobile application(s), in a manner which facilitates comparison of two or more images, or, e.g., the comparison of portion(s) of two or more images. For example, as is discussed in detail elsewhere herein, automated system(s) can provide for two images, e.g., one or both of such images being saved images, to be compared side by side. In aspects, such two images can be, e.g., an original image and a morphed image, presenting a predicted aesthetic appearance with a given feature set applied. In aspects, portions of images, e.g., the left half of one image and the right half of the other image, can be presented side by side, such that, e.g., if each image is of a human face, a complete face is presented with one half representing, e.g., an original (current) aesthetic appearance of the face and the other half representing, e.g., a predicted appearance should the features of a feature set resulting in the predicted appearance be applied in an aesthetic modification procedure. In aspects, as discussed below and shown in FIG. 22, a user can maintain a first image, e.g., an original image, indicated in FIG. 22 as “A” while comparing “A” to a plurality of morphed images (portions thereof), indicated as “B” in FIG. 22, wherein one morphed image (B) is first selected and shown side by side with image A, then a second morphed image (B) is selected and presented with image A, etc. Such a presentation of images, e.g., captured and retrieved images, can facilitate a system user comparing “before” with predicted “after” image(s) prior to the performance of an aesthetic modification procedure, allowing a subject/patient, medical professional, or both, to select the feature set which, if applied in the performance of the aesthetic modification procedure, is predicted to lead to a desired aesthetic appearance.

### System Access & SAAS Model(s)

According to aspects, automated system(s), e.g., mobile application(s) of this disclosure are accessed by one or more user(s). In aspects, system(s) provided herein comprise one or more controlled access function(s). In aspects, system(s) comprise a plurality of access level(s), each of which provide(s) access to a different one or more, e.g., a different set of, feature(s) of the system(s), e.g., application(s). In aspects, access to system(s), e.g., application(s), or, e.g., to feature(s) or function(s) thereof may be controlled by one or more user setting(s), e.g., the status of user account(s).

In one particular aspect, automated system(s), e.g., mobile application(s), are accessible according to a Software as a Service (SAAS) model. In aspects, system(s), e.g., application(s) herein are accessible only to subscriber(s), e.g., those that have a paid (or, in aspects, non-paid) subscription thereto. According to aspects, for example, feature(s) of automated system(s) described herein may be available to one or more access or subscriber levels, while features (s) of such systems (s), e.g., application(s) may be unavailable to such access or subscriber level(s). As a more specific example, in aspects, access to one or more feature(s) or set(s) of feature(s) may be available to medical professional user(s), but not potential patient(s) interested in aesthetic modification procedure(s). In another specific example, medical professional user(s), or, e.g., certain medical professional subscriber level(s) may have access to one or more parameter option(s), e.g., parameter input option(s), that one or more other medical professional user(s) or medical professional subscriber level(s) do not, such as one or more parameters related to lip lift calculation(s). In certain aspects, an all-access level (e.g., which may be referred to as a “Pro” version, is available, e.g., is available to certain subscriber(s). System(s) herein can comprise administrative accessibility configuration(s) allowing for an administrator/system controller to control access to one or more level(s) of access, control the accessibility of one or more feature(s) within one or more level(s), and the like. Such an administrative accessibility configuration can, in aspects, further allow such an administrator/system controller to establish, delete, modify, otherwise administer user account(s) (such as lock or unlock user account(s), and the like, or combination(s) thereof.

According to aspects, automated system(s), e.g., mobile application(s), can comprise or provide access to a payment processing functionality(ies). In aspects, a payment processing function can be any payment processing function capable of providing secure (e.g., as in identification-protected) payment processing (payment method input, amount input, contact information input, and the like) functionality. In aspects, a payment processing function can be any payment processing function capable of accepting payment(s). In aspects, a payment processing function can be any payment processing function capable of accepting payment(s) and, upon accepting such a payment, providing access to, or communicating with a function capable of providing access to, one or more system access level(s), feature(s), or applicable functionality associated with such a payment. In aspects, successful payment processing yields access to particular feature(s) of the automated system(s), e.g., mobile application(s).

In aspects, automated system(s), e.g., mobile application(s), disclosed here comprise one or more authentication mechanism(s) for accessing the system(s), e.g., application(s), or, e.g., for accessing particular feature(s) or function(s) of the system(s), e.g., application(s). According to aspects, one authentication mechanism may be present to log into the system(s), e.g., application(s) themselves. In aspects, the same or different mechanisms may be present to log into one or more feature(s) or function(s) of the system(s), e.g., application(s). In aspects, the same or different authentication mechanisms may be present to verify an e.g. account version, granting access to one or more applicable feature(s) thereof.

In aspects, authentication mechanism(s) can comprise, e.g., a requirement for a username, password, account number, or other unique identifier (such as, e.g., a social security number, personal identification number (PIN), etc.), biological indicator such as facial recognition, fingerprint identification, retinal scan, and the like.

According to certain aspects, automated system(s), e.g., mobile application(s), provided herein comprise payment processing and subscription access, e.g., leveled subscription access functionality(ies). In aspects, automated system(s) disclosed here comprise integrated payment gateway(s). In aspects, such payment gateway(s) can be any suitable payment gateway allowing for the performance of payment transaction(s). In aspects, payment transaction(s) lead to the provision of access to one or more feature(s) of the automated system(s). In aspects, payment transaction(s) lead to the provision of access to one or more feature(s) or feature level(s) of the automated system(s) according to a software as a service (SAAS) model. In aspects, automated system(s) can comprise any suitable payment processing platform or gateway. In aspects, a suitable payment processing gateway can be, e.g., Stripe, Braintree, PayPal, and the like. In aspects, automated system(s) described herein comprise one or more of Stripe, Braintree, or PayPal. In aspects, a suitable payment processing gateway can be, e.g., one or more of Stripe, Braintree, PayPal, or any one or more payment processing gateway(s) comprising one, some, most, generally all, substantially all, essentially all, or all feature(s) and function(s) of one, two, or each of Stripe, Braintree, and PayPal.

In aspects, automated system(s) herein, e.g., mobile application(s) disclosed herein, comprise a user account management framework. In aspects, a user account management framework provides user authentication functionality, account management functionality (e.g., verifying account versions, granting access to applicable feature(s) associated with account(s), etc.), or both. In aspects, automated system(s) can comprise any suitable account management framework(s) providing user authentication functionality, account management functionality, or both, such as, e.g., frameworks such as Firebase Authentication, OAuth, and the like. In aspects, automated system(s) comprise one or more of Firebase Authentication, OAuth, or one or more frameworks comprising one, some, most, generally all, substantially all, essentially all, or all feature(s) and function(s) of one or both of Firebase Authentication and OAuth.

In aspects, automated system(s) comprise one, some, or each of Stripe, Braintree, PayPal, Firebase Authentication, OAuth, or one or more gateway(s) or framework(s) comprising one, some, most, generally all, substantially all, essentially all, or all of the feature(s) of Stripe, Braintree, PayPal, Firebase Authentication, and OAuth.

### Exemplary Mobile Application(s)

According to certain aspects, this disclosure provides automated system(s) in the form of mobile application(s), e.g., mobile application(s) that operate on a portable device. In aspects, portable device(s) can be, e.g., mobile telephone(s), smartphone(s), tablet(s), mobile computer(s), laptop(s), ultra mobile personal computer(s) (UMPC(s), personal data assistant(s) (PDA(s)), and the like. In certain specific aspects, mobile application(s) herein can operate on mobile telephone(s) such as smartphone(s) and tablet(s). In aspects, such smartphone(s), tablet(s), and the like can be Apple (iOS) or Android device(s). In aspects, system(s) function on both Android and iOS platforms.

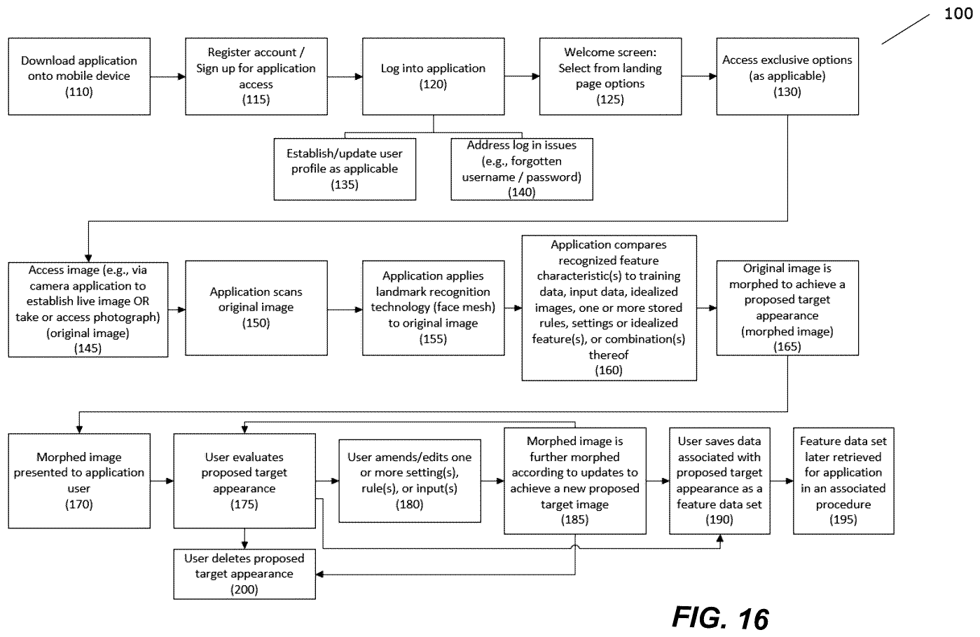

While FIG. 16 accompanying this Application describes one exemplary embodiment of a mobile application provided by this disclosure, mobile application(s) provided herein comprise one or more feature(s), function(s), or computer-aided system component(s) described herein.

For ease of description, the term “screen” is used to describe a particular presentation of the mobile application provided by the mobile application on the screen the mobile device to the mobile device user. In aspects, automated system(s) provided herein are mobile application(s) suitable for use on a smart device such as a smartphone or tablet.

In one aspect, mobile application(s) provided herein can comprise, e.g., a screen presenting the mobile application to the application user (“user”). Such a screen can comprise, e.g., a welcome message, introducing the user to the application and, e.g., inviting the user to get started—e.g., to log in, create an account, and the like. In aspects, such a screen may be a first screen presented to a user upon opening the application.

In one aspect, mobile application(s) provided herein can comprise, e.g., a screen providing the ability for the user to log into the application. In aspects, such a screen provides data input option(s) such as, e.g., one or more fields for capturing identifying information provided by the user. For example, such identifying information can be, e.g., an email address, telephone number, account number, patient identification number, personal identification number, password, and the like, or combination(s) of any or all thereof. In aspects, such a screen may provide the ability to utilize biometric identifiers such as, e.g., facial recognition, retinal scan(s), fingerprint identification, and the like.

According to aspects, mobile application(s) can comprise one or more screens directed to reestablishing access if, for example, a user has trouble signing into the application, such as one or more screens relating to a forgotten username, password, or both. In aspects, mobile application(s) can comprise an authentication function wherein, e.g., a verification code can be provided to the user outside of the application, such as, e.g., by text message or email, and entered into the application in order to aid in reestablishing access. In aspects, one or more authentication function(s) can be provided on screen(s).

In aspects, mobile application(s) provided herein can comprise, e.g., a screen providing the ability for the user to create an account within the application, e.g., as a new user of the application (accessing the application for the first time). In aspects, such a screen provides data input option(s) such as, e.g., one or more fields for establishing or capturing identifying information provided by the user, such as, e.g., name, contact information (email, telephone number, address, and the like), access password(s), etc. and combination(s) of any or all thereof.

In one aspect, mobile application(s) provided herein can comprise, e.g., one or more screen(s) presenting to the user a privacy policy related to use of the application, terms and conditions related to the application, or other informational content. In aspect(s), a link to such a privacy policy page, terms and conditions page, or to any one, some, most, generally all, substantially all, essentially all, or all other pages of the application, is available on another page. That is, a link to one, some, generally all, substantially all, essentially all, or all pages of the application is/are available on one, some, generally all, substantially all, essentially all, or all other pages of the application.

In certain aspects, mobile application(s) can comprise one or more options for presenting the user with a user profile, such a profile comprising one or more pieces of user-associated identification information, e.g., their name, date of birth, physical attributes such as height, weight, gender, ethnicity, location (city, state, country, street address), contact information, application access status or application access level, etc.

In aspects, a user profile screen is an element of mobile application(s) herein. In aspects, mobile application(s) can comprise one or more feature(s) allowing the user to edit one or more pieces of information associated with their profile. In aspect(s), screen(s) for editing, reviewing, saving, or deleting such profile information are available within the mobile application(s) herein.

According to aspects, mobile application(s) herein can comprise, e.g., a screen presenting one or more options to a user, such as one or more types of aesthetic modification procedure(s). In aspects, an aesthetic modification procedure presented on such a screen can include, e.g., an upper lip aesthetic modification procedure, such as, e.g., an upper lip lift, e.g., a subnasal upper lip lift, e.g., a subnasal upper lip lift comprising use of a bull's horn incision technique, such as a deep plane release upper lip aesthetic modification procedure, e.g., a deep plane release upper lip aesthetic modification procedure comprising use of a bull's horn incision technique. In aspects, an aesthetic modification procedure presented on such a screen can include, e.g., a corner lip lift, a nasal base suspension, a muscle suspension, neurotoxin injection(s) into depressor(s), volumization procedure(s), e.g., volumization of a triangle inferior to the commissure, facelift, or combination(s) of any or all thereof. In certain aspects, mobile application(s) can comprise a screen presenting options to explore a deep plane release upper lip aesthetic modification procedure and an evaluation of whether or not a corner lip lift may augment the effect(s) of such an upper lip lift procedure.

According to aspects, depending on the level of access provided to the user according to the user's account settings (e.g., the presence or absence of a paid subscription to the application or, e.g., the level of subscription maintained by the user), one or more screen(s) of the application can present one or more feature(s) of the application, such as, e.g., specific content related to marking system(s) described herein. In aspects, such content can be any one or more characteristic(s) of any one or more marker(s) of marking system(s) provided herein,

According to aspects, mobile application(s) can comprise a screen upon which a live camera image of the user is shown. In aspects, mobile application(s) can comprise a screen upon which a photograph of a human face, e.g., the face of the user, is shown. In aspects, such a screen can comprise scanning the image by a landmark detection system/feature of the application and the application of, e.g., the addition of landmark detection indicator(s) to the image. In aspects, such landmark detection indicator(s) can be referred to as a facial mesh (as, e.g., when applied, it can appear as a web of lines placed upon the image, marking a plurality of recognized points on the image).

According to aspects, mobile application(s) can comprise a screen providing a user to modify the image, e.g., “morph” the image, according to one or more inputs, based on the landmark(s) detected by the landmark detection feature. In aspects, one or more specific areas of the image, e.g., the areas of the image represented by marker(s) of marking system(s) presented herein, e.g., one or more of the specific areas identified by radial marker(s) of marking system(s) described herein, e.g., one, some, most, generally all, substantially all, essentially all, or all of marker(s) 1, 2A, 2B, 3A, 3B, 4A, 4B, 5A, 5B, H-A, H-B, LE-A, LE-B, may be adjusted via adjustment feature(s) presented allowing for the user to interact with the application. In aspects, one or more available adjustment feature(s) can allow a user to morph the image at one or more locations using adjustment mechanism(s) such as scale adjustment (e.g., slider(s)) or direct data entry (e.g., regarding intensity of adjustment at such location). For example, mobile application(s) provided here can comprise a screen wherein an original image is present of a human face, wherein a landmark detection system of the application has been applied and a plurality of landmark(s) have been detected and which may be indicated by a visible or invisible set of landmark markings or guides, is presented; and, further, wherein one or more adjustment mechanism(s) are present to adjust the appearance of the image at one or more locations, such as locations represented by one or more of marker(s) 1, 2A, 2B, 3A, 3B, 4A, 4B, 5A, 5B, H-A, H-B, LE-A, and LE-B. In aspects, the intensity of morphing of the image at any one or more locations can be individually controlled by the user via an input mechanism on such a screen.

In aspects, mobile application(s) provided herein can comprise a screen presenting a comparison between an original image (e.g., original photograph of a face, e.g., the users face) and a morphed image (e.g., the appearance of the face as modified by the changes made to the photograph using the adjustment mechanism(s) described here. In aspects, the comparison can be two images, presented side-by-side or one above the other. In aspects, the comparison can be a single image, wherein approximately one half of the image is, e.g., half of the original photograph (half of the photograph of the original human face) and the other half of the image is, e.g., the opposite half of the human face, but as morphed by the user within the application. In aspects, such screen(s) described here allow for a user to assess possible intervention(s) associated with an aesthetic modification procedure will look like and to compare, e.g., before and anticipated “after” appearance(s). As provided elsewhere herein, in certain aspects, automated system(s) provided herein can save a plurality of morphed images, e.g., any number of original image(s), morphed image(s), or both created by or introduced to the system(s) based on input(s). As also provided elsewhere herein, in aspects, automated system(s) can save one or more original image(s), e.g., a plurality of original image(s) of a human face, such as, e.g., a plurality of original image(s) of a human face from a plurality of angles (e.g., frontal view, left-side profile view, right-side profile view, etc. According to certain aspects, automated system(s), such as, e.g., mobile application(s) disclosed herein can comprise a screen providing the ability to compare half of an original image, e.g., the left-side of an original image of a frontal view of a human face, with half of a morphed image, e.g., the right-side of a morphed image of a frontal view of the same human face with input(s) applied, such that the human subject can see the predicted difference between “before” and “after” the application of a proposed feature set or input(s) resulting in the predicted outcome represented by the morphed image. In aspects, screen(s) of mobile application(s) can present to a user on a viewable screen (interface), e.g., a portion of an original image and a plurality of morphed images (having been derived using varying input(s)) from which the user can select, allowing the user to easily compare an original image or portion thereof to a predicted outcome according to particular input(s), represented by morphed image(s). See, e.g., FIG. 22, wherein a portion of two images are shown: Image (or portion of image) “A” is shown on the left and may represent, e.g., an original image. Image (or portion of image) “B” is shown on the right and may represent, e.g., a morphed image. In aspects, such an interface may allow, e.g., a user to compare an original image or portion thereof to predicted outcome(s) represented by morphed image(s) in series, e.g., comparing one predicted outcome to an original image, then a different predicted outcome, then, e.g., yet a still different predicted outcome, in order to identify the preferred outcome and thus the preferred requirements which should be applied in an aesthetic modification procedure in order to achieve the predicted outcome. See, e.g., in FIG. 22, “Option Image 1,” “Option Image 2,” “Option Image 3,” and, e.g., “Option Image 4” shown at the bottom of the exemplary user interface. In aspect(s), an automated system, e.g., a mobile application, may comprise a screen, such as a screen exemplified in FIG. 22, wherein a user can “swipe” (run one's finger across a screen thereby selecting screen(s) or feature(s) shown therein or, e.g., moving from screen to screen or option to option thereon) through a series of available image(s), e.g., thumbnail image(s), e.g., as represented by Option Images 1-4 shown. In aspects, such image(s) can be, e.g., various original images, various morphed image(s), or a mixture thereof. In aspects, one image or portion thereof can remain static on an interface, e.g., screen, of an automated system, e.g., mobile application, while the user swipes or scrolls through a plurality of presented options for the user to select. In one specific example, an interface can present a portion, e.g., one half of an original image, e.g., showing the left side of a human face, and the user can “swipe” or scroll through a plurality of morphed images, e.g., morphed images of the same human face (each morphed according to input(s)), wherein upon stopping, selecting, or stopping and selecting any one morphed image places a portion, e.g., the right half of that morphed image adjacent to the left half of the original image. This side-to-side comparison allows system user(s) to compare an original image to a predicted outcome and thus facilitates the selection of a/the desired (target) feature set(s) which are predicted to achieve the desired (target) aesthetic result(s) when applied in an aesthetic modification procedure. In a specific example, this comparison allows a system user to identify the differences in resulting appearance when varying amount(s) of an upper lip, e.g., various amount(s) at various locations, e.g., locations identified in marking system(s) disclosed herein, are removed in an upper lip lift procedure.

According to aspects, the mobile application(s) provided here comprise option(s) for saving any data (e.g., raw data, associated images, or both) associated with morphed images. In aspects, for example, if a user does not like the outcome/effect(s) of the image morphing, the user can elect not to save the resulting morphed image data. Alternatively, if a user likes the outcome/effect(s) of the image morphing or, e.g., wishes to share the results of image morphing with other(s), the user can elect to save the resulting morphed image data. In aspects, screen(s) of application(s) provided herein present the user with option(s) for saving data.

In aspects, screen(s) of mobile application(s) can comprise branding element(s) such as logo(s), color scheme(s), font(s), imagery, and the like, which provide a recognizable aesthetic or recognizable appearance to the application(s).

In aspects, application(s) herein are developed using tool(s) allowing for the creation of natively compiled application(s) for mobile and web use from a single codebase. In alternative aspects, multiple codebases may be appropriate, applicable, and otherwise implemented. In aspects, a single codebase allows for use across a plurality of platforms. In aspects, a suitable tool for such application(s) is a mobile user interface framework such as a framework developed by Google. In aspects, such a framework is an open-source framework. In aspects, such a tool is Flutter or a tool providing some, most, generally all, substantially all, essentially all, or all of the feature(s) and functionality of Flutter or tool(s) offering at least some, most, generally all, substantially all, essentially all, all, or more feature(s) and functionality as provided by Flutter.

### Computer-Aided Method(s)

In aspects, disclosure herein provides method(s), computerized method(s), or computer system facilitate(d)/implemented or aided method(s) of establishing target effect(s), e.g., target aesthetic appearance(s), resulting from one or more aesthetic modification procedure(s), such as, e.g., an upper lip lift, a subnasal upper lip lift, a deep plane release upper lip lift, a corner lip lift, a nasal base suspension, a muscle suspension, or any combination of any or all thereof (e.g., subnasal and corner lip lift procedure).

According to aspects, computer-aided method(s) described herein comprise generating or acquiring an original image. In typical aspects, the image is an at least partial image of a human face, e.g., including the upper lip of a human face. In aspects, automated system(s) herein are capable of generating or acquiring such an original image. In aspects, the method(s) herein include a step for pre-operative photography to generate such an original image.

In aspects, disclosed here are method(s) of establishing target effect(s), e.g., target aesthetic appearance(s), e.g., effect(s) and appearance(s) (e.g., of a human face, e.g., of the upper lip of a human face) resulting from one or more aesthetic modification procedure(s) described herein, wherein the method comprises (1) establishing a starting aesthetic appearance; (2) using automated system(s) and feature(s) thereof to establish the required characteristic(s) of one or more marker(s) of marking system(s) herein which, when applied to the starting aesthetic appearance yield a target aesthetic appearance (such as, e.g., a target final lip height, target teeth show, target slope of the upper vermillion border, or combination(s) thereof); and (3) applying such characteristic(s) in an aesthetic modification procedure, wherein the application of such characteristic(s) in the aesthetic modification procedure results in the establishment of the targeted effect(s), e.g., targeted aesthetic appearance(s). In aspects, such method(s) described here can comprise the use of one or more automated system(s) and feature(s)/function(s) thereof described herein.

According to certain specific aspects, automated system(s) provided herein can communicate with one or more instrument(s), e.g., one or more robotic instrument(s), capable of applying marking(s) of marking system(s) provided herein, to human skin. In aspects, such markings may be physical markings (made, e.g., using an ink), e.g., topically applied or, e.g., via temporary tattoo technique(s). In aspects, such marking(s) may be made via any mechanism(s) which do not result in permanent visibility on the subject(s) face after more than about 1 month, more than ˜3 weeks, more than ˜2 weeks, more than ˜1 week, more than ˜5 days, more than ˜3 days, or, e.g., for more than ˜1 day, (e.g., for more than about 12 hours, or more than about 6 hours) after completion of associated aesthetic procedure(s).

In aspects, automated method(s) disclosed herein comprise storing data describing, related to, or which otherwise, when applied, yield specific aesthetic effects. In aspects, In aspects, such automated method(s) comprise storing data describing, related to, or which otherwise when applied, or, e.g., when compiled, collected, then applied, yield a target aesthetic effect, or, e.g., when compared to a subject image, provide suggested intervention(s)/modification(s) to be made in a procedure to attain the specific aesthetic effect(s)/target aesthetic appearance(s) which have been predetermined as being “optimal” or, e.g., providing for particularly attractive, pleasant, pleasing, or otherwise desirable appearance. As one example, in aspects, such automated method(s) comprise storing data describing, related to, or which otherwise, when applied, yield a target vermillion slope. In particular, for example, in aspects, such automated method(s) comprise storing data describing, related to, or which otherwise when applied or combined/compiled and applied yield a consistent target vermillion slope or suggested intervention(s)/modification(s) to be made based upon a comparison to a subject image to yield a consistent target vermillion slope, the vermillion slope being a consistent height difference along the upper vermillion border (020) between points associated with adjacent radial marker(s), such as, e.g., 2A (008A) and 3A (010A), and 3A (010A) and 4A (012A), 2B (008B) and 3B (010B), 3B (010B) and 4B (012B), or any or all thereof.

According to aspects, data with respect to idealized upper lip feature(s), e.g., lip shape(s), can be stored in computer system(s). In aspects, data related to idealized lip shape(s) can be established based on the size(s), relationship(s) between, or both, of facial feature(s), such as, for example, face shape and size, nose shape and size, cheek shape and size, chin shape and size, etc. or any or all thereof can be stored in computer system(s). In aspects, for example, data related to an idealized upper lip symmetry, an idealized upper final lip height, an idealized symmetry or balance of nasal feature(s), an idealized symmetry or balance between nasal feature(s) and lip feature(s), or any combination thereof can be stored in computer system(s). In aspects, computer system(s) can comprise software capable of modifying specific attributes of idealized feature profiles based upon one or more factors, such as, e.g., patient age, face size, patient or practitioner aim(s)/goal(s), or any combination of any or all thereof. In aspects, data related to idealized target effect(s) such as lip shape, e.g., lip slope, are established based on mathematical calculations directed to height differences between two or more points along the upper vermillion border (020), wherein height differences between two or more points along the upper vermillion border (020) are consistent across multiple pairs of neighboring points along the upper vermillion border (020) (as is, e.g., described elsewhere herein). In aspects, data related to idealized target effect(s) such as lip shape, e.g., lip slope, are established so as to address uniformity, e.g., symmetry, of nasal structure(s), upper lip anatomy, or both.

In aspects, automated method(s) of establishing target effect(s), e.g., target aesthetic appearance(s), resulting from one or more aesthetic modification procedure(s), such as, e.g., an upper lip lift, a subnasal upper lip lift, a deep plane release upper lip lift, a corner lip lift, a nasal base suspension, a muscle suspension, or any combination of any or all thereof (e.g., a subnasal and corner lip lift procedure, such as a deep plane release subnasal and corner lip lift procedure) can comprise capturing, importing, or accessing one or more digital image(s) of a subject. In aspects, digital image(s) can be imported using any suitable data import or data access/retrieval technology. In aspects, digital image(s) can be captured by routine photography or by, e.g., scanning technology(ies), including, e.g., technology(ies) utilizing facial recognition technology(ies).

In aspects, method(s) comprise comparing digital image(s) of a subject prior to an intervention and making such image(s) available to computer(s), computer program(s)/software program(s), artificial intelligence algorithm(s), or combination(s) thereof for analysis. In aspects, such analysis comprises, e.g., automated comparison of the image(s) of the subject(s) to stored profile(s), wherein stored profile(s) include data comprising or establishing standard(s) considered aesthetically ideal, attractive, or otherwise desirable. In aspects, such an analysis comprises use of artificial intelligence system(s). In aspects, such an analysis comprises use of neural network(s), wherein such analytical program(s) learn over time and become more and more savvy with regard to nuanced action(s) to suggest or otherwise require in order to attain target aesthetic effect(s)/appearance(s). Means for comparison of facial elements are known in the art and any suitable one or combination of such methods can be applied to the methods and systems described in the system. Those of skill in the art will also be able to modify such systems to be suitable for use with the particular methods provided herein, as adapted to the digital/software environment.

According to aspects, method(s) comprise an automated analysis of the result of such an automated comparison as described above, wherein the analysis yields an output comprising suggested or required modification(s) to be made in aesthetic modification procedure(s) in order to achieve target effect(s)/a target aesthetic appearance.

In certain aspects, data for use in creating standard(s), e.g., idealized standard(s), described in this section, e.g., data related to target aesthetic effect(s) or used in algorithm(s) to establish such target effect(s) (such as target aesthetic effect(s) described herein) and in calculations or method(s) of establishing parameter(s) of marking system(s) as presented herein, are present within a computer software package or, e.g., a software application (“an app”), for use in conjunction with aesthetic modification procedure(s), such as procedure(s) provided herein.

According to aspects, the disclosure provides computer-aided or controlled (automated) method(s) of performing procedures comprising the use of marking system(s) and mathematical analysis and mathematically based parameters.

In aspects, automated method(s) described herein comprise use of robotics (robotic devices/robotic systems) to perform procedure(s) according to suggested or required action(s) derived from automated analysis(es), such as the analysis(es) described in this section. In aspects, elements of an actual procedure are performed at least in part by a robotic device or system that carries out surgical procedural step(s) according to output from the other elements of a method of the disclosure.

In aspects, the disclosure provides method(s) of automating use of marking system(s) provided herein, e.g., marking system(s) comprising one or more of the characteristic(s) provided herein. In aspects, such method(s) comprise(s) step(s) or feature(s) described in this section.

According to certain aspects, the disclosure provides method(s) of automating one or more evaluation and characterization step(s), e.g., one or more evaluation and characterization step(s) of method(s) comprising use or development of marking system(s); one or more step(s) in method(s) incorporating use of marking system(s); or both. In aspects, such method(s) comprise(s) step(s) or feature(s) described in this section.

In aspects, the disclosure provides method(s) of using computer system(s), computer or otherwise automated application(s), or combination(s) of software-based system(s)/application(s), artificial intelligence-based system(s)/application(s), or both, with or without use of neural network(s) facilitating machine learning, to automate the evaluation and characterization process(es) associated with method(s) described herein. In aspects, the disclosure provides method(s) of using computer system(s), computer or otherwise automated application(s), or combination(s) of software-based system(s)/application(s), artificial intelligence-based system(s)/application(s), or both, with or without use of neural network(s) facilitating machine learning, to automate the development of marking system(s) provided herein which are suitable for acting upon in order to attain one or more target aesthetic effect(s).

According to certain specific embodiments, the disclosure provides programming the concepts provided herein, e.g., marking system(s) provided herein, concept(s) founding the establishment of marking system(s) provided herein, mathematical relationship(s) upon which various element(s) of marking system(s) are established, the combination of various decision point(s) contributing to procedure(s) herein (e.g., deep plane release surgical procedure(s) comprising subnasal and corner lip lift modification(s)), or combination(s) of some, most, generally all, or all thereof, into morphing software.

In aspects, the computerized methods provided herein also or alternatively comprise applying the principles of the methods of the disclosure to a particular setting, such as the optimization of appearance in a model of facial change, which can then be submitted to an artificial intelligence/machine learning system, such as a generative artificial intelligence system, such as, for example, one or more neural networks, which can be trained to optimize for one or more of the various optimal characteristics described herein, for application of those to an individual's pre-surgery facial features, and the like. The application of image-based neural network/machine learning methods is known in the art and, generally, any suitable method can be applied in such aspects. Thus, for example, a suitable neural network can be trained using outcome data from application of the methods described herein, can be provided the algorithms described herein as starting/default parameters, or both, and can be optimized based on user feedback (patient and/or healthcare provider), iterative runs of the neural network process, and the like, so as to produce further optimized and patient-specific parameters for presentation in modeling, for guidance in performance of surgical methods, and further improvement on the various parameters described in this disclosure.

### Security & Regulatory Considerations

According to aspects, automated system(s) provided herein comprise sensitive subject matter data, such as, e.g., identifying information related to human subject(s). In aspects, such sensitive subject matter data which may be captured by, housed within, processed by, or transmitted by (or combination(s) thereof) automated system(s) disclosed herein can comprise, e.g., identifying information including image(s) of a person or people.

According to aspect(s), automated system(s) herein provide security measure(s) for securing sensitive data associated therewith. In aspects, system(s) herein meet or exceed all applicable regulatory standard(s) for data compliance present at the time of this Applicant and at the time of commercial operation of such system(s).

### Outcomes

In aspects, marking system(s) provided herein, and (1) method(s) of their use, (2) method(s) incorporating them; (3) method(s) comprising their development; (4) procedure(s) incorporating them; and, e.g., (5) procedure(s) incorporating method(s) comprising their development, collectively “marking system(s) and associated procedure(s),” yield one or more identifiable outcomes which are detectably or significantly improved compared to outcomes resulting from method(s), procedure(s), or both which are performed without use of such marking system(s). In aspects, the disclosure provides method(s) of performing procedure(s) comprising marking system(s) provided herein. In aspects, the disclosure provides method(s) of performing procedure(s) comprising incorporation of one or more steps of method(s) provided herein, comprising incorporation of one or more marker(s) of marking system(s) provided herein, or both. In aspects, procedure(s) incorporating one or more steps of method(s) provided herein, incorporating one or more marker(s) of marking system(s) provided herein, or both yield one or more identifiable outcome(s) which are detectably or significantly improved compared to outcomes resulting from procedure(s) which are performed without use of such marking system(s) or related method(s).

### Exaggeration & “Snarling”

In aspects, marking system(s) provided herein, and (1) method(s) of their use, (2) method(s) incorporating them; (3) method(s) comprising their development; (4) procedure(s) incorporating them; and, e.g., (5) procedure(s) incorporating method(s) comprising their development (marking system(s) and associated method(s)) yield one or more identifiable outcomes which are detectably or significantly improved compared to outcomes resulting from method(s), procedure(s), or both which are performed without use of such marking system(s), wherein one of the one or more identifiable outcome(s) is a detectable or significant reduction in the frequency of an undesirable, unexpected, or otherwise non-targeted exaggerated appearance. In aspects, marking system(s) and associated method(s) described here result in a detectable or significant reduction in the frequency of a resulting exaggerated appearance in a population of patients having a subnasal lift procedure incorporating the method compared to the frequency of an undesirable exaggerated appearance resulting in a population of patients having a subnasal lift procedure not incorporating the system(s), method(s), or combination thereof, as determined by the professional(s) performing the procedure(s), the subject(s) undergoing the procedure(s), a group of medical professionals trained in the art tasked with assessing the outcome of the procedure(s), one or more proactive or retroactive studies performed by or overseen by one or more skilled professionals in art, or any combination of any or all thereof.

In certain aspects, lip types having central peaking and lateral drooping are especially at risk of iatrogenic exaggeration. In aspects, certain ethnic variations, such as Asian lips, are detectably or significantly predisposed to central exaggeration. In aspects, such a predisposition requires a laterally biased excision, removing more laterally than centrally. In certain aspects, marking system(s) and associated method(s) described here detectably or significantly reduce the frequency of an exaggerated appearance resulting from procedure(s) incorporating system(s), method(s), or both described herein in population(s) of patient(s) having central peaking and lateral drooping, being of an ethnicity detectably or significantly predisposed to central exaggeration as is known in the art, or combinations thereof, compared to procedure(s) lacking the same, as determined by the professional(s) performing the procedure(s), the subject(s) undergoing the procedure(s), a group of medical professionals trained in the art tasked with assessing the outcome of the procedure(s), one or more proactive or retroactive studies performed by or overseen by one or more skilled professionals in art, or any combination of any or all thereof.

In aspects, marking system(s) and associated method(s) yield one or more identifiable outcomes which are detectably or significantly improved compared to outcomes resulting from method(s), procedure(s), or both which are performed without use of such marking system(s), wherein one of the one or more identifiable outcome(s) is a detectable or significant reduction in the frequency of an undesirable, unexpected, or otherwise non-targeted exaggeration in the slope of the upper vermillion border (020) yielding an unsatisfactory result in a population of patients having a subnasal lift procedure incorporating use of the method compared to the frequency of an exaggeration in the slope of the upper vermillion border (020) yielding an unsatisfactory result in a population of patients having a subnasal lift procedure not incorporating use of the method as determined by the professional(s) performing the procedure(s), the subject(s) undergoing the procedure(s), a group of medical professionals trained the art tasked with assessing the outcome of the procedure(s), one or more proactive or retroactive studies performed by or overseen by one or more skilled professionals in art, or any combination of any or all thereof.

According to aspects, one mechanism provided by marking system(s) and associated method(s) described here which detectably or significantly reduce(s) the frequency of an undesirable outcome from procedure(s) incorporating the system(s) or method(s) described here, such as exaggeration characterizable as “bowing” or “snarled” lip (“snarling” appearance) is the detectable or significant relatively higher lifting at location(s) marked by one or more of radial marker(s) 2A (008A), 2B (008B), 4A (012A), and 4B (012B) (including, a combination of any or all thereof or all thereof) of marking system(s) compared to location(s) marked by radial marker(s) 3A (010A), 3B (010B), or both. In certain aspects, lifting at location(s) indicated by 3A (010A), 3B (010B), or both excessively relative to location(s) marked by radial marker(s) 2A (008A), 2B (008B), 4A (012A), and 4B (012B) (including a combination of any or all thereof or all thereof) can cause a detectable or significant undesirable effect characterizable as an angry or snarled appearance. According to certain aspects, marking system(s) and associated method(s) provide a length of radial marking(s) 3A (010A), 3B (010B), or both which yield(s) an application of a lift along radial marking(s) 3A (010A), 3B (010B), or both which is detectably or significantly lower relative to the lift along one or more other radial marking(s), detectably or significantly relieving an angry, scowling, or snarled appearance. In aspects, marking system(s) and associated method(s) establish radial marking(s) 3A (010A), 3B (010B), or both to have length(s) yielding an application of a lift along them which is detectably or significantly lower relative to the lift along one or more other radial marking(s) resulting in the upper lip lift procedure, e.g., subnasal upper lip lift procedure, e.g., subnasal and corner lip lift procedure, detectably or significantly relieving an appearance characterizable as angry, scowling, or snarled. In aspects, marking system(s) and associated method(s) establish radial marking(s) 3A (010A), 3B (010B), or both to have length(s) yielding an application of a lift along them which is detectably or significantly greater relative to the lift along one or more other radial marking(s) results in the upper lip lift procedure, e.g., subnasal upper lip lift procedure, e.g., subnasal and corner lip lift procedure detectably or significantly increasing or creating an appearance characterizable as angry, scowling, or snarled.

### Incisor Show

In aspects, incisor display is measured as the difference in height between the border of the 2 central incisors and the lower vermillion wet-dry border at the stomion. If the lip border is exactly at the level of the central incisors, this is designated as “0” incisor display. A lip that is 2 mm longer than the teeth would have “˜2 mm” display, whereas teeth that are longer than the upper lip would have “+2 mm” incisor display.

According to certain aspects, marking system(s) and associated method(s) provide a guide for yielding an incisor display of the human subject resulting from an upper lip lift procedure, e.g., subnasal upper lip lift procedure, e.g., subnasal and corner lip lift procedure of between about 1 mm and about 8 mm, such as, e.g., ˜1 mm-˜7 mm, ˜1 mm-˜6 mm, ˜1 mm-˜6 mm, ˜1 mm-˜4 mm, ˜1 mm-˜3 mm, ˜1 mm-˜2 mm, such as, e.g., ˜2 mm-˜8 mm, ˜3 mm-˜8 mm, ˜4 mm-˜8 mm, ˜5 mm-˜8 mm, ˜6 mm-˜8 mm, or ˜7 mm-˜8 mm, as in, for example, ˜2 mm-˜7 mm, or ˜3 mm-˜6 mm, such as ˜2 mm-˜5 mm, or, e.g., ˜2 mm-˜4 mm. In aspects, marking system(s) and associated method(s) provide a guide for yielding an incisor display of the human subject resulting from an upper lip lift procedure, e.g., subnasal upper lip lift procedure, e.g., subnasal and corner lip lift procedure of less than about 8 mm, ≤˜7 mm, ≤˜6 mm, ≤˜5 mm, ≤˜4 mm, ≤˜3 mm, or ≤˜2 mm.

In some aspects, in patient(s) presenting with medium-density skin type(s), a deep plane release upper lip lift procedure, without use of muscle suspension, excising about 4 mm-5 mm of the upper lip results in a +0 incisor show; excising about 6 mm of the upper lip results in a +1 incisor show; excising about 7 mm of the upper lip results in a +2 incisor show; excising about 8 mm of the upper lip results in a +3 incisor show; excising about 9 mm of the upper lip results in a +4 incisor show; and excising about 10 mm of the upper lip results in a +5 incisor show; excising about 11 mm of the upper lip results in a +6 incisor show. Such excisions are exemplary and may be used as a guideline. In aspects, the presence of detectable or significant amount(s), e.g., excess, temporary or permanent lip filler(s) can cause such values to change.

### Upper Vermillion Slope(s)

According to aspect(s), marking system(s) and associated method(s) provided herein yield one or more slopes, e.g., one or more of slopes 28A, 26A, 24A, 22A, 22B, 24B, 26B, and 28B, of the upper vermillion border (identified in FIG. 2N) of between about 1 and about 3, such as a slope of about 2. In aspects, marking system(s) and associated method(s) provided herein yield slopes 28A and 28B which are at least mostly, at least generally, at least substantially, at least essentially, are essentially, or are the same (e.g., within about 0.5, 0.4, 0.3, 0.2, or 0.1 of one another). In aspects, marking system(s) and associated method(s) provided herein yield slopes 26A and 26B which are at least mostly, at least generally, at least substantially, at least essentially, are essentially, or are the same (e.g., within about 0.5, 0.4, 0.3, 0.2, or 0.1 of one another). In aspects, marking system(s) and associated method(s) provided herein yield slopes 24A and 24B which are at least mostly, at least generally, at least substantially, at least essentially, are essentially, or are the same (e.g., within about 0.5, 0.4, 0.3, 0.2, or 0.1 of one another). In aspects, marking system(s) and associated method(s) provided herein yield slopes 22A and 22B which are at least mostly, at least generally, at least substantially, at least essentially, are essentially, or are the same (e.g., within about 0.5, 0.4, 0.3, 0.2, or 0.1 of one another). In aspects, marking system(s) and associated method(s) provided herein yield slopes CP-A and CP-B which are at least mostly, at least generally, at least substantially, at least essentially, are essentially, or are the same (e.g., within about 0.5, 0.4, 0.3, 0.2, or 0.1 of one another). In aspects, some, most, generally all, substantially all, essentially all, or all (each) of slopes 28A, 26A, 24A, 22A, 22B, 24B, 26B, and 28B, of the upper vermillion border are at least mostly, at least generally, at least substantially, at least essentially, essentially, or are the same. In aspects, some, most, generally all, substantially all, essentially all, or all (each) of slopes 28A, 26A, 24A, 22A, 22B, 24B, 26B, and 28B, of the upper vermillion border are within about 0.5, 0.4, 0.3, 0.2, or within 0.1 of one another.

### Predictability, Consistency, & Frequency of Corrective Procedure(s)

In aspects, marking system(s) and associated method(s) provided herein yield one or more identifiable outcomes which are detectably or significantly more predictable compared to outcomes resulting from method(s), procedure(s), or both which are performed without use of such marking system(s). In aspects, marking system(s) and associated method(s) detectably or significantly improve upon or otherwise increase the predictability of attaining one or more target aesthetic effect(s), e.g., the predictability of attaining a target aesthetic appearance, compared to method(s), procedure(s) or both which are performed without such system(s) and associated method(s).

In aspects, marking system(s) and associated method(s) provided herein yield one or more identifiable outcomes which are detectably or significantly more consistent compared to outcomes resulting from method(s), procedure(s), or both which are performed without use of such marking system(s). In aspects, marking system(s) and associated method(s) detectably or significantly improve upon or otherwise increase the consistency in attaining one or more target aesthetic effect(s), e.g., the predictability of attaining a target aesthetic appearance, compared to method(s), procedure(s) or both which are performed without such system(s) and associated method(s).

In aspects, marking system(s) and associated method(s) provided herein yield one or more identifiable outcomes which are detectably or significantly more predictable, consistent, or both, compared to outcomes resulting from method(s), procedure(s), or both which are performed without use of such marking system(s), such that the frequency of the need to perform a revision procedure to modify result(s) is detectably or significantly reduced. In aspects, marking system(s) and associated method(s) detectably or significantly improve upon or otherwise reduce the frequency of revision procedure(s) performed to modify a result by at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, or, e.g., at least about 5% compared to method(s), procedure(s) or both which are performed without such system(s) and associated method(s).

In aspects, marking system(s) and associated method(s) provided herein yield one or more identifiable outcomes which are detectably or significantly more predictable, consistent, or both, compared to outcomes resulting from method(s), procedure(s), or both which are performed without use of such marking system(s), such that patient satisfaction with the procedure(s) incorporating the marking system(s), associated method(s), or both is at least about 5% greater, at least about 10% greater, at least about 15% greater, at least about 20% greater, at least about 30% greater, at least about 40% greater, at least about 50% greater, at least about 60% greater, at least about 65% greater, at least about 70% greater, at least about 80% greater, at least about 90% greater, at least about 95% greater than that of patients undergoing procedure(s) lacking incorporation of the marking system(s) and associated method(s) described herein, as determined by an appropriately powered and administered study(ies), such as survey(s) or trial(s), conducted by and overseen by practitioner(s) recognized as skilled in the art.

### Aging

In aspects, marking system(s) and associated method(s) provided herein yield a lip design resulting from an upper lip lift procedure, e.g., subnasal upper lip lift procedure, e.g., subnasal and corner lip lift procedure which detectably or significantly ages better than an upper lip lift procedure performed without the application of the method, wherein degree of aging is assessed by measuring the change in relaxed skin tension lines (RSTLs) over time, the extent of drooping around the upper alveolus over time, measuring the degree of the subfacial lip(s) and lateral mouth hanging from the anterior cheek and face relative to the central subnasal lop more densely attached to the pyriform and supported by the front teeth over time, or combination(s) thereof. In aspects such result(s) is/are determined by the professional(s) performing the procedure(s), the subject(s) undergoing the procedure(s), a group of medical professionals trained the art tasked with assessing the outcome of the procedure(s), one or more proactive or retroactive studies performed by or overseen by one or more skilled professionals in art, or any combination of any or all thereof over a period of time lasting from between about 3 years to about 70 years after the performance of the procedure, such as a period of time lasing at least about 3 years from the time the procedure was conducted, e.g., a period of time of ≥4 years, ≥5 years, ≥6 years, ≥7 years, ≥8 years, ≥9 years, ≥10 years, ≥12 years, ≥14 years, ≥16 years, ≥18 years, ≥20 years, ≥25 years, ≥30 years, ≥35 years, ≥40 years, ≥45 years, ≥50 years, ≥55 years, ≥60 years, ≥65 years, or, e.g., ≥70 years. In certain aspects, marking system(s) and associated method(s) provided herein yield a lip design resulting from the upper lip lift procedure, e.g., subnasal upper lip lift procedure, e.g., subnasal and corner lip lift procedure, which detectably or significantly avoids acceleration of sign(s) of aging and exaggeration of appearance over a period of at least about 3 years, at least about 5 years, at least about 10 years, at least about 25 years, or more compared to procedure(s) performed without incorporation of the marking system(s) and associated method(s) described here.

### Marked-Tip Adjustable Spacing Device(s)

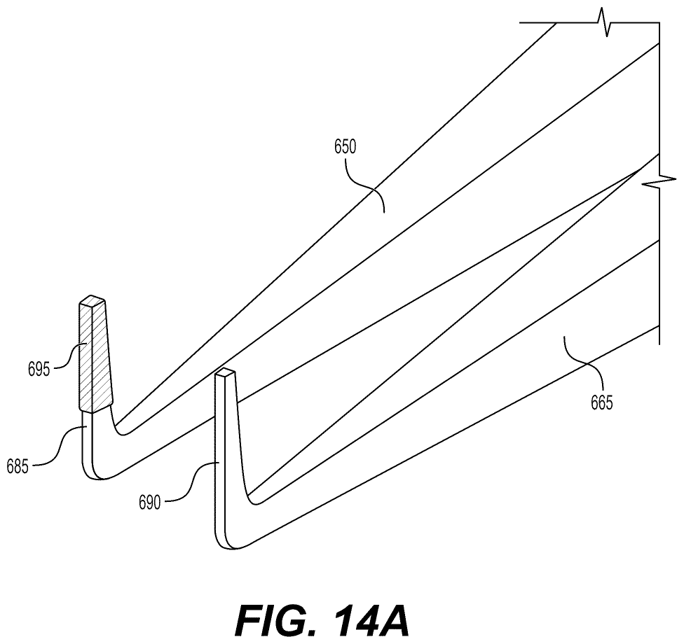

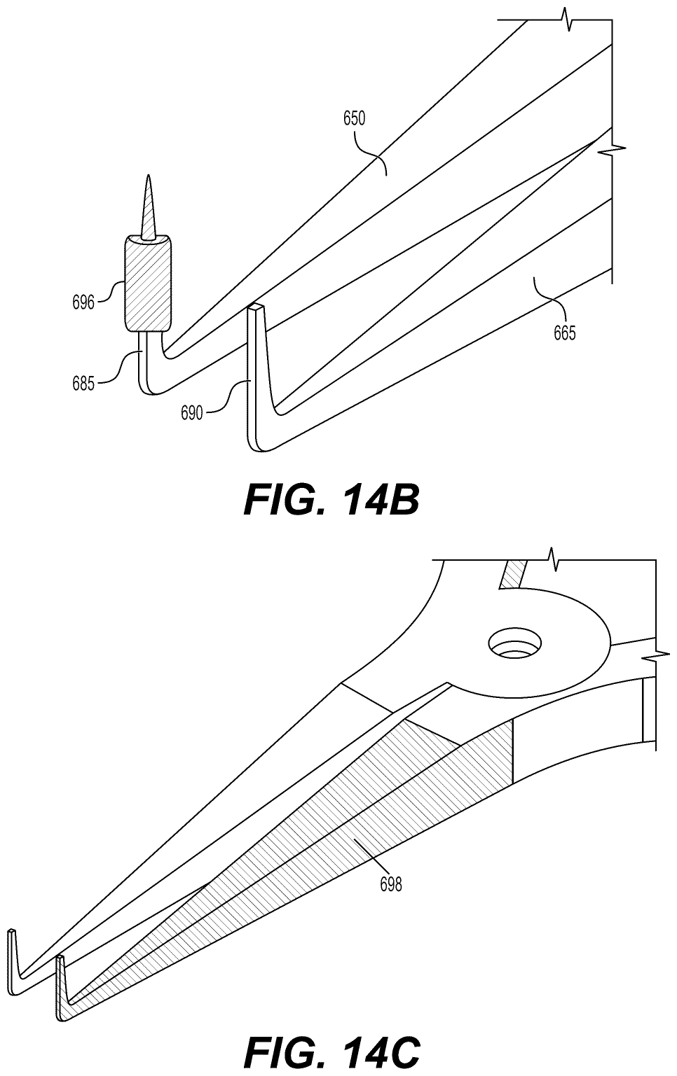

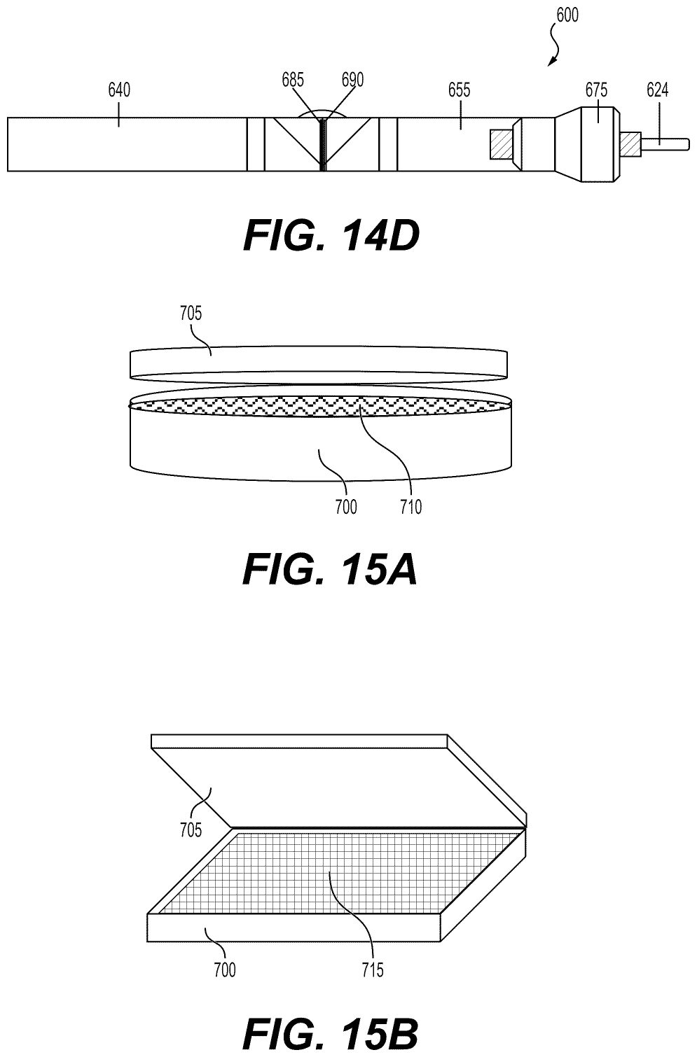

Marked-tip adjustable spacing device(s) (MT-ASD(s)) are described here. FIGS. 14A, 14B, 14C, and 14D are referenced and described here to aid the reader in understanding the disclosure.

According to certain aspects, the disclosure provides a means for stretching, measuring, marking, or combination(s) of stretching, measuring, and marking the skin for use in the production of marking system(s), for use in the performance of method(s) associated with marking system(s) provided herein, or both.

In aspects, a means for stretching, measuring, or marking the skin, or combination(s) thereof, provided by the disclosure can be any means suitable for use in establishing such marking system(s) and performing such associated method(s) described herein, such as, e.g., spring-loaded device(s) with an associated marking device; 2 part device(s) wherein a component part is capable of applying a stretch to the skin while a second component part is capable of applying a marking on the skin; specially designed caliper(s) such as caliper(s) comprising sliding mechanism(s) or caliper(s) comprising pivot point(s); skin stretching device(s) known in the art modified to incorporate a marking device, traction-based skin stretching device(s), etc.

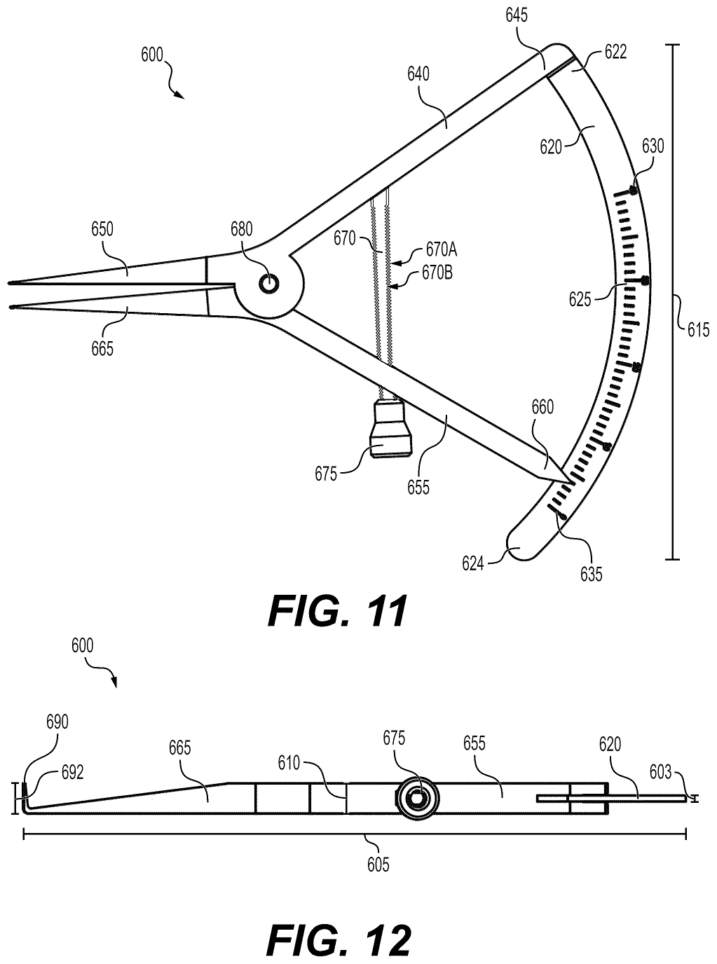

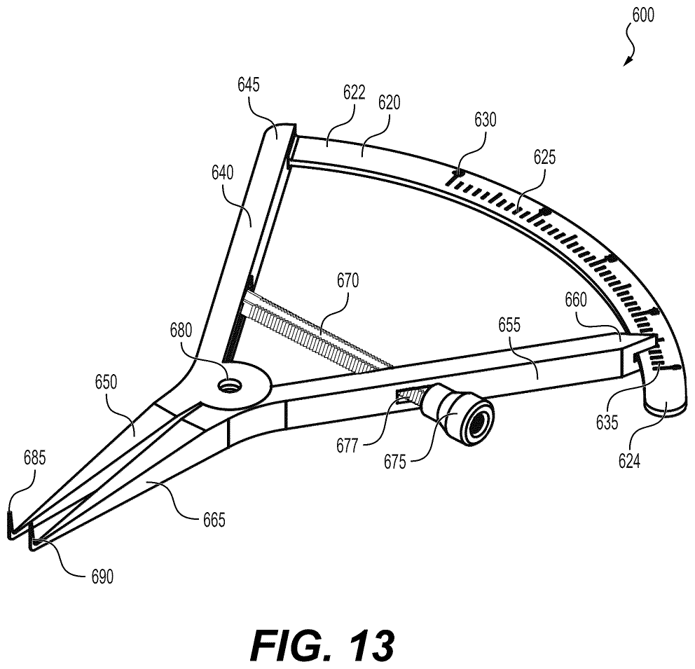

In certain aspects, a means for stretching, or a means for both stretching and marking the skin provided by the disclosure is a marked-tip adjustable spacing device, (“MT-ASD”). An exemplary MT-ASD (600) is provided in FIG. 11, FIG. 12, FIG. 13, FIG. 14A (in part), FIG. 14B (in part), FIG. 14C (in part), and FIG. 14D. In aspects, such a tool can be used to establish marking system(s) provided herein, can be used in method(s) comprising marking system(s) provided herein, can be used in procedure(s) comprising use of marking system(s) provided herein, or combination(s) of any or all thereof. In aspects, an MT-ASD comprises one or more “settings,” a setting indicating the distance at which the device is set to apply a marking, stretch the skin, measure a location, or combination(s) thereof. In aspects, the setting is indicated by a scale as described herein.

In aspects, an MT-ASD provided herein is a device which can be held by a medical practitioner performing or preparing to perform an aesthetic modification procedure in a position at least generally, at least substantially, at least essentially, essentially, or completely parallel to a subject's (patient's) face (e.g., to a subject(s) upper lip), while component(s)/elements of the MT-ASD make contact with the subject(s) face and allow for the practitioner to stretch, measure, mark, or combination(s) thereof, the subject's (patient's) face. In certain aspects, the MT-ASDs provided herein are specially designed to apply marking(s) of marking system(s) described herein, to be utilized in the performance of specific procedure(s) described herein, or both.

An MT-ASD provided herein is an adjustable device, allowing for the control of stretch, measurement, or marking application(s) applied by the device. MT-ASD(s) described herein typically comprise a first component (herein, such a component of an MT-ASD may be referred to as an element) and a second component (element), each having a length, wherein the two components are movable relative to one another. In aspects, while maintaining one of two such components in a fixed position, the other may be moved, and vice versa. However, in certain embodiments, one such component is fixed relative to at least another element of the device and, thus, may herein be referred to as a “fixed frame element.” In such embodiments, the second component is not fixed relative to such other element and thus may be herein referred to as a “movable frame element.” Herein, the term “frame” is used as a way to describe such element(s) as participating in the establishment of the size, shape, and character of the MT-ASD(s). Further, such element(s) may support the presence of one or more additional device component(s)/element(s) thereon/therewith. In certain aspects, the first and second movable components, e.g., a fixed frame element and a movable frame element, each rotate about a fixed point.

In aspects, each of the first and second movable components (e.g., the fixed frame element and the movable frame element) comprises an end suitable for making contact with the skin, an end comprising a marking device, or both. In aspects, each of the first and second movable components (e.g., the fixed frame element and the movable frame element) comprises a marking end. In aspects, the marking end is the end suitable for making contact with the skin. In aspects, when each of the first and second movable components ends suitable for making contact with the skin, or comprising a marking device, are in contact with the skin, movement of the first and second components relative to one another in a first direction reduces stretch of the skin, while movement of the first and second components relative to one another in a second direction increases stretch of the skin. Further, in aspects, when each of the first and second movable components ends suitable for making contact with the skin, or comprising a marking device, are in contact with the skin, movement of the first and second components relative to one another in a first direction reduces the distance between marking ends, while movement of the first and second components relative to one another in a second direction increases the distance between marking ends of the device.

Provided herein are MT-ASD(s) designed for establishing marking(s) of marking system(s) of this disclosure to mammalian skin, e.g., human skin, e.g., to the face of a human, e.g., to the upper lip of a human subject. In aspects, the MT-ASD(s) are suitable for use by medical professional(s) in the performance of aesthetic modification procedure(s) to measure distance(s) related to, and further to apply, marking(s) of marking system(s) or portion(s) of marking(s) of marking system(s) to the skin. MT-ASD(s) provided here can measure and apply such marking(s) while applying no stretch, a gentle stretch, or, e.g., a significant stretch to the skin.

Specific exemplary characteristic(s) and function(s) of MT-ASD(s) provided by the disclosure are described here. FIG. 13 provides an exemplary

### MT-ASD Size

In aspects, the disclosure provides MT-ASD(s) (600) having a length (605). In aspects, the length (605) of an MT-ASD is the dimension of the device spanning from the marking tip(s) (685), (690) to the furthest point on the opposite end of the device, in a straight line. In aspects, the length (605) of the device is the longest dimension of the device. See, e.g., length (605) in, e.g., FIG. 12.

In aspects, MT-ASD(s) of this disclosure can comprise any length allowing the device(s) to successfully perform their designed function(s). In aspects, MT-ASD(s) of this disclosure have a length (605) of no more than about 130 mm, such as, e.g., ≤˜125 mm, ≤˜120 mm, ≤˜115 mm, ≤˜110 mm, ≤˜105 mm, ≤˜100 mm, ≤˜95 mm, ≤˜90 mm, ≤˜85 mm, or ≤˜80 mm.

In aspects, MT-ASD(s) of this disclosure have a length (605) of between about 80 mm and about 125 mm, such as, e.g., ˜80 mm-˜120 mm, ˜80 mm-˜115 mm, ˜80 mm-˜110 mm, or, e.g., ˜85 mm-˜130 mm, ˜90 mm-˜130 mm, ˜95 mm-˜130 mm, ˜100 mm-˜130 mm, ˜105 mm-˜130 mm, or ˜110 mm-˜130 mm, such as, for example, ˜85 mm-˜125 mm, ˜130 mm-˜120 mm, ˜95 mm-˜115 mm, ˜100 mm-˜115 mm, ˜105 mm-˜115 mm, ˜110 mm-˜115 mm, or, e.g., ˜111 mm.

In aspects, the disclosure provides MT-ASD(s) (600) having a width (615). In aspects, the width (615) of an MT-ASD is the dimension of the device perpendicular to, but sharing a plane with, the device length (605). See, width (615) in, e.g., FIG. 11.

In aspects, MT-ASD(s) of this disclosure can comprise any width allowing the device(s) to successfully perform their designed function(s). For example, in aspects, MT-ASD(s) of this disclosure can comprise any width allowing the device(s) to providing a minimum marking distance of zero and a maximum marking distance of, e.g., ˜40 mm, ˜38 mm, ˜36 mm, ˜34 mm, ˜32 mm, or about 30 mm, such as, e.g., about 35 mm. In aspects, device(s) herein comprise a width of no more than about 80 mm, such as, e.g., ≤˜75 mm, ≤˜70 mm, ≤˜65 mm, ≤˜60 mm, ≤˜55 mm, or ≤˜50 mm.

In aspects, MT-ASD(s) of this disclosure have a width (615) of between about 50 mm and about 80 mm, such as, e.g., ˜50 mm-˜75 mm, ˜55 mm-˜75 mm, ˜60 mm-˜75 mm, or, e.g., ˜55 mm-˜80 mm, or ˜50 mm-˜80 mm, such as, e.g., ˜52 mm-˜80 mm, ˜54 mm-˜75 mm, ˜56 mm-˜70 mm, ˜58 mm-˜65 mm, or, e.g., ˜60 mm.

In aspects, the disclosure provides MT-ASD(s) (600) having a height (610). In aspects, the height (610) of an MT-ASD is the widest dimension of the device perpendicular to the plane of the device defined by its length (605) and its width (615). See, height (610) in, e.g., FIG. 12.

In aspects, MT-ASD(s) of this disclosure can comprise any height allowing the device(s) to successfully perform their designed function(s). In aspects, MT-ASD(s) of this disclosure have a height (610) of no more than about 10 mm, such as, e.g., ≤˜9 mm, ≤˜8 mm, ≤˜7 mm, ≤˜6 mm, ≤˜5 mm, ≤˜4 mm, or ≤˜3 mm.

In aspects, MT-ASD(s) of this disclosure have a height (610) of between about 3 mm and about 10 mm, such as, e.g., ˜3 mm-˜9 mm, ˜3 mm-˜8 mm, ˜3 mm-˜7 mm, ˜3 mm-˜6 mm, or ˜3 mm-˜5 mm, e.g., ˜3.5 mm-˜10 mm, or ˜4 mm-˜10 mm, as in, e.g., ˜3.2 mm-˜9 mm, ˜3.4 mm-˜8 mm, ˜3.6 mm-˜7 mm, ˜3.8 mm-˜6 mm, ˜3.9-˜5 mm, ˜4 mm-˜4.5 mm, or, e.g., about 4.1 mm, about 4.2 mm, or about 4.15 mm.

### MT-ASD Marking, Stretching, Measuring Distance

According to aspects, MT-ASD(s) of this disclosure provide the ability to measure an apply marking(s) or portion(s) thereof having a particular length or, e.g., distance between two points. That is, in aspects, MT-ASD(s) comprise a range of measurement/marking application, wherein a maximum distance is the maximum distance between the two marking tips (685) and (690) of the device which can be achieved by the device and wherein a minimum distance is the minimum distance between the two marking tip(s) (685) and (690) achievable by the device. Marking tip(s) (685) and (690) are described herein and are shown in, e.g., FIGS. 12, 13, 14A, and 14B. In aspects, a maximum distance between the pair of marking tips (685) and (690) establishes a maximum stretch which can be applied to skin by the device, a maximum measured distance provided by the device, and maximum distance between two points which can be marked by the device. In aspects, a minimum distance between the pair of marking tips (685) and (690) reflects no stretch applied to skin by the device, a measured distance of 0 (e.g., 0 mm) provided by the device, and minimum distance between two points which can be marked by the device (e.g., a distance of zero, e.g., 0 mm).

In aspects, MT-ASD(s) provided herein can comprise any maximum distance between the pair of marking tips (685) and (690) suitable for performing the tasks associated with device use. According to aspects, MT-ASD(s) herein provide a maximum distance between the pair of marking tips (685) and (690) of about 50 mm, such as, e.g., a maximum distance of about 48 mm, ˜46 mm, ˜44 mm, ˜42 mm, ˜40 mm, ˜38 mm, ˜36 mm, ˜34 mm, ˜32 mm, ˜30 mm, ˜28 mm, ˜26 mm, ˜24 mm, ˜22 mm, or, e.g., a maximum distance of about 20 mm.

In aspect, MT-ASD(s) provided herein comprise a maximum distance between the pair of marking tips (685) and (690) of about 20 mm-about 50 mm, such as, e.g., ˜22 mm-˜50 mm, ˜24 mm-˜50 mm, ˜26 mm-˜50 mm, ˜28 mm-˜50 mm, ˜30 mm-˜50 mm, ˜32 mm-˜50 mm, or ˜34 mm-˜50 mm, such as ˜20 mm-˜48 mm, ˜20 mm-˜46 mm, ˜20 mm-˜44 mm, ˜20 mm-˜42 mm, ˜20 mm-˜40 mm, ˜20 mm-˜38 mm, or ˜20 mm-˜36 mm, as in, e.g., ˜22 mm-˜48 mm, ˜24 mm-˜46 mm, ˜26 mm-˜44 mm, ˜28 mm-˜42 mm, ˜30 mm-˜40 mm, ˜31 mm-˜39 mm, ˜32 mm-˜38 mm, ˜33 mm-˜37 mm, ˜34 mm-˜36 mm, or, e.g., about 35 mm.

In aspects, MT-ASD(s) provided herein can comprise any minimum distance between the pair of marking tips (685) and (690) suitable for performing the tasks associated with device use. According to aspects, MT-ASD(s) herein provide a minimum distance between the pair of marking tips (685) and (690) of less than about 10 mm, such as, e.g., ≤˜8 mm, ≤˜6 mm, ≤˜4 mm, ≤˜2 mm, ≤˜1 mm, ≤˜0.5 mm, ≤˜0.1 mm, ≤˜0.05 mm, ≤˜0.01 mm, ≤˜0.005 mm, or ≤˜0.001 mm.

In aspect, MT-ASD(s) provided herein comprise a maximum distance between the pair of marking tips (685) and (690) of about 0 mm to about 3 mm, such as, e.g., ˜0 mm-˜2 mm, ˜0 mm-˜1 mm, or ˜0 mm-˜0.5 mm, such as, e.g., 0 mm. In aspects, the minimum marking distance is zero mm.

Accordingly, for example, MT-ASDs herein provide a measurement, skin stretch, marking distance or combination thereof of, e.g., between about 0 mm and about 50 mm, such as, e.g., ˜0 mm-˜45 mm, ˜0 mm-˜40 mm, ˜0 mm-˜35 mm, ˜0 mm-˜30 mm, ˜0 mm-˜25 mm, ˜0 mm-˜20 mm, ˜0 mm-˜15 mm, ˜0 mm-˜10 mm, ˜0 mm-˜9 mm, ˜0 mm-˜8 mm, ˜0 mm-˜7 mm, ˜0 mm-˜6 mm, ˜0 mm-˜5 mm, ˜0 mm-˜4 mm, ˜0 mm-˜3 mm, ˜0 mm-˜2 mm, ˜0 mm-˜1 mm, or ˜0 mm-˜0.5 mm, such as, e.g., ˜0.5 mm-˜50 mm, ˜1 mm-˜50 mm, ˜2 mm-˜50 mm, ˜3 mm-˜50 mm, ˜4 mm-˜50 mm, ˜5 mm-˜50 mm, ˜6 mm-˜50 mm, ˜7 mm-˜50 mm, ˜8 mm-˜50 mm, ˜9 mm-˜50 mm, ˜10 mm-˜50 mm, ˜15 mm-˜50 mm, ˜20 mm-˜50 mm, ˜25 mm-˜50 mm, ˜30 mm-˜50 mm, ˜35 mm-˜50 mm, ˜40 mm-˜50 mm, or ˜45 mm-˜50 mm. In certain aspects, MT-ASDs herein provide a measurement, skin stretch, marking distance, or combination thereof of between about 0 mm and about 50 mm, such as between about 0 mm and about 40 mm.

### MT-ASD Material(s)

MT-ASD(s) provided by this disclosure can comprise, e.g., can be made of a single type of material. In aspects, MT-ASD(s) comprise two or more component(s)/element(s) which differ in the material from which they are made. In aspects, MT-ASD(s) provided herein comprise at least 2, such as at least 3, 4, or at least 5 different materials.

In aspects, MT-ASD(s) can comprise any material suitable for allowing the MT-ASD(s) to perform their intended function. In aspects, a suitable material is a washable material. In aspects, a suitable material is a material capable of being sterilized, such as, e.g., by autoclaving. In aspects, a suitable material is a material suitable for contact with human skin without undesirable damage to the skin, reaction by the skin, or the like.

In aspects, MT-ASD(s) herein can comprise a polished finish. In aspects, MT-ASD(s) can comprise a material which is at least generally, at least substantially, at least essentially, is essentially, or is corrosion resistant.

In aspects, a suitable material is or comprises (e.g., generally comprises) a metallic material, such as, e.g., steel, e.g., stainless steel, or titanium.

In aspects, a suitable material is a non-metal material. In aspects, such a non-metal material is a plastic. In aspects, a non-metal material is suitable for maintaining, at least temporarily, a colorant, and as such is referred to herein as a non-metal colorant application material. For example, in aspects, the disclosure provides an MT-ASD for use in marking human skin, the MT-ASD comprising modifiable tips, e.g., coated tip(s) (see, e.g., FIG. 14A, element labeled as (695)), marking end(s) of frame elements (see, e.g., FIG. 14C, element labeled as (698)), or both, made of, coated with, or covered by a non-metal material capable of maintaining an effective amount of colorant, such that when such a colorant is present on the material, the MT-ASD applies visible markings to human skin when the MT-ASD is in use.

In aspects, marking device(s) can comprise, e.g., a rubber tip or tip made of a synthetic material, a rubber material, a fibrous material such as, e.g., cotton, polyester, paper, etc. capable of receiving a colorant, either temporarily or permanently, when such a colorant is placed upon it. See, for example, FIG. 14B, wherein element (696) exemplifies a tip similar to that of a typical children's coloring marker, pen, or the like is embodied. Note that FIG. 14B for sake of simplicity illustrates such a tip on only one marking tip/marking end of the MT-ASD. In most aspects, the same type of tip would be present on each marking tip of an MT-ASD or, e.g., each of the two tip(s) would have at least generally, at least substantially, at least essentially, essentially, or the same length. In certain aspect, one or more tip(s) can vary from one or more other tip(s) in type, functionality, length, or combination(s) thereof. In one example, in aspects, marking device(s) comprise a rubber tip, wherein prior to use, ink or other marking material is added to the rubber tip such that when the device is placed on the skin and the skin is stretched, the marking device(s) provide a marking on the skin. In aspects, marking device(s) of MT-ASD(s) comprise tip(s) which are selectively capable of providing marking(s). In aspects, a tip of an MT-ASD, e.g., a non-metal material tip, is sufficiently smooth, soft, malleable, or flexible so as to not detectably or significantly damage skin when the MT-ASD is used to apply a visible marking to human skin in embodiment(s) where such a tip is in contact with the skin when stretch is applied.

### Exemplary Component(s) of MT-ASD(s)

### Fixed Frame Element (640)

According to aspects, MT-ASD(s) provided herein comprise two movable components, the two movable components being movable relative to one another. As previously described, in aspects, while maintaining one of two such components in a fixed position, the other may be moved, and vice versa. However, in some aspects, one such component is fixed relative to at least another element of the device and, thus, may herein be referred to as a “fixed frame element.” In such embodiments, the second component is not fixed relative to such other element and thus may be herein referred to as a “movable frame element.”

According to aspects, MT-ASD(s) herein comprise a fixed frame element (640). In aspects, a fixed frame element has a fixed frame element fixed end (645) and a fixed frame element marking end (650). Fixed frame element marking end (650) can comprise a marking tip (685). See, e.g., FIGS. 11, 13, and 14D).

In aspects, fixed frame element(s)/component(s) (640) of MT-ASD(s) herein comprise a length, wherein their length, in aspects, defines the length of MT-ASD(s). In aspects, the length of a fixed frame element (640) is at least substantially the same, at least essentially the same, is essentially the same, or is the same as the length of a movable frame element (655). According to aspects, a fixed frame element has a width which is significantly less than its length (e.g., is less than about 10% of its length). In aspects, a fixed frame element (640) comprises a location along its length at which a pivot point (680) is positioned. In aspects, two or more components of an MT-ASD rotate or pivot about pivot point (680). In aspects, fixed frame element (640) pivots about pivot point (680). See pivot point (680) in, e.g., FIGS. 11, 12, and 13. In aspects, a pivot point can comprise, e.g., a pin, a screw, or the like which allows for two or more device components to rotate about it, e.g., by extending through opening(s) in such device component(s).

In aspects, a fixed frame element (640) comprises fixed frame element fixed end (645) which is fixedly attached to one or more other component(s) of a device, such as, e.g., an end of a scale display component (620). In aspects, a fixed frame element (640) comprises a marking end (650) which can comprise, e.g., a marking tip (685). See exemplary marking end (650) in, e.g., FIGS. 11, 13, and FIGS. 14A and 14B. In aspects, a marking tip (685) of a fixed frame element (640) extends out of the plane of the fixed frame element (640), e.g., marking tip (685) extends perpendicularly to the fixed frame element (640).

In certain aspects, a fixed frame element (640) can be made of a single material. In other aspects, one or more feature(s) or element(s) of a fixed frame element (640) can be made of multiple material(s). Material(s) of fixed frame element (640) can be any material described herein. In certain aspects, a fixed frame element marking end (650), the marking tip thereof (685), or both, can be made of a non-metal colorant application material as described elsewhere herein.

According to certain embodiments, a fixed frame element (640) comprises one or more openings within it, such as, e.g., at least an opening at the location of pivot point (680) and, e.g., optionally one or more openings or partial openings along its length. In aspects, an opening or partial opening along its length facilitates an interface with, or facilitates a connection to, a variable width spacer component (670) (discussed elsewhere herein). Such an opening or partial opening along its length can be, e.g., a complete opening or a partial opening such as a slot or track.

In certain aspects, a fixed frame element (640) is at least mostly, at least generally, at least substantially, at least essentially, is essentially, or is a straight component. In aspects, along its length, a fixed frame element (640) comprises a bend or, e.g., is angled. In aspects, such an angle can be an angle of, e.g., between about 5 degrees and about 45 degrees, such as, e.g., ˜5°-˜40°, ˜5°-˜35°, ˜5°-˜30°, ˜5°-˜25°, ˜5°-˜20°, ˜5°-˜15°, or, e.g., ˜5°-˜10°, such as, e.g., ˜10°-˜45°, ˜15°-˜45°, ˜20°-˜45°, ˜25°-˜45°, ˜30°-˜45°, ˜35°-˜45°, or ˜40°-˜45°, as in, e.g., ˜10°-˜40°, ˜15°-˜35°, or ˜20°-˜30° or ˜15°-˜35° or ˜20°-˜35°. In aspects, such an angle is also present in a movable frame element (655), however the angle of the movable frame element (655) represents a mirror image of that of the fixed frame element (640). See for example FIG. 13.

In certain aspects, a fixed frame element (640) can comprise a thickness (height) which is uniform across its length (absent, e.g., the length of the marking tip(s)). In other aspects, a fixed frame element (640) can comprise a height (thickness) which is non-uniform across its length, such as, e.g., comprising one or more areas which have a height which is less than about 95% of, e.g., ≤˜90%, ≤˜85%, ≤˜80%, ≤˜75%, ≤˜70%, ≤˜65%, ≤˜60%, ≤˜55%, ≤˜50%, ≤˜45%, ≤˜40%, ≤˜35%, ≤˜30%, ≤˜25%, ≤˜20%, ≤˜15%, ≤˜10%, or, e.g., ≤˜5% that of the largest height of the element.

In aspects, each of first and second movable components, e.g., each of fixed frame element (640) and movable frame element (655) comprise an angle, wherein the angle of each component is the same, and the direction of each angle represents a mirror image of the other.

### Movable Frame Element (655)

Again, as provided above, MT-ASD(s) provided herein can comprise two movable components, the two movable components being movable relative to one another and while maintaining one of two such components in a fixed position, the other may be moved, and vice versa. In some aspects, one such component is not fixedly attached to an element to which a fixed frame element (640) is fixedly attached. Accordingly, this second movable component may be referred to herein as a “movable frame element.”

According to aspects, MT-ASD(s) herein comprise a movable frame element (655). In aspects, a movable frame element has a movable frame element indicator end (660) and a movable frame element marking end (655). Movable frame element marking end (655) can comprise a marking tip (690). See, e.g., FIGS. 11, 12, 13, and 14D.

In aspects, movable frame element(s)/component(s) (655) of MT-ASD(s) herein comprise a length, wherein their length, in aspects, defines the length of MT-ASD(s). In aspects, the length of a movable frame element (655) is at least substantially the same, at least essentially the same, is essentially the same, or is the same as the length of a fixed frame element (640). According to aspects, a movable frame element has a width which is significantly less than its length (e.g., is less than about 10% of its length). In aspects, a movable frame element (655) comprises a location along its length at which a pivot point (680) is positioned. In aspects, movable frame element (655) pivots about pivot point (680).

In aspects, a movable frame element (655) comprises a movable frame element indicator end (624) which, as the device is manipulated (e.g., manipulated to select a desired setting, e.g., measurement/marking/stretch distance), is capable of moving along the scale (625) of the scale display component (620). In aspect, the position of the movable frame element (655), or more specifically the position of the movable frame element indicator end (624) of the movable frame element (655) indicates the marking/measurement/stretch distance present between the pair of tips (685) and (690). In aspects, a movable frame element (655) comprises a marking end (665) which can comprise, e.g., a marking tip (690). In aspects, a marking tip (690) of a movable frame element (655) extends out of the plane of the movable frame element (655), e.g., marking tip (690) extends perpendicularly to the movable frame element (655). See, e.g., FIGS. 11, 12, and 13 for exemplary scale display component (620), and FIGS. 11 and 13 for exemplary movable frame element indicator end (624) and its interface to/with scale (625).

In certain aspects, a movable frame element (655) is at least mostly, at least generally, at least substantially, at least essentially, is essentially, or is a straight component. In aspects, along its length, a movable frame element (655) comprises a bend or, e.g., is angled. In aspects, such an angle can be an angle of, e.g., between about 5 degrees and about 45 degrees, such as, e.g., ˜5°-˜40°, ˜5°-˜35°, ˜5°-˜30°, ˜5°-˜25°, ˜5°-˜20°, ˜5°-˜15°, or, e.g., ˜5°-˜10°, such as, e.g., ˜10°-˜45°, ˜15°-˜45°, ˜20°-˜45°, ˜25°-˜45°, ˜30°-˜45°, ˜35°-˜45°, or ˜40°-˜45°, as in, e.g., ˜10°-˜40°, ˜15°-˜35°, or ˜20°-˜30° or ˜15°-˜35° or ˜20°-˜35°. In aspects, such an angle is also present in a fixed frame element (640), however the angle of the movable frame element (640) represents a mirror image of that of the movable frame element (655). See for example FIG. 13.

In certain aspects, a movable frame element (655) can comprise a thickness (height) which is uniform across its length (absent, e.g., the length of the marking tip(s)). In other aspects, a movable frame element (655) can comprise a height (thickness) which is non-uniform across its length, such as, e.g., comprising one or more areas which have a height which is less than about 95% of, e.g., ≤˜90%, ≤˜85%, ≤˜80%, ≤˜75%, ≤˜70%, ≤˜65%, ≤˜60%, ≤˜55%, ≤˜50%, ≤˜45%, ≤˜40%, ≤˜35%, ≤˜30%, ≤˜25%, ≤˜20%, ≤˜15%, ≤˜10%, or, e.g., ≤˜5% that of the largest height of the element.

In certain aspects, a movable frame element (655) can be made of a single material. In other aspects, one or more feature(s) or element(s) of a movable frame element (655) can be made of multiple material(s). Material(s) of movable frame element (655) can be any material described herein. In certain aspects, a movable frame element marking end (665), the marking tip thereof (690), or both, can be made of a non-metal colorant application material as described elsewhere herein. Movable frame element marking end (665) is shown in, e.g., FIGS. 11-13 and FIGS. 14A and 14B.

According to certain embodiments, a movable frame element (655) comprises one or more openings within it, such as, e.g., at least an opening at the location of pivot point (680) and, e.g., optionally one or more openings or partial openings along its length. In aspects, movable frame element (655) comprises movable frame element opening (677) facilitating an interface with, or facilitating a connection to, a variable width spacer component (670). In aspects, movable frame element opening (677) passes completely through movable frame element (655). In aspects, one or more MT-ASD component(s) are positioned within movable frame element opening (677), such as, e.g., at least a portion of a variable width spacer component (670). See, e.g., FIGS. 11 and 13.

### Scale Display Component (620)

In aspects, MT-ASD(s) of this disclosure comprise a scale display component (element) (620). In aspects, the scale display component can be any component capable of providing a scale which reflects a device setting (e.g., a span/distance between the pair of marking tips (685) and (690)). See, e.g., FIGS. 11-13, and, e.g., FIG. 14D (in part).

According to aspects, a scale display component comprises a first end (622) and a second end (624). In aspects, a scale display component is fixedly attached to one or more other device component(s), such as, e.g., a frame element, e.g., a fixed frame element (640). In aspects, a scale display component comprises a first end (622) fixedly attached to a fixed frame element (640), which the second end (624) is not fixedly attached to any other device component. In aspect, one or more device component(s) can move about or along a scale display component (620). In aspects, a movable frame element (655) can move about or along a scale display component, establishing the marking/measuring/stretching distance of the device. In aspects, such movement is controlled at least in part by a variable width spacer component (670) and element(s) thereof, described elsewhere herein.

In aspects, a scale display component (620) spans a width representative of the device width. In aspects, a scale display component spans a width which is less than the device width. In aspects, when the device is established at one setting (e.g., at one marking/measuring/stretching distance), the maximum dimension of the scale display component (620) establishes the maximum width of the MT-ASD. In aspects, when the device is established at another setting (e.g., another marking/measuring/stretching distance), the maximum dimension of the scale display component (620) is less than the maximum width of the MT-ASD.

In certain aspects, a scale display component (620) has a height (603). See, e.g., FIG. 12. In aspects, the height of the scale display component is less than the height of the MT-ASD. In aspects, the height of the scale display component is less than about 95%, ≤˜90%, ≤˜85%, ≤˜80%, ≤˜75%, ≤˜70%, ≤˜65%, ≤˜60%, ≤˜55%, ≤˜50%, ≤˜45%, ≤˜40%, ≤˜35%, ≤˜30%, ≤˜25%, or ≤˜20% that of the MT-ASD.

In aspects, the scale display component (620) is an at least substantially straight element, being oriented in at least substantially one direction. In aspects, the scale display component (620) is characterizable as being curved, e.g., having an arced shape.

In certain aspects, such a scale display component (620) comprises a scale (625). In aspects, the scale has a low value end (635) and a high value end (630). See, e.g., FIGS. 11 and 13. In aspects, the highest value identified at the high value end (630) reflects the maximum distance which can be established by the device between the pair of marking tips (685), (690). In aspects, the lowest value identified at the low value (635) end reflects the minimum distance which can be established between the pair of marking tips (685), (690). In aspects, the high value end (630) of scale (625) is positioned closer to the scale display first end (622), which may be fixedly attached to another device component (e.g., a fixed frame element (640), while the low value end (635) of scale (625) is positioned closer to the scale display second end (624) which lacks a fixed attachment to any other component of the MT-ASD. In alternative embodiments, not shown, MT-ASD(s) comprise a digital scale component.

In aspects, a scale (625) can comprise any range of values using any unit(s) suitable for allowing the device to achieve its intended function(s). In aspects, a single scale is provided, with values provided in a single unit. In aspects, multiple scales are present such a plurality of units may be used. In aspects, unit(s) of scale(s) (625) of MT-ASD(s) herein must be sufficiently small so as to facilitate careful control over marking(s) made by the device. In aspects, the MT-ASD has a scale in millimeters. In certain specific aspects, the device has a scale ranging from, e.g., 0 mm to about 50 mm, ˜45 mm, ˜40 mm, or, e.g., about ˜35 mm. In aspects, the device can be set to any value within such a range.

In aspects, the high value end (630) of scale (625) is a value of about 20 mm-about 50 mm, such as, e.g., ˜22 mm-˜50 mm, ˜24 mm-˜50 mm, ˜26 mm-˜50 mm, ˜28 mm-˜50 mm, ˜30 mm-˜50 mm, ˜32 mm-˜50 mm, or ˜34 mm-˜50 mm, such as ˜20 mm-˜48 mm, ˜20 mm-˜46 mm, ˜20 mm-˜44 mm, ˜20 mm-˜42 mm, ˜20 mm-˜40 mm, ˜20 mm-˜38 mm, or ˜20 mm-˜36 mm, as in, e.g., ˜22 mm-˜48 mm, ˜24 mm-˜46 mm, ˜26 mm-˜44 mm, ˜28 mm-˜42 mm, ˜30 mm-˜40 mm, ˜31 mm-˜39 mm, ˜32 mm-˜38 mm, ˜33 mm-˜37 mm, ˜34 mm-˜36 mm, or, e.g., about 35 mm.

In aspects, the low value end (635) of scale (625) is a value of between about 0 mm and about 10 mm, such as a value of about 0 mm.

According to aspects, at least one component of the MT-ASD is capable of being positioned at different locations of the scale display component (620) and at different position(s) along the scale (625). In aspects, a movable frame element (655) is capable of being positioned at different locations of the scale display component (620), and, e.g., in particular aspects, the movable frame element indicator end (660) is capable of being positioned at different position(s) along the scale (625).

In aspects a scale (625) is present on a single side, e.g., a single face, of a scale display component (620). In alternative aspects, a scale (625) is present on multiple side(s), e.g., multiple faces, of a scale display component (640). In aspect(s) scale(s) on multiple face(s) of an MT-ASD can be the same or different.

### Variable Width Spacer (670)

According to aspects, MT-ASD(s) comprise a mechanism for adjusting the marking, stretching, or measurement distance capable of being applied by the device. In aspects, MT-ASD(s) comprise multiple component(s) independently participating in the marking, stretching, and measurement functionality(ies) of an MT-ASD. In such aspects, an adjustment mechanism can, in aspects, independently control the marking, stretching, or measurement distance(s) independently. In certain aspects, a single adjustment mechanism controls the marking, stretching, and measurement distance(s) of MT-ASD(s) in embodiment(s) where such functionality(ies) are provided by shared device component(s), such as paired marking tips (685), (690).

According to certain aspects, an adjustment mechanism capable of adjusting the marking, stretching, measurement distance(s) of device(s) herein can be any suitable mechanism allowing the device(s) herein to accomplish their intended function. In aspects, an adjustment mechanism is a mechanism providing an interface, e.g., a direct or indirect interface, between a fixed frame element (640) and a movable frame element (655). In aspects, one portion of such a mechanism interfaces with a fixed frame element (640) while another portion of such a mechanism interfaces with a movable frame element (655). In aspects, adjustment of the adjustment mechanism changes the position of the fixed frame element (640) relative to the movable frame element (655), and vice versa. In aspects, the adjustment mechanism is a screw mechanism. In aspects, the mechanism is a variable width spacer (670). In aspects, the variable width spacer (670) comprises an adjustment mechanism (675). See an exemplary variable width spacer (670) in, e.g., FIGS. 11, 13, and (in part) 14D.

According to aspects, MT-ASD(s) herein comprise a variable width spacer (670) having a portion which passes through a movable frame element (655) and having another portion which interfaces with a fixed frame element (640). In aspect, the interface with fixed frame element (640) is a constant interface, meaning that the variable width spacer (670) cannot be disconnected from a fixed frame element (640); however, it is not a fixed interfaced in that the variable width spacer (670) is movable relative to a fixed frame element (640). For example, in aspects, a variable width spacer (670) comprises a feature allowing for the interface angle between the variable width spacer (670) and the fixed frame element (640) to change according to device setting(s). In aspects, the interface angle between a variable width spacer (670) and a fixed frame element (640) approaches 90 degrees as the device approaches its maximum marking distance. In aspects, the interface angle between a variable width spacer (670) and a fixed frame element (640) (in the direction of the marking end (650) of the fixed frame element) gets smaller as the MT-ASD approaches its minimum marking distance (e.g., a marking distance of zero).

In certain aspects, a variable width spacer (670) is movable relative to a fixed frame element (640), a movable frame element (655), or both. In aspects, a variable width spacer (670) is rotatable relative to a fixed frame element (640) and a movable frame element (655).

According to certain aspects, a variable width spacer (670) comprises a variable width spacer outer element (670A) and a variable width spacer inner element (670B). In aspects, variable width spacer inner element (670B) is movable relative to variable width spacer outer element (670A). See, e.g., FIG. 11. According to aspects, the variable width spacer (670), a variable width spacer outer element (670A), a variable width spacer inner element (670B), or any combination thereof comprise more or less of their total lengths on one side of movable frame element (655) than another at different settings of the device. That is, in aspects, as setting(s) of an MT-ASD are modified, more or less of one or more of a variable width spacer (670), a variable width spacer outer element (670A), or a variable width spacer inner element (670B) pass through movable frame element opening (677). In aspects, a device user interfaces with a variable width spacer (670) via an adjustment interface (675). See an exemplary adjustment interface in, e.g., FIGS. 11 and 13. In aspects, modification of a device setting using a variable width spacer (670) is made via use of an adjustment interface (675). In aspects, a user interfaces with an adjustment interface (675) to modify the position of a variable width spacer (670) which, in turn, establishes a device setting.

In one specific aspect, a variable width spacer (670) comprises a screw. In aspects, the setting adjustment mechanism of an MT-ASD described herein is a screw mechanism. In aspects, the screw operates as a variable width spacer inner element (670B). In aspects, a user interfaces with an adjustment interface (675) to modify the position of a variable width spacer (670), variable width spacer inner element (670B), or both, which, in turn, establishes a device setting.

According to aspects, a variable width spacer (670) comprises an adjustment interface (675) providing a user with an ability to modify a device setting. In aspects, an adjustment interface (675) can be any interface facilitating the modification of a variable width spacer (670) or element(s) thereof resulting in the changing of a device setting. In aspects, such an adjustment interface (675) can be, e.g., a digital interface. In aspects, such an adjustment interface (675) can be an element which can be gripped by a user and pushed, pulled, or turned. In aspects, the variable width spacer (670) comprises a thumb screw mechanism and the adjustment interface is a thumb screw knob grippable by, e.g., two fingers (e.g., a thumb and second finger) of a human hand. In one exemplary use, an MT-ASD user grips the adjustment interface (675) and, e.g., rotates the adjustment interface (675). In aspects, the rotation changes the length of the variable width spacer (670) or length(s) of element(s) of the variable width spacer (670) (such as one or both of a variable width spacer outer element (670A) and a variable width spacer inner element (670B) positioned between a fixed frame element (640) and a movable frame element (655). In aspects, the rotation changes the length of the variable width spacer (670) or length(s) of element(s) of the variable width spacer (670) (such as one or both of a variable width spacer outer element (670A) and a variable width spacer inner element (670B)) positioned outside of a movable frame element (655). In aspects, rotation of the adjustment interface (675) rotate(s) the variable width spacer (670), and, if present, one or both of a variable width spacer outer element (670A) and a variable width spacer inner element (670B). In aspects, rotation in one direction moves marking tip (685) of a fixed frame element and marking tip (690) of a movable frame element closer together, while rotation in the opposite direction moves the pair of marking tips (685) and (690) closer together.

According to aspect, the adjustment interface (675) provides for sufficiently fine movement of the variable width spacer (670) to facilitate the millimetric (and, e.g., sub-millimetric) control of the distance between the pair of marking tips (685) and (690). In aspects, the distance between end(s) of the movable components (e.g., fixed frame element (640) and movable frame element (655), each comprising tip(s) suitable for making contact with and marking the skin can be selectively adjusted by the user. In aspects, the selective adjustment is accomplished by the use of a variable width spacer (670) which can be adjusted via an adjustment interface (675). In aspects, the distance between the two tips can be as low as 0 mm (e.g., when the tip(s) are adjacent to one another, such as, e.g., when the MT-ASD is in a closed position) and as wide as 60 mm, such as between about 0 mm and about 55 mm, ˜0 mm-˜50 mm, ˜0 mm-˜45 mm, or, e.g., ˜0 mm-˜40 mm. In aspects, the widest distance between tips can be, e.g., 40 mm or, e.g., about 35 mm. Accordingly, variable width spacer(s) (670) of MT-ASD(s) herein facilitate settings across such range(s). In aspects, a variable width spacer (670), e.g., thumbscrew mechanism, stably maintains tips apart at a desired/selected setting.

### Pivot Point (680)

According to certain embodiment(s), MT-ASD(s) comprise a pivot point (680) about which two or more device component(s) rotate. See, e.g., FIGS. 11-13.

According to aspects, a pivot point (680) of a device is positioned at a point where a fixed frame element (640) and a movable frame element (655) at least partially overlap. In aspects, at such point, the two frame elements (640) and (655) are movably connected to one another.

According to aspects, a shared pivot point (680) is positioned in a location along the length of each of a fixed frame element (640) and a movable frame element (655) identifiable as a vertex, wherein on either side of such pivot point (680) the fixed frame element (640) has portion(s) pointing in different directions and the movable frame element (655) has portion(s) pointing in different directions as is visible in FIG. 11. In aspects, pivot point (680) is characterizable as a hinge which is only movable via adjustment of the variable width spacer (670).

### Marking Tip(s) (685), (690)

Herein, the term “marking tip” is frequently used to describe the portion(s) of MT-AST(s) applying markings. In common aspects, such a feature also participate(s) in measurement function(s) (e.g., identifying a starting or ending point of a measurement). In common aspects, such a feature also participate(s) in stretching function(s), e.g., the application of a stretch to skin. As such, where the term(s) “marking tip” or “marking tip(s)” (or similar as clear by context) are used herein, the reader should interpret such language as encompassing terms such as “stretching tip(s),” “measurement tip(s),” and the like when such functionality is applicable.

According to aspects, MT-ASD(s) comprise two features capable of moving toward and away from one another according to a device setting to (a) increase or reduce skin stretch; (b) determine the distance between two points; (c) establish a distance between two points; (d) apply two marking(s) (e.g., point(s)) to a surface, such as, e.g. human skin, such mark(s) being a desired distance apart; or (e) combination(s) thereof. According to specific aspects, MT-ASD(s) comprise a pair of pointed tips that move away from each other (“open”) and move toward one another (“close”) according to device setting(s).

In aspects, the end of the first movable component (e.g., fixed frame element (640)), the marking end (650) of fixed frame element (640), the end of the second movable component (e.g., movable frame element (655)), the marking end (665) of movable frame element (655), or combination(s) thereof, each suitable for making contact with the skin, comprise a marking device. In aspects, marking device(s), e.g., a marking tip of a fixed frame element (685), a marking tip of a movable frame element (690), or both extend beyond the length of the first and second movable component(s). In aspects, in at least one position, marking device(s), e.g., a marking tip of a fixed frame element (685), a marking tip of a movable frame element (690), or both do not extend beyond the length of the first and second movable component(s). In aspects, marking device(s), e.g., a marking tip of a fixed frame element (685), a marking tip of a movable frame element (690), or both can selectively extend beyond the length of the first and second movable component(s). In aspects, marking device, e.g., a marking tip of a fixed frame element (685), a marking tip of a movable frame element (690), or both can be present in a first storage position and a second ready-for-use position. In aspects, changing marking device(s), e.g., a marking tip of a fixed frame element (685), a marking tip of a movable frame element (690), or both from a storage position to a ready-for-use position or vice versa requires input by a user of the MT-ASD with which such marking device(s) are associated. In aspects, input by a user is the extension of the marking device(s), e.g., a marking tip of a fixed frame element (685), a marking tip of a movable frame element (690), or both beyond the length of the first or second movable component(s) (e.g., beyond the length of the fixed frame element (640), movable frame element (655), or both). Exemplary positioning of marking device(s) e.g., marking tip(s) of fixed frame element(s) (685) and marking tip(s) of movable frame element(s) (690) is further described below. See also, e.g., FIGS. 12, 13, 14A, 14B, and 14D.

In aspects, marking device(s), e.g., marking tip(s) (e.g., (685), (690)) are present at the end of the first movable component (e.g., fixed frame element (640)), second movable component (e.g., movable frame element (655), or both and are suitable for making contact with the skin. In certain aspects, marking tip(s) (e.g., (685), (690)) are provided as blunt tips, comprising a shape which is not sharp or pointed. In alternative aspects, marking tip(s) comprise a sharp or pointed shape. In certain aspects, one tip may comprise a shape which is detectably or significantly different than the other, such as detectably or significantly more blunted in shape or more pointed in shape, than the other.

In aspects, marking device(s), e.g., marking tip(s) are available for use at all times but require modification prior to being able to effectively place a mark upon the skin.

In aspects, a marking device can be, e.g., a tip (e.g., a marking tip (685) of a fixed frame element (640), a marking tip (690) of a movable frame element (655), or both). In aspects, such tip(s) can comprise any suitable material for contacting human skin, such as, e.g., a polyester tip, e.g., comprising polyester fibers such as polyethylenterephalate or acrylic fibers (e.g., polyacrylonitrile). In aspects, such a tip is a felt tip or a tip made of highly compressed synthetic fibers or porous ceramics. In aspects, such a tip can be similar to that of a typical pen, e.g. comprising a roller ball/ball point. In aspects, such a tip can be made out of a marking material such as lead, or mixtures of materials such as those used in typical cosmetics such as eyebrow or lip marking pencils (e.g., thickeners waxes, oils, colorants, etc.) In aspects, a particularly suitable material is a material capable of marking directly onto human skin. In aspects, a particularly suitable material is a material capable of maintaining, at least temporarily, a sufficient amount of colorant such that the colorant can be applied by such a tip to human skin.

In aspects, a marking tip, e.g., a marking tip of a fixed frame element (640), a marking tip of a movable frame element (655), or both, is/are made of a non-metal material, e.g., a non-metal colorant application material. In aspects, a marking tip, e.g., a marking tip of a fixed frame element (640), a marking tip of a movable frame element (655), or both, is coated with of a non-metal material (695), e.g., a non-metal colorant application material. In aspects, marking tip(s) (e.g., (685), (690)) comprise a cap. In aspects, the cap is removable. In aspects, the cap is made of a non-metal material, e.g., a non-metal colorant application material. In aspects, the cap is made of a silastic material. In aspects, the disclosure herein provides cap(s), e.g., non-metallic caps which can be positioned on marking tip(s), e.g., marking tip(s) (685), (690), or both. In aspect(s), such a cap or, e.g., a coating, as described here, is a silastic cap or coating. In aspects, such a material can maintain a sufficient amount of colorant to apply a suitable amount of colorant to human skin. In particular aspects, a non-metallic material, e.g., a non-metal colorant application material (when discussed herein, such material can be present as a tip cap or a coating or, e.g., can form the body of, e.g., a marking end as discussed herein), is sufficiently flexible so as to not detectably or significantly damage mammalian skin when the device is used to stretch the skin, apply a visible marking to the skin, or both. According to certain embodiments, a marking tip of a fixed frame element (640), a marking tip of a movable frame element (655), or both, comprise a pen-like or marker-like tip or similar such tip which does not require a separate step of acquiring colorant prior to application of mark(s) to human skin (such a tip is exemplified as element (696) in FIG. 14B shown on a single tip of an exemplary device). Marking devices(s), e.g., marking tip(s) (685), (690), or both of MT-ASD(s) described herein, comprise a length. In aspects, the length of marking tip(s) (685), (690), or both define the height of device; that is, in aspects, there is no location of a device having a span in the same direction as the marking tip(s) (685), (690), or both, which extends beyond that of the marking tip(s). According to aspects, the marking tip of a fixed frame element (685), the marking tip of a movable frame element (690), or both, has/have a length which is no greater than about 10 mm, such as, e.g., ≤˜9 mm, ≤˜8 mm, ≤˜7 mm, ≤˜6 mm, ≤˜5 mm, ≤˜4 mm, or ≤˜3 mm. In aspects, the marking tip of a fixed frame element (685), the marking tip of a movable frame element (690), or both, has/have a length which is between about 3 mm and about 10 mm, such as, e.g., ˜3 mm-˜9 mm, ˜3 mm-˜8 mm, ˜3 mm-˜7 mm, ˜3 mm-˜6 mm, or ˜3 mm-˜5 mm, such as, e.g., ˜4 mm-˜10 mm, as in, e.g., ˜3 mm-˜9 mm, ˜3.2 mm-˜8 mm, ˜3.4 mm-˜7 mm, ˜3.6 mm-˜6 mm, ˜3.8 mm-˜5 mm, or about 4 to about 4.5 mm, such as, e.g., about 4 mm, or, e.g., about 4.15 mm.

According to particular aspects of the invention, fixed frame element (640) and movable frame element (655) at least generally, at least substantially, at least essentially, or essentially share the same plane.

In aspects, each of the first and second movable components, e.g., each of the fixed frame element (640) and movable frame element (655) comprises an angle, wherein the angle of each component is the same, and the direction of each angle represents a mirror image of the other. However, at least generally all, at least substantially all, or at least essentially all of each of the fixed frame element (640) and movable frame element (655) share the same plane except for any marking tip(s) associated therewith, which do not share such a plane, and in fact typically extend in a direction perpendicular to such plane, in a direction pointing 90 degrees away from such a plane.

In aspects, marking device(s) of such elements, e.g., a marking tip of a fixed frame element (640), a marking tip of a movable frame element (655), or both, do not share this plane. In aspects, the central axis of each marking tip, e.g., a marking tip of a fixed frame element (640), a marking tip of a movable frame element (655), or both, of the device(s) herein points in a direction perpendicular to the plane shared by the frame elements. See FIG. 12, wherein the marking tip (690) extends vertically 90 degrees from the plane of the remainder of the device. According to aspects, when in a closed position, tip(s) of an MT-ASD, e.g., a marking tip, e.g., a marking tip of a fixed frame element (640) and a marking tip of a movable frame element (655) are not pointing toward or angled toward one another. In aspects, a marking tip, e.g., a marking tip of a fixed frame element (640) and a marking tip of a movable frame element (655) are adjacent one another at such a setting. In aspects, a marking tip, e.g., a marking tip of a fixed frame element (640) and a marking tip of a movable frame element (655), are adjacent to one another across the entirety of their length(s) when the MT-ASD is in a fully closed position, representing the setting at which a marking distance of 0 is established. According to specific aspects, marking tips extend at a right angle from the plane established by the remainder of the body of the MT-ASD, e.g., extending in a direction establishing a right angle to the plane established by a fixed frame element (640), movable frame element (655).

According to one specific embodiment, the disclosure provides a marked-tip adjustable spacing device (MT-ASD) (600) for use in marking human skin, comprising a minimum marking distance and a maximum marking distance. In aspects, the MT-ASD comprises two frame elements (640), (655), each having at least approximately the same size and shape. In aspects, the frame elements are both elongated elements. In aspects, the two frame elements (640), (655) exist in a single, e.g., shared, device plane. That is, in aspects, the two frame elements are parallel to one another. In aspects, one frame element is movable (655) relative to the other (640) about a fixed point (680). According to aspects, each frame element ((640), (655)) comprises a marking end ((650), (665), respectively). In aspects, each marking end ((650), (665)) comprises a marking tip ((685), (690), respectively). In aspects, the marking tip extends from each of the two marking ends ((650) and (665)) of the frame elements ((640), (655), respectively), to form a pair of marking tips. In aspects, each marking tip has a length (692). See, e.g., FIG. 12. In aspects, each marking tip has the same length (692). In aspects, the length of the marking tips (685), (690) extends from each of the two frame elements ((640), (655), respectively) in a direction perpendicular to the device plane. In aspects, the pair of marking tips (685), (690) is positioned parallel to one another at all times during use of the device. Accordingly, in aspects, the length of the marking tips (692) is less than the device width (615) or is at least about the same, at least generally the same, at least substantially the same, at least essentially the same, is essentially the same, or is the same as the device width (615). According to certain embodiments, the length of the marking tips (692) is greater than the device width (615) but is within about 50%, such as within ˜45%, ˜40%, ˜35%, ˜30%, ˜25%, ˜20%, ˜15%, ˜10%, ˜5%, or, e.g., is within about 1% of the device width. In aspects, marking tip(s) (685) and (690) are in physical contact with one another across the entirety of their length when the MT-ASD (600) is established at its minimum marking distance position. In aspects, marking tip(s) (685), (690), or both are coated with, covered by, comprise a cap made of, or are made of a non-metal colorant application material (695); or marking end(s) (650), (665), or both, and marking tip(s) (685), (690), or both are coated with, covered by, or comprise a cap made of (as applicable), or are made of a non-metal colorant application material (695). Non-metal colorant application material is capable of maintaining an effective amount of a colorant (710) such that when the colorant (710) is present on one or both of marking tip(s) (685) and (690), the MT-ASD (600) applies visible markings to human skin when the MT-ASD (600) is in use. Colorant(s) and associated container(s) thereof, and kit(s) comprising the same are described elsewhere herein.

According to aspects, an MT-ASD is capable of applying one or more markings on the skin at a time, such as, e.g., two markings on the skin at a time. In aspects, an MT-AST applies two markings to the skin at a time when used in an upper lip lift procedure, e.g., subnasal upper lip lift procedure, e.g., subnasal and corner lip lift procedure. According to certain exemplary aspects, an MT-ASD is used in method(s) herein, e.g., method(s) of marking the skin, to mark the central (medial) line of marking system(s), e.g., radial marker 1 (006), one or more of the paramedial (philtral) lines of marking system(s), e.g., radial markers 2A (008A), 2B (008B), or both, or combination(s) thereof, for reference in an upper lip lift procedure, e.g., subnasal upper lip lift procedure, e.g., subnasal and corner lip lift procedure. In aspects, an MT-ASD is used in method(s) here, e.g., method(s) of marking the skin, to place the central (medial) line of marking system(s), e.g., radial marker 1 (006), one or more of the paramedial (philtral) lines of marking system(s), e.g., radial markers 2A (008A), 2B (008B), or both, or combination(s) thereof, for reference in an upper lip lift procedure, e.g., subnasal upper lip lift procedure, while applying a detectable or significant stretch to the upper lip to which the marking lines are applied.

According to certain aspects, ends comprising marking device(s) of first and second movable components of an MT-ASD are angled relative to one or more other component(s) of the first and second movable components, such as, e.g., a main body of each of the first and second movable component(s), the main bodies of the two movable components being, at least in one aspect, at least generally, at least substantially, at least essentially, essentially, or completely straight within a single plane. In certain aspects, ends of first and second movable components can comprise at least generally the same, at least substantially the same, at least essentially the same, essentially the same, or the same relative angle. In aspects, the end of a first movable component and the end of the second movable component, each suitable for marking the skin or comprising a marking device, can comprise a detectably or significantly different angle. In aspects, angle(s) of end(s) of first and second movable component(s) of an MT-ASD can comprise any angle relative to one or more component(s) of the first and second movable component(s), respectively, such as, e.g., an angle of about ˜10°, ˜15°, ˜20°, ˜25°, ˜30°, ˜35°, ˜40°, ˜45°, ˜50°, ˜55°, ˜60°, ˜65°, ˜70°, ˜75°, ˜80°, ˜85°, or, e.g., ˜90°. In aspects, the angle(s) of the end(s) of the first and second movable component(s) of an MT-ASD are positioned at about 90° angles relative to the main body of each of the respective first and second movable components. In aspects, marking tips (685), (690), or both of marking tips (685) and (690), e.g., marking tips of a fixed frame element (640), a movable frame element (655), or both, extend away from each of the main body of each of fixed frame element (640), movable frame element (655), or both, respectively, at an angle of between about 85° and about 95°, such as, e.g., at an angle of ˜85°-˜94°, ˜85°-˜93°, ˜85°-˜92°, ˜85°-˜91°, or ˜85°-˜90°, as in, e.g., ˜86°-˜95°, ˜87°-˜95°, ˜88°-˜95°, ˜89°-˜95°, or, e.g., ˜90°-˜95°, such as, e.g., ˜86°-˜94°, ˜87°-˜93°, ˜88°-˜92°, ˜89°-˜91°, or, e.g., at an angle of about 900.

In aspects, the disclosure provides the MT-ASD (600) of one or more of aspects 130-136, wherein each of the marking tips (685), (690) extends at least substantially in the same, single direction, wherein the direction is a direction perpendicular to the device plane. (ASPECT 137.). The invention also provides components corresponding to these parts of the devices as stand-alone devices, which may be adapted to engage a caliper and provide the functionality of the illustrated caliper devices.

According to aspects, the disclosure provides method(s) of establishing a marking system on the face of a subject in preparation for an aesthetic modification procedure wherein the method(s) comprise use of MT-ASD(s) comprising one or more characteristic(s) provided herein. According to certain aspects, the disclosure provides method(s) of performing an upper lip lift procedure, e.g., a subnasal and corner lip lift procedure, comprising the use of MT-ASD(s) comprising one or more of the characteristic(s) provided herein.

In aspects, to use MT-ASD(s) described herein, the tip(s) of the MT-ASD are dipped into or pressed onto a source of colorant, e.g., an ink pad, to load the tip(s) with sufficient colorant (e.g., ink) to make a marking on the skin when placed in contact therewith. In aspects, the MT-AST is adjusted such that there is a specifically defined distance between the tips as selected by the user. In aspects, the MT-ASD is then positioned and placed upon a subject's skin so as to establish two markings, made by the MT-ASD tips, on the subject's skin, a defined distance apart from one another.

According to certain aspects, MT-ASDs of the disclosure comprise one or more features of MT-ASD(s) provided in Figure(s) 11-13, and 14A-14D.

### Associated Colorant(s) and Colorant Container(s)

According to aspects, disclosed herein are MT-ASD(s) which apply a colorant (710) to human skin (e.g., in the form of one or more marking(s) or portion(s) thereof).

In certain aspects, MT-ASD(s) alone do not comprise a detectable or significant amount of colorant, e.g., a sufficient amount of colorant to apply a visible mark to human skin. In such embodiment(s), colorant must be added or otherwise applied to MT-ASD(s) prior to their ability to apply visible mark(s) to the skin. According to certain alternative aspects, MT-ASD(s) comprise a colorant, e.g., a store of colorant, such that the MT-ASD is in a ready-to-use state vis-à-vis its ability to apply visible mark(s) to the skin without the requirement to add or apply colorant to the device prior to use. In such aspects, e.g., which may be exemplified by an embodiment of FIG. 14B, MT-ASD(s) may comprise a marker-like or pen-like tip, e.g., a felt-tip or ball-point type tip (696) which comprises a sufficient amount of colorant so as to be capable of applying visible mark(s) to skin. In such embodiments, MT-ASD(s) can comprise a sufficient store of colorant to keep such a tip in a ready-to-use state.

In certain embodiments, marking device(s) of MT-ASD(s), e.g., marking tip(s) of fixed frame element(s), marking tip(s) of movable frame element(s) or both (e.g., (elements (685), (690), or both, as exemplified in the figure(s) herewith) are dipped in a colorant (710) prior to use. In aspects, marking device(s) of MT-ASDs are made of a suitable material for maintaining a sufficient amount of colorant thereon to facilitate the transfer of a visible amount of colorant to the target location, such as the skin of a human face.

According to aspects, colorant(s) can be any non-permanent inks, dyes, or the like as described herein. In aspects, a suitable dye can be, e.g., a triarylmethane dye. In aspects, gentian violet, also referred to as crystal violet, methyl violet 10B, or hexamethyl pararosaniline, is a suitable dye.

According to aspects, a suitable colorant can be housed in a colorant container (710) prior to it being added to an MT-ASD. In aspects, a colorant (710) can be present in such a container in free liquid form. See, for example, FIG. 15A, wherein exemplary colorant (710) is housed in liquid form in container (700). Therein, container (700) is shown as comprising lid (705), which can aid in maintaining liquid colorant (710) within container (700) without spilling.

In aspects, a colorant (710) can be present in a container (700) but maintained without spilling by the presence of a matrix or substrate (715) (see, e.g., FIG. 15B) which holds a sufficient amount of colorant (710) such that when an MT-ASD contacts substrate (715), a sufficient amount of colorant (710) is transferred to an MT-ASD (e.g., marking tip(s) of an MT-ASD) to facilitate the application of visible colorant mark(s) to the human skin by the MT-ASD. Such a substrate can be any substrate, e.g., a pad made of an absorbent material such as felt(s), foam(s), and the like.

In aspects, the disclosure herein provides MT-ASDs to which ink, e.g., gentian violet, is added using a pad-like container, e.g., an ink-pad-like container such as that provided in, e.g., FIG. 15B. In aspects, such container(s) described here are refillable. In aspects, a matrix or substrate (715) can be refillable, e.g., liquid colorant can be added when the matrix or substrate no longer comprises a sufficient amount of colorant. In aspects, when tip(s) of MT-ASD(s) are touched upon or pressed upon the matrix and an insufficient volume of colorant is captured by the tip(s) to apply suitably visible marking(s) to skin, more colorant in liquid form can be added to the matrix or substrate such that when tip(s) of MT-ASD(s) are touched upon or pressed upon the matrix or substrate, a sufficient amount of colorant is maintained on the tip(s) for the tip(s) to be able to apply marking(s) to the skin.

According to certain aspects, container(s) comprising colorant can comprise a fastening mechanism for attaching the container to one or more other objects. In aspects, such a fastening mechanism can be, e.g., any mechanism suitable for attaching the container to, e.g., a piece of clothing or cover/covering worn by, e.g., a medical professional participating in the performance of an aesthetic modification procedure or for example to a piece of clothing or cover/covering worn by a patient undergoing an aesthetic modification procedure. In aspects, such a fastening mechanism can be, e.g., any mechanism suitable for attaching the container to a device, machine, tool, piece of furniture, and the like which may be present during the performance of an aesthetic modification procedure in which an MT-ASD is being used (and for which the presence of the colorant container may be useful for applying colorant to the MT-ASD a plurality of times). In aspects, a fastening mechanism can be, e.g., a clip, snap, adhesive, hook-and-loop (e.g., Velcro), or any other similar or suitable fastening mechanism. In aspects, such a mechanism is a hook-and-loop fastening mechanism. In aspects, disclosed herein are colorant containers, e.g., colorant containers comprising a matrix/substrate for storing colorant, wherein the colorant container(s) comprise a hook-and-loop fastening mechanism. In aspects, colorant container(s) disclosed herein comprise a hook-and-loop fastening mechanism on one or more exterior surfaces(s) of the container, such as, e.g., on a lid, on the bottom of the container, or both.

### MT-ASD Kit(s)

According to aspects, disclosed herein are kit(s) comprising one or more MT-ASD(s) (600) and one or more colorant container(s) (700) each comprising colorant (710), such colorant optionally present in a colorant matrix (715).

In aspects, such MT-ASD(s) can comprise any one or more of the characteristic(s), feature(s), or functionality(ies) described herein. In aspects, colorant(s) and colorant container(s) can comprise any of the characteristic(s), feature(s), or functionality(ies) described herein. According to aspects, kit(s) comprising MT-ASD(s) can comprise one or more features of MT-ASD(s) provided in Figure(s) 11-13, and 14A-14D, one or more feature(s) of container(s) provided in Figure(s) 15A and 15B, or combination(s) thereof.

In aspects, kit(s) comprising a plurality of MT-ASD(s) can comprise at least one MT-ASD having at least one detectably or significantly different characteristic, feature, or function than that of at least one other MT-ASD in the kit. In aspects, kit(s) comprising a plurality of sources of colorant can comprise at least one source of colorant which has at least one detectably or significantly different characteristic, feature, or function than that of at least one other source of colorant in the kit.

### DETAILED DESCRIPTION OF SELECT FIGURES

The figures provided herein provide non-limiting embodiment(s), application(s), or related principle(s) of disclosure provided herein. Figures of this Application are provided to support the understanding of aspects of the disclosure, principles related to the disclosure, or practice of aspects of the disclosure.

Many figures are described elsewhere, throughout this specification, in the context of disclosure relevant to depiction(s) provided by such figures. Description of such figures is not repeated here.

However, select figures not described elsewhere are described here.

Any particular element(s) of system(s), step(s) of methods, condition(s) of process(es), and the like employed/depicted in the Figures and in the following detailed description thereof or disclosure elsewhere herein, are merely intended to further illustrate aspects of the disclosure. Specific element(s) of system(s), step(s) of method(s), condition(s) of process(es), and the like, such as their presence, position/order, and the like, and the general implications thereof, can be combined with or in aspects replaced by any other relevant part of this disclosure.

Readers should understand that the disclosure is not in any way limited by the Figures, or any description thereof.

### FIG. 16

The disclosure herein provides, in aspects, automated system(s), e.g., mobile application(s), for use in establishing guidelines such as measurement(s), feature characteristic(s), etc. as described herein, that are required to be applied in an aesthetic modification procedure in order to achieve target effect(s), e.g., target aesthetic appearance(s), (e.g., of a human face, e.g., of the upper lip of a human face) in the performance of one or more such aesthetic modification procedure(s). FIG. 16 provides one exemplary use flow (100) for an exemplary mobile application device provided by this disclosure used to identify such required guideline(s).

A mobile application (“application”) according to this disclosure can be made available for download from one or more application platforms or stores, such as, e.g., the Apple application store, or, e.g., equivalent(s) on other platform(s).

In a first step, a device user, here a “device” being a mobile device, such as, e.g., a smartphone or tablet, downloads the application onto the mobile device (110). Such download is typically facilitated by wireless transmission.

Once the application is available to the user on the mobile device, the user establishes a user account, e.g., signs up for application access by registering an account (115). In aspects, account registration can comprise the collection of one or more types of user-associated identification data, such as that described elsewhere herein. User-associated identification data can be used to authenticate the user's access to the application and can represent element(s) of the user's profile.

Upon creation of an account, a user can subsequently log into the application (120). Logging into the application typically comprises providing at least one, e.g., at least two or at least three pieces of identifying information, which are captured as input by the application and authenticated by comparing the input to expected input stored by the application. Successful login is achieved when successfully authenticated user-identifying input is provided to the application.

Once in the application, a user can create (e.g., establish a new) or update an existing user profile (135). A user profile may contain user-associated identification information, user preferences, or both. If a user should experience challenges accessing the application due to forgotten/misplaced login information, the user may use mechanism(s) within the application to address such login issue(s) (140), such as, e.g., to retrieve or reset one or both of a username and password or other required access information.

Once logged in, a user may arrive at a welcome screen (125) serving as a landing screen/page from which one or more option(s) may be available. In certain aspects, such a page may represent one, some, most, generally all, substantially all, essentially all, or all of the possible function(s) or feature(s) available in the application. In aspects one, some, most, generally all, substantially all, essentially all, or all feature(s)/function(s) are unavailable on such a screen unless the user has a paid account associated with access to one or more such feature(s)/function(s) (e.g., as would be typical in a software as a service (SAAS) model. According to certain aspects, one level of access (an all access or exclusive level, (130)) is available providing access to all feature(s)/function(s) of the application to certain users meeting the criteria for accessing such level—e.g., to user(s) having an appropriate subscription level and being current on payment for the same.

Within the application, users can access an image (145), either via, in aspects, a camera, camera-related application, or both, on the mobile device, wherein the camera, camera-related application, or both are in communication with the application such that images provided by the camera, camera-related application, or both are accessible by the application, or also or alternatively via a photograph. In aspects, the photograph can be taken by the camera, camera-related application, or both, or, e.g., the photograph can be retrieved from the mobile device (e.g., as stored locally or as stored in a cloud-based storage service accessible by the mobile device). In aspects, the application is in sufficient communication with the mobile device to be able to retrieve an image that can then be utilized and otherwise manipulated by the application. The image of (145) is herein referred to as an original image.

Once an image, e.g., an image of a human face, e.g., the face of the user or potential patient, is available to the application, the application scans the original image (150) to identify and apply specific landmarks using landmark recognition technology (155) to the original image. In aspects, the landmark recognition technology can be any landmark recognition technology as described elsewhere herein. In aspects, such a technology can create what may be referred to as a “mesh,” or “face mesh” as applied to a face, as, in certain embodiments, landmark recognition technology applied to a photo can look like a “web” or mesh of position indicator(s) placed upon the photo.

The application then compares (160) recognized feature characteristic(s) to training data, input data (data inputted or otherwise provided by application user(s), not shown), idealized image(s), one or more stored rule(s), one or more stored setting(s) or idealized feature(s), or combination(s) thereof. From this comparison, the original image is morphed to achieve a proposed target appearance (165). The proposed target appearance (165) is referred to here as a morphed image.

The morphed image is presented to the user (170). The user then evaluate(s) (175) the proposed target appearance and determines whether or not the morphed image achieves a target aesthetic appearance desired by the user. If the user determines that the morphed image fails to meet a target aesthetic appearance, and the user wishes not to save or further amend the morphed image, the user deletes (200) the proposed target appearance. If the user determines that the morphed image achieves a target aesthetic appearance desired by the user, the user saves (190) data associated with the proposed target appearance (morphed image) as a feature data set. If the user determines that the morphed image fails to meet a target aesthetic appearance however the user wishes to further amend the morphed image, the user amends/edits (180), either by manipulating one or more application setting(s)/feature(s), rules, or input(s) or, e.g., by allowing one or more AI algorithm(s) to amend the morphed image. Upon this additional morphing, the application generates (185) an updated morphed image to achieve a new proposed target image which can then be re-evaluated (175), saved (190), deleted (200), or again amended (180), until a satisfactory image is attained or until the user ends use of the application. Once a morphed image presenting a proposed target appearance is achieved, the image and associated data can be saved (190) as, e.g., a feature data set. The feature data set can be retrieved (195) when needed to use/application in an associated aesthetic modification procedure.

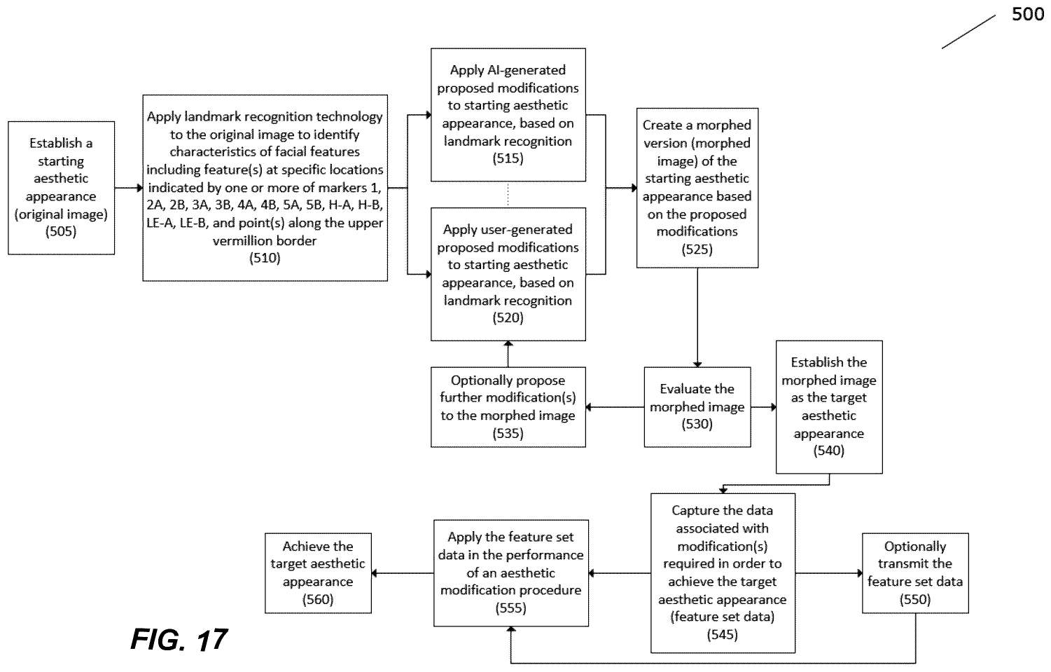

### FIG. 17

Provided herein are automated system(s) for establishing target effect(s), e.g., target aesthetic appearance(s) (e.g., of a human face, e.g., of the upper lip of a human face) using feature(s), characteristic(s), and principle(s) of marking system(s) described herein, which are anticipated to result from one or more aesthetic modification procedure(s) in which such marking system feature(s), characteristic(s), or principle(s) established by the system(s) are applied. Further, provided herein are method(s) of using such system(s) to achieve targeted result(s). To aid the reader in this general understanding, FIG. 17 presents an embodiment of method(s) (500) of achieving a target aesthetic result comprising the use of automated system(s) described herein.

A first starting, original aesthetic appearance is established (505). This can be by the capture of an original image, described elsewhere herein. This is established within an automated system participating in the method(s) described here. The original image is of a human, e.g., a human face, e.g., at least the upper lip and at least part of the nose of the human face. The human face is that of a person interested in undergoing an aesthetic modification procedure such as, e.g., an upper lip lift, a corner lip lift, or both. The automated system is operated by a user. The user is typically the person interested in undergoing the aesthetic modification procedure(s) or a medical professional anticipated to participate in such a procedure if/when performed.

Landmark recognition technology (e.g., a facial feature image recognition artificial intelligence model) is applied (510) to the original image to identify characteristic(s) of facial feature(s), including feature(s) at specific locations indicated by marker(s) described herein, such as, e.g., any one or more of 1, 2A, 2B, 3A, 3B, 4A, 4B, 5A, 5B, H-A, H-B, LE-A, LE-B, and point(s) along the upper vermillion border.

AI-generated proposed modifications are then applied (515) to the original image, based on the landmark recognition technology applied in step (510). Also or alternatively, a user of the automated system(s) applies (520) proposed modification(s) to the original image, based on the landmark recognition technology applied in step (510), via one or more interface(s) with the automated system(s). Such a proposed modification can be, e.g., an adjustment of morphing intensity setting(s), specifying feature(s) data (e.g., the length of marker(s) or segment(s) of marker(s) of marking system(s) described herein and indicated as modifiable within the automated system(s)), and the like.

Upon receipt of additional instructional data provided in (515) and also or alternatively (520), the automated system creates (525) a morphed version of the original image (a morphed image) based on the proposed modifications.

The morphed image is then presented to the user for evaluation (530). Upon evaluation, the user can delete the image and associated data (not shown) or, e.g., in one aspect, propose (535) additional modifications to the morphed image. Such modifications can again be AI-generated (as in 515), user-generated (as in 520), or both. In pursuing additional modifications, upon receipt of the same, the system then again creates (525) a new morphed image. Upon evaluation (530) of a presented image, the user can identify the proposed aesthetic appearance presented in the morphed image as suitable and establish (540) the morphed image as the target aesthetic appearance.

When a morphed image is established as a target aesthetic appearance, the data associated with the image (including, e.g., the image itself) can be captured/saved (545). This data includes all data associated with the modification(s) required to achieve the target aesthetic appearance. This data is captured as a feature data set.

A feature data set can optionally be transmitted (550), such as, for example, transmitted to an individual (e.g., medical professional), to a system (e.g., one or more systems associated with the performance of an aesthetic modification procedure), or the like. Feature data set(s) can be applied (555) in the performance of an aesthetic modification procedure, upon which the target aesthetic appearance is achieved (560).

The method(s) described by this figure utilize automated system(s) comprising at least the feature(s) and function(s) described in this particular example. System(s) may comprise one or more additional feature(s) and function(s) described elsewhere herein.

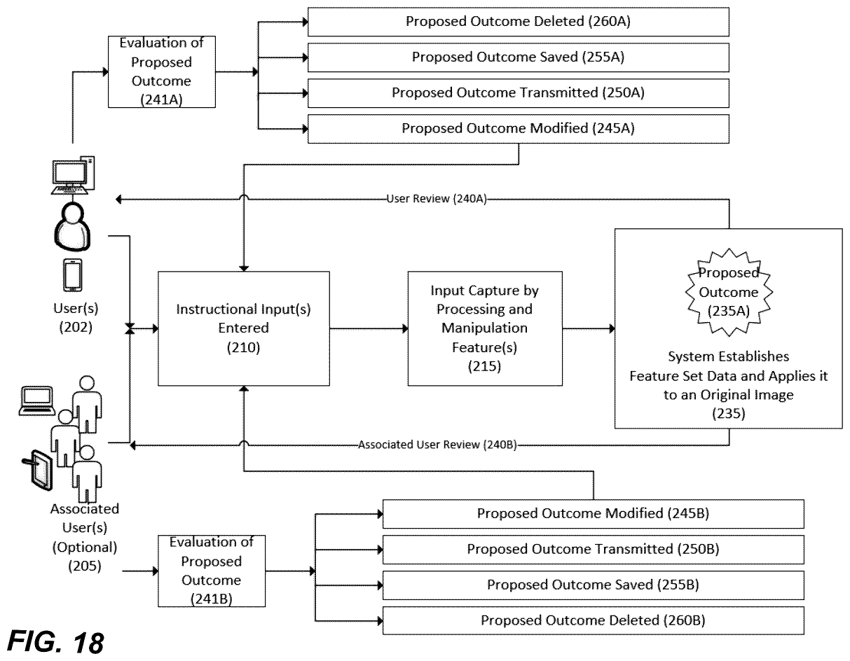

### FIG. 18

FIG. 18 presents an additional representation (200) of method(s) of achieving a target aesthetic result comprising the use of automated system(s) described herein. The process starts with an existing (original) aesthetic appearance image (not shown; e.g., of a human face), present on one or more device(s) housing the automated system and accessible to user(s) (202), associated user(s) (205), or both.

User(s) (202) or optional associated user(s) (205) enter (210) one or more instructional input(s) into an automated system. Such instructional input(s) can be, e.g., related to one or more characteristic(s) of marking system(s) described herein, such as, e.g., the total length or segment length associated with one or more of the marker(s) of marking system(s) provided herein, e.g., one or more of the total length(s), segment length(s), or positioning of, e.g., one or more of marker(s) 1, 2A, 2B, 3A, 3B, 4A, 4B, 5A, 5B, H-A, H-B, LE-A, LE-B, vermillion slope at one or more location(s), height difference(s) in point(s) where adjacent marker(s) intersect with the upper vermillion border, and the like. In aspects, automated system(s) can comprise, without the need for user input, one or more rules, exemplary input(s) related to or representing any of the input(s) described in this paragraph, or any guideline(s) for achieving target aesthetic appearance(s) disclosed herein.

The entered data is captured by a set of features/functions of the automated system associated with data processing and data manipulation (215). Such exemplary feature(s)/function(s) are exemplified in other figure(s) and disclosure herein and include such feature(s) and function(s) as, e.g., memory & processing function(s), visual guide(s) & measurement(s) generation and application technology(ies), landmark recognition technology(ies), image morphing technology(ies), artificial intelligence-based calculations, machine learning function(s), data editing capability(ies), and other such feature(s) and function(s) described elsewhere herein.

Upon processing, manipulating, or otherwise addressing (215) applied data, the system establishes and applies (235) a feature data set to the original image. The feature data set data comprises guideline(s) which could be applied in an aesthetic modification procedure on the subject matter of the original image (e.g., human face). The application of the feature data set to the original image results in the generation of the proposed outcome (235A). Proposed outcome (235A) is typically presented as a morphed version of the original image. The morphed version of the original image presents what the subject matter of the original image (e.g., human face) would look like if the guidelines of the feature set data were applied in an aesthetic modification procedure performed on such subject matter.

The proposed outcome (235A) is then presented ((240A), (240B), or both) to the user (as in 240A), associate user(s) (as in 240B), or both.

Upon receipt of the proposed outcome (235A) by the user, associate user(s), or both, the proposed outcome (235A) is evaluated ((241A), (241B), or both) by the user (as in 241A), associate user (as in 241B), or both.

Upon evaluation, the user or associate user can delete ((260A), (260B)) the proposed outcome (235A); save ((255A), (255B)) the proposed outcome (235A); transmit ((250A), (250B)), or propose additional modifications ((245A), (245B)) to the proposed outcome (235A). If modification(s) are to be proposed, one or more instructional input(s) are entered (210) by one or both of user(s) and associate user(s), and the process can iterate. In aspects, the process can iterate until a proposed outcome is established as a target aesthetic appearance and, e.g., saved, transmitted, or both.

As described elsewhere herein, automated system(s) disclosed herein can comprise control(s) preventing user(s) and associated user(s) from providing conflicting instruction(s) to the system. For example, one or more action(s) taken by an associate user may be first presented to a user as a proposed action, at which time the user can accept or reject such proposal(s) or, e.g., action(s) taken by a user can be, e.g., identified as one set of instructions, leading to, for example, the generation of one proposed outcome, while action(s) take by an associated user can be, e.g., identified as a separate set of instruction, leading to, for example, the generation of another proposed outcome. In aspects, a plurality of proposed outcome(s) can be maintained by system(s) and one, some, most, generally all, substantially all, essentially all, or all (each) presented to user(s), associated user(s), or both.

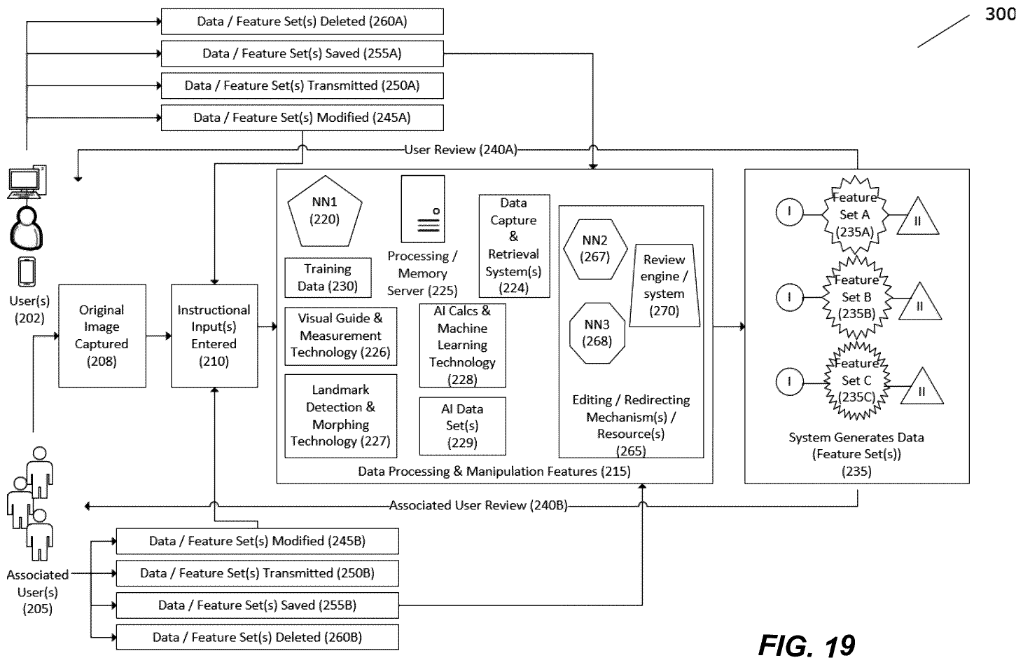

### FIG. 19

Provided herein are automated system(s) for establishing target effect(s), e.g., target aesthetic appearance(s) (e.g., of a human face, e.g., of the upper lip of a human face) using feature(s), characteristic(s), and principle(s) of marking system(s) described herein, which are anticipated to result from one or more aesthetic modification procedure(s) in which such marking system feature(s), characteristic(s), or principle(s) established by the system(s) are applied. FIG. 19 provides an example of feature(s) and function(s) which may be present in such system(s) (300).

System(s) provided herein provide access to user(s) (202), and optionally associated user(s) (205). User(s) (202), associated user(s) (205), or both can access the system via any one or more suitable interface(s). As exemplified, an interface can be a computer, such as a laptop or desktop computer, or mobile device such as, e.g., a smartphone or tablet, e.g., where an interface is delivered as a web page or a web application or similar application delivered via the internet, LAN, SDWAN, VPN, or another virtual network, etc. In aspects, user(s) (202), associated user(s) (205), or both are required to comply with authorization protocol(s) or tools and the system includes authorization engine(s) or other authorization methods/components (not shown).

According to aspects, associated user(s) (205), can also similarly access the system, optionally only with authorization as discussed briefly above, through such an interface or a different interface. Associated user(s) can include, for example, one or more medical professional(s) (e.g., one or more physician(s) or surgeon(s) associated with the performance of a pending procedure or, e.g., one or more medical professional(s) associated with a subject patient(s) medical care), the subject patient, one or more individual(s) associated with the subject patient, and the like. In aspects, one or more associated users can provide content either to the training set or instructional prompts. For example, an associated user might input an instructional prompt that applies to an individual subject procedure or alternatively to procedure(s) generally, in one or more situations (such as in generating a specific target outcome (e.g., a specific radial marker excision height segment length, a final lip height segment length, etc., or such as in generating a generalized target outcome (e.g., a target upper vermillion slope at a specific location for any target subject meeting one or more defined criteria such as race, gender, facial feature(s), etc.) Associated user(s) can also represent administrators that can change operational setting(s) of the system, such as by directly training neural network(s), via changing weights, providing the neural network with training experiences, or otherwise, or change other aspects of the system. In aspects, access to one or more feature(s) or function(s) differentiate(s) an associated user from a user.

To begin use of the system, an original image is captured (208). The original image represents the starting aesthetic appearance, which the system will modify (morph) to result in a target aesthetic appearance.

A user (202), associated user (205), or both, can submit instructional prompt data, (210) to the system via interface(s). Instructional prompt data can be input by direct submission (e.g., directly entering a value into a field), by user selection of system-loaded options presented via the interface, or both, or, e.g., is also or alternatively inputted by automatic operation of one or more functions of the system. The system can comprise data which, without user/associated user input, can optionally be (according to direction from the user/associated user) or which can automatically be (without direction from the user/associated user) applied to the original image. Together, such data can be referred to collectively as instructional data, as it instructs morphing software regarding how to modify the image.

The original image and instructional data are then addressed by data processing and manipulation feature(s) (215) of the system.

Data processing and manipulation feature(s) (215) of system(s) herein can comprise one or more neural network(s). Instructional data can be, or primarily/generally or essentially includes information/data that a neural network (e.g., neural network (220) or other(s)) is/are trained to recognize and prioritize in the generation of draft system-generated recommendation(s) (e.g., recommended marking system feature(s) used to morph an image).

Instructional data/content can include anything that the system recognizes as priority context for the generation of recommendation(s)/recommended feature set(s). Examples can include, for example, specific subject patient race, gender, age, skin type, etc., or e.g., specific marking system component characteristic(s), such as, e.g., a total radial marking 1 length, an excision height segment length of radial marking 1, a final lip height segment length of radial marking 1, etc.

Data processing and manipulation feature(s) (215) of system(s) herein can comprise a capable server comprising processing and memory components (225), and one or more trained neural network(s) (220). The processing and memory components of the system can be actual on-site componentry/systems, virtual (e.g., cloud-based) processing or memory systems, such as are available through commercially available and known platforms such as Microsoft Azure, Amazon Web Services, Google Cloud Platform, and the like, or a combination thereof. Similarly, neural network(s) of the system can be locally based, accessible through platform(s), such as, e.g., OpenAI, Microsoft Azure, HuggingFace, etc., or a mixture thereof, provided that at least some of the neural network(s) have any required training specified here for carrying out the functions/steps of the system/method. Other suitable platform(s) for providing functionality described here are exemplified herein.

Neural network(s) of data processing and manipulation feature(s) (215), e.g., neural network (220) (and optionally other(s), such as (267) and (268), if present, is/are trained with training data (230). Training data (230) can include, for example, photographs of positive facial feature(s) (e.g., photographs comprising feature(s) which may be identifiable as a target outcome of applied procedure(s)) photographs of negative facial feature(s) (e.g., photographs comprising feature(s) which may be identifiable as outcome(s) to be avoided in performance of procedure(s)), options for specific feature indicator(s), such as, e.g., optional total length(s) of radial marker(s) 1 (002), 2A (008A), 2B (008B), 3A (010A), 3B (010B), 4A (012A), 4B (012B), 5A (014A), 5B (014B), etc. or options for lengths of one or more of the excision segment length or final lip length of any one or more thereof; or, e.g., optional target slope(s) 1A (022A), 1B (022B), 2A (024A), 2B (024B), 3A (026A), 3B (026B), 4A (028A), 4B (028B), etc.; or, e.g., optional target height differences 1A (032A), 1B (032B), 2A (034A), 2B (034B), 3A (036A), 3B (036B), 4A (038A), 4B (038B); or, e.g., one or more features of a type I corner lift marking A (042A), type I corner lift marking B (042B), type II corner lift marking A (044A), type II corner lift marking B (044B); or, e.g., one or more feature(s) of horizontal indicator H-A (046A) or H-B (046B), and the like. Exemplary training data platform(s) are described elsewhere herein.

Data processing and manipulation feature(s) (215) of system(s) herein can comprise one or more visual guides and measurement technology(ies) (226). Such visual guide and measurement technology(ies) (226) can be capable of drawing visual guide(s), applying a measurement to or taking a measurement of element(s) of an image (e.g., photograph), or combination(s) thereof. Such visual guide and measurement technology(ies) (226) can be capable of building one or more user interface component(s) for displaying visual guide(s) and measurement(s).

Data processing and manipulation feature(s) (215) of system(s) herein can comprise one or more landmark detection and morphing technology(ies) (227).

Landmark detection technology(ies) can comprise a vision framework providing face detection and landmark detection capabilities. Landmark detection technology(ies) can comprise one or more library(ies) comprising one or more facial landmark detection algorithms. Landmark detection technology(ies) can comprise one or more framework(s) providing landmark detection capability(ies), e.g., facial landmark detection capability(ies), which provide pre-trained model(s), support real-time processing, or which provide pre-trained model(s) and support real-time processing. Facial landmark detection algorithms can provide facial landmark detection and tracking capability(ies), landmark detection algorithms such as those provided by one or more library(ies) or toolkit(s), or both. Morphing technology(ies) (image morphing technology(ies)) can provide one or more tools for image processing and morphing effect(s). Morphing technology(ies) can be capable of creating augmented reality experience(s) for visualizing one or more before and after effect(s) of an applied set of rules or actions, such as, e.g., before and after effects of a proposed lip lift procedure. Exemplary landmark detection and morphing technology(ies) are described elsewhere herein.

Data processing and manipulation feature(s) (215) of system(s) herein can comprise one or more AI-based calculation technology(ies) (228). AI-based calculation technology(ies) (228) can enable integration of pre-trained deep learning models for such AI-based calculation(s). AI-based calculation technology(ies) (228) may be characterizable as machine learning framework(s) and can be capable of training, deploying, or training and deploying customized deep learning model(s). Particular such AI-based calculation technology(ies) (228) can be specifically tailored for particular application(s), such as lip lift measurements. AI-based calculation technology(ies) (228) can enable interoperability between a plurality of deep learning frameworks.

Data processing and manipulation feature(s) can comprise or can be trained by one or more artificial intelligence (AI) data sets (229). AI data sets (229) can be used, e.g., to train deep learning models for facial analysis task(s). Artificial intelligence data set(s) (229) may provide landmark annotations, e.g., facial, or specifically lip landmark annotations, for a plurality, e.g., tens, hundreds, thousands, or, e.g., tens of thousands of feature(s), e.g., faces, in real-world images. AI data set(s) (229) can be used for training and evaluating facial landmark detection model(s).

Using steps/engines as will be described below, data processing and manipulation feature(s) (215) cooperate to generate one or more draft system-generated feature set(s) (235). FIG. 19 provides the one or more form(s) as feature sets A, B, and C ((235A), (235B), and (235C), respectively). Such feature set(s) can comprise data in one or more forms (exemplified by the indicators “I” and “II” associated with each of exemplified feature set(s) (235A), (235B), and (235C). For example, data associated with data set(s) can be, e.g., data which can be directly interpreted by a practitioner, data which can be employed manually by a practitioner in an associated procedure, data which can be interpreted by automated system(s) for automated use in a procedure, images, etc. In aspects, data in feature set(s) can be generated by the employment of one or more neural network(s) (e.g., (220), (267), (268)). In aspects, the employment of one or more neural network(s) provide(s) a distribution of each of the featured elements that the neural network or other component(s) of the system determine to include within a draft system-generated feature set. In certain aspects, such a distribution can be selected from by a selection engine (not shown) and thereafter incorporated into the applicable draft system-generated feature set. Distribution(s) and feature selection mechanism(s) of system(s) can be, in aspects, such that in most, generally all, or substantially all cases/iterations the feature sets generated by the system differ from each other by at least one or more characteristic(s). E.g., draft feature sets A, B, and C are expected to be distinct according to at least one characteristic. For example, system-generated draft feature sets A, B, and C may share a common draft radial marking 1 total length but may vary in the proposed excision height segment length of, e.g., radial marker(s) 2A, 2B, 3C, or 3B, etc.

These draft system-generated feature sets A, B, and C (e.g., (235A), (235B, (235C)) can comprise various types of data, or, e.g., can provide various types of data in various form(s) (shown as (“I”) and (“II”)). For example, one type of data may be raw data, in form(s) interpretable by a human system user, interpretable by an automated system (e.g., computer software), or both. For example, another type of data may be, e.g., image(s), e.g., computer-modified, or computer-generated morphed images of a subject patient with, e.g., each of the proposed system-generated draft features sets applied, wherein such image(s) provide an expected outcome appearance of the subject patient according to each proposed feature set.

Draft system-generated feature set(s) (235) are presented to the user (202), associated user(s) (205), or both, via their respective interface(s), wherein the user (202), associated user (205), or both can review ((240A), (240B), respectively) the feature set(s) (235) and rank/evaluate, edit, save, or, e.g., transmit the feature set(s). Data processing and manipulation feature(s) (215) can comprise or be associated with a communication server (not shown), such as, e.g., an email server, or can interface with any well-known email system, such as Gmail, Outlook365, and the like, to facilitate transmission of feature set(s) for, for example, further evaluation by system or non-system user(s) or, e.g., to facilitate transmission of a feature set once the user determines such a feature set is ready for applying to a specific procedure.

Data processing and manipulation features (215) of system(s) can comprise editing/redirection mechanism(s)/resources(s) (265), for promoting (e.g., detectably or significantly enhancing) the usefulness of system-generated feature set(s), the accuracy of system-generated feature set(s) in yielding target aesthetic appearance characteristic(s), the reproducibility of target aesthetic appearance characteristic(s) resulting from the system-generated feature set(s), and the like, or combination(s) thereof, of system-generated feature sets over future iterations/uses.

For example, system(s), e.g., (300), can comprise or be associated with one or more additional neural networks (267) which rank(s) system-generated feature sets on a scale or other rating system as compared to the content of original instructional data or by some other standard, which make(s) adjustment(s) to the instructional data, adjust(s) any present neural network weights/architecture/operation, and the like, or combination(s) thereof, in response to variances from any standard set by the user, associated user(s), or both, or which is/are programmed into the system.

In one example, a neural network (267) can rank proposed feature set(s) on a scale, and a separate neural network (268) can adjust one or more instructional prompts or pieces of instructional data (not shown). In related aspects of the disclosure, the disclosure also or alternatively provides for method(s)/system(s) comprising one or more neural network(s) that directly edit draft feature set(s) prepared by the system (not shown).

Also, or alternatively, the editing/redirecting resources can include review engines, systems, etc., (270), which facilitate user/associated user review of system-generated feature set(s), such as in a supervised learning model/environment, wherein such human assessment of system-generated feature set(s) (or other output evaluation) is used to change the instructional data, modify the operation conditions of the neural network(s) (e.g., weights or operation of some of the neurons in some layer(s) thereof), or both.

Editing/redirecting mechanism(s)/resource(s) (265) can comprise a third neural network or third type of neural network (268) which automatically interprets the content of the system-generated feature set(s) (235), and uses the content of such feature set(s) to generate draft feature set-derived system-generated instructional data inputs, and thereafter recommends changes to the instructional data to the user, automatically changes/amends the instructional data to incorporate such draft feature set-derived instructional data inputs, or a combination thereof. The instructional data content can be, in aspects, relatively constrained (e.g., comprising less than 500, less than 300, less than 200, or less than 100 elements) or can be quite broad (e.g., comprising greater than 500 elements), In aspects, a small amount of instructional data content, when prioritized through adequate training of neural network(s) (e.g., one or more of neural network(s) (220), (267), and (268)) can result in a feature set system(s)/method(s) that is/are significantly more accurate/trustworthy in the generation of draft feature set(s) (235), particularly over a number of iterations.

Feature set data (235) is typically presented to user(s) (202), associated user(s) (205), or both, for review ((240A) and (240B), respectively) as morphed image(s), e.g., original image(s) modified according to instructional input(s). Morphed images reflect an aesthetic appearance which would result if such feature set data (235) were applied to the subject of the original image in an aesthetic modification procedure. Upon review, ((240A), (240B)), respectively) user(s) (202), associated user(s) (205), or both can modify ((245A) and (245B), respectively) data associated with feature set(s) by way of adjusting instructional input(s) (210), at which time the process can iterate. Upon review, ((240A), (240B), respectively) user(s) (202), associated user(s) (205), or both can transmit ((250A), (250B), respectively) feature set(s) data. Upon review ((240A), (240B), respectively), user(s) (202), associated user(s) (205), or both can save ((255A), (255B), respectively) feature set(s) data. Data can, in aspects, be captured by data capture & retrieval system(s) (224) of data processing and manipulation feature(s) (215). Upon review, ((240A), (240B), respectively) user(s) (202), associated user(s) (205), or both can delete ((260A), (260B), respectively) feature set(s) data.

### FIG. 20

As described, herein are automated system(s) for establishing target effect(s), e.g., target aesthetic appearance(s) (e.g., of a human face, e.g., of the upper lip of a human face) using feature(s), characteristic(s), and principle(s) of marking system(s) described herein, which are anticipated to result from one or more aesthetic modification procedure(s) in which such marking system feature(s), characteristic(s), or principle(s) established by the system(s) are applied. Requirement(s) for achieving a target aesthetic appearance, e.g., requirement(s) for the characteristic(s) of marker(s) of marking system(s) and component(s) thereof described herein, can be saved by system(s) as feature set(s) as described elsewhere herein. FIG. 20 provides one simplified example of a process of querying (400) system(s) to retrieve saved feature set(s).

The process of querying (400) system(s) to retrieve saved feature set(s) presented in FIG. 20 begins with such feature set(s) being stored within one or more data processing and manipulation feature(s) (215) of system(s) disclosed herein. User(s) (202), associated user(s) (205), or both enter a query (205) into the automated system. Such a query can be via an interface with the system(s), such as, e.g., via a screen of a mobile application. For example, such a query can be via an interface with the system, such that the user, e.g., a prospective recipient of an aesthetic modification procedure, can show a medical professional a desired outcome of an aesthetic modification procedure intervention. Such a query can be, e.g., via an interface with the system associated with one or more ancillary system(s), such as, e.g., a system associated with a medical professional(s) practice, a medical professional(s) instrumentation/equipment. For example, a medical professional can query the system to retrieve the feature set data required to perform an aesthetic modification procedure intervention. In another example, feature set data saved by the automated system(s) can be queried by an instrument, device, or ancillary system associated with the performance of the aesthetic modification system, such as, e.g., an automated marking system, an automated surgical system (e.g., a system/instrument/device comprising robotics), and the like.

The data processing and manipulation feature(s) (215) of system(s) receive the entered query and locate (410) the queried feature set(s) data. Upon its identification/location, the system retrieves (415) the queried feature set data (420). The retrieved queried feature set data (420) is presented for user review (240A), associated user review (240B), or both.

Upon review, the queried feature set(s) data (420) can be deleted ((260A) and (260B), by user(s)/associated user(s), respectively), saved ((255A) and (255B), by user(s)/associated user(s), respectively), transmitted ((250A) and (250B), by user(s)/associated user(s), respectively, or modified ((245A) and (245B), by user(s)/associated user(s), respectively). Transmission can comprise transmission via any suitable means, such as, e.g., by email, by SMS, or by other data transfer across media such as wi-fi, Bluetooth, Ethernet, satellite, etc. Transmission can be, e.g., to a human recipient or, e.g., transmission can be directly to a device/instrument, or system, e.g., a device/instrument or system utilized in the performance of an aesthetic modification procedure wherein the feature set data is used in the performance of the aesthetic modification procedure. In aspects, saved or modified queried data/feature set(s) are addressed by the data processing and manipulation feature(s) (215) of the automated system(s) herein.

### REPRESENTATIVE EXPERIMENTS/EMBODIMENTS (“EXAMPLES”)

The following detailed exemplary expository descriptions or experiments involving embodiments, applications, or related principles, of or otherwise related to the disclosure (“Examples”) are provided to assist readers in further understanding aspects of the disclosure or principles related to the disclosure or practice of aspects of the disclosure.

Any particular materials, methods, steps, and conditions employed/described in the following Examples, and any results thereof, are merely intended to further illustrate aspects of the disclosure. These Examples reflect exemplary embodiments of the disclosure, and the specific methods, findings, principles of such Examples, and the general implications thereof, can be combined with any other part of this disclosure. However, readers should understand that the disclosure is not limited by these Examples or any part thereof.

### Example 1

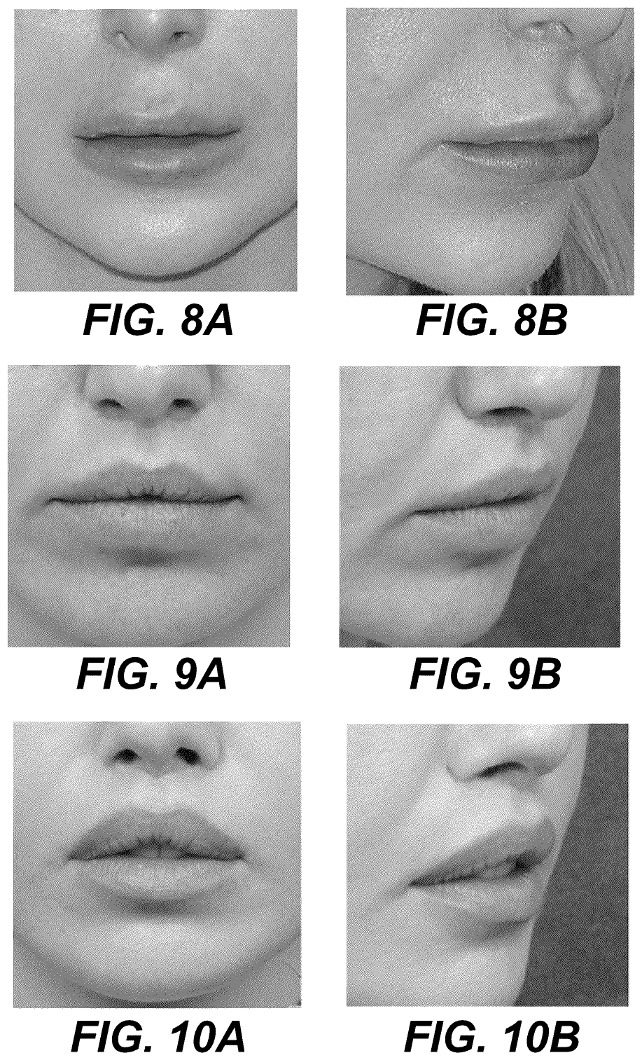

A 31-year-old female subject presented with excess lip filler seeking additional lip modification. FIGS. 8A and 8B provided herein are before photographs of the patient having received lip fillers (front (8A) and slight profile (8B) views).

A classically designed, deep plane lip lift utilizing uniform excision height was performed according to the prior art. FIGS. 9A and 9B provide photographs of the same patient shown in FIGS. 8A and 8B, resulting from the classically designed, uniform excision deep plane lip lift (front (9A) and slight profile (9B) views).

Upon completion, the patient felt imbalanced, and a second procedure according to the design parameter(s) identified herein (e.g., with a target effect of a 2 mm height difference along the vermillion slope of neighboring radial marker(s) as described herein) was performed according to the principle(s) of the disclosure described herein. The target effect(s) were to gain an improved uniformity of exposure along the entirety of the lip. FIGS. 10A and 10B provide photographs of the same patient shown in FIGS. 8A, 8B, 9A, and 9B, resulting from the application of procedure(s) comprising the marking system(s) provided by the present disclosure (front (10A) and slight profile (10B) views).

This example illustrates the nuanced and improved outcome(s) resulting from the application of system(s) and method(s) provided herein.

### Construction and Terms

### Construction Principles and Description of Select Terms

This section offers guidelines and resources intended to aid readers in understanding this disclosure.

### General Terms and Principles

The intended audience for this disclosure (“readers”) are persons having at least ordinary skill(s) in the practice of technologies discussed or used herein. Readers may also be called “skilled persons,” and such technologies and related publicly available prior knowledge are collectively referred to as “the art.” Terms such as “understood,” “known,” and “ordinary meaning” refer to the general knowledge of skilled persons.

The purpose of this document (“disclosure”) is to describe a number of new technologies (sometimes just called the “technology” and each discrete embodiment of which sometimes being called “a technology”) and to provide readers with the ability to practice such technologies (e.g., by exemplification, by description of elements thereof and relationship(s) of such elements to each other, and possibly other means). Readers will understand from this disclosure whether the technology/technologies include different forms, e.g., objects (such as systems, compositions, or devices), methods, or both, as will be clear from the disclosure. Disclosed technologies may be associated with surprising, unexpected, or otherwise inventive properties and, accordingly, in many cases, a described technology may reflect an invention.

The term “uncontradicted” means not contradicted by this disclosure, logic, or plausibility, the latter two elements being based on the knowledge of skilled persons.

Disclosed here are several different but related exemplary aspects (variations) of the technology(ies) (also referred to as, e.g., “cases,” “facets,” “respects,” or “embodiments”). The disclosure encompasses all such aspects as described individually and as can be arrived at by any combination of such individual aspects. Thus, uncontradicted, any reference to “aspects” (e.g., “according to aspects” or “in an aspect”) will be understood as referring to according to any of the other suitable aspects of the technology(ies) described herein. In this respect, the breadth and scope of the disclosure should not be limited by any exemplary aspect(s)/embodiment(s) herein. No language in this disclosure should be construed as indicating any element/step is essential to the practice of any technology or group of technologies provided by this disclosure unless such a requirement is explicitly stated. Uncontradicted, any aspect(s) described in any part of this disclosure can be combined with any other aspect(s) in any other part. Readers will discern that the term “aspect” also is used sometimes to refer to portions of this disclosure and, in such respects is used in a manner similar to “subject” or “topic.”

Uncontradicted, all technical/scientific terms used here should be read, at least in one aspect, to have the same meanings as commonly understood by skilled persons, regardless of any narrower examples or descriptions provided here (including any term introduced initially in quotations). However, readers will also recognize that some aspects can be characterized by the inclusion of elements, steps, features, characteristics, etc., associated with specific descriptions provided here and that such specific disclosures represent distinct embodiments of the disclosure apart from the corresponding aspect that is provided by interpreting the relevant aspect using any broader commonly used terminology or concept. Uncontradicted, disclosure of any aspect using known terms, which terms are narrowed by example or otherwise, implicitly discloses one or more related aspects in which the applicable terms are alternatively interpreted using the broadest reasonable interpretation of skilled persons.

Uncontradicted, the term “or” means “and/or” here, regardless of any inclusion of the actual phrase “and/or” (e.g., phrases such as “A, B, or C” and “A, B, and/or C” each simultaneously discloses aspects including (1) all of A, B, and C; (2) A and C; (3) A and B; (4) B and C; (5) only A; (6) only B; and (7) only C (and also support sub-groupings, such as “A or B,” “A or C,” etc.)). Uncontradicted the use of a modifier/term such as “or both” in connection with elements (e.g., “element A, element B, or both”) does not mean or imply that elements listed only as “A or B” do not include combinations of A and B.

For conciseness, symbols are used where appropriate. E.g., “&” is used for “and,” & “˜” for “about.” Symbols such as < and > are given their ordinary meaning (e.g., “<” means “less than or equal to” & “>” means “greater than or equal to”). A slash “/” between terms here can represent “or” (“A/B” means “A or B”) or identify synonyms of an element, depending on context. The inclusion of “(s)” after an element or a step indicates that ≥1 of such an element is present, step performed, and the like. E.g., “element(s)” refers to both 1 element and >2 elements, with the understanding that each thereof is an independent aspect of the disclosure.

Uncontradicted, the term “also” means “also or alternatively.” Uncontradicted, the terms “here” & “herein” mean “in this disclosure.” The abbreviation “i.a.” (alternatively “ia” or “ia”) means “inter alia” or “(possibly) among other things.” “Also known as” is abbreviated “aka,” “AKA,” “a.k.a” (and can also or alternatively mean “is otherwise referred to here,” even if the relationship between the terms is not well known). The similar abbreviation “aka/ac” means “also known as or otherwise called” is sometimes alternatively used to stress this point about the nature of the acronym. Uncontradicted, the term “elsewhere” means “elsewhere herein.”

Use of the abbreviation “etc.” (or “et cetera”) in association with a list of elements/steps means any or all suitable combinations of the recited elements/steps or any known equivalents of such recited elements/steps for achieving the function(s) of such elements/steps known in the art. Readers should interpret phrases like “and the like” similarly.

Uncontradicted, terms such as “and combinations,” “or combinations,” and “combinations thereof,” etc., regarding listed elements/steps, means any or all possible/suitable combinations of the associated elements/steps. Thus, e.g., uncontradicted, a phrase like “combination of any thereof” refers to any or all combinations.

Aspects may be described as suitable for method(s)/use(s) disclosed herein. Uncontradicted, terms such as “suitable” or “suitability” mean acceptable, appropriate, or, in aspects practical for performing a particular function/achieving particular state(s)/outcome(s), and typically means effective, practical, and non-deleterious/harmful to associated valuable subject matter (human health, resource state, etc.). E.g., uncontradicted, the term “suitable” means appropriate, acceptable, or in contexts sufficient, or providing at least generally or substantially all an intended function (of the element or overall whole of the aspect), without causing or imparting significant negative/detrimental impact. Uncontradicted, each method step, component/ingredient, or result element of the technical aspects of this disclosure should be understood to implicitly be mostly, generally only, substantially only, or only of an amount, degree, or character suitable in connection with its intended function, the intended function of the associated whole, or both. In some respects, suitability can be demonstrated through scientific studies and to a degree of significance through suitable tests/measures such as scientific tests, well-controlled and adequate studies (e.g., clinical studies), adequately powered trials, etc.

Steps, elements, compositions, devices, components, and the like also or alternatively can be characterized as being “effective.” Uncontradicted, any disclosed element is to be construed as being effective for its intended purpose and present in an effective amount, and any step performed is to be understood as being performed/applied effectively, such as in an effective amount or an effective number of times, etc. Uncontradicted efficacy can be judged by evaluating the element(s) ability to perform or contribute to the described function(s) or characteristic(s) associated with the component, device, step, etc., the overall aspect, or both in any manner disclosed here, known in the art, or both. For example, when applied to effects in organisms, such as people, efficacy and similar terms uncontradicted should be interpreted to at least implicitly disclose efficacy that can be measured (1) in a treated subject, (2) in a majority of subjects in a population, (3) in a statistically significant number of subjects in a population, (4) generally all subjects in a population, (5) substantially all subjects in a population, or (6) in a statistically significant number of or more of a typical or average subject of the class of subjects treated. Object elements or steps are, uncontradicted, understood to be implicitly present in “effective amount,” and, uncontradicted, any described class of object or step in connection with a device, system, composition, or method, is understood to be present in the associated whole or performed in association with the associated entire method in an effective amount, effective way, or having effective characteristic(s), which generally means, an amount that the described object/component or step is effective for the described function(s) associated with the element, associated whole, or both. A “step” is not necessarily a general “step for” performing a function.

Uncontradicted, heading(s) (e.g., “Construction and Terms”) and subheadings used here are included for convenience and do not limit the scope of any aspect(s). Uncontradicted, aspect(s), step(s), or element(s) described under one heading can apply to other aspect(s) or step(s)/element(s) here.

Ranges of values here represent each value falling within a range within an order of magnitude of the smallest endpoint of the range, without having to write each value of the range explicitly. E.g., a recited range of 1-2 implicitly discloses each of 1.0, 1.1, 1.2, . . . 1.9, and 2.0, and 10-100 implicitly discloses each of 10, 11, 12, . . . 98, 99, and 100). Uncontradicted, all ranges include the range's endpoints, regardless of how a range is described. E.g., “between 1-5” includes 1 and 5 in addition to 2, 3, and 4 (and all numbers between such numbers within an order of magnitude of such endpoints, e.g., 1.0, 1.1, . . . 4.9, and 5.0). For the avoidance of doubt, any number within a range, regardless of the order of magnitude of the number, is covered by the range (e.g., a range of 2-20 covers 18.593). Uncontradicted, readers will understand that any two values in a range provided as a list herein can be combined as endpoints to form a range defining a more particular aspect of the disclosure (e.g., if a list of values 1, 2, 3, 4, and 5 of element X is provided, readers will understand that the disclosure implicitly discloses an aspect comprising 2-4 X, 3-5 X, and 1-3 X, etc.

Terms of approximation (e.g., “about,” “˜,” or “approximately”) can be used here (1) to refer to a set of related values or (2) where a precise value is difficult to define (e.g., due to limits of measurement). Uncontradicted, all exact values provided here simultaneously/implicitly disclose corresponding approximate values and vice versa (e.g., disclosure of “about 10” provides explicit support for the use of 10 exactly in such aspect/description). Ranges described with approximate value(s) include all values encompassed by each approximate endpoint, regardless of presentation (e.g., “about 10-20” has the same meaning as “about 10-about 20”). The scope of value(s) encompassed by an approximate term typically depends on the context of the disclosure, criticality or operability, statistical significance, understanding of the art, etc. In the absence of guidance here or in the art for an element, terms such as “about” when used in connection with an element should be interpreted as ±10% of the indicated value(s) and implicitly disclosing ±5%, ±2%, ±1%, and ±0.5%. Two or more values may be characterized as approximately similar if they are considered to be about the same on such bases.

Aspects may be associated with the description of a change or difference. In some cases, similarity or difference can be assessed as similar or not (i.e., statistically similar or different). In cases, a difference or change means a “sizable” change or difference, which means a change or difference that is beyond what would be considered substantially the same or approximately the same (approximately or about in such contexts being either defined in the art or +/−10% or being recognized by readers as not having different characteristics or outcomes that are substantially different with respect to intended function) (e.g., a change of ≥12.5%, ≥15%, ≥20%, etc., such as 12.5%-50%, 12.5%-33%, 15-45%, etc., or 15-150%, 20-200%, 30-300%, etc.). In aspects, a change or difference in element(s) can be characterized as a “major” change or difference, which means a change or difference that is an increase or decrease (1) of at least 33% and can be a change of at least 50%, at least 75%, at least 100%, at least 150%, at least 200% (2×), e.g., at least 0.5×-5×, 10×, or 20× or (2) by one or more (e.g., 2 or 3) order(s) of magnitude. Uncontradicted, elements, compositions, outcomes, etc., described as different herein or described as different in any of these ways provide implicit support for corresponding aspects in which the applicable change/difference is characterized as one of the other differences. The modifier “constrained” means that a value, such as a sizable value, a detectable value, or a major value is limited to 50% or less of a whole (e.g., 45% or less, 40% or less, 37.5% or less, or 35% or less of a whole). Uncontradicted, any disclosure of such a term herein provides implicit support for an otherwise corresponding aspect in which the scope of the applicable value can be characterized as a constrained value.

This disclosure includes aspects of the technology that are associated with particular characteristics, such as amounts of components (or ranges thereof). In cases, several such characteristics of varying scope may be provided. Readers will understand that each such characteristic can be associated with particular properties that distinguish such aspects from other aspects, and, accordingly, each such range can be viewed as critical to a particular aspect of the technology, even if the associated results, properties, functions, etc., associated with such aspects are not directly/explicitly communicated in association with any such characteristics.

Lists of aspects, elements, steps, and features are sometimes employed for conciseness. Unless indicated, each member of each list should be viewed as an independent aspect. Each aspect defined by any individual member of a list can have and often will have, nonobvious properties vis-à-vis aspects characterized by other members of the list.

Uncontradicted, the terms “a” and “an” and “the” and similar referents encompass both the singular and the plural form of the referenced element, step, or aspect. Uncontradicted, terms in the singular implicitly convey the plural and vice versa herein (in other words, disclosure of an element/step implicitly discloses the corresponding use of such/similar elements/steps and vice versa). Hence, e.g., a passage regarding an aspect including X step supports a corresponding aspect including several X steps. Uncontradicted, any mixed use of a referent such as “a” in respect of one element/step or characteristic and “one or more of” concerning another element/step or characteristic in a paragraph, sentence, aspect, or claim, does not change the meaning of such referents. Thus, for example, if a paragraph describes a composition comprising “an X” and “one or more Ys,” the paragraph should be understood as providing disclosure of “one or more Xs” and “one or more Ys.”

“Significant” and “significantly” mean results/characteristics that are statistically significant using ≥1 appropriate test(s)/trial(s) in the given context (e.g., p≤0.05/0.01). “Detectable” means measurably present/different using known detection tools/techniques. The acronym “DOS” (or “DoS”) means “detectable(ly) or significant(ly).” The term “measurably” means at a measurable level and, uncontradicted, comprises at a suitable measurable level/amount. The term detectable provides implicit disclosure for aspects that are “measurable,” and the term “measurable” implicitly supports aspects where the measured or measurable element is “detectable.” Uncontradicted, any aspect including an element described as “similar” to another element implicitly discloses, at least as one aspect, where the similarity comprises statistical similarity. Uncontradicted, any reference to a comparison, change, or other relationship between elements (e.g., a result) characterized by similarity or detectability also implicitly discloses changes or comparisons where the difference is approximately/about the same (e.g., within +/−10% of each other).

Uncontradicted, any value provided here that is not accompanied by a unit of measurement (e.g., a weight of 50 or a length of 20), either any previously provided unit for the same element/step or the same type of element/step will apply, or, in cases where no such disclosure exists, the unit most used in association with such an element/step in the art applies.

Uncontradicted, the terms “including,” “containing,” “comprising,” and “having” mean “including, but not limited to,” or “including, without limitation.” Uncontradicted, use of terms such as comprising and including regarding elements/steps means including any detectable number or amount of an element or including any detectable performance of a step/number of steps (with or without other elements/steps). Uncontradicted, “a” means one or more, even when terms such as “one or more” or “at least one” are used in association with the referent “a.”

For conciseness, description of an aspect “comprising” or “including” an element, concerning a collection/whole (e.g., a system, device, or composition), implicitly provides support for any detectable amount/number, such as, e.g., between a detectable or measurable amount and about 33%, such as, e.g., ≥˜1%, ≥˜5%, ≥˜10%, ≥˜20%, ≥˜25%, or ≥˜33%, as in, for example, ˜0.00001%-˜33%, ˜1%-˜33%, ˜5%-˜33%, ˜10%-˜33%, ˜15%-˜33%, ˜20%-˜33%, ˜25%-˜33%, or, e.g., ˜30%-˜33%.

In certain respects, description of an aspect “comprising” or “including” an element concerning a collection/whole implicitly provides support for amounts greater than about 33%, such as, e.g., ˜50%-˜75%, such as, e.g., ≥˜50%, ≥˜51%, ≥˜51%, ≥˜66%, or ≥˜70%, such as, e.g., ˜55%-˜75%, ˜60%-˜75%, or ˜70%-˜75%, such as, e.g., ˜35%, ˜40%, or ˜45%.

In still further respects, description of an aspect “comprising” or “including” an element concerning a collection/whole implicitly provides support for amounts greater than about 80%, such as, e.g., ˜80%, ˜85%, ≥˜90%, ˜93%, ≥˜95%, ≥˜99%, or ˜100% of the whole/collection being made up of the element, as in, e.g., ˜77% or more, or essentially all of the whole/collection being made up of the element (i.e., that the collection consists essentially of the referenced element). Similarly, a method described as including a step concerning an effect/outcome implicitly provides support for the referenced step, providing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the effect/outcome, representing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the steps/effort performed, or both. Explicit listing of percentages of elements in connection with particular aspects does not limit or contradict such implicit disclosure. Uncontradicted, readers should interpret terms such as “essentially all” or “essentially” consistent with the concept of “consisting essentially of.”

Uncontradicted, terms such as “comprising” when used in connection with a step of a method provide implicit support for performing the step once, ≥2 times, or until an associated function/effect is achieved.

Further, readers will understand that uncontradicted, use of terms such as “comprising” or “including” provides aspects for which the referenced element (or step(s), etc.) is “generally” present, “substantially” present, “essentially” present, or is preset. In certain alternative aspects, “comprising” or “including” can refer to something that is mostly present, about equally present, or is present in another amount, such as about 40%, about 50%, etc. In certain aspects, accordingly, the use of “comprising” and “including” provides support for referenced element(s) to be present in “significant” amounts (e.g., a statistically significant amount) or in DOS amounts, in “some” amount, or, e.g., a “predominate” amount.

Uncontradicted, any disclosure of an object or method (e.g., composition, device, or system) “comprising” or “including” element(s) provides implicit support for an alternative corresponding aspect that is characterized by the object consisting of that element or “consisting essentially of” that element (excluding anything that would “materially affect” the “basic and novel characteristic(s)” of any inventive aspect of this disclosure). Uncontradicted, any specific use of phrases such as “consists of” and “consists essentially of” herein does not modify this construction principle.

Readers will understand that the disclosure of concentrations/amounts/numbers of different elements/components acts as a disclosure of compositions/devices characterized by relationships in such amounts formed between them. Accordingly, uncontradicted, any disclosure of amounts/concentrations/numbers that reflects a suitable relationship between elements/components provides an implicit disclosure of a composition/device that varies from the specifically disclosed amounts/concentrations/numbers, but which retains the relationship. For example, if the disclosure provides 1 unit of A and 3 units of B, readers will understand that this means that the disclosure provides a corresponding aspect characterized by the inclusion of suitable amounts of A and B, wherein such amounts are present in a ratio of about 1 part A to about 3 parts B.

Readers will understand the “basic and novel characteristic(s)” of an invention provided in this disclosure and the scope of what constitutes a “material effect” (or “material effect”) of such “basic and novel characteristics” will vary with the specific applicable aspect at issue. Uncontradicted, the basic and novel characteristics of any inventive aspect include the specific recited and associated elements of an aspect and exclude any other element that significantly detracts from the intended function(s) of the recited elements, that introduce significant new functions that are unrelated to the intended function(s), that significantly reduce the performance of the function(s), or that significantly negatively change other characteristics of performing such function(s) (e.g., by increasing the cost of performing the functions in energy, money, or both). Uncontradicted, the basic and novel characteristics also include at least significantly retaining the suitability, effectiveness, or both, of recited elements or the overall aspect. Accordingly, a material effect can be an effect that reduces, diminishes, eliminates, counteracts, cancels, or prevents one or more of such functions in one or more respects (e.g., delaying onset, reducing scope, reducing duration, reducing output, reducing the level of applicability, reducing effect, or combinations thereof). In an aspect, a material effect is one that changes such functions by making such functions impractical, difficult to obtain, or materially more expensive or otherwise costly in terms of inputs. From this and the other guidance provided herein, readers can understand the scope of an aspect that is defined by consisting essentially of a collection of elements.

Uncontradicted, the term “one” means a single type, single iteration/copy/thing, of a recited element or step, or both, which will be clear from the context of the relevant disclosure. For example, the referent “one” used with respect to a component of a composition/article or system can refer to one type of element (which may be present in numerous copies, as in the case of an ingredient in a composition) one unit of the element, or both. Similarly, “one” component, a “single” component, or the “only component” of a system typically means 1 type of element (which may be present in numerous copies), 1 instance/unit of the element, or both. Further, “one” step of a method typically means performing one type of action (step), one iteration of a step, or both. Uncontradicted, a disclosure of “one” element provides support for both, but uncontradicted, any claim to any “one” element means one type of such an element (e.g., a type of component of a composition/system/article).

Uncontradicted, the term “some” means ≥2 copies/instances or ≥5% (e.g., ≥7.5%, ≥12.5%, ≥17.5%, ≥27.5%, or ≥37.5%) of a listed collection/whole is or is made up of an element. Regarding methods, some means ≥5% of an effect, effort, or both is made up of or is attributable to a step (e.g., as in “some of the method is performed by step Y”) or indicates a step is performed ≥2 times (e.g., as in “step X is repeated some number of times”). Terms such as “considerable amount” or “considerable portion” mean at least 1%, 2%, or 2.5%, but less than most of a whole, such as 2.5-25%, e.g., 5-25%, 5-20%, 7.5-22.5%, 10-20%, 2.5-10%, 2.5-12.5%, 5-15%, etc. Terms such as “sizable portion” mean 10-50% and in aspects 15-50%, 20-50%, or 25-50%, or subranges thereof (e.g., 15-45%, 20-40%, 25-45%, etc.). Terms such as “predominately,” “most,” or “mostly” (and “primarily” when not used to refer to an order of events or “mainly”) means detectably >50% (e.g., mostly comprises, predominately includes, etc., mean >50%) (e.g., a system that mostly includes element X is composed of >50% of element X). The term “generally” means >75% (e.g., generally consists of, generally associated with, generally comprises, etc., means ≥75%) (e.g., a method that generally consists of step X means that 75% of the effort or effect of the method is attributable to step X). “Substantially” or “nearly” means ≥95% (e.g., nearly all, substantially consists of, etc., mean ≥95%) (e.g., a collection that nearly entirely is made up of element X means that at least 95% of the elements in the collection are element X). Terms such as “generally free” of an element or “generally lacking” an element mean comprising ≤25-% of an element, and terms such as “substantially free” of an element mean comprising ≤˜5% of an element. Uncontradicted, any aspect described as “generally comprising” or “generally consisting” of an element implicitly discloses an element that “substantially comprises” the element. The same principle applies to any disclosure where an aspect is described as being “generally free” of an element.

Uncontradicted, phrases such as “substantially identical” or “substantially similar” may be used to refer to element(s)/component(s)/ingredient(s)/thing(s) (e.g., composition, system, device, etc.) or step(s)/method(s) that have the same or about the same characteristic(s) or achieve the same or about the same result(s), typically in a similar way, as a referenced element/thing or step/method or otherwise do not meaningfully differ in intended result and manner of achieving such a result or are otherwise recognized in the art as not differing or not differing substantially in the relevant context (e.g., by being considered equivalents). Uncontradicted, readers will understand that a “substantially identical” or “substantially similar” element/thing or step/method when compared to a comparator thing/element or method/step means that the referenced element/thing or step/method exhibits such a similar function as a comparator at identical, approximately identical, or statistically similar amounts as the comparator thing or method when applied under similar conditions of use. Again, where statistical, approximate, or other measured comparisons are not possible, readers will understand the phrase as encompassing those things known as being identical or substantially identical to the referenced element/step or are described as such herein.

Uncontradicted, any aspect described concerning an optionally present element(s)/step(s) also provides implicit support for corresponding aspect(s) in which one, some, most, generally all, nearly all, essentially all, or all such element(s) are lacking/step(s) not performed, in respect of the relevant aspect. E.g., disclosure of a system comprising element X implicitly also supports a system lacking element X. That is, readers will understand that any element, feature, step, or characteristic of any aspect of the technology recited herein as being present in an aspect also implicitly provides support for the element, feature, step, or characteristic as being excluded from a corresponding/similar aspect implicitly disclosed by the explicit positive disclosure and vice versa. Uncontradicted, changes to tense or presentation of terms (e.g., using “comprises predominately” in place of “predominately comprises”) do not change the meaning of the corresponding term/phrase.

Uncontradicted, all methods provided here can be performed in any suitable order regardless of presentation (e.g., a method comprising steps A, B, and C can be performed in the order C, B, and A; B and A and C simultaneously, etc.). Uncontradicted, elements of a composition/device/system can be assembled in any suitable manner by any suitable method. In general, any methods and materials similar or equivalent to those described here can be used in the practice of embodiments in at least the broadest version of the relevant aspect. Uncontradicted, the use of ordinal numbers such as “first,” “second,” “third,” and so on is primarily, though not exclusively, intended to distinguish respective elements rather than to limit the disclosure to a particular order of those elements, importance, or configuration.

Any elements associated with a function can be alternatively described as “means for” performing a function in a composition/device/system or a “step for” performing a part of a method, and parts of this disclosure refer to “equivalents,” which means known equivalents known in the art for achieving a referenced function associated with disclosed mean(s)/step(s). However, no element of this disclosure or claim should be interpreted as limited to a “means-plus-function” or “step-plus-function” construction unless such intent is clearly indicated by the use of the terms “means for” or “step for.” Terms such as “configured to” or “adapted to” do not indicate “means-plus-function” interpretation but, rather, describe element(s)/step(s) configured to, designed to, selected to, or adapted to achieve a certain performance, characteristic, property, or the like using teachings provided here or in the art.

As used herein, the word “exemplary” means “serving as an example, instance, or illustration.” Any embodiment described herein as “exemplary,” “representative,” or “illustrative,” etc., should not necessarily be construed as preferred or advantageous over other embodiments. Any embodiment described herein as “exemplary” is not necessarily to be construed as preferred or advantageous over other embodiments.

Except where explicitly indicated or clearly indicated by context, terms such as “improved” or “better” mean DOS increased (e.g., sizably, constrained sizably, majorly, or constrained sizably, increased, etc.). In some respects, as will be clear from context or knowledge, terms such as “improved” or “better” mean DOS “reduced,” such as concerning reducing negative elements of a method or composition or performance characteristic (as in, e.g., with a device/system). Uncontradicted, terms such as “enhanced,” “improved,” “better,” and the like are used synonymously.

Any reference to a trademark name (product trademark (™)) incorporates all publicly known information about that product as of the filing date of this Application. Further, reference to a trademarked name is intended to refer to those product(s) as recognizable to readers as of the date of filing of this disclosure that are recognized as equivalents to such product(s), such as product(s) that perform the same function/are recognized as operationally the same (operate on the same key principles or by way of the same feature(s)); comprise the same key characteristic(s), component(s), mechanism(s), etc. Readers should recognize, however, that simplified and alternative product(s) or element(s) from those provided herein could be incorporated, used, or provided, e.g., in aspects, modification(s) of the product(s) specifically described herein which maintain the spirit of this disclosure are incorporated or can be considered as “means” type equivalents of a referenced product/element.

Regarding figures, graphs, and the like disclosed in this Application, such figures, graphs, and the like are exemplary, and their disclosure concurrently provides disclosure of anything statistically significantly close to or which is “about” the same as the disclosure—e.g., as the data, feature(s)/characteristic(s), and demonstrated performance characteristic(s) provided. For example, any/all data points provided in, e.g., graph(s) disclosed in this Application incorporate data point(s) within a range of statistical similarity and points which are about the same (e.g., within a reasonable fraction of an order of magnitude) as those exemplified. Graph(s) or figure(s) provided herein should be understood to include, where such feature(s) are present, data providing similar patterns, shapes, curves, peaks, etc.

All references (e.g., publications, patent applications, and patents) cited herein are hereby incorporated by reference as if each reference were individually and specifically indicated to be incorporated by reference and set forth in its entirety herein. Uncontradicted, any suitable principles, methods, or elements of such references (collectively “teachings”) can be combined with or adapted to aspects. However, citation/incorporation of patent documents is limited to the technical disclosure thereof and does not reflect any view regarding the validity, patentability, etc., thereof. Uncontradicted, in the event of any conflict between this disclosure and the teachings of such documents, the content of this disclosure takes precedence regarding interpreting aspects of the disclosure. Numerous references are cited here to concisely incorporate known information and aid skilled persons in putting aspects into practice. While efforts have been made to include the most relevant references for such purposes, readers will understand that not every aspect of every cited reference will apply to every aspect of this disclosure or the technology.

While elements disclosed in such incorporated references can be combined with aspects of the disclosure provided herein, readers will understand that the described technology is intended to stand apart from such disclosures and, accordingly, uncontradicted, in aspects, any element(s) of the objects or methods of any such references can be considered to be excluded from the scope of what is presented as new technology here (e.g., if reference A discloses object or element B, any aspect that is not directed to object or element B can be characterized by, as one aspect, the lack of object or element B).

All original claims contained in this disclosure, when filed, are incorporated into this specification as if they were a part of the description.

### Terms and Principles Relating to Systems and Methods of the Disclosure

Uncontradicted, the term “component” refers to a device, system, or a part of a device or system (e.g., a device in a system), that performs one or more defined functions (alone or together with other components, devices, etc.) and that has a definable composition, construction, or other characteristic(s).

Uncontradicted, an element that is “configured to” or “adapted to” means an element (e.g., a component) that is designed to perform a function suitably/effectively. In the context of functions performed by processor components or components thereof (e.g., software engines), the term typically means that the processor is programmed to perform the function.

Uncontradicted, a “system” comprises an electronic device/collection of devices that is composed of interrelated/interacting components/devices (including, e.g., separate networked device(s)/component(s)), including (1) memory component(s) comprising computer-readable media including preprogrammed instructions for carrying out Function(s) (“engines”, artificial intelligence model(s) (AIM(s), or both) and (2) a computer/processing unit (processor) capable of reading/executing such instructions causing Function(s) to be performed. Typically, systems are characterized by the inclusion of one or more specially trained neural network(s) (NN(s)). AIMs/NNs are typically specially trained (or some, most, generally all of such NN(s) are specially trained) to perform specific Functions described herein (e.g., providing a distribution of semantic elements or interpreting content of a message and recommending instructional prompts based on the semantic content of the interpreted message). Terms such as “computing unit,” “computer,” and the like, typically mean a device comprising physical computer-readable media and a processor that processes (“reads”) information in such media. The media can comprise informative data and also functional information (modifiable/programmable computer-executable instructions (CEI)). Such instructions comprise specialized Engines and perform Functions. Computerized devices in communication with each other form a data network (“network”). Components in operation typically interact/communicate on a recurring basis (typically a regular or continuous basis), usually using common communication protocols over digital interconnections for the purpose of sharing data, functions, or other resources. Networks typically comprise physical components, e.g., routers, switches, and the like, described elsewhere. Components of networks are sometimes described as “clients” and “servers.” Client and server devices/components/Units are generally remote from each other and typically interact through a communication/data network. The relationship of client and server typically arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated in a user device, e.g., a result of user interaction, can likewise be relayed to a server.

Systems will comprise processors (sometimes called processor unit(s), processor systems, processor components, and the like). Processors are understood in the art and typically the term is understood to mean device(s)/system(s) that can, i.a., execute (“read” or “run”) CEIs (engines, algorithms, applications, code, etc.) stored in memory. A system can comprise multiple processors/processor units/components, which can be physically or functionally separate from each other. Such separate processing device(s)/system(s) can form a discrete/inter-operating processor, or a system can comprise multiple processing unit(s) that at least some, most, or all of the time operate separately from each other. Systems can include different types of processors, such as, e.g., a stream data processor for handling intake of streaming data delivered from another part of the system or associated network. Processors typically comprise physical components, at some level, but processing can be “virtual,” and elements of processing can be performed at a software/engine level. Processors can, for example, comprise a workflow/architecture that can be characterized as comprising massively parallel processes/very large workflow (e.g., as such terms are understood in the art in connection with leading cloud-based systems, such as Microsoft Azure systems). E.g., in aspects, the number of cores/processors in a multi-core MA is ≥˜10, e.g., ≥˜20, ≥˜50, ≥˜100, ≥˜200, ≥˜500, or ≥˜1,000, such as ≥˜2,000, ≥5,000, ≥10,000, ≥12,500, or ≥˜15,000 cores (e.g., ˜1,000-20,000 cores, 1,000-15,000 cores, 3,000-18,000 cores, 2,000-16,000 cores, or ˜2,500-15,000 cores). In aspects, the processor of a system is primary, generally only, or entirely composed of cloud-based processor capabilities/functions. In aspects, a processor operates based on a scalable, massively parallel, and distributed processor system. Massively parallel process (MPP) functions/methods can be achieved by use of large system step functions, such as Amazon Web Services (AWS) Step Functions. Microsoft Azure services also include MPP functions. In aspects, one, some, most, or all processor(s) of a system can be characterized as being “highly available” or comprising “highly available” workflow(s). In aspects, a system or component of a system exhibits ≥˜97%, ≥˜98%, ≥˜99% availability (accessibility and standard/optimal operability) over a period (e.g., per quarter, year, 3-year period, 5-year period, etc.), and in more particular aspects, a highly available system exhibits ≥˜99.8%, ≥˜99.9%, ≥˜99.95%, or ≥˜99.999% availability. High availability can be achieved by any suitable method(s) including normal means, message queues, lambda retry functions (e.g., in AWS), or a combination thereof. General resources/structures for high availability comprise application of component redundancy, component monitoring and management, failover (node/process substitution/routing), use of distributed replicated volume(s), load balancing, or a combination thereof. Processors can employ, in aspects, parallel processing, which can comprise an architecture of or perform distributed parallel processing, non-distributed parallel processing, or both. “Distributed processing” typically means processing performed on physically separate but networked machines. Non-distributed parallel processing can be performed on interconnected and co-located cores. Parallel processing systems can comprise systems classifiable as clusters, grids, cloud, or a combination thereof. Systems and associated networks can comprise/use routers, which can comprise networking/communication equipment configured to provide internal and external communications. Routers can include packet-switching and/or routing device(s)/unit(s) (including switches and/or gateways) configured to provide network communications between system/network component(s). Routers can be selected for capability to handle, configured for, or both, to handle the data demands of network, data storage, latency, availability, and throughput of system/network and other factors that may contribute to the cost, speed, fault-tolerance, resiliency, efficiency, and/or other design goals of the network/system architecture. Router(s) of the system/network can comprise security capabilities, such as VPN, firewall, etc. and can also include multiprotocol label switching capabilities. In aspects, some, most, generally all, essentially all, or all routing, switching, and similar functions are performed by software defined wide-area network components/units (SD-WAN appliances) (which can be on-demand components).

In aspects, system engine(s)/component(s)/unit(s)/system(s) are mostly, generally, essentially, or entirely based in public cloud networks/remote server devices (e.g., integrated/linked server cluster(s)) that can be used for outsourced computation, data storage, communication, and service hosting operations. These server(s) may be virtualized (i.e., server(s) may be or comprise virtual machines). Examples of public cloud networks include Amazon Web Services (AWS), Google Cloud services, and Microsoft Azure Services. A system can comprise a network management platform and server cluster(s) supporting public cloud networks, providing load balancing, redundancy, high availability, scalability, etc.

In aspects, system can comprise virtual machines/servers (emulations comprising memory, processing, and communication resources), which can be embodied by computer device(s), server cluster(s), etc., which typically are managed by a centralized server device, application, etc., which acts as a system controller, and can be accessed selectively by authorized system administrators. Virtual machine systems through Microsoft, VMWare, and the like are known in the art and can be adapted to such applications.

Although system/network can be/comprise computers, typically systems comprise/have memory and processing capabilities that far exceed those of typical general-purpose laptops, mobile phones, etc., as well as comprising specialized instructions/systems for performing the particular processes described herein. Devices that can access a network with a system, or otherwise access a system, can, however, comprise laptops, mobile smart phones, etc.

Systems of the invention include the ability to store data, including computer-executable instructions (CEI) (code) (e.g., engines). Systems can include any suitable type of memory component(s), system(s), etc. Various forms of memory systems are well known in the art. E.g., hardware components of a system's memory can use any suitable type of memory media. E.g., in aspects memory can comprise dynamic RAM or Flash memory. In aspects, memory comprises disk-based storage memory or a hybrid structure disk-based storage and DRAM/flash memory (e.g., using disk storage for “colder” data with DRAM or flash being used for “hotter” data).

In general, memory component(s)/system(s), which can also be referred to as memory unit(s), may include computer-executable instructions encoding kernel(s), and/or applications. A kernel can be an operating system, including modules for memory management, scheduling and management of processes, input/output, and communication, or device drivers that allow the operating system to communicate with the hardware modules/components (e.g., memory units, networking interfaces, ports, and buses) of the relevant computing device/system. Applications may be one or more user-space software programs, such as web browsers or email clients, as well as any software libraries used by these programs.

Memory may also store data used by these and other programs and applications. Data storage/memory unit(s) of a system or other network device can comprise/be in data storage arrays that can include drive array controllers configured to manage read and write access to groups of hard disk drives, solid state drives, etc. Drive array controllers, alone or in conjunction with system component(s) (server device(s)), also can be configured to manage backup or redundant copies of data stored in data repository(ies) (DR(s)) of a system (also sometimes called memory unit(s)) to protect against data failures, etc., e.g., failures that prevent system component(s) from accessing parts/units of memory/data storage. As discussed elsewhere, DR(s) can include any suitable form of data repository(ies), including any suitable database(s), such as a structured query language (SQL) database, no-SQL databases, data lakes, and the like. Various types of data structures can store information (e.g., analytical data) in such a database, including but not limited to tables, arrays, lists, trees, and tuples. Furthermore, databases or other DR(s) in data storage can be monolithic or distributed across multiple physical devices.

Data repositories (“DRs”) can be classified based on how data is primarily, substantially only, or only received (ingested), stored, and accessed/utilized (consumed) in/from the DR. Examples of known DRs include databases, data warehouses, and data lakes. Typically, each DR in a device or system's memory can be distinguished based on characteristics in these other ways from other DRs in the overall memory of the device/system. E.g., skilled persons can distinguish a database DR from a data lake DR based on, i.a., the structure of the DR, the type of data stored in the DR, etc.

In specific facets, DR(s) are stored in whole or in part and processor function(s) are employed via a cloud platform such as Microsoft Azure, AWS, or Google Cloud. Distributed processor system(s)/component(s) that can make up some, most, generally all, or all parts of a system processor can use distributed memory, whereby, e.g., processors can be endowed with chunks of the memory space and communicate via message-passing or similar methods. Each processor can in such aspects have direct access to its local memory and indirect access to nonlocal or remote chunks of memory. In aspects, some, most, generally all or all of system memory is based on a cloud computing platform/paradigm (e.g., DR/data store as a Service/Storage (DaaS) Platform or an Infrastructure as a Service (IaaS) or Platform as a Service (PaaS) Platform, comprising processing and possibly other functions in addition to memory and memory-supportive functions only). In aspects, cloud-based system memory element(s) of a system is or are based on a distributed system, scalable on demand (or automatically) system, or both. In aspects, distributed file systems that form some, most, generally all, or all of the system memory store data across many separate servers. In some facets, most, generally all, or substantially all the system memory is not distributed.

In aspects, the system memory unit(s) comprises a cloud-based memory comprising an internet-scale file system (e.g., Google File System (GFS)). Examples of cloud-based storage systems having these, and other features discussed here include Amazon Simple Storage Service (S3), Nirvanix Cloud Storage, OpenStack Swift, and Windows Azure Binary Large Object (Blob) storage. A system memory unit can comprise a file management system/layer, which defines some or most of the architecture for other data management levels in the Memory Unit (e.g., a Hadoop Distributed File System (HDFS) or similar system with capability to partition and replicate data sets to nodes where they are more likely to be consumed by applications directed by Mapping Functions (mappers)). Such file systems typically comprise a configurable data set replication function, detectably or significantly minimizing the impact of component/device failure in the system memory unit hardware architecture or operating system/software. In aspects, function(s) or component(s) of system memory unit(s) also can comprise a data management system (DMS), which can include a database management system (DBMS). In aspects, elements of the system memory unit(s) can comprise or operate with an execution tool to detectably or significantly (DoS) distribute computational load among components of a memory system and which can act as an API for the memory and can include or relate to other system component(s)/function(s), such as data inspection, data auditing, or data visualization functions directed to system memory. System processor(s) also typically comprises a query system for information extraction from DR(s).

Generally, any method described herein can be adapted to provide a corresponding system and vice versa. Accordingly, disclosure of any method simultaneously implicitly discloses a corresponding system that can carry out the indicated Functions and a disclosure of a system that comprises certain engine(s) or other components (e.g., certain trained NN(s)) implicitly discloses a corresponding method that comprises the steps for performing Functions corresponding to such engine(s), NN(s), or other component(s). Thus, uncontradicted, terms like “system” implicitly mean/disclose a corresponding “system or (i.e., and/or) method”, and terms like “method” likewise implicitly disclose a corresponding “method or system.”

The computer/software units/components of networks and systems can be characterized on the basis of “function(s)” that it/they perform. A “Function” or “function” is a computer-implemented action performed by a system component based on both preprogrammed computer-readable and -executed instruction(s), e.g., in response to input(s). A Function also can describe the result of step(s) of a method. The step(s) of such methods/elements of such Function(s) can comprise algorithms, methods, and the like described below. A function can, depending on context, be selectively performed, conditionally performed, or both, or can be performed at all times during operation or can be an inherent feature of a part or component.

Uncontradicted, the term “engine” (sometimes also referred to as an “Engine” or “data engine”) refers to computer/processor-executable software program/application (code, algorithm, etc.) or other components of or executed by a computer system which is configured to perform specified function(s), based on computer-executable/readable instructions (“CET”) contained in a memory (such CEI typically make up much, most, generally all, or all of the engine), or an engine can refer to any other component(s)/system(s) that perform similar functions on a recurring basis (either automatically, conditionally/situationally, on command/selectably, or a combination thereof). Typically, an engine processes input(s) and generates output(s). An engine typically can be implemented on computer(s) located in one or more locations. In aspects, multiple components of an engine are installed and running on one or more computer(s); multiple instances of an engine are installed and running one or more computers; or both. The operation of an engine typically performs function(s), which can represent step(s) in method(s). Such corresponding aspects are implicitly described by any explicit description of an engine or a function (e.g., description of a system/component comprising an Engine for performing Function(s) implicitly discloses a method including performance of the Function as step(s)). An engine that receives User input and provides output, i.a., to an end User can be called an “application.” Engines can make up part of, or also be described as, or can comprise “programs,” code,” “algorithms,” or similar elements known in the art for the control of electronic computer systems.

Engines/programs typically can encoded/written in any form of programming language (code), including compiled or interpreted languages, or declarative or procedural languages; and it can be deployed in any form, including as a standalone program or as a module, component, subroutine, or other unit suitable for use in a computing environment (e.g., Python, Java, C++, Ruby, C#, Go, JavaScript, R, Swift, and the like, which are known in the art). A program may, but need not, correspond to a file in a file system. A program/Engine can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program/Engine can be deployed to be executed on one computer or on multiple computers that are located at one site or distributed across multiple sites and interconnected by a data communication network. Programs/engines also can be described as “instructions” or “computer-implemented instructions,” “processor-implemented instructions”, “computer-readable instructions,” “computer-implemented data engines,” etc. Features/characteristics of engines are further described below. Functions can also be performed by more advanced systems/components in the context of methods/systems, such as neural network(s).

“Input” generally is understood in the art and can refer to/mean data that is provided to a system or system component (such as a neural network, engine, or the like), from any external source thereto. Typically here input refers to data provided by a user, but input may also originate from a medical professional, a system manager, or other source. “Output” typically refers to data generated by the system and delivered, directly or indirectly, to one or more users or other individuals (here, medical professionals). Output can also refer to activities performed under the control of a system or by a system (an output application). In typical aspects, output in methods/systems of this invention include draft messages generated, at least in part (e.g., mostly, generally entirely, or entirely) by the system.

An “interface” is understood in the art, and typically refers to any collection of components, engines, etc., that provide for interactions between a user and a computer, a network, or a system. In aspects, the term “interface” is used to refer to a user interface implemented in the form of a web page or similar media that can be viewed/navigated on several devices via the internet (e.g., using standard web browser(s)) (a “web interface” or “software interface”). Such an interface typically comprises a preprogrammed layout specific to the user, user class, or both. A device that displays/accesses an interface, such as a specialized web interface, can be considered a device of a network. In aspects, devices that access a network comprise fixed/specialized interface hardware. As such, network devices are sometimes referred to as devices/interfaces; however, uncontradicted, any disclosure of one such aspect implicitly discloses the other and vice versa.

A network, system, or interface can comprise output unit(s), such as display unit(s). A display unit can be any suitable device/component for visually displaying information to users, such as a computer monitor/screen, etc. In aspects, display units can comprise or be interactive (e.g., touch screen devices). Interfaces also can relay or receive information in audio format. In this respect, the term display is to be implicitly understood as meaning a “sensory output” component of an interface, device, etc.

Terms such as “data” and “information” are used interchangeably here and are only limited to a specific form when so indicated by explicit definition/statement or clear context. Interrelated data, as recognized by the system or a system component (through programming or otherwise), can be described as a “Record” or “record.” Records can include, e.g., attributes (characteristics) and values (measurements/attributes). In connection with this invention, relevant records comprise, mostly comprise, or at least generally consist of (directly or when translated) natural language messages. E.g., a record can include the body of some, most, or all of an email or other message that is suitable for use with the systems/methods provided here. Records and other data can be stored in the memory of the system or a device, etc. In cases, components/systems for the collection of large amounts of data may be referred to as a “data repository” (examples of which include data lakes, data warehouses, databases, and the like). Systems of the invention can include such memory storage devices/systems, and methods can include storing data generated by the systems/methods, e.g., method outcome data (described elsewhere) in such storage systems for later application.

Uncontradicted, data can be relayed, transmitted, etc. (“delivered”) through any suitable mode/mechanism and terms such as relayed, transmitted, conveyed, etc. are used interchangeably unless otherwise specified. The terms upload and download can be used in relation to the direction of data flow to (into) and from (out of) a referenced system, device, or other source. Electronic information relayed between components or over a network is typically relayed in packets, which can comprise other information such as identification data, source data, destination data, size data, persistence information (time to live (TTL)), composition data (e.g., flags), etc., as known in the art and discussed elsewhere. Packets can be organized into “streams.” Data also can be relayed via frames/chunks, or other known units/forms. Use of such terms can, accordingly, be considered exemplary herein, such forms being typically interchangeable or replaceable by data in other formats suitable for data transmission, storage, or both. Methods can include, for example, receiving data from one or more system components, e.g., from one or more neural networks, via streaming data delivery (and systems can comprise, e.g., streaming data processor(s)), and, in other aspects, data can be received in a batch mode, or methods/systems can operate in both modes, either somewhat concurrently, or by selection, or under different preprogrammed conditions.

The term “training” in connection with neural networks or other AIMs is understood in the art as comprising determining better/optimal operating parameters/conditions for a neural network to perform function(s). Typically, training comprises determining and applying the best set of weights for maximizing accuracy of an NN (adjusting weights in iteration(s) of use/performance), There are a variety of methods known in the art for evaluating neural networks, such as backpropagation. Uncontradicted, the term “training set” is used as understood in the art. Typically, the “training set” (and the term “training set data”) refers to any and all data used to train a neural network. The terms “initial training set” and “initial training set data” (ITSD) are used in this disclosure to refer to training set data provided before the initial use of a neural network or before the neural network is retrained in a significant re-training event. In aspects, a significant re-training event is one in which the system is provided with an amount of training that approximates 50% or more (e.g., ≥75%, ≥90%, ≥100%, etc.) of the prior initial training set. The “initial” descriptor can also be applied to other inputs, such as prompt data.

Neural networks of systems are typically artificial intelligence systems (also called models and vice versa), wherein such systems continuously learn with further uses/iterations/applications. As such, in a typical system/method, training data can be being added to the overall training set (the initial training set plus any new training set inputs) with each operation of the system by operation of one or more system components (e.g., one or more neural networks that modifies inputs to the systems, weights of a first neural network, etc.). For example, in aspects, messages generated by an iteration of the system can be used as additional training data for subsequent iterations of the system. Neural networks also may undergo some amount of training by human system administrators or by operation of system components as described herein. In aspects where a system is specifically trained to “onboard” a new user, adapt to a new type of recipient, deal with new triggers, etc., methods can specifically include performing several rounds of collecting training data before considering a system fully functional or ready for intended/regular use, and the training data is intentionally intended to increase during such steps of the method. In general, any description of a NN provides implicit support for a corresponding system/method in which a different type of AIM is used in place of the NN.

Neural networks generally comprise a number of networked neurons in multiple layers forming an architecture. Often, a neural network neuron operates through a perceptron, which, in turn, typically consists of input values, weights, and a bias, a weighted sum, and activation function. An activation function decides whether a neuron should be activated or not, and sometimes is referred to as an activator. In aspects, neural networks can comprise multi-layer perceptrons (MLPs). In aspects, neural networks comprise non-linear activators, such as sigmoid activation functions/logistic function activators, or non-sigmoid activators. In aspects, most or generally all of the activators/neurons in one or more neural network(s) are not sigmoid activators. In aspects, most of the activators or generally all of the activators in one or more neural networks are also or alternatively not Tanh (hyperbolic function) activators. Examples of non-linear activators/neurons include, e.g., Rectified Linear Activation Function (ReLU) activators. Neural networks, with, e.g., primarily ReLU activators, are often classified as ReLU neural networks or rectifier neural networks and can be incorporated in systems of the invention in some respects. In aspects, some, most, or at least generally all of the activators of one or more neural networks are known variants of ReLU activators, such as Softplus (Soft Plus), Softmax, Swish, Leaky ReLU, ELU (exponential linear unit), Gaussian Error Linear Unit (GELU), and SELU (scaled exponential linear unit) activators. In aspects, visible layers of a neural network are generally free or substantially free of ReLU activators and ReLU variant activators. In aspects, hidden layers generally lack or substantially lack sigmoid or Tanh activators. In aspects, visible layers comprise sigmoid or Tanh activators. Aspects of activators and other elements of neural network architecture/operation are described in, e.g., Goodfellow, DEEP LEARNING, 2016, ISBN-10: 0262035618.

The term “artificial neural network” (ANN) generally is synonymous with the term “neural network,” but sometimes the term ANN is used to describe MLP NNs or other types of NNs that are not recurrent NNs (RNNs) or convolutional NNs (CNNs).

In aspects, neural network(s) of a system can comprise/be characterized as feedforward neural networks. Other NN architectures that can characterize NN(s) of systems, in aspects, include radial basis network, deep feed forward, long/short term memory (LSTM), gated recurrent unit (GRU), auto encoder (AE), variational AE, denoising AE, sparse AE, Markov chain, Hopfield Network, Boltzman Machine (BM), restricted BM, deep belief network, deep convolutional network, deconvolutional network, generative adversarial network, liquid state machine, extreme learning machine, echo state network, deep residual network, Kohonen Network, support vector machine, and Neural Turing Machine architectures/types, or any other similarly known architectures in the art that are suitable for the applicable NN.

In aspects, one, some, most, or at least generally all of the NNs are RNNs. However, in other aspects, at least most, at least generally all, or at least substantially all of the NNs of the system are non-RNN neural networks. RNNs and other relevant concepts used herein, such as natural language models, are described in, e.g., U.S. Pat. No. 11,144,725. Some of these concepts and others (e.g., various layers of operation, cloud computing systems, tokens, NLP functions, and the like) are further described in US 20210342380 and US 20210182662. System elements/methods described in such references that are adapted to systems/methods herein can be combined with any systems/methods of the invention.

In aspects, neural network(s) can comprise convolutional neural networks (CNNs). CNNs are similar to feedforward networks, but they are usually utilized for recognition of data, such as pattern recognition, e.g., image recognition, rather than, e.g., for natural language processing functions. In other aspects, some, most, generally all, or at least substantially all of the NNs of the system are not classifiable as CNNs.

In aspects, NN(s) can comprise an adaptive-resonance (ART) neural network (described in, e.g., G. A. Carpenter & S. Grossberg, “Neural dynamics of category learning and recognition: Structural invariants, reinforcement, and evoked potentials”, in M. L. Commons, R. J. Hermstein, S. M. Kosslyn, & D. B. Mumford, (Eds.), 9 Computational and clinical approaches to pattern recognition and concept formation. Quantitative analyses of behavior (Hillsdale, N.J., Lawrence Erlbaum Associates, 1990)).

In aspects, the trainable neural networks of the invention can be considered “deep” neural networks. Uncontradicted, a “deep” neural network in the context of this invention is a neural network containing at least three layers, typically where at least one layer is considered a “hidden” layer, one an input layer, and one an output layer. Often, NNs will include many hidden layers. Such concepts are well known in the art. In aspects, a neural network can include ≥5, 10, 20, 30, 50, 100, 200, 250, 300, 500, 700, 800, 1000, or even more layers. In general, any suitable number of layers can be used in neural networks of the invention. Each layer of an NN can comprise any suitable number of neurons. The number of neurons in, e.g., an input layer can be selected based on known factors, such as the number of inputs, provided that in language Neurons can be of any suitable type, including multi-valued neurons. Neural network(s) can include, in aspects, complex-valued neural network(s) (CVNN(s)) or NNs having CVNN-like functioning/properties (see, e.g., Bassey et al., arXiv:2101.12249, Jan. 28, 2021).

In aspects, some, most, generally all, or all NNs of a system are capable of processing sequential input data, such as natural language (which is not possible with certain other types of NNs). In aspects, some, most, or generally all of the NNs in a system are capable of processing sequential data out of order (e.g., as in the case of a transformer neural network).

Neural network(s) in systems of the invention can include one or more attention layers or other “attention” elements known in the art. Neural network(s) can, for example, comprise a transformer architecture (and can be characterized as transformer neural network(s)) (see, e.g., Vaswani et al., 2017, arXiv:1706.03762). In aspects, some, most, at least generally all, or at least substantially all of the NNs of systems are characterizable as comprising/having a transformer architecture/operation. In aspects, any, some, most, or all of the transformer NN(s) are transformers with additional memory and retrieval units as known in the art (e.g., as described in arxiv.org/abs/2202.06991 and arxiv.org/abs/2203.08913).

A neural network (NN) can be referred to as a “model” or with respect to a “model.” The term “model” is sometimes applied to NNs, in general (i.e., in the sense that a NN can be considered an analytical model, computational model, machine learning model, etc.). In another sense, the term “model” in the art refers to either the function or the type of function performed by a neural network. Descriptions of prior art disclosures, however, may use “model” in a different manner. Skilled persons will recognize this variability in the meaning of this term and be able to work with it, given the context of its use.

Neural networks are “intelligent” systems and can “learn” or “evolve” with training/learning, including learning/training that occurs through use that increases the overall training set available to the NN, feedback, or both. Methods of learning/training NNs are known in the art. Uncontradicted, training of an NN herein comprises performance of various techniques that typically result, usually indirectly, in detectable or significant changes in weight(s) of neuron(s) of an NN, usually also or alternatively leading to a detectably or significantly change in output, in terms of output of the NN or of the system, in respect of a given analysis/function, input, or both. Typically, such changes in weights occur from further training of the neural network or changing the parameters of such training (e.g., applying different rewards to an NN for complying with a standard, such as output of another NN). Skilled readers will understand that in this and several other respects, systems/methods of the invention embody “deep learning” approaches to information processing/analysis. Because NN(s) “learn” and “evolve,” NN(s) (or models) can be described as either immature or mature. While such terms are relative, they will be generally understood in the art at least in clear cases (e.g., immature NN(s)/model(s) meaning models in the first number of iterations of a method/use of a system and mature NN(s)/model(s) meaning when an NN approaches convergence (e.g., generally or substantially producing substantially identical results in each use/iteration). As can be seen from various parts of this disclosure, in aspects, systems of the invention comprise ≥2 types of neural networks, each type of neural network performing a particular function.

Uncontradicted, any description of an engine can be performed by a neural network, another artificial intelligence (AI) component (e.g., a Random Forest machine learning element), or a non-AI/non-neural network engine (e.g., a typical program that carries out algorithm(s)). Typically, an “engine” is also recognized as a type of electronic/computerized component/device comprising physical media comprising instructions in computer-readable media (CRM) executed (read and carried out) by processor(s) to perform function(s). The term “protocol” is sometimes used to refer to an engine (e.g., engines that perform multi-step operations). The term protocol can also refer to a series of steps performed by engine(s). Engines also often can use/implement algorithm(s), which are also understood as, in cases, being similar to the latter type of protocol.

Terms such as “computing unit,” “computer,” and the like typically mean a device comprising physical computer-readable media and a processor that processes (“reads”) information in such media. Computer-readable media (CRM) can comprise informative data and also functional information (modifiable/programmable computer-executable instructions (CEI)). Memory can comprise, mostly comprise, physical, transferable, and reproducible computer-readable media (PTRCRM) containing stored instructions (engine(s)) and non-functional data (input, analytical data, in-process data, stored record data, output, etc.). Examples of memory systems are described elsewhere. Systems/devices described herein can be considered special-purpose computing systems as such systems typically require specialized engine(s) as exemplified below. In aspects, devices that make up Systems are also further characterized by possessing certain device characteristics, such as massively parallel processing capabilities. Alternative processor components are described elsewhere. Although a system/network can comprise any suitable number/type of computer(s), systems can comprise/have memory and processing capabilities that far exceed those of typical general-purpose laptops, mobile phones, etc., as well as comprising specialized instructions/systems for performing the particular processes described herein. Devices that can access a network with a system, or otherwise access a system, can, however, comprise laptops, mobile smartphones, etc.

Terms such as “machine learning” (ML) and “artificial intelligence” (AI) mean any suitable method/system in which one or more functions apply one or more machine learning models to information, typically resulting in computer modification of the function by the operation of the machine learning model(s). ML/AI methods can involve system administrators either in terms of training, supervision, or both. In aspects, ML is carried out by neural networks. In other aspects, ML is carried out by other, non-neural network AI model(s).

Computerized devices in communication with each other over distance, and the data connections between such devices, can be said to form a “network.” In aspects, some, most, or all of a system can also be considered a network. Network components typically interact/communicate on a recurring basis (typically a regular or continuous basis), usually using common communication protocols over digital interconnections for the purpose of sharing data, functions, or other resources. Networks can comprise other physical components, e.g., routers, switches, and the like, described elsewhere or known in the art, or suitable virtual counterparts thereof. Some computer device components of networks are sometimes described as “clients” and “servers.” E.g., in embodiments, a server transmits data, e.g., an HTML page, to a user device, e.g., for purposes of displaying data to and receiving user input from a user interacting with the device, which acts as a client. Data generated in a user device, e.g., a result of user interaction, can likewise be relayed to a server. Elements of network systems provided herein that can be alternatively described as clients and servers will be clear to readers. The presence of other network elements described herein is understood as implicitly described in aspects relating to network systems. Additional network elements can include firewalls, authentication protocols, virtual private network elements, etc. Any element described herein as a system or being performed by a system can be performed by a single device where suitable and vice versa.

Generally, any “method” (uncontradicted, meaning a “method of the invention”) described here can be adapted to provide a corresponding system and vice versa. Thus, disclosure of any method including steps simultaneously implicitly discloses a corresponding system comprising components (e.g., engines) that can perform corresponding functions and any disclosure of a system that comprises certain engine(s) or other components (e.g., certain trained NN(s)) implicitly discloses a corresponding method that comprises the steps for performing the functions performed by such engine(s), NN(s), or other component(s).

In certain contexts, herein, the term “element” may be used to reference a part, component, mechanism, device, system, or assembly. For example, disclosure herein may use the phrase “the X element” of the “Y component.” Readers will recognize from other portions of this disclosure that the term “element” also can be generally used herein to describe a thing (an object, such as a device, composition, system, etc.) or a part of a thing.

Although aspects exemplified here are described with reference to flow charts/block diagrams, any portion or combination of each block, combination of blocks, functions, etc., can be combined, separated into separate operations, or performed in other orders, as would be suitable in the context of a system or method of the invention. References to “modules,” “units,” “steps,” etc., in this disclosure typically are made for the convenience of the reader and are not intended to limit implementation of any method or system. Any portion or combination of any block, module, etc. can be implemented as computer-executable (program) instructions (e.g., software), hardware (e.g., combinatorial logic, Application Specific Integrated Circuits (ASICs), Field-Programmable Gate Arrays (FPGAs), processor(s), or other hardware), firmware, or any combination thereof (CT). In other words, flowcharts, block diagrams, etc. reflected in the Figures, illustrate the architecture, functionality, and operation of possible implementations of systems/methods. Each block in flowchart/block diagram(s) may represent a device, component, module, segment, CEI, or portion of CEI, for implementing the specified step(s)/function(s). In aspects, step(s) of methods or arrangement of function(s) described with respect to such blocks may occur in an order different from that set forth in the Figures. E.g., two blocks shown in succession may, in fact, be executed substantially concurrently, or the blocks may sometimes be executed in the reverse order, depending upon the functionality involved. Readers will note that each block of block diagrams or flowchart illustrations, and combinations of blocks in block diagrams/flowchart illustrations, can be implemented by special-purpose hardware-based systems that perform the specified functions or acts or carry out combinations of special-purpose hardware and computer instructions.

Different programming techniques can be employed, e.g., procedural or object-oriented approaches in system(s)/method(s). Any particular routine can execute on a single processor, multiple processors, or even in multiple devices, as suitable. Data can be stored in a single storage medium or distributed through multiple storage mediums and may reside in a single database or multiple databases (or other data storage techniques). Thus, although the steps, operations, or computations may be presented in a specific order, this order may be changed in alternative aspects and any of the specifically disclosed routines/workflows provided here can comprise rearrangement, repeating, skipping, combining, or any combination thereof (CT) of one or more step(s)/function(s) to provide the indicated output. In embodiments, to the extent multiple steps are shown as sequential in the Figures, some combination of such steps in alternative embodiments may be performed at the same time. Where suitable, any sequence of operations described herein can be interrupted, suspended, or otherwise controlled by another process, e.g., an operating system, kernel, etc. The routines can operate in an operating system environment or as stand-alone routines. Functions, routines, methods, steps, and operations described herein can be performed in hardware, software, firmware, or any CT (combination thereof), e.g., as described herein.

For these and other reasons, the illustrative description of aspects in the Figures and described here with respect to the Figures should not be used to limit the scope of the disclosure or any claimed or claimable invention based thereon as provided by other portions of this disclosure or the disclosure read as a whole in view of the art.

What is claimed is:

1. A method of generating predicted outcome imagery of a surgical aesthetic modification procedure comprising
(1) providing a user with a computerized device comprising (a) an interface that facilitates the collection of user input and transmission of the user input to a computer component comprising a computer component processor component that performs one or more functions on the input and returns output to the user through the interface; (b) a user interface processing component that performs one or more functions locally on the computerized device; and (c) a memory component that stores data and system instructions to be carried out by the user interface processing component, the computer component processor component, or both,
(2) prompting the user to provide user-identifying information through the interface and via the user interface processing component, the computer component processor component, or both, collecting the user-identifying information, and storing the user-identifying information in the memory component,
(3) prompting the user to provide, via the interface, the computer component with one or more image data files, each image data file corresponding to a photographic image of at least a portion of the user's face that is the subject of a potential surgical aesthetic modification procedure,
(4) subjecting each of the one or more image data files to analysis by a facial feature recognition artificial intelligence (FFRAI) module to identify and locate a plurality of mapped facial features in the photographic image and associating the facial features with the image data file,
(5) through the operation of the user interface processing component, computer component processor component, or both, applying a face image morphing function, the image morphing function carried out in part by a face imaging morphing artificial intelligence model (FIMAIM) that is trained to generate morphed facial images based on expected parameters of a surgical aesthetic modification procedure, wherein the face image morphing function modifies one or more of the image data files according to one or more preprogrammed instructions or one or more artificial intelligence models in a manner resulting in one or more initial morphed facial image files each expected to correspond with the outcome of performing the surgical aesthetic modification procedure according to the one or more preprogrammed instructions or the one or more artificial intelligence models,
(6) the user causing the interface processing component, computer component processor component, or both, to modulate one or more parameters associated with one or more selected facial feature elements from among the mapped facial features to generate one or more secondarily morphed facial image files,
(7) through the interface permitting the user to evaluate each of the initial morphed facial image files and each of the one or more of the secondarily morphed facial image files, and
(8) storing information regarding the user's evaluation in the memory component.

2. The method of claim 1, wherein the FFRAI module comprises at least one facial feature recognition neural network (FFRNN) that is trained to recognize facial features.

3. The method of claim 1, wherein the FIMAIM comprises at least one facial image morphing neural network (FIMNN), and the method comprises training the FIMNN based on user evaluation, medical expert evaluation, or a combination thereof.

4. The method of claim 1, wherein the method comprises performing a side-by-side comparison of at least two images selected from among an unmodified photographic image of the user's face provided by the user input, an initially morphed image file image, and one or more secondarily morphed image file images.

5. The method of claim 4, wherein the method comprises evaluating the secondarily morphed image files by a morphing function evaluation artificial intelligence model (MFEAI), which optionally comprises one or more morphing function evaluation neural networks (MFENN(s)).

6. The method of claim 1, wherein the method comprises providing the user with the option of selecting among a plurality of surgical aesthetic modification procedures or a combination of two or more surgical aesthetic modification procedures, wherein each surgical aesthetic modification procedure among the plurality of surgical aesthetic modification procedures corresponds to a surgical aesthetic modification procedure performed within a discrete portion of the face, and the method optionally comprises subjecting the image input provided by the user to a plurality of facial feature recognition artificial intelligence models, wherein each facial feature recognition artificial intelligence model of the plurality of facial feature recognition artificial intelligence models is trained to recognize one or more different facial features associated with a distinct part of the face.

7. The method of claim 4, wherein the method comprises providing the user with the option of selecting among a plurality of surgical aesthetic modification procedures or a combination of two or more surgical aesthetic modification procedures, wherein each surgical aesthetic modification procedure among the plurality of surgical aesthetic modification procedures corresponds to a surgical aesthetic modification procedure performed within a discrete portion of the face, and the method optionally comprises subjecting the image input provided by the user to a plurality of facial feature recognition artificial intelligence models, wherein each facial feature recognition artificial intelligence model of the plurality of facial feature recognition artificial intelligence models is trained to recognize one or more different facial features associated with a distinct part of the face.

8. The method of claim 1, wherein the method comprises the user interface processing component, the computer component processor component, or both, carrying out one or more functions to evaluate whether the user's image data file input is sufficient to perform an effective evaluation and prompting the user to provide additional or alternative image input if the one or more functions determine the image input is insufficient to provide an effective evaluation.

9. The method of claim 6, wherein the method comprises the user interface processing component, the computer component processor component, or both, carrying out one or more functions to evaluate whether the user's image data file input is sufficient to perform an effective evaluation and prompting the user to provide additional or alternative image input if the one or more functions determine the image input is insufficient to provide an effective evaluation.

10. The method of claim 7, wherein the method comprises the user interface processing component, the computer component processor component, or both, carrying out one or more functions to evaluate whether the user's image data file input is sufficient to perform an effective evaluation and prompting the user to provide additional or alternative image input if the one or more functions determine the image input is insufficient to provide an effective evaluation.

11. The method of claim 10, wherein the one or more functions are performed at least in part by the operation of one or more artificial intelligence models that are trained in evaluating the sufficiency of user image input.

12. The method of claim 6, wherein the method comprises prompting the user to provide one or more images for each of two or more distinct areas of the face through the interface and facilitating the association of the user image data file input with at least two different areas of the face.

13. The method of claim 7, wherein the method comprises prompting the user to provide one or more images for each of two or more distinct areas of the face through the interface and facilitating the association of the user image data file input with at least two different areas of the face.

14. A method of directing a surgical aesthetic modification procedure, the method comprising
(1) providing a user with a computerized device comprising (a) an interface that facilitates the collection of user input and transmission of the user input to a computer component comprising a computer component processor component that performs one or more functions on the input and returns output to the user through the interface; (b) a user interface processing component that performs one or more functions locally on the computerized device; and (c) a memory component that stores data and system instructions to be carried out by the user interface processing component, the computer component processor component or both,
(2) prompting the user to provide user-identifying information through the interface and via the user interface processing component, the computer component processor component, or both collecting the user-identifying information and storing the user-identifying information in the memory component,
(3) prompting the user to provide, via the interface, the computer component with one or more image data files, each image data file corresponding to a photographic image of at least a portion of the user's face that is the subject of a potential surgical aesthetic modification procedure,
(4) subjecting each of the one or more image data files to an analysis by a facial feature recognition artificial intelligence module (FFRAI) to identify and locate a plurality of mapped facial features in the photographic image and associating the facial features with the image data file,
(5) through the operation of the user interface processing component, computer component processor component, or both, applying a face image morphing function that modifies one or more of the image data files according to one or more preprogrammed instructions or one or more artificial intelligence models in a manner resulting in one or more initial morphed facial image files each expected to correspond with the outcome of performing the surgical aesthetic modification procedure according to the one or more preprogrammed instructions or the one or more artificial intelligence models,
(6) permitting the user to cause the user interface processing component, computer component processor component, or both, to modulate one or more parameters associated with one or more selected facial feature elements from among the mapped facial features to generate one or more secondarily morphed facial image files,
(7) through the interface, permitting the user to evaluate the initial morphed facial image files and each of the one or more of the secondarily morphed facial image files to generate user evaluation data,
(8) through the operation of the computer component processor component using the user evaluation data to generate procedure-related output that provides a medical practitioner with information that assists the medical practitioner in conducting the surgical aesthetic modification procedure,
(9) the medical practitioner carrying out the surgical aesthetic modification procedure on the user, based at least in part on the procedure-related output, and
(10) conducting a post-surgical aesthetic modification procedure evaluation and using the post-procedure evaluation to modify one or more AI models or other functions carried out by the processor component.

15. The method of claim 14, wherein the surgical aesthetic modification procedure-related output comprises facial image data corresponding to the user's face and providing one or more markings for guiding a plastic surgeon in applying the surgical aesthetic modification procedure.

16. The method of claim 15, wherein the surgical aesthetic modification procedure-related output comprises augmented reality output, such as a display of one or more markings for guiding a plastic surgeon in applying the surgical aesthetic modification procedure in an augmented reality output.

17. The method of claim 14, wherein the method comprises the computer component executing one or more functions that evaluate the user evaluation and providing a system-driven evaluation of the user evaluation to the medical practitioner.

18. The method of claim 17, wherein the system-driven evaluation of the user evaluation comprises a proposed modification of the user evaluation.

19. The method of claim 18, wherein the operation of the computer component processor component facilitates a review of the user input, the system-driven evaluation, or both by the medical practitioner by relaying output and one or more prompts or one or more directions to a computer device used by the medical practitioner that is in communication with the computer component.

20. The method of claim 15, wherein the method further comprises the medical practitioner carrying out the surgical aesthetic modification procedure on the user based at least in part on the procedure-related output.
